(19) United States
(12) Patent Application Publication
(10) Pub. No.: US 2004/0127470 A1
(43) Pub. Date: Jul. 1, 2004

(54) Methods and compositions for the prevention or treatment of neoplasia comprising a Cox-2 inhibitor in combination with an epidermal growth factor receptor antagonist

(75) Inventor: Jaime Masferrer, Ballwin, MO (US)

Correspondence Address: Charles E. Dunlap, Nelson Mullins Riley & Scarborough, LLP, P.O. Box 11070, Columbia, SC, 29211-1070, US

(73) Assignee: Pharmacia Corporation, St. Louis, MO (US)

(21) Appl. No.: 10/651,916

(22) Filed: Aug. 29, 2003

Related U.S. Application Data
(63) Continuation-in-part of application No. 09/470,951, filed on Dec. 22, 1999, now abandoned.
(60) Provisional application No. 60/113,786, filed on Dec. 23, 1998.

(51) Int. Cl. A61K 31/60; A61K 31/415; A61K 31/19

(52) U.S. Cl. 514/165; 514/406; 514/471; 514/420; 514/569; 514/570

(57) ABSTRACT

The present invention relates to a novel method of preventing and/or treating neoplasia disorders in a subject that is in need of such prevention or treatment by administering to the subject at least one Cox-2 inhibitor in combination with an EGF receptor antagonist. Compositions, pharmaceutical compositions and kits are also described.

### CROSS REFERENCE TO RELATED PATENTS AND PATENT APPLICATIONS

This application is a continuation-in-part of U.S. patent application Ser. No. 09/470,951 filed Dec. 22, 1999, which claims the benefit of U.S. provisional patent application Serial No. 60/113,786 filed Dec. 23, 1998, both of which are incorporated herein by reference in their entireties.

### BACKGROUND OF THE INVENTION

(1) Field of the Invention

The present invention relates generally to compositions and methods for the prevention or treatment of neoplasia and neoplasia-related disorders, and more particularly to the prevention or treatment of neoplasia and neoplasia-related disorders by the administration of one or more enzyme inhibitors and receptor antagonists.

(2) Description of Related Art

A neoplasm, or tumor, is an abnormal, unregulated, and disorganized proliferation of cell growth. A neoplasm is malignant, or cancerous, if it has properties of destructive growth, invasiveness and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphotics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

Cancer is now the second leading cause of death in the United States and over 8,000,000 persons in the United States have been diagnosed with cancer. In 1995, cancer accounted for 23.3% of all deaths in the United States. See U.S. Dept. of Health and Human Services, National Center for Health Statistics, Health United States 1996-97 and Injury Chartbook 117 (1997).

Cancer is not fully understood on the molecular level. It is known that exposure of a cell to a carcinogen such as certain viruses, certain chemicals, or radiation, leads to DNA alteration that inactivates a “suppressive” gene or activates an “oncogene”. Suppressive genes are growth regulatory genes, which upon mutation, can no longer control cell growth. Oncogenes are initially normal genes (called prooncogenes) that by mutation or altered context of expression become transforming genes. The products of transforming genes cause inappropriate cell growth. More than twenty different normal cellular genes can become oncogenes by genetic alteration. Transformed cells differ from normal cells in many ways, including cell morphology, cell-to-cell interactions, membrane content, cytoskeletal structure, protein secretion, gene expression and mortality (transformed cells can grow indefinitely).

Cancer is now primarily treated with one or a combination of three types of therapies: surgery, radiation, and chemotherapy. Surgery involves the bulk removal of diseased tissue. While surgery is sometimes effective in removing tumors located at certain sites, for example, in the breast, colon, and skin, it cannot be used in the treatment of tumors located in other areas, such as the backbone, nor in the treatment of disseminated neoplastic conditions such as leukemia.

Chemotherapy involves the disruption of cell replication or cell metabolism. It is used most often in the treatment of breast, lung, and testicular cancer.

The adverse effects of systemic chemotherapy used in the treatment of neoplastic disease is most feared by patients undergoing treatment for cancer. Of these adverse effects, nausea and vomiting are the most common and severe side effects. Other adverse side effects include cytopenia, infection, cachexia, mucositis in patients receiving high doses of chemotherapy with bone marrow rescue or radiation therapy, alopecia (hair loss), cutaneous complications See M. D. Abeloff, et al: Alopecia and Cutaneous Complications, p. 755-56, in Abeloff, M. D., Armitage, J. O., Lichter, A. S., and Niederhuber, J. E. (eds) Clinical Oncology. Churchill Livingston, N.Y., (1992) for cutaneous reactions to chemotherapy agents, such as pruritis, urticaria, and angioedema, neurological complications, pulmonary and cardiac complications in patients receiving radiation or chemotherapy, and reproductive and endocrine complications.

The adverse side effects induced by chemotherapeutic agents and radiation therapy have become of major importance to the clinical management of cancer patients.

Chemotherapy-induced side effects significantly impact the quality of life of the patient and may dramatically influence patient compliance with treatment. Additionally, adverse side effects associated with chemotherapeutic agents are generally the major dose-limiting toxicity (DLT) in the administration of these drugs. For example, mucositis is a major dose limiting toxicity for several anticancer agents, including the antimetabolite cytotoxic agents 5-FU, methotrexate, and antitumor antibiotics, such as doxorubicin. Many of these chemotherapy-induced side effects, if severe, may lead to hospitalization, or require treatment with analgesics for the treatment of pain.

Historically, physicians have treated inflammation-related disorders with a regimen of nonsteroidal anti-inflammatory drugs (NSAIDS), such as, for example, aspirin and ibuprofen. Of particular interest is the recent discovery that NSAID use has been associated with the prevention and treatment of several types of cancer. See Thun, M., et al., J. National Cancer Inst. 94(4):252-266 (2002). Undesirably, however, some NSAIDS are known to cause gastrointestinal (GI) bleeding or ulcers in patients undergoing consistent long-term regimens of NSAID therapy. See Henry, D., et al., Lancet 337:730 (1991).

A reduction of unwanted side effects of common NSAIDS was made possible by the discovery that two cyclooxygenases are involved in the transformation of arachidonic acid as the first step in the prostaglandin synthesis pathway. These enzymes exist in two forms and have been termed cyclooxygenase-1 (Cox-1) and cyclooxygenase-2 (Cox-2). See Needleman, P. et al., J. Rheumatol. 24, Suppl.49:6-8 (1997).

Cox-1 is a constitutive enzyme responsible for the biosynthesis of prostaglandins in the gastric mucosa and in the kidney. Cox-2 is an enzyme that is produced by an inducible gene that is responsible for the biosynthesis of prostaglandins in inflammatory cells. Inflammation causes the induction of Cox-2, leading to the release of prostanoids (prostaglandin E2), which sensitize peripheral nociceptor terminals and produce localized pain hypersensitivity, inflammation, and oedema. See Samad, T., et al., Nature 410(6827):471-5 (2001).

Many common NSAIDs are now known to be inhibitors of both Cox-1 and Cox-2. Accordingly, when administered in sufficiently high levels, these NSAIDs not only alleviate the inflammatory consequences of Cox-2 activity, but also inhibit the beneficial gastric maintenance activities of Cox-1.

Research into the area of arachidonic acid metabolism has resulted in the discovery of compounds that selectively inhibit the cyclooxygenase-2 enzyme to a greater extent than the activity of Cox-1. The Cox-2 selective inhibitors are believed to offer advantages that include the capacity to prevent or reduce inflammation while avoiding harmful side effects associated with the inhibition of Cox-1. Thus, Cox-2 selective inhibitors have shown great promise for use in therapies—especially in therapies that require maintenance administration, such as for pain and inflammation control.

Of particular importance, for the present invention is that overexpression of Cox-2 has been documented in several premalignant and maliganant tissues. See Subbaramaiah, K. and Dannenberg, A. J. Trends Pharmacol Sci, 24:96-102 (2003). This increase in expression is thought to be a product of stimulation of protein kinase C (PKC) signaling, which stimulates the activity of mitogen-activated protein kinase (MAPK), enhancing transcription of Cox-2 by nuclear factors. Additionally, enhanced stability of Cox-2 mRNA transcripts in cancer cells due to augmented binding of the RNA-binding protein HuR, as well as activation of extracellular signal related kinase 1/2 (ERK 1/2) and p38, contributes to increased expression of Cox-2. Id.

Recently, several additional chemotherapeutic agents have reported to have efficacy in treating or preventing neoplasia-related disorders and include the Epidermal Growth Factor Receptor (EGFR or EGF receptor) antagonists. There are several lines of evidence in support of EGFR as a target for neoplasia therapy. Coexpression of high levels of EGFR and its ligands leads to a transformed cellular phenotype, the expression of EGFR is increased in many epithelial tumors and tumor-derived cell lines, and this overexpression correlates with a poor clinical outcome in a number of neoplasia-related malignancies. See Mendelsohn J., et al., Oncogene 19:6550-6565 (2000).

Tarceva™ (erlotinib) is a small molecule designed to selectively target the human epidermal growth factor receptor (HER1) pathway, also known as EGFR or the EGF receptor, which is critical to cell growth in many cancers. HER1/EGFR is a key component of the HER signaling pathway, which is often involved in the formation and growth of numerous cancers. In order for a tumor to grow, tumor cell receptors must be able to link with certain enzymes, one of them being tyrosine kinase. Tarceva™ is designed to inhibit specifically the tyrosine kinase activity of HER1/EGFR, thereby blocking the signaling pathway and inhibiting tumor cell growth. Tarceva® antagonizes the activity of tyrosine kinase before it can join with the cell, thereby shutting down tumor growth.

An important advantage of targeted agents like Cox-2 inhibitors and EGFR antagonists like Tarceva™ is that they are not associated with common chemotherapy side effects, such as nausea, vomiting, hair loss and reduction in normal blood counts—side effects that occur frequently with conventional chemotherapeutic agents.

Unfortunately, even with the multitude of chemotherapeutic agents that are now available or in clinical trials for the treatment or prevention of neoplasia, it is still a disorder that defies most attempts at eradication. At best, remission of an existing neoplasia disorder is the only available prognosis. In addition, conventional chemotherapeutic agents have the marked disadvantage of causing a wide array of dehabilitating side effects.

From the foregoing, it can be seen that a need still exists for improved methods and therapeutic compositions to treat neoplasia and neoplasia-related disorders. It would also be useful to provide an improved method and composition for reducing the symptoms associated with neoplasia. Likewise, methods and compositions that improve patient outcomes following radiation and chemotherapy treatment regimens for neoplasms would also be desirable. Also, methods and compositions that reduce dosages or reduce unwanted side effects that are often associated with conventional treatments for neoplasia or neoplasia-related disorders are desirable. Finally, methods and compositions that improve the efficacy of treating neoplasia or a neoplasia-related disorder that is considered resistant or intractable to known methods of therapy alone would also be desirable.

### SUMMARY OF THE INVENTION

Briefly, therefore, the present invention is directed to a novel method for preventing or treating a neoplasia disorder in a subject that is in need of such prevention or treatment comprising administering to the subject a Cox-2 inhibitor in combination with an EGF receptor antagonist.

In one embodiment, the present invention is directed to a novel method for preventing or treating a pathological condition or physiological disorder characterized by or associated with neoplasia in a subject that is in need of such therapy, the method comprising administering to the subject a Cox-2 inhibitor in combination with an EGF receptor antagonist.

In another embodiment, the present invention is directed a novel therapeutic composition comprising a Cox-2 inhibitor and an EGF receptor antagonist.

In yet another embodiment, the present invention is directed to a pharmaceutical composition for preventing or treating a neoplasia-related disorder in a subject that is in need of such prevention and treatment, the pharmaceutical composition comprising a Cox-2 inhibitor, an EGF receptor antagonist, and a pharmaceutically acceptable carrier.

The present invention is also directed to a kit for the purpose of preventing or treating a neoplasia disorder in a subject that is in need of such prevention or treatment, the kit comprising one dosage form comprising a Cox-2 inhibitor and a second dosage form comprising an EGF receptor antagonist.

The present invention is also directed to a novel method of preventing or treating a pathological condition or physiological disorder characterized by or associated with neoplasia in a subject that is in need of such therapy comprising administering to the subject a Cox-2 inhibitor and an EGF receptor-modulating amount of an EGF receptor antagonist.

The present invention is also directed to a novel method that comprises treating a subject with a therapeutically effective amount of a combination comprising two or more agents. The first agent is an antiangiogenesis agent selected from a first group of antiangiogenesis agents consisting of:

a matrix metalloproteinase inhibitor (MMP),

a cyclooxygenase-2 inhibitor (Cox-2),

an alpha v beta 3 inhibitor, and

a pBATT.

The additional agent, or agents, is selected from the group of antineoplastic agents, or therapeutic approaches consisting of:

an antiangiogenesis agent, other than the agent selected from the first group,

an antineoplastic agent, other than an antiangiogenesis agent,

an adjunctive agent,

an immunotherapeutic agent,

a device,

a vaccine,

an analgesic agent, and

a radiotherapeutic agent.

In some embodiments, the antineoplastic agent is an EGF receptor antagonist.

The present invention is also directed to a novel method of preventing or treating neoplasia disorders and neoplasia disorder-related complications in a subject that is in need of such prevention or treatment comprising administering to the subject a Cox-2 inhibitor in combination with an EGF receptor antagonist, wherein the Cox-2 inhibitor and EGF receptor antagonist are administered to the subject in combination with one or more antineoplastic agents, wherein-the antineoplastic agent is other than a Cox-2 inhibitor or an EGF receptor antagonist.

Among the several advantages found to be achieved by the present invention, therefore, may be noted the provision of improved methods and therapeutic compositions for the prevention or treatment of neoplasia disorders such as colon cancer, lung cancer and breast cancer. Other advantages achieved by the present invention include improved methods and compositions for reducing both the inflammation and the pain associated with neoplasia disorders. Still other advantages achieved by the present invention include methods and compositions that improve patient responses following acute neoplasia episodes. In addition, the present invention provides methods and compositions that reduce dosages or reduce unwanted side effects of conventional treatments for neoplasia disorders are desirable. Finally, the present invention provides methods and compositions that improve the efficacy of treating a neoplasia disorder that is considered resistant or intractable to known methods of therapy alone.

### DETAILED DESCRIPTION OF PREFERRED EMBODIMENTS

In accordance with the present invention it has been discovered that the treatment or prevention of neoplasia disorders, including such neoplasia disorders as cancer, is provided by a combination therapy comprising a Cox-2 inhibitor and an EGF receptor antagonist.

For purposes of the present invention, the novel combination therapy is useful for the purpose of preventing or treating neoplasia disorders and neoplasia disorder-related complications in a subject that is in need of such prevention or treatment, and involves administering to the subject at least one Cox-2 inhibitor and one or more EGF receptor antagonists.

The present invention also encompasses a method for inhibiting the growth of neoplasia, including a malignant tumor or cancer, by exposing the neoplasia to an inhibitory or therapeutically effective amount or concentration of at least one of the disclosed Cox-2 inhibitor compounds in combination with at least one of the disclosed EGF receptor antagonists. This method may be used therapeutically, in the treatment of neoplasia, including cancer, or in comparison tests such as assays for determining the activities of related analogs as well as for determining the susceptibility of a subject's cancer to one or more of the compounds according to the present invention.

The administration of the novel combination therapy of Cox-2 inhibitors and EGF receptor antagonists described herein is unexpectedly effective therapy for the prevention and treatment of neoplasia. Such administration is for preventing and treating the symptoms of neoplasia while reducing or avoiding the disadvantages and side effects associated with current treatment strategies.

In a preferred embodiment, the present invention also encompasses methods and compositions that improve subject outcomes following radiation and chemotherapy treatment regimens for neoplasia. In another preferred embodiment, the present invention encompasses methods and compositions that reduce dosages or reduce unwanted side effects in conventional treatments for neoplasia or neoplasia-related disorders. In yet another preferred embodiment, the present invention also encompasses methods and compositions that improve the efficacy of treating neoplasia or a neoplasia-related disorder that is considered resistant or intractable to known methods of therapy alone.

In a preferred embodiment, the treatment or prevention of neoplasia can be accomplished by administering to a subject suffering from or needing prevention of a neoplasia a Cox-2 inhibitor and an EGF receptor antagonist.

Preferably, the amount of a single dosage of a combination comprising a Cox-2 inhibitor and an EGF receptor antagonist is a therapeutically effective amount of the combination.

As used herein, the phrases “combination therapy”, “co-administration”, “co-administering”, “administration with”, “administering”, “combination”, or “co-therapy”, when referring to use of a Cox-2 inhibitor in combination with an EGF receptor antagonist, are intended to embrace administration of each agent in a sequential manner in a regimen that will provide beneficial effects of the drug combination, and is intended as well to embrace co-administration of these agents in a substantially simultaneous manner. Thus, the Cox-2 inhibitor and EGF receptor antagonist may be administered in one therapeutic dosage form, such as in a single capsule, tablet, or injection, or in two separate therapeutic dosage forms, such as in separate capsules, tablets, or injections.

Sequential administration of such treatments encompasses both relatively short and relatively long periods between the administration of each of the drugs of the present method. However, for purposes of the present invention, the second drug is administered while the first drug is still having an efficacious effect on the subject. Thus, the present invention takes advantage of the fact that the simultaneous presence of the combination of a Cox-2 inhibitor and EGF receptor antagonist in a subject has a greater efficacy than the administration of either agent alone.

Preferably, the second of the two drugs is to be given to the subject within the therapeutic response time of the first drug to be administered. For example, the present invention encompasses administration of a Cox-2 inhibitor to the subject and then the later administration of an EGF receptor antagonist, as long as the EGF receptor antagonist is administered to the subject while the Cox-2 inhibitor is still present in the subject at a level, which, in combination with the level of the EGF receptor antagonist, is therapeutically effective, and vice versa. As used herein, the term “therapeutic response time” means the duration of time that a compound is present or detectable at any level within a subject's body.

In one embodiment, the Cox-2 inhibitor and EGF receptor antagonist are administered in one therapeutic dosage form, such as in a single capsule, tablet, or injection.

In other embodiments, the Cox-2 inhibitor and EGF receptor antagonist are administered in two separate therapeutic dosage forms, such as in separate capsules, tablets, or injections.

In one embodiment, the present invention encompasses a method for preventing a neoplasia disorder and neoplasia disorder-related complication in a subject that is in need of such prevention, and involves administering to the subject at least one Cox-2 inhibitor and one or more EGF receptor antagonists.

As used herein, the term “prevention” refers to any reduction, no matter how slight, of a subject's predisposition or risk for developing a neoplasia or neoplasia-related disorder. For purposes of prevention, the subject is any subject, and preferably is a subject that is at risk for, or is predisposed to, developing a neoplasia or neoplasia-related disorder or a neoplasia-related complication.

As used herein, a subject that is “predisposed to” or “at risk for,” both of which are used interchangeably herein, includes any subject at risk for developing a neoplasia-related disorder or any neoplasia-related complication. For example, after treatment, many neoplasia disorders subside into remission, meaning that the disease is present, but inactive within the subject and is thus, capable of re-developing at a later time, which makes the subject at risk for developing a neoplasia-related disorder or complication. The subject may also be at risk due to genetic predisposition, diet, lifestyle, age, exposure to radiation, exposure to neoplasia-causing agents, and the like.

In another embodiment, the present invention encompasses a method for treating a neoplasia disorder and neoplasia disorder-related complication in a subject that is in need of such treatment, and involves administering to the subject at least one Cox-2 inhibitor and one or more EGF receptor antagonists.

As used herein, the terms “treating” or “to treat,” refer to any reduction in the symptoms of a neoplasia disorder, no matter how slight of any of the neoplasia diseases or disorders described herein.

Without being bound by this or any other theory, it is believed that a therapy comprising a Cox-2 inhibitor and an EGF receptor antagonist is efficacious for preventing or treating neoplasia disorders and neoplasia disorder-related complications. Moreover, in one embodiment, the combination of a Cox-2 inhibitor and an EGF receptor antagonist provides synergistic effects, which reduce the symptoms associated with neoplasia disorders and neoplasia disorder-related complications to a greater extent than would be expected based on the administration of either one alone. The term “synergistic” refers to the combination of a Cox-2 inhibitor and an EGF receptor antagonist as a combined therapy having an efficacy for the prevention and treatment of neoplasia disorders that is greater than the sum of their individual effects.

The synergistic effects of certain embodiments of the present invention's combination therapy encompass additional unexpected advantages for the treatment or prevention of neoplasia disorders. Such additional advantages include, but are not limited to, lowering the required dose of EGF receptor antagonists, reducing the side effects of EGF receptor antagonists, and rendering those antagonists more tolerable to subjects in need of neoplasia disorder therapy.

The combination therapy of the present invention also provides for the treatment of neoplasia disorder-related complications that may arise indirectly from having a neoplasia disorder. For example, if a subject is suffering from a neoplasia disorder-related complication, such as pain and/or chronic pain, the treatment of the underlying neoplasia disorder, such as colon cancer, by the methods and compositions of the present invention will likewise improve the symptoms of the associated complication.

In still other embodiments, the treatment or prevention of a neoplasia disorder in a subject in need of such treatment or prevention is provided by methods and combinations using two or more components with at least one component being an antiangiogenesis agent.

The method comprises treating a subject with a therapeutically effective amount of a combination comprising two or more agents. The first agent is an antiangiogenesis agent selected from a first group of antiangiogenesis agents consisting of:

a matrix metalloproteinase inhibitor (MMP),

a cyclooxygenase-2 inhibitor (Cox-2),

an alpha v beta 3 inhibitor, and

a pBATT.

The additional agent, or agents, are selected from the group of antineoplastic agents, or therapeutic approaches consisting of:

an antiangiogenesis agent, other than the agent selected from the first group,

an antineoplastic agent, other than an antiangiogenesis agent;

an adjunctive agent,

an immunotherapeutic agent,

a device,

a vaccine,

an analgesic agent, and

a radiotherapeutic agent.

For purposes of the present invention, the term “antiangiogenesis agent” encompasses, among others, Cox-2 inhibitors, and, in particular Cox-2 selective inhibitors. For purposes of the present invention, the term “antineoplastic agent” encompasses, among others, EGF receptor antagonists. Therefore, in one embodiment, the antiangiogenesis agent is a Cox-2 inhibitor and the antineoplastic agent is an EGF receptor antagonist.

The present invention is also directed to a novel a method of preventing or treating neoplasia disorders and neoplasia disorder-related complications in a subject that is in need of such prevention or treatment comprising administering to the subject a Cox-2 inhibitor in combination with an EGF receptor antagonist, wherein the Cox-2 inhibitor and EGF receptor antagonist is administered to the subject in combination with one or more antineoplastic agents and the antineoplastic agent is other than a Cox-2 inhibitor and other than a EGF receptor antagonist.

As used herein, the terms “neoplasia” and “neoplasia disorder”, which used interchangeably, refer to new cell growth that results from a loss of responsiveness to normal growth controls, e.g. to “neoplastic” cell growth. Neoplasia is also used interchangeably herein with the term “cancer” and for purposes of the present invention; cancer is one subtype of neopiasia. Neopiasia is also used interchangeably herein to describe a “benign” or non-malignant growth of cells. As used herein, the term “neoplasia disorder” also encompasses other cellular abnormalities, such as hyperplasia, metaplasia and dysplasia. The terms neoplasia, metaplasia, dysplasia and hyperplasia can be used interchangeably herein and refer generally to cells experiencing abnormal cell growth.

Both of the terms, “neoplasia” and “neoplasia disorder”, refer to a “neoplasm” or tumor, which may be benign, premalignant, metastatic, or malignant. Also encompassed by the present invention are benign, premalignant, metastatic, or malignant neoplasias. Also encompassed by the present invention are benign, premalignant, metastatic, or malignant tumors. Thus, all of benign, premalignant, metastatic, or malignant neoplasia or tumors are encompassed by the present invention and may be referred to interchangeably, as “neoplasia,” “neoplasms” or “neoplasia-related disorders.” Tumors are generally known in the art to be a mass of neoplasia or “neoplastic” cells. Although, it is to be understood that even one neoplastic cell is considered, for purposes of the present invention to be a neoplasm or alternatively, neoplasia.

The methods and combinations of the present invention may be used for the treatment or prevention of neoplasia disorders including acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangiocarcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intraepithelial neoplasia, interepithelial squamous cell neopiasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiatied carcinoma, uveal melanoma, verrucous carcinoma, vipoma, well differentiated carcinoma, and Wilm's tumor.

The methods and compositions of the present invention provide one or more benefits. Combinations of antiangiogenesis inhibitors with the compounds, compositions, agents and therapies of the present invention are useful in treating and preventing neoplasia disorders. Preferably, the antiangiogenic agent or agents and the compounds, compositions, agents and therapies of the present invention are administered in combination at a low dose, that is, at a dose lower than has been conventionally used in clinical situations for each of the individual components administered alone.

A benefit of lowering the dose of the compounds, compositions, agents and therapies of the present invention administered to a mammal includes a decrease in the incidence of adverse effects associated with higher dosages. For example, by lowering the dosage of a chemotherapeutic agent such as methotrexate, a reduction in the frequency and the severity of nausea and vomiting will result when compared to that observed at higher dosages. Similar benefits are contemplated for the compounds, compositions, agents and therapies in combination with the antiangiogenesis agents of the present invention.

By lowering the incidence of adverse effects, an improvement in the quality of life of a patient undergoing treatment for cancer is contemplated. Further benefits of lowering the incidence of adverse effects include an improvement in patient compliance, a reduction in the number of hospitalizations needed for the treatment of adverse effects, and a reduction in the administration of analgesic agents needed to treat pain associated with the adverse effects.

The use of the antiangiogenesis agents TNP-470 and minocycline in combination with cyclophasphamide, CDDP, or thiotepa have been observed to substantially increase the tumor growth delay in one pre-clinical solid tumor model. See Teicher, B. A. et al., Breast Cancer Research and Treatment 36:227-236 (1995). Additionally, improved results were observed when these antiangiogenesis agents were used in combination with cyclophosphamide and fractionated radiation therapy. See Teicher, B. A. et al., European Journal of Cancer 32A(14): 2461-2466 (1996).

WO 9803516 A describes phosphinate compounds in combination with cytotoxic anticancer agents for the treatment of cancer; diseases characterized by matrix metalloproteinase activity; diseases involving the production of tumor necrosis factor (TNF); or for inhibition of matrix metalloproteinase (MMP) or the production of TNF; in mammals, including humans.

WO9748685 describes metalloprotease (MMP) inhibitors in combination with current chemotherapy and/or radiation for systemic chemotherapy of cancer.

Kumar and Armstrong describe antiangiogenesis therapy used as an adjunct to chemotherapy, radiation therapy, or surgery. See Kumar, C C, and Armstrong, L., Tumor-induced angiogenesis: a novel target for drug therapy?, Emerging Drugs 2:175-190 (1997).

The present invention further includes kits comprising a Cox-2 inhibitor, a MMP inhibitor, an integrin antagonist and an antineoplastic agent.

The term “treatment” refers to any process, action, application, therapy, or the like, wherein a mammal, including a human being, is subject to medical aid with the object of improving the mammal's condition, directly or indirectly.

The term “inhibition,” in the context of neoplasia, tumor growth or tumor cell growth, may be assessed by delayed appearance of primary or secondary tumors, slowed development of primary or secondary tumors, decreased occurrence of primary or secondary tumors, slowed or decreased severity of secondary effects of disease, arrested tumor growth and regression of tumors, among others. In the extreme, complete inhibition, is referred to herein as prevention or chemoprevention.

The term “prevention” includes either preventing the onset of clinically evident neoplasia altogether or preventing the onset of a preclinically evident stage of neoplasia in individuals at risk. Also intended to be encompassed by this definition is the prevention of initiation for malignant cells or to arrest or reverse the progression of premalignant cells to malignant cells. This includes prophylactic treatment of those at risk of developing the neoplasia.

The phrase “therapeutically-effective” is intended to qualify the amount of each agent that will achieve the goal of improvement in neoplastic disease severity and the frequency of neoplastic disease over treatment of each agent by itself, while avoiding adverse side effects typically associated with alternative therapies.

A “therapeutic effect” or “therapeutic effective amount” is intended to qualify the amount of an anticancer agent required to relieve to some extent one or more of the symptoms of a neoplasia disorder, including, but is not limited to: 1) reduction in the number of cancer cells; 2) reduction in tumor size; 3) inhibition (i.e., slowing to some extent, preferably stopping) of cancer cell infiltration into peripheral organs; 3) inhibition (ie., slowing to some extent, preferably stopping) of tumor metastasis; 4) inhibition, to some extent, of tumor growth; 5) relieving or reducing to some extent one or more of the symptoms associated with the disorder; and/or 6) relieving or reducing the side effects associated with the administration of anticancer agents.

The phrase “combination therapy” (or “co-therapy”) embraces the administration of an antiangiogenesis inhibitor and optionally an antineoplastic agent as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other two therapeutic agents of the combination may be administered orally. Alternatively, for example, all three therapeutic agents may be administered orally or all three therapeutic agents may be administered by intravenous injection. The sequence in which the therapeutic agents are administered is not narrowly critical. “Combination therapy” also can embrace the administration of the therapeutic agents as described above in further combination with other biologically active ingredients (such as, but not limited to, a second and different antineoplastic agent) and non-drug therapies (such as, but not limited to, surgery or radiation treatment). Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

The phrases “low dose” or “low dose amount”, in characterizing a therapeutically effective amount of the antiangiogenesis agent and the antineoplastic agent or therapy in the combination therapy, defines a quantity of such agent, or a range of quantity of such agent, that is capable of improving the neoplastic disease severity while reducing or avoiding one or more antineoplastic-agent-induced side effects, such as myelosupression, cardiac toxicity, alopecia, nausea or vomiting.

The phrase “adjunctive therapy” encompasses treatment of a subject with agents that reduce or avoid side effects associated with the combination therapy of the present invention, including, but not limited to, those agents, for example, that reduce the toxic effect of anticancer drugs, e.g., bone resorption inhibitors, cardioprotective agents; prevent or reduce the incidence of nausea and vomiting associated with chemotherapy, radiotherapy or operation; or reduce the incidence of infection associated with the administration of myelosuppressive anticancer drugs.

The phrase an “immunotherapeutic agent” refers to agents used to transfer the immunity of an immune donor, e.g., another person or an animal, to a host by inoculation. The term embraces the use of serum or gamma gobulin containing performed antibodies produced by another individual or an animal; nonspecific systemic stimulation; adjuvants; active specific immunotherapy; and adoptive immunotherapy. Adoptive immunotherapy refers to the treatment of a disease by therapy or agents that include host inoculation of sensitized lymphocytes, transfer factor, immune RNA, or antibodies in serum or gamma globulin.

The phrase a “device” refers to any appliance, usually mechanical or electrical, designed to perform a particular function.

The phrase a “vaccine” includes agents that induce the patient's immune system to mount an immune response against the tumor by attacking cells that express tumor associated antigens (TAAs).

The phrase “multi-functional proteins” encompass a variety of pro-angiogenic factors that include basic and acid fibroblast growth factors (bFGF and aFGF) and vascular permeability factor/vascular endothelial growth factor (VPFNEGF). See Bikfalvi, A. et al., Endocrine Reviews 18:26-45 (1997). Several endogenous antiangiogenic factors have also been characterized as multi-functional proteins and include angiostatin (O'Reilly, et al., Cell (Cambridge, Mass.) 79(2): 315-328, 1994), endostatin (O'Reilly, et al., Cell (Cambridge, Mass.) 88(2): 277-285, 1997), interferon .alpha. (Ezekowitz, et al., N. Engl. J Med., May 28, 326(22) 1456-1463, 1992), thrombospondin (Good, et al, Proc Natl Acad Sci USA 87(17): 6624-6628, 1990; Tolsma, et al., J Cell Biol 122(2): 497-511, 1993), and platelet factor 4 (PF4) (Maione, et al., Science 247:(4938): 77-79, 1990).

The phrase “analgesic agent” refers to an agent that relieves pain without producing anesthesia or loss of consciousness generally by altering the perception of nociceptive stimuli.

The phrase a “radiotherapeutic agent” refers to the use of electromagnetic or particulate radiation in the treatment of neoplasia.

The term “pBATT” embraces or “Protein-Based Anti-Tumor Therapies,” refers to protein-based therapeutics for solid tumors. The pBATTs include proteins that have demonstrated efficacy against tumors in animal models or in humans. The protein is then modified to increase its efficacy and toxicity profile by enhancing its bioavailability and targeting.

“Angiostatin” is a 38 kD protein comprising the first three or four kringle domains of plasminogen and was first described in 1994. See O'Reilly, M. S. et al., Cell (Cambridge, Mass.) 79(2): 315-328 (1994). Mice bearing primary (Lewis lung carcinoma-low metastatic) tumors did not respond to angiogenic stimuli such as bFGF in a corneal micropocket assay and the growth of metastatic tumors in these mice was suppressed until the primary tumor was excised. The factor responsible for the inhibition of angiogenesis and tumor growth was designated mouse angiostatin. Angiostatin was also shown to inhibit the growth of endothelial cells in vitro.

Human angiostatin can be prepared by digestion of plasminogen by porcine elastase (O'Reilly, et al., Cell 79(2): 315-328, 1994) or with human metalloelastase (Dong, et al., Cell 88:801-810, 1997). The angiostatin produced via porcine elastase digestion inhibited the growth of metastases and primary tumors in mice. O'Reilly, et al., (Cell 79(2):315-328, 1994) demonstrated that human angiostatin inhibited metastasis of Lewis lung carcinoma in SCID mice. The same group (O'Reilly, M. et al., Nat Med. (N.Y.) 2(6):689-692, 1996) subsequently showed that human angiostatin inhibited the growth of the human tumors PC3 prostate carcinoma, clone A colon carcinoma, and MDA-MB breast carcinoma in SCID mice. Human angiostatin also inhibited the growth of the mouse tumors Lewis lung carcinoma, T241 fibrosarcoma and M5076 reticulum cell carcinoma in C57BI mice. Because these enzymatically-prepared angiostatins are not well characterized biochemically, the precise composition of the molecules is not known.

Angiostatins of known composition can be prepared by means of recombinant DNA technology and expression in heterologous cell systems. Recombinant human angiostatin comprising Kringle domains one through four (K1-4) has been produced in the yeast Pichia pastoris. See Sim, et al., Cancer Res 57:1329-1334 (1997). The recombinant human protein inhibited growth of endothelial cells in vitro and inhibited metastasis of Lewis lung carcinoma in C57BI mice. Recombinant murine angiostatin (K1-4) has been produced in insect cells. See Wu, et al., Biochem Biophys Res Comm 236:651-654 (1997). The recombinant mouse protein inhibited endothelial cell growth in vitro and growth of primary Lewis lung carcinoma in vivo. These experiments demonstrated that the first four kringle domains are sufficient for angiostatin activity but did not determine which kringle domains are necessary.

Cao, et al., J. Biol. Chem. 271:29461-29467 (1996), produced fragments of human plasminogen by proteolysis and by expression of recombinant proteins in E. coli. These authors showed that kringle one and to a lesser extent kringle four of plasminogen were responsible for the inhibition of endothelial cell growth in vitro. Specifically, kringles 1-4 and 1-3 inhibited at similar concentrations, while K1 alone inhibited endothelial cell growth at four-fold higher concentrations. Kringles two and three inhibited to a lesser extent. More recently Cao, et al., J. Biol. Chem. 272:22924-22928 (1997), showed that recombinant mouse or human kringle five inhibited endothelial cell growth at lower concentrations than angiostatin (K1-4). These experiments demonstrated in vitro angiostatin-like activity, but did not address in vivo action against tumors and their metastases.

PCT publication WO 95/29242 discloses purification of a protein from blood and urine by HPLC that inhibits proliferation of endothelial cells. The protein has a molecular weight between 38 kilodaltons and 45 kilodaltons and an amino acid sequence substantially similar to that of a murine plasminogen fragment beginning at amino acid number 79 of a murine plasminogen molecule. PCT publication WO 96/41194, discloses compounds and methods for the diagnosis and monitoring of angiogenesis-dependent diseases. PCT publication WO 96/35774 discloses the structure of protein fragments, generally corresponding to kringle structures occurring within angiostatin. It also discloses aggregate forms of angiostatin, which have endothelial cell inhibiting activity, and provides a means for inhibiting angiogenesis of tumors and for treating angiogenic-mediated diseases.

“Endostatin” is a 20-kDa (184 amino acid) carboxy fragment of collagen XVIII, is an angiogenesis inhibitor produced by a hemangioendothelioma. See O'Reilly, M. et al., Cell, 88(2):277-285 (1997) and WO 97/15666. Endostatin specifically inhibits endothelial proliferation and inhibits angiogenesis and tumor growth. Primary tumors treated with non-refolded suspensions of E. coli-derived endostatin regressed to dormant microscopic lesions. Toxicity was not observed and immunohistochemical studies revealed a blockage of angiogenesis accompanied by high proliferation balanced by apoptosis in tumor cells.

“Interferon .alpha.” (IFN.alpha.) is a family of highly homologous, species-specific proteins that possess complex antiviral, antineoplastic and immunomodulating activities (Extensively reviewed in the monograph “Antineoplastic agents, interferon alfa”, American Society of Hospital Pharmacists, Inc., 1996). Interferon .alpha. also has anti-proliferative, and antiangiogenic properties, and has specific effects on cellular differentiation (Sreevalsan, in “Biologic Therapy of Cancer”, pp. 347-364, (eds. V. T. DeVita Jr., S. Hellman, and S. A. Rosenberg), J. B. Lippincott Co, Philadelphia, Pa., 1995).

Interferon .alpha. is effective against a variety of cancers including hairy cell leukemia, chronic myelogenous leukemia, malignant melanoma, and Kaposi's sarcoma. The precise mechanism by which IFN.alpha. exerts its anti-tumor activity is not entirely clear, and may differ based on the tumor type or stage of disease. The anti-proliferative properties of IFN.alpha., which may result from the modulation of the expression of oncogenes and/or proto-oncogenes, have been demonstrated on both tumor cell lines and human tumors growing in nude mice. See Gutterman, J. U., Proc. Natl. Acad. Sci., USA 91:1198-1205 (1994).

Interferon is also considered an anti-angiogenic factor, as demonstrated through the successful treatment of hemangiomas in infants (Ezekowitz, et al., N. Engl. J. Med., 326(22) 1456-1463,1992) and the effectiveness of IFN.alpha. against Kaposi's sarcoma (Krown, Semin. Oncol. 14(2 Suppl 3): 27-33,1987). The mechanism underlying these anti-angiogenic effects is not clear, and may be the result of IFN.alpha. action on the tumor (decreasing the secretion of pro-angiogenic factors) or on the neo-vasculature. IFN receptors have been identified on a variety of cell types. See Navarro, et al., Modern Pathology 9(2): 150-156 (1996).

U.S. Pat. No. 4,530,901, by Weissmann, describes the cloning and expression of IFN-.alpha.-type molecules in transformed host strains. U.S. Pat. No. 4,503,035, Pestka, describes an improved processes for purifying 10 species of human leukocyte interferon using preparative high performance liquid chromatography. U.S. Pat. No. 5,231,176, Goeddel, describes the cloning of a novel distinct family of human leukocyte interferons containing in their mature form greater than 166 and no more than 172 amino acids.

U.S. Pat. No. 5,541,293, by Stabinsky, describes the synthesis, cloning, and expression of consensus human interferons. These are non-naturally occurring analogues of human (leukocyte) interferon-.alpha. assembled from synthetic oligonucleotides. The sequence of the consensus interferon was determined by comparing the sequences of 13 members of the IFN-.alpha. family of interferons and selecting the preferred amino acid at each position. These variants differ from naturally occurring forms in terms of the identity and/or location of one or more amino acids, and one or more biological and pharmacological properties (e.g., antibody reactivity, potency, or duration effect) but retain other such properties.

“Thrombospondin-1” (TSP-1) is a trimer containing three copies of a 180 kDa polypeptide. TSP-1 is produced by many cell types including platelets, fibroblasts, and endothelial cells (Frazier, Curr Opin Cell Biol. 3(5): 792-799, 1991) and the cDNA encoding the subunit has been cloned (Hennessy, et al., 1989, J Cell Biol 108(2): 729-736; Lawler and Hynes, J Cell Biol 103(5): 1635-1648, 1986). Native TSP-1 has been shown to block endothelial cell migration in vitro and neovascularization in vivo. See Good, et al., Proc. Natl. Acad. Sci. USA 87(17): 6624-6628 (1990). Expression of TSP-1 in tumor cells also suppresses tumorigenesis and tumor-induced angiogenesis. See Sheibani and Frazier, Proc. Natl. Acad. Sci. USA 92(15) 6788-6792 (1995); and Weinstat-Saslow, et al., Cancer Res 54(24):6504-6511, 1994). The antiangiogenic activity of TSP-1 has been shown to reside in two distinct domains of this protein. See Tolsma, et al., J Cell Biol 122(2):497-511 (1993). One of these domains consists of residues 303 to 309 of native TSP-1 and the other consists of residues 481 to 499 of TSP-1. Another important domain consists of the sequence CSVTCG that appears to mediate the binding of TSP-1 to some tumor cell types. See Tuszynski and Nicosia, Bioessays 18(1):71-76 (1996). These results suggest that CSVTCG, or related sequences, can be used to target other moieties to tumor cells. Taken together, the available data indicate that TSP-1 plays a role in the growth and vascularization of tumors. Subfragments of TSP-1, then, may be useful as antiangiogenic components of chimeras and/or in targeting other proteins to specific tumor cells. Subfragments may be generated by standard procedures (such as proteolytic fragmentation, or by DNA amplification, cloning, expression, and purification of specific TSP-1 domains or subdomains) and tested for antiangiogenic or anti-tumor activities by methods known in the art. See Tolsma, et al., J. Cell Biol. 122(2): 497-511 (1993); and Tuszynski and Nicosia, Bioessays 18(1): 71-76 (1996).

The combination of Cox-2 inhibitors and matrix metalloproteinase inhibitors may be used in conjunction with other treatment modalities, including, but not limited to surgery and radiation, hormonal therapy, antiangiogenic therapy, chemotherapy, immunotherapy, and cryotherapy. The present invention may be used in conjunction with any current or future therapy.

The following discussion highlights some agents in this respect, which are illustrative, not limitative. A wide variety of other effective agents also may be used.

Surgery and Radiation

In general, surgery and radiation therapy are employed as potentially curative therapies for patients under 70 years of age who present with clinically localized disease and are expected to live at least 10 years.

For example, approximately 70% of newly diagnosed prostate cancer patients fall into this category. Approximately 90% of these patients (65% of total patients) undergo surgery, while approximately 10% of these patients (7% of total patients) undergo radiation therapy. Histopathological examination of surgical specimens reveals that approximately 63% of patients undergoing surgery (40% of total patients) have locally extensive tumors or regional (lymph node) metastasis that was undetected at initial diagnosis. These patients are at a significantly greater risk of recurrence. Approximately 40% of these patients will actually develop recurrence within five years after surgery. Results after radiation are even less encouraging. Approximately 80% of patients who have undergone radiation as their primary therapy have disease persistence or develop recurrence or metastasis within five years after treatment. Currently, most of these surgical and radiotherapy patients generally do not receive any immediate follow-up therapy. Rather, they are monitored frequently for elevated Prostate Specific Antigen (“PSA”), which is the primary indicator of recurrence or metastasis.

Thus, there is considerable opportunity to use the present invention in conjunction with surgical intervention.

Hormonal Therapy

Hormonal ablation is the most effective palliative treatment for the 10% of patients presenting with metastatic prostate cancer at initial diagnosis. Hormonal ablation by medication and/or orchiectomy is used to block hormones that support the further growth and metastasis of prostate cancer. With time, both the primary and metastatic tumors of virtually all of these patients become hormone-independent and resistant to therapy. Approximately 50% of patients presenting with metastatic disease die within three years after initial diagnosis, and 75% of such patients die within five years after diagnosis. Continuous supplementation with NAALADase inhibitor based drugs are used to prevent or reverse this potentially metastasis-permissive state.

Among hormones, which may be used in combination with the present inventive compounds, diethylstilbestrol (DES), leuprolide, flutamide, cyproterone acetate, ketoconazole and amino glutethimide are preferred.

Immunotherapy

The antiangiogenic inhibitors of the present invention may also be used in combination with monoclonal antibodies in treating cancer. For example, monoclonal antibodies may be used in treating prostate cancer. A specific example of such an antibody includes cell membrane-specific anti-prostate antibody.

The present invention may also be used with immunotherapies based on polyclonal or monoclonal antibody-derived reagents, for instance. Monoclonal antibody-based reagents are most preferred in this regard. Such reagents are well known to persons of ordinary skill in the art. Radiolabelled monoclonal antibodies for cancer therapy, such as the recently approved use of monoclonal antibody conjugated with strontium-89, also are well known to persons of ordinary skill in the art.

Antiangiogenic Therapy

The antiangiogenic inhibitors of the present invention may also be used in combination with other antiangiogenic agents in treating cancer. Antiangiogenic agents include but are not limited to MMP inhibitors, integrin antagonists, Cox-2 inhibitors, angiostatin, endostatin, thrombospondin-1, and interferon alpha. Examples of preferred antiangiogenic agents include, but are not limited to vitaxin, marimastat, Bay-12-9566, AG-3340, metastat, celecoxib, rofecoxib, JTE-522, EMD-121974, and D-2163 (BMS-275291).

Cryotherapy

Cryotherapy recently has been applied to the treatment of some cancers. Methods and compositions of the present invention also could be used in conjunction with an effective therapy of this type.

All of the various cell types of the body can be transformed into benign or malignant neoplasia or tumor cells and are contemplated as objects of the invention. A “benign” tumor cell denotes the non-invasive and non-metastasized state of a neoplasm. In man, the most frequent neoplasia site is lung, followed by colorectal, breast, prostate, bladder, pancreas, and then ovary. Other prevalent types of cancer include leukemia, central nervous system cancers, including brain cancer, melanoma, lymphoma, erythroleukemia, uterine cancer, and head and neck cancer. Examples 1 through 9 are provided to illustrate contemplated therapeutic combinations, and are not intended to limit the scope of the invention.

The phrase “integrin antagonist” includes agents that impair endothelial cell adhesion via the various integrins. Integrin antagonists induce improperly proliferating endothelial cells to die, by interfering with molecules that blood vessel cells use to bridge between a parent blood vessel and a tumor.

Adhesion forces are critical for many normal physiological functions. Disruptions in these forces, through alterations in cell adhesion factors, are implicated in a variety of disorders, including cancer, stroke, osteoporosis, restenosis, and rheumatoid arthritis. See Horwitz, Scientific American 276:(5):68-75 (1997).

Integrins are a large family of cell surface glycoproteins, which mediate cell adhesion and play central roles in many adhesion phenomena. Integrins are heterodimers composed of noncovalently linked alpha and beta polypeptide subunits. Currently eleven different alpha subunits have been identified and six different beta subunits have been identified. The various alpha subunits can combine with various beta subunits to form distinct integrins.

One integrin known as aᵥb₃ (or the vitronectin receptor) is normally associated with endothelial cells and smooth muscle cells. Aᵥb₃ integrins can promote the formation of blood vessels (angiogenesis) in tumors. These vessels nourish the tumors and provide access routes into the bloodstream for metastatic cells.

The aᵥb₃ integrin is also known to play a role in various other disease states or conditions including tumor metastasis, solid tumor growth (neoplasia), osteoporosis, Paget's disease, humoral hypercalcemia of malignancy, angiogenesis, including tumor angiogenesis, retinopathy, arthritis, including rheumatoid arthritis, periodontal disease, psoriasis, and smooth muscle cell migration (e.g. restenosis).

Tumor cell invasion occurs by a three step process: 1) tumor cell attachment to extracellular matrix; 2) proteolytic dissolution of the matrix; and 3) movement of the cells through the dissolved barrier. This process can occur repeatedly and can result in metastases at sites distant from the original tumor.

The aᵥb₃ integrin and a variety of other aᵥ-containing integrins bind to a number of Arg-Gly-Asp (RGD) containing matrix macromolecules. Compounds containing the RGD sequence mimic extracellular matrix ligands and bind to cell surface receptors. Fibronectin and vitronectin are among the major binding partners of aᵥb₃ integrin. Other proteins and peptides also bind the aᵥb₃ ligand. These include the disintegrins (Pfaff, et al., Cell Adhes. Commun. 2(6): 491-501, 1994), peptides derived from phage display libraries (Healy, J., et al., Protein Pept. Lett. 3(1): 23-30, 1996; Hart, S. L., et al., J. Biol. Chem. 269(17): 12468-12474, 1994) and small cyclic RGD peptides (Pfaff, et al., J. Biol. Chem., 269(32): 20233-20238, 1994). The monoclonal antibody LM609 is also an aᵥb₃ integrin antagonist. See Cheresh, et al., J. Biol. Chem., 262(36):17703-17711 (1987).

Aᵥb₃ inhibitors are being developed as potential anti-cancer agents. Compounds that impair endothelial cell adhesion via the aᵥb₃ integrin induce improperly proliferating endothelial cells to die.

The aᵥb₃ integrin has been shown to play a role in melanoma cell invasion. See Seftor, et al., Proc. Natl. Acad. Sci. USA, 89:1557-1561 (1992). The aᵥb₃ integrin expressed on human melanoma cells has also been shown to promote a survival signal, protecting the cells from apoptosis. See Montgomery, et al., Proc. Natl. Acad. Sci. USA, 91:8856-8860 (1994).

Mediation of the tumor cell metastatic pathway by interference with the aᵥb₃ integrin cell adhesion receptor to impede tumor metastasis would be beneficial. Antagonists of aᵥb₃ have been shown to provide a therapeutic approach for the treatment of neoplasia (inhibition of solid tumor growth) because systemic administration of aᵥb₃ antagonists causes dramatic regression of various histologically distinct human tumors. See Brooks, et al., Cell 79:1157-1164 (1994).

The adhesion receptor identified as integrin aᵥb₃ is a marker of angiogenic blood vessels in chick and man. This receptor plays a critical role in angiogenesis or neovascularization. Angiogenesis is characterized by the invasion, migration and proliferation of smooth muscle and endothelial cells by new blood vessels. Antagonists of aᵥb₃ inhibit this process by selectively promoting apoptosis of cells in the neovasculature. The growth of new blood vessels, also contributes to pathological conditions such as diabetic retinopathy (Adonis, et al., Amer. J. Ophthal., 118: 445-450, 1994) and rheumatoid arthritis (Peacock, et al., J. Exp. Med., 175:, 1135-1138, 1992). Therefore, aᵥb₃ antagonists can be useful therapeutic targets for treating such conditions associated with neovascularization. See Brooks, et al., Science 264:569-571 (1994).

The aᵥb₃ cell surface receptor is also the major integrin on osteoclasts responsible for the attachment to the matrix of bone. Osteoclasts cause bone resorption and when such bone resorbing activity exceeds bone-forming activity, osteoporosis (a loss of bone) results, which leads to an increased number of bone fractures, incapacitation and increased mortality. Antagonists of aᵥb₃ have been shown to be potent inhibitors of osteoclastic activity both in vitro (Sato, et al., J. Cell. Biol., 111: 1713-1723, 1990) and in vivo (Fisher, et al., Endocrinology, 132: 1411-1413, 1993). Antagonism of aᵥb₃ leads to decreased bone resorption and therefore assists in restoring a normal balance of bone forming and resorbing activity. Thus, it would be beneficial to provide antagonists of osteoclast aᵥb₃, which are effective inhibitors of bone resorption and therefore are useful in the treatment or prevention of osteoporosis.

PCT Int. Appl. WO 97/08145 by Sikorski, et al., discloses meta-guanidine, urea, thiourea or azacyclic amino benzoic acid derivatives as highly specific aᵥb₃ integrin antagonists.

PCT Int. Appl. WO 96/00574 Al 960111 by Cousins, R. D. et. al., describe preparation of 3-oxo-2,3,4,5-tetrahydro-1H-1,4-benzodiazepine and 2-benzazepine derivatives and analogs as vitronectin receptor antagonists.

PCT Int. Appl. WO 97/23480 A1 970703 by Jadhav, P. K. et al. describe annelated pyrazoles as novel integrin receptor antagonists. Novel heterocycles including 3-[1-[3-(imidazolin-2-ylamino)propyl]indazol-5-ylcarbonylamino]-2-(benzyl oxycarbonylamino)propionic acid, which are useful as antagonists of the avb3 integrin and related cell surface adhesive protein receptors.

PCT Int. Appl. WO 97/26250 A1 970724 by Hartman, G. D. et al., describe the preparation of arginine dipeptide mimics as integrin receptor antagonists. Selected compounds were shown to bind to human integrin aᵥb₃ with EIB <1000 nM and claimed as compounds, useful for inhibiting the binding of fibrinogen to blood platelets and for inhibiting the aggregation of blood platelets.

PCT Int. Appl. WO 97/23451 by Diefenbach, B. et. al. describe a series of tyrosine-derivatives used as alpha v-integrin inhibitors for treating tumors, osteoporosis, osteolytic disorder and for suppressing angiogenesis.

PCT Int. Appl. WO 96/16983 A1 960606 by Vuori, K. and Ruoslahti, E. describe cooperative combinations of aᵥb₃ integrin ligand and second ligand contained within a matrix, and use in wound healing and tissue regeneration. The compounds contain a ligand for the aᵥb₃ integrin and a ligand for the insulin receptor, the PDGF receptor, the IL-4 receptor, or the IGF receptor, combined in a biodegradable polymeric (e.g. hyaluronic acid) matrix.

PCT Int. Appl. WO 97/10507 A1 970320 by Ruoslahti, E; and Pasqualini, R. describe peptides that home to a selected organ or tissue in vivo, and methods of identifying them. A brain-homing peptide, nine amino acid residues long, for example, directs red blood cells to the brain. Also described is use of in vivo panning to identify peptides homing to a breast tumor or a melanoma.

PCT Int. Appl. WO 96/01653 A1 960125 by Thorpe, Philip E.; Edgington, Thomas S. describes bifunctional ligands for specific tumor inhibition by blood coagulation in tumor vasculature. The disclosed bispecific binding ligands bind through a first binding region to a disease-related target cell, e.g. a tumor cell or tumor vasculature; the second region has coagulation-promoting activity or is a binding region for a coagulation factor. The disclosed bispecific binding ligand may be a bispecific (monoclonal) antibody, or the two ligands may be connected by a (selectively cleavable) covalent bond, a chemical linking agent, an avidin-biotin linkage, and the like. The target of the first binding region can be a cytokine-inducible component, and the cytokine can be released in response to a leukocyte-activating antibody; this may be a bispecific antibody which crosslinks activated leukocytes with tumor cells.

The Vitaxin used in the therapeutic combinations of the present invention can be prepared in the manner set forth in WO 98/33,919.

Some preferred integrin antagonists that may be used in the present invention are listed in the following references hereby each individually incorporated by reference, herein: U.S. Pat. No. 5,773,644; U.S. Pat. No. 5,773,646; U.S. patent application Ser. No. 092/89,140; U.S. Pat. No. 5,852,210; U.S. Pat. No. 5,843,906; U.S. patent application Ser. No. 091/41,547; U.S. Pat. No. 5,952,381; U.S. patent application Ser. No. 092/88,742; U.S. patent application Ser. No. 600/03,277; U.S. patent application Ser. No. 087/13,555; U.S. patent application Ser. No. 092/15,229; U.S. patent application Ser. No. 090/34,758; U.S. patent application Ser. No. 092/61,822; WO 98/33919.

The phrase “matrix metalloproteinase inhibitor” or “MMP inhibitor” includes agents that specifically inhibit a class of enzymes, the zinc metalloproteinases (metalloproteases). The zinc metalloproteinases are involved in the degradation of connective tissue or connective tissue components. These enzymes are released from resident tissue cells and/or invading inflammatory or tumor cells. Blocking the action of zinc metalloproteinases interferes with the creation of paths for newly forming blood vessels to follow. Examples of MMP inhibitors are described in Golub, L M, Inhibition of Matrix Metalloproteinases: Therapeutic Applications (Annals of the New York Academy of Science, Vol 878). Robert A. Greenwald and Stanley Zucker (Eds.), June 1999), and is hereby incorporated by reference.

Connective tissue, extracellular matrix constituents and basement membranes are required components of all mammals. These components are the biological materials that provide rigidity, differentiation, attachments and, in some cases, elasticity to biological systems including human beings and other mammals. Connective tissues components include, for example, collagen, elastin, proteoglycans, fibronectin and laminin. These biochemicals makeup, or are components of structures, such as skin, bone, teeth, tendon, cartilage, basement membrane, blood vessels, cornea and vitreous humor.

Under normal conditions, connective tissue turnover and/or repair processes are controlled and in equilibrium. The loss of this balance for whatever reason leads to a number of disease states. Inhibition of the enzymes responsible loss of equilibrium provides a control mechanism for this tissue decomposition and, therefore, a treatment for these diseases.

Degradation of connective tissue or connective tissue components is carried out by the action of proteinase enzymes released from resident tissue cells and/or invading inflammatory or tumor cells. A major class of enzymes involved in this function are the zinc metalloproteinases (metalloproteases).

The metalloprotease enzymes are divided into classes with some members having several different names in common use. Examples are: collagenase I (MMP-1, fibroblast collagenase; EC 3.4.24.3); collagenase II (MMP-8, neutrophil collagenase; EC 3.4.24.34), collagenase III (MMP-13), stromelysin 1 (MMP-3; EC 3.4.24.17), stromelysin 2 (MMP-10; EC 3.4.24.22), proteoglycanase, matrilysin (MMP-7), gelatinase A (MMP-2, 72 kDa gelatinase, basement membrane collagenase; EC 3.4.24.24), gelatinase B (MMP-9, 92 kDa gelatinase; EC 3.4.24.35), stromelysin 3 (MMP-11), metalloelastase (MMP-12, HME, human macrophage elastase) and membrane MMP (MMP-14). MMP is an abbreviation or acronym representing the term Matrix Metalloprotease with the attached numerals providing differentiation between specific members of the MMP group.

The uncontrolled breakdown of connective tissue by metalloproteases is a feature of many pathological conditions. Examples include rheumatoid arthritis, osteoarthritis, septic arthritis; corneal, epidermal or gastric ulceration; tumor metastasis, invasion or angiogenesis; periodontal disease; proteinuria; Alzheimer's Disease; coronary thrombosis and bone disease. Defective injury repair processes also occur. This can produce improper wound healing leading to weak repairs, adhesions and scarring. These latter defects can lead to disfigurement and/or permanent disabilities as with post-surgical adhesions.

Matrix metalloproteases are also involved in the biosynthesis of tumor necrosis factor (TNF) and inhibition of the production or action of TNF and related compounds is an important clinical disease treatment mechanism. TNF-α, for example, is a cytokine that at present is thought to be produced initially as a 28 kD cell-associated molecule. It is released as an active, 17 kD form that can mediate a large integer of deleterious effects in vitro and in vivo. For example, TNF can cause and/or contribute to the effects of inflammation, rheumatoid arthritis, autoimmune disease, multiple sclerosis, graft rejection, fibrotic disease, cancer, infectious diseases, malaria, mycobacterial infection, meningitis, fever, psoriasis, cardiovascular/pulmonary effects such as post-ischemic reperfusion injury, congestive heart failure, hemorrhage, coagulation, hyperoxic alveolar injury, radiation damage and acute phase responses like those seen with infections and sepsis and during shock such as septic shock and hemodynamic shock. Chronic release of active TNF can cause cachexia and anorexia. TNF can be lethal.

TNF-α convertase is a metalloproteinase involved in the formation of active TNF-α. Inhibition of TNF-α convertase inhibits production of active TNF-α. Compounds that inhibit both MMPs activity have been disclosed in, for example PCT Publication WO 94/24140. Other compounds that inhibit both MMPs activity have also been disclosed in WO 94/02466. Still other compounds that inhibit both MMPs activity have been disclosed in WO 97/20824.

There remains a need for effective MMP and TNF-α convertase inhibiting agents. Compounds that inhibit MMPs such as collagenase, stromelysin and gelatinase have been shown to inhibit the release of TNF. See Gearing, et al., Nature 376:555-557 (1994). McGeehan, et al., Nature 376:558-561 (1994) also reports such findings.

MMPs are involved in other biochemical processes in mammals as well. Included is the control of ovulation, post-partum uterine involution, possibly implantation, cleavage of APP β-Amyloid Precursor Protein) to the amyloid plaque and inactivation of α₁-protease inhibitor (α₁-PI). Inhibition of these metalloproteases permits the control of fertility and the treatment or prevention of Alzheimer's Disease. In addition, increasing and maintaining the levels of an endogenous or administered serine protease inhibitor drug or biochemical such as α₁-PI supports the treatment and prevention of diseases such as emphysema, pulmonary diseases, inflammatory diseases and diseases of aging such as loss of skin or organ stretch and resiliency.

Inhibition of selected MMPs can also be desirable in other instances. Treatment of cancer and/or inhibition of metastasis and/or inhibition of angiogenesis are examples of approaches to the treatment of diseases wherein the selective inhibition of stromelysin (MMP-3), gelatinase (MMP-2), or collagenase III (MMP-13) are the relatively most important enzyme or enzymes to inhibit especially when compared with collagenase I (MMP-1). A drug that does not inhibit collagenase I can have a superior therapeutic profile.

Inhibitors of metalloproteases are known. Examples include natural biochemicals such as tissue inhibitor of metalloproteinase (TIMP), α₂-macroglobulin and their analogs or derivatives. These are high molecular weight protein molecules that form inactive complexes with metalloproteases. An integer of smaller peptide-like compounds that inhibit metalloproteases have been described. Mercaptoamide peptidyl derivatives have shown ACE inhibition in vitro and in vivo. Angiotensin converting enzyme (ACE) aids in the production of angiotensin II, a potent pressor substance in mammals and inhibition of this enzyme leads to the lowering of blood pressure.

Thiol group-containing amide or peptidyl amide-based metalloprotease (MMP) inhibitors are known as is shown in, for example, WO 95/12389. Thiol group-containing amide or peptidyl amide-based metalloprotease (MMP) inhibitors are also shown in WO 96/11209. Still further Thiol group-containing amide or peptidyl amide-based metalloprotease (MMP) inhibitors are shown in U.S. Pat. No. 4,595,700. Hydroxamate group-containing MMP inhibitors are disclosed in a number of published patent applications that disclose carbon back-boned compounds, such as in WO 95/29892. Other published patents include WO 97/24117. Additionally, EP 0 780 386 further discloses hydroxamate group-containing MMP inhibitors. WO 90/05719 disclose hydroxamates that have a peptidyl back-bones or peptidomimetic back-bones. WO 93/20047 also discloses hydroxamates that have a peptidyl back-bones or peptidomimetic back-bones. Additionally, WO 95/09841 discloses disclose hydroxamates that have peptidyl back-bones or peptidomimetic back-bones, and WO 96/06074 further discloses hydroxamates that have peptidyl back-bones or peptidomimetic back-bones. Schwartz, et al., Progr. Med. Chem. 29.271-334 (1992) also discloses disclose hydroxamates that have peptidyl back-bones or peptidomimetic back-bones. Furthermore, Rasmussen, et al., Pharmacol. Ther. 75(1): 69-75 (1997) discloses hydroxamates that have peptidyl back-bones or peptidomimetic back-bones. Also, Denis, et al., Invest. New Drugs 15(3): 175-185 (1997) discloses hydroxamates that have a peptidyl back-bones or peptidomimetic back-bones as well.

One possible problem associated with known MMP inhibitors is that such compounds often exhibit the same or similar inhibitory effects against each of the MMP enzymes. For example, the peptidomimetic hydroxamate known as batimastat is reported to exhibit IC₅₀ values of about 1 to about 20 nanomolar (nM) against each of MMP-1, MMP-2, MMP-3, MMP-7, and MMP-9. Marimastat, another peptidomimetic hydroxamate was reported to be another broad-spectrum MMP inhibitor with an enzyme inhibitory spectrum very similar to batimastat, except that marimastat exhibited an IC₅₀ value against MMP-3 of 230 nM. See Rasmussen, et al., Pharmacol. Ther. 75(1):69-75 (1997).

Meta analysis of data from Phase I/II studies using marimastat in patients with advanced, rapidly progressive, treatment-refractory solid tumor cancers (colorectal, pancreatic, ovarian, prostate), indicated a dose-related reduction in the rise of cancer-specific antigens used as surrogate markers for biological activity. The most common drug-related toxicity of marimastat in those clinical trials was musculoskeletal pain and stiffness, often commencing in the small joints in the hands, spreading to the arms and shoulder. A short dosing holiday of 1-3 weeks followed by dosage reduction permits treatment to continue. See Rasmussen, et al., Pharmacol. Ther. 75(1):69-75 (1997). It is thought that the lack of specificity of inhibitory effect among the MMPs may be the cause of that effect.

In view of the importance of hydroxamate MMP inhibitor compounds in the treatment of several diseases and the lack of enzyme specificity exhibited by two of the more potent drugs now in clinical trials, it would be beneficial to use hydroxamates of greater enzyme specificity. This would be particularly the case if the hydroxamate inhibitors exhibited limited inhibition of MMP-1 that is relatively ubiquitous and as yet not associated with any pathological condition, while exhibiting quite high inhibitory activity against one or more of MMP-2, MMP-9 or MMP-13 that are associated with several pathological conditions.

The Marimastat used in the therapeutic combinations of the present invention can be prepared in the manner set forth in WO 94/02,447.

The Bay-12-9566 used in the therapeutic combinations of the present invention can be prepared in the manner set forth in WO 96/15,096.

The AG-3340 used in the therapeutic combinations of the present invention can be prepared in the manner set forth in WO 97/20,824.

The Metastat used in the therapeutic combinations of the present invention can be prepared in the manner set forth in U.S. Pat. No. 5,837,696.

The D-2163 used in the therapeutic combinations of the present invention can be prepared in the manner set forth in WO 97/19,075.

One component of the present invention is an antiangiogenesis inhibitor such as, for example, a Cox-2 inhibitor. Studies indicate that prostaglandins synthesized by cyclooxygenases play a critical role in the initiation and promotion of cancer. Moreover, Cox-2 is overexpressed in neoplastic lesions of the colon, breast, lung, prostate, esophagus, pancreas, intestine, cervix, ovaries, urinary bladder, and head & neck. In several in vitro and animal models, Cox-2 inhibitors have inhibited tumor growth and metastasis.

In addition to cancers perse, Cox-2 is also expressed in the angiogenic vasculature within and adjacent to hyperplastic and neoplastic lesions indicating that Cox-2 plays a role in angiogenesis. In both the mouse and rat, Cox-2 inhibitors markedly inhibited bFGF-induced neovascularization. The utility of Cox-2 inhibitors as chemopreventive, antiangiogenic and chemotherapeutic agents is described in the literature (Koki, et al., Potential utility of Cox-2 inhibitors in chemoprevention and chemotherapy. Exp. Opin. Invest. Drugs (1999) 8(10) pp. 1623-1638, hereby incorporated by reference). Amplification and/or overexpression of HER-2/nue (ErbB2) occurs in 20-30% of human breast and ovarian cancers as well as in 5-15% of gastric and esophageal cancers and is associated with poor prognosis. Additionally, it has been recently discovered in vitro that Cox-2 expression is upregulated in cells overexpressing the HER-2/neu oncogene. (Subbaramaiah, et al., Increased expression of Cox-2 in HER-2/neu-overexpressing breast cancer. Cancer Research (submitted 1999), hereby incorporated by reference). In this study, markedly increased levels of PGE₂ production, Cox-2 protein and mRNA were detected in HER-2/neu transformed ammary epithelial cells compared to a non-transformed partner cell line. Products of Cox-2 activity, i.e., prostaglandins, stimulate proliferation, increase invasiveness of malignant cells, and enhance the production of vascular endothelial growth factor, which promotes angiogenesis. Further, HER-2/neu induces the production of angiogenic factors such as vascular endothelial growth factor.

Consequently, the administration of a Cox-2 inhibitor in combination with an anti HER-2/neu antibodies such as trastuzumab (Herceptin®) and other therapies directed at inhibiting HER-2/neu is contemplated to treat cancers in which HER-2/neu is overexpressed.

Also, it is contemplated that Cox-2 levels are elevated in tumors with amplification and/or overexpression of other oncogenes including but not limited to c-myc, N-myc, L-myc, K-ras, H-ras, N-ras. Products of Cox-2 activity stimulate cell proliferation, inhibit immune surveillance, increase invasiveness of malignant cells, and promote angiogenesis. Consequently, the administration of a Cox-2 inhibitor in combination with an agent or agents that inhibits or suppresses oncogenes is contemplated to prevent or treat cancers in which oncogenes are overexpressed.

Accordingly, there is a need for a method of treating or preventing cancer in a patient that overexpresses Cox-2 and/or an oncogene. Methods for the production of anti-ErbB2 antibodies are described in WO 99/31140.

Specific Cox-2 inhibitors are useful for the treatment of cancer (WO98/16227) and in several animal models reduce angiogenesis driven by various growth factors (WO98/22101). Anti-angiogenesis was achieved with a Cox-2 inhibitor in rats implanted with bFGF, vascular endothelium growth factor (VEGF) or carrageenan, proteins with well-known angiogenic properties. (Masferrer, et al., 89th Annual Meeting of the American Association for Cancer Research, March 1998.)

The phrase “cyclooxygenase-2 inhibitor” or“Cox-2 inhibitor” or “cyclooxygenase-II inhibitor” or “Cox-II inhibitor” includes agents that specifically inhibit a class of enzymes, Cox-2, with less significant inhibition of Cox-1.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC₅₀ value for inhibition of Cox-1, divided by the IC₅₀ value for inhibition of Cox-2 (Cox-1 IC₅₀/Cox-2 IC₅₀). A Cox-2 selective inhibitor is any inhibitor for which the ratio of Cox-1 IC₅₀ to Cox-2 IC₅₀ is greater than 1. In preferred embodiments, this ratio is greater than 2, more preferably greater than 5, yet more preferably greater than 10, still more preferably greater than 50, and more preferably still greater than 100.

As used herein, the term “IC₅₀” refers to the concentration of a compound that is required to produce 50% inhibition of cyclooxygenase activity. Preferred Cox-2 selective inhibitors of the present invention have a cyclooxygenase-2 IC₅₀ of less than about 1 μM, more preferred of less than about 0.5 μM, and even more preferred of less than about 0.2 μM.

Preferred Cox-2 selective inhibitors have a Cox-1 IC₅₀ of greater than about 1 μM, and more preferably of greater than 20 μM. Such preferred selectivity may indicate an ability to reduce the incidence of common NSAID-induced side effects.

Also included within the scope of the present invention are compounds that act as prodrugs of Cox-2 selective inhibitors. As used herein in reference to Cox-2 selective inhibitors, the term “prodrug” refers to a chemical compound that can be converted into an active Cox-2 selective inhibitor by metabolic or simple chemical processes within the body of the subject. One example of a prodrug for a Cox-2 selective inhibitor is parecoxib, which is a therapeutically effective prodrug of the tricyclic Cox-2 selective inhibitor valdecoxib. An example of a preferred Cox-2 selective inhibitor prodrug is parecoxib sodium. A class of prodrugs of Cox-2 inhibitors is described in U.S. Pat. No. 5,932,598. The Cox-2 selective inhibitor of the present invention can be, for example, the Cox-2 selective inhibitor meloxicam, Formula B-1 (CAS registry number 71125-38-7), or a pharmaceutically acceptable salt or prodrug thereof.

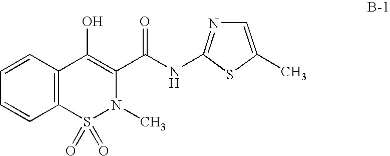

In another embodiment of the invention the Cox-2 selective inhibitor can be the Cox-2 selective inhibitor RS 57067, 6-[[5-(4-chlorobenzoyl)-1,4-dimethyl-1H-pyrrol-2-yl]methyl]-3(2H)-pyridazinone, Formula B-2 (CAS registry number 179382-91-3), or a pharmaceutically acceptable salt or prodrug thereof.

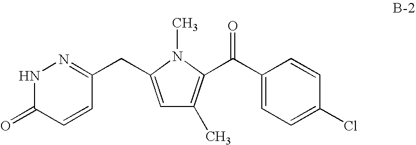

In a another embodiment of the invention the Cox-2 selective inhibitor is of the chromene/chroman structural class that is a substituted benzopyran or a substituted benzopyran analog, and even more preferably selected from the group consisting of substituted benzothiopyrans, dihydroquinolines, or dihydronaphthalenes having the structure of any one of the compounds having a structure shown by general Formulas I, II, III, IV, V, and VI, shown below, and possessing, by way of example and not limitation, the structures disclosed in Table 1, including the diastereomers, enantiomers, racemates, tautomers, salts, esters, amides and prodrugs thereof.

Benzopyrans that can serve as a Cox-2 selective inhibitor of the present invention include substituted benzopyran derivatives that are described in U.S. Pat. No. 6,271,253. One such class of compounds is defined by the general formula shown below in formulas I:

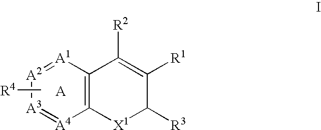

wherein X¹ is selected from O, S, CR^{c} R^{b} and NR^{a};

wherein R^{a} is selected from hydrido, C₁-C₃-alkyl, (optionally substituted phenyl)-C₁-C₃-alkyl, acyl and carboxy-C₁-C₆-alkyl;

wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{b}R^{c} forms a 3-6 membered cycloalkyl ring;

wherein R¹ is selected from carboxyl, aminocarbonyl, C₁-C₆-alkylsulfonylaminocarbonyl and C₁-C₆-alkoxycarbonyl;

wherein R² is selected from hydrido, phenyl, thienyl, C₁-C₆-alkyl and C₂-C₆-alkenyl;

wherein R³is selected from C₁-C₃-perfluoroalkyl, chloro, C₁-C₆-alkylthio, C₁-C₆-alkoxy, nitro, cyano and cyano-C₁-C₃-alkyl;

wherein R⁴is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₁-alkoxycarbonyl, formyl, C₁-C₆-haloalkylcarbonyl and C₁-C₆-alkylcarbonyl; and

wherein the A ring atoms A¹, A², A³ and A⁴ are independently selected from carbon and nitrogen with the proviso that at least two of A¹, A², A³ and A⁴ are carbon;

or wherein R⁴ together with ring A forms a radical selected from naphthyl, quinolyl, isoquinolyl, quinolizinyl, quinoxalinyl and dibenzofuryl;

or an isomer or pharmaceutically acceptable salt thereof.

Another class of benzopyran derivatives that can serve as the Cox-2 selective inhibitor of the present invention includes a compound having the structure of formula II:

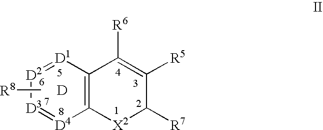

wherein X² is selected from O, S, CR^{c}R^{b} and NR^{a};

wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁-C₆-alkyl;

wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl;

or wherein CRC R^{b} form a cyclopropyl ring;

wherein R⁵ is selected from carboxyl, aminocarbonyl, C₁-C₆-alkylsulfonylaminocarbonyl and C₁-C₆-alkoxycarbonyl;

wherein R⁶ is selected from hydrido, phenyl, thienyl, C₂-C₆-alkynyl and C₂-C₆-alkenyl;

wherein R⁷ is selected from C₁-C₃-perfluoroalkyl, chloro, C₁-C₆-alkylthio, C₁-C₆-alkoxy, nitro, cyano and cyano-C₁-C₃-alkyl;

wherein R⁸ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, —O(CF₂)₂ O—, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl), C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and

wherein the D ring atoms D¹, D², D³ and D⁴ are independently selected from carbon and nitrogen with the proviso that at least two of D¹, D², D³ and D⁴ are carbon; or

wherein R⁸ together with ring D forms a radical selected from naphthyl, quinolyl, isoquinolyl, quinolizinyl, quinoxalinyl and dibenzofuryl;

or an isomer or pharmaceutically acceptable salt thereof.

Other benzopyran Cox-2 selective inhibitors useful in the practice of the present invention are described in U.S. Pat. Nos. 6,034,256 and 6,077,850. The general formula for these compounds is shown in formula III:

Formula III is:

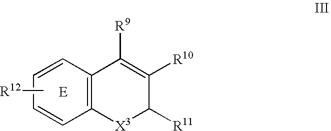

wherein X³ is selected from the group consisting of O or S or NR^{a};

wherein R^{a} is alkyl;

wherein R⁹ is selected from the group consisting of H and aryl;

wherein R¹⁰ is selected from the group consisting of carboxyl, aminocarbonyl, alkylsulfonylaminocarbonyl and alkoxycarbonyl;

wherein R¹¹ is selected from the group consisting of haloalkyl, alkyl, aralkyl, cycloalkyl and aryl optionally substituted with one or more radicals selected from alkylthio, nitro and alkylsulfonyl; and

wherein R¹² is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or

wherein R¹² together with ring E forms a naphthyl radical; or an isomer or pharmaceutically acceptable salt thereof; and

including the diastereomers, enantiomers, racemates, tautomers, salts, esters, amides and prodrugs thereof.

A related class of compounds useful as Cox-2 selective inhibitors in the present invention is described by Formulas IV and V:

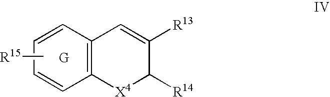

wherein X⁴ is selected from O or S or NR^{a};

wherein R^{a} is alkyl;

wherein R¹³ is selected from carboxyl, aminocarbonyl, alkylsulfonylaminocarbonyl and alkoxycarbonyl;

wherein R¹⁴ is selected from haloalkyl, alkyl, aralkyl, cycloalkyl and aryl optionally substituted with one or more radicals selected from alkylthio, nitro and alkylsulfonyl; and

wherein R¹⁵ is one or more radicals selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl;

or wherein R¹⁵ together with ring G forms a naphthyl radical;

or an isomer or pharmaceutically acceptable salt thereof.

Formula V is:

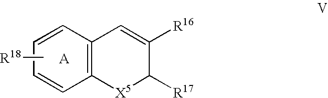

wherein:

X⁵ is selected from the group consisting of O or S or NR^{b};

R^{b} is alkyl;

R¹⁶ is selected from the group consisting of carboxyl, aminocarbonyl, alkylsulfonylaminocarbonyl and alkoxycarbonyl;

R¹⁷ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

R¹⁸ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R¹⁸ together with ring A forms a naphthyl radical;

or an isomer or pharmaceutically acceptable salt thereof.

The Cox-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting of lower haloalkyl, lower cycloalkyl and phenyl; and

R¹⁸ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, and lower alkylcarbonyl; or

wherein R¹⁸ together with ring A forms a naphthyl radical;

or an isomer or pharmaceutically acceptable salt thereof.

The Cox-2 selective inhibitor may also be a compound of Formula V, wherein:

X⁵ is selected from the group consisting of oxygen and sulfur;

R¹⁶ is carboxyl;

R¹⁷ is lower haloalkyl; and

R¹⁸ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl; or wherein R¹⁸ together with ring A forms a naphthyl radical;

or an isomer or pharmaceutically acceptable salt thereof.

The Cox-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, and trifluoromethyl; and

R¹⁸ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethyalamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or

wherein R² together with ring A forms a naphthyl radical;

or an isomer or pharmaceutically acceptable salt thereof.

The Cox-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting trifluoromethyl and pentafluoroethyl; and

R¹⁸ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or wherein R¹⁸ together with ring A forms a naphthyl radical;

or an isomer or prodrug thereof.

The Cox-2 selective inhibitor of the present invention can also be a compound having the structure of Formula VI:

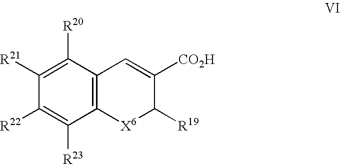

wherein:

X⁶ is selected from the group consisting of O and S;

R¹⁹ is lower haloalkyl;

R²⁰ is selected from the group consisting of hydrido, and halo;

R²¹ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6-membered nitrogen-containing heterocyclosulfonyl;

R²² is selected from the group consisting of hydrido, lower alkyl, halo, lower alkoxy, and aryl; and

R²³ is selected from the group consisting of the group consisting of hydrido, halo, lower alkyl, lower alkoxy, and aryl;

or an isomer or prodrug thereof.

The Cox-2 selective inhibitor can also be a compound of having the structure of Formula VI, wherein:

X⁶ is selected from the group consisting of O and S;

R¹⁹ is selected from the group consisting of trifluoromethyl and pentafluoroethyl;

R²⁰ is selected from the group consisting of hydrido chloro, and fluoro;

R²¹ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, and morpholinosulfonyl;

R²² is selected from the group consisting of hydrido, methyl, ethyl, isopropyl, tert-butyl, chloro, methoxy, diethylamino, and phenyl; and

R²³ is selected from the group consisting of hydrido, chloro, bromo, fluoro, methyl, ethyl, tert-butyl, methoxy, and phenyl;

or an isomer or prodrug thereof.

TABLE 1
Examples of Chromene Cox-2 Selective Inhibitors

| Compound | |
|---|---|
| Number | Structural Formula |
| B-3 | 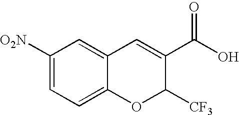 |
| | 6-Nitro-2-trifluoromethyl-2H-1- |
| | benzopyran-3-carboxylic acid |
| B-4 | 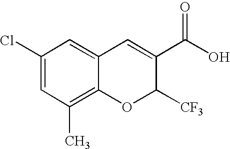 |
| | 6-Chloro-8-methyl-2-trifluoromethyl- |
| | 2H-1-benzopyran-3-carboxylic acid |
| B-5 | 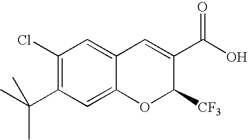 |
| | ((S)-6-Chloro-7-(1,1-dimethylethyl)-2-(trifluoromethyl- |
| | 2H-1-benzopyran-3-carboxylic acid |
| B-6 | 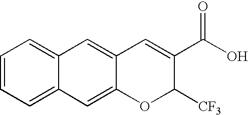 |
| | 2-Trifluoromethyl-2H-naphtho[2,3-b] |
| | pyran-3-carboxylic acid |
| B-7 | 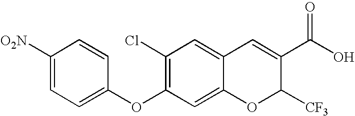 |
| | 6-Chloro-7-(4-nitrophenoxy)-2-(trifluoromethyl)-2H-1- |
| | benzopyran-3-carboxylic acid |
| B-8 | 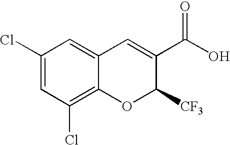 |
| | ((S)-6,8-Dichloro-2-(trifluoromethyl)-2H-1- |
| | benzopyran-3-carboxylic acid |
| B-9 | 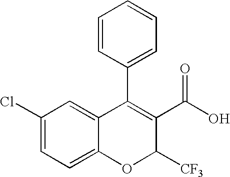 |
| | 6-Chloro-2-(trifluoromethyl)-4-phenyl-2H- |
| | 1-benzopyran-3-carboxylic acid |
| B-10 | 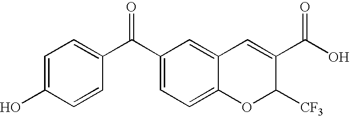 |
| | 6-(4-Hydroxybenzoyl)-2-(trifluoromethyl)-2H- |
| | 1-benzopyran-3-carboxylic acid |
| B-11 | 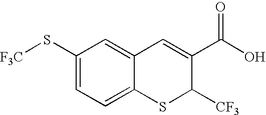 |
| | 2-(Trifluoromethyl)-6-[(trifluoromethyl)thio]- |
| | 2H-1-benzothiopyran-3-carboxylic acid |
| B-12 | 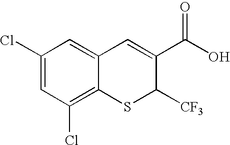 |
| | 6,8-Dichloro-2-trifluoromethyl-2H-1- |
| | benzothiopyran-3-carboxylic acid |
| B-13 | 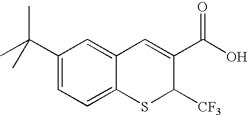 |
| | 6-(1,1-Dimethylethyl)-2-(trifluoromethyl)- |
| | 2H-1-benzothiopyran-3-carboxylic acid |
| B-14 | 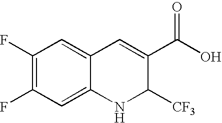 |
| | 6,7-Difluoro-1,2-dihydro-2-(trifluoromethyl)-3- |
| | quinolinecarboxylic acid |
| B-15 | 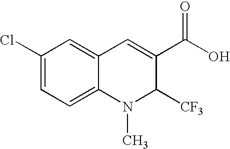 |
| | 6-Chloro-1,2-dihydro-1-methyl-2- |
| | (trifluoromethyl)-3-quinolinecarboxylic acid |
| B-16 | 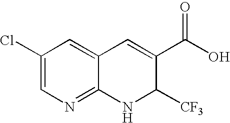 |
| | 6-Chloro-2-(trifluoromethyl)-1,2-dihydro |
| | [1,8]naphthyridine-3-carboxylic acid |
| B-17 | 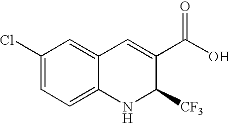 |
| | ((S)-6-Chloro-1,2-dihydro-2-(trifluoromethyl)- |
| | 3-quinolinecarboxylic acid |
| B-18 | 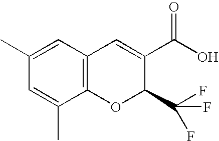 |
| | (2S)-6,8-dimethyl-2-(trifluoromethyl)-2H- |
| | chromene-3-carboxylic acid |
| B-19 | 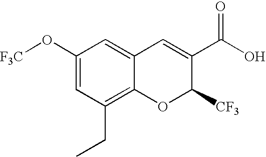 |
| | (2S)-8-ethyl-6-(trifluoromethoxy)-2- |
| | (trifluoromethyl)-2H-chromene-3-carboxylic acid |
| B-20 | 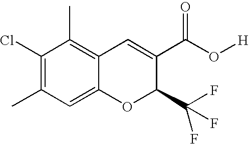 |
| | (2S)-6-chloro-5,7-dimethyl-2- |
| | (trifluoromethyl)-2H-chromene-3-carboxylic acid |

Examples of specific compounds that are useful for the Cox-2 selective inhibitor include (without limitation):

a1) 8-acetyl-3-(4-fluorophenyl)-2-(4-methylsulfonyl)phenyl-imidazo(1,2-a)pyridine;

a2) 5,5-dimethyl-4-(4-methylsulfonyl)phenyl-3-phenyl-2-(5H)-furanone;

a3) 5-(4-fluorophenyl)-1-[4-(methylsulfonyl)phenyl]-3-(trifluoromethyl)pyrazole;

a4) 4-(4-fluorophenyl)-5-[4-(methylsulfonyl)phenyl]-1-phenyl-3-(trifluoromethyl)pyrazole;

a5) 4-(5-(4-chlorophenyl)-3-(4-methoxyphenyl)-1H-pyrazol-1-yl)benzenesulfonamide

a6) 4-(3,5-bis(4-methylphenyl)-1H-pyrazol-1-yl)benzenesulfonamide;

a7) 4-(5-(4-chlorophenyl)-3-phenyl-1H-pyrazol-1-yl)benzenesulfonamide;

a8) 4-(3,5-bis(4-methoxyphenyl)-1H-pyrazol-1-yl)benzenesulfonamide;

a9) 4-(5-(4-chlorophenyl)-3-(4-methylphenyl)-1H-pyrazol-1-yl)benzenesulfonamide;

a10) 4-(5-(4-chlorophenyl)-3-(4-nitrophenyl)-1H-pyrazol-1-yl)benzenesulfonamide;

b1) 4-(5-(4-chlorophenyl)-3-(5-chloro-2-thienyl)-1H-pyrazol-1-yl)benzenesulfonamide;

b2) 4-(4-chloro-3,5-diphenyl-1H-pyrazol-1-yl)benzenesulfonamide

b3) 4-[5-(4-chlorophenyl)-3-(trifluoromethyl)-1H-pyrazol-1-yl]benzenesulfonamide;

b4) 4-[5-phenyl-3-(trifluoromethyl)-1H-pyrazol-1-yl]benzenesulfonamide;

b5) 4-[5-(4-fluorophenyl)-3-(trifluoromethyl)-1H-pyrazol-1-yl]benzenesulfonamide;

b6) 4-[5-(4-methoxyphenyl)-3-(trifluoromethyl)-1H-pyrazol-1-yl]benzenesulfonamide;

b7) 4-[5-(4-chlorophenyl)-3-(difluoromethyl)-1H-pyrazol-1-yl]benzenesulfonamide;

b8) 4-[5-(4-methylphenyl)-3-(trifluoromethyl)-1H-pyrazol-1-yl]benzenesulfonamide;

b9) 4-[4-chloro-5-(4-chlorophenyl)-3-(trifluoromethyl)-1H-pyrazol-1-yl]benzenesulfonamide;

b10) 4-[3-(difluoromethyl)-5-(4-methylphenyl)-1H-pyrazol-1-yl]benzenesulfonamide;

c1) 4-[3-(difluoromethyl)-5-phenyl-1H-pyrazol-1-yl]benzenesulfonamide;

c2) 4-[3-(difluoromethyl)-5-(4-methoxyphenyl)-1H-pyrazol-1-yl]benzenesulfonamide;

c3) 4-[3-cyano-5-(4-fluorophenyl)-1H-pyrazol-1-yl]benzenesulfonamide;

c4) 4-[3-(difluoromethyl)-5-(3-fluoro-4-methoxyphenyl)-1H-pyrazol-1-yl]benzenesulfonamide;

c5) 4-[5-(3-fluoro-4-methoxyphenyl)-3-(trifluoromethyl)-1H-pyrazol-1-yl]benzenesulfonamide;

c6) 4-[4-chloro-5-phenyl-1H-pyrazol-1-yl]benzenesulfonamide;

c7) 4-[5-(4-chlorophenyl)-3-(hydroxymethyl)-1H-pyrazol-1-yl]benzenesulfonamide;

c8) 4-[5-(4-(N,N-dimethylamino)phenyl)-3-(trifluoromethyl)-1H-pyrazol-1-yl]benzenesulfonamide;

c9) 5-(4-fluorophenyl)-6-[4-(methylsulfonyl)phenyl]spiro[2.4]hept-5-ene;

c10) 4-[6-(4-fluorophenyl)spiro[2.4]hept-5-en-5-yl]benzenesulfonamide;

d1) 6-(4-fluorophenyl)-7-[4-(methylsulfonyl)phenyl]spiro[3.4]oct-6-ene;

d2) 5-(3-chloro-4-methoxyphenyl)-6-[4-(methylsulfonyl)phenyl]spiro[2.4]hept-5-ene;

d3) 4-[6-(3-chloro-4-methoxyphenyl)spiro[2.4]hept-5-en-5-yl]benzenesulfonamide;

d4) 5-(3,5-dichloro-4-methoxyphenyl)-6-[4-(methylsulfonyl)phenyl]spiro[2.4]hept-5-ene;

d5) 5-(3-chloro-4-fluorophenyl)-6-[4-(methylsulfonyl)phenyl]spiro[2.4]hept-5-ene;

d6) 4-[6-(3,4-dichlorophenyl)spiro[2.4]hept-5-en-5-yl]benzenesulfonamide;

d7) 2-(3-chloro-4-fluorophenyl)-4-(4-fluorophenyl)-5-(4-methylsulfonylphenyl)thiazole;

d8) 2-(2-chlorophenyl)-4-(4-fluorophenyl)-5-(4-methylsulfonylphenyl)thiazole;

d9) 5-(4-fluorophenyl)-4-(4-methylsulfonylphenyl)-2-methylthiazole;

d10) 4-(4-fluorophenyl)-5-(4-methylsulfonylphenyl)-2-trifluoromethylthiazole;

e1) 4-(4-fluorophenyl)-5-(4-methylsulfonylphenyl)-2-(2-thienyl)thiazole;

e2) 4-(4-fluorophenyl)-5-(4-methylsulfonylphenyl)-2-benzylaminothiazole;

e3) 4-(4-fluorophenyl)-5-(4-methylsulfonylphenyl)-2-(1-propylamino)thiazole;

e4) 2-[(3,5-dichlorophenoxy)methyl)-4-(4-fluorophenyl)-5-[4-(methylsulfonyl)phenyl]thiazole;

e5) 5-(4-fluorophenyl)-4-(4-methylsulfonylphenyl)-2-trifluoromethylthiazole;

e6) 1-methylsulfonyl-4-[1,1-dimethyl-4-(4-fluorophenyl)cyclopenta-2,4-dien-3-yl]benzene;

e7) 4-[4-(4-fluorophenyl)-1, 1-dimethylcyclopenta-2,4-dien-3-yl]benzenesulfonamide;

e8) 5-(4-fluorophenyl)-6-[4-(methylsulfonyl)phenyl]spiro[2.4]hepta-4,6-diene;

e9) 4-[6-(4-fluorophenyl)spiro[2.4]hepta-4,6-dien-5-yl]benzenesulfonamide;

e10) 6-(4-fluorophenyl)-2-methoxy-5-[4-(methylsulfonyl)phenyl]-pyridine-3-carbonitrile;

f1) 2-bromo-6-(4-fluorophenyl)-5-[4-(methylsulfonyl)phenyl]-pyridine-3-carbonitrile;

f2) 6-(4-fluorophenyl)-5-[4-(methylsulfonyl)phenyl]-2-phenyl-pyridine-3-carbonitrile;

f3) 4-[2-(4-methylpyridin-2-yl)-4-(trifluoromethyl)-1H-imidazol-1-yl]benzenesulfonamide;

f4) 4-[2-(5-methylpyridin-3-yl)-4-(trifluoromethyl)-1H-imidazol-1-yl]benzenesulfonamide;

f5) 4-[2-(2-methylpyridin-3-yl)-4-(trifluoromethyl)-1H-imidazol-1-yl]benzenesulfonamide;

f6) 3-[1-[4-(methylsulfonyl)phenyl]-4-(trifluoromethyl)-1H-imidazol-2-yl]pyridine;

f7) 2-[1-[4-(methylsulfonyl)phenyl-4-(trifluoromethyl)-1H-imidazol-2-yl]pyridine;

f8) 2-methyl-4-[1-[4-(methylsulfonyl)phenyl-4-(trifluoromethyl)-1H-imidazol-2-yl]pyridine;

f9) 2-methyl-6-[1-[4-(methylsulfonyl)phenyl-4-(trifluoromethyl)-1H-imidazol-2-yl]pyridine;

f10) 4-[2-(6-methylpyridin-3-yl)-4-(trifluoromethyl)-1H-imidazol-1-yl]benzenesulfonamide;

g1) 2-(3,4-difluorophenyl)-1-[4-(methylsulfonyl)phenyl]-4-(trifluoromethyl)-1H-imidazole;

g2) 4-[2-(4-methylphenyl)-4-(trifluoromethyl)-1H-imidazol-1-yl]benzenesulfonamide;

g3) 2-(4-chlorophenyl)-1-[4-(methylsulfonyl)phenyl]-4-methyl-1H-imidazole;

g4) 2-(4-chlorophenyl)-1-[4-(methylsulfonyl)phenyl]-4-phenyl-1H-imidazole;

g5) 2-(4-chlorophenyl)-4-(4-fluorophenyl)-1-[4-(methylsulfonyl)phenyl]-1H-imidazole;

g6) 2-(3-fluoro-4-methoxyphenyl)-1-[4-(methylsulfonyl)phenyl-4-(trifluoromethyl)-1H-imidazole;

g7) 1-[4-(methylsulfonyl)phenyl]-2-phenyl-4-trifluoromethyl-1H-imidazole;

g8) 2-(4-methylphenyl)-1-[4-(methylsulfonyl)phenyl]-4-trifluoromethyl-1H-imidazole;

g9) 4-[2-(3-chloro-4-methylphenyl)-4-(trifluoromethyl)-1H-imidazol-1-yl]benzenesulfonamide;

g10) 2-(3-fluoro-5-methylphenyl)-1-[4-(methylsulfonyl)phenyl]-4-(trifluoromethyl)-1H-imidazole;

h1) 4-[2-(3-fluoro-5-methylphenyl)-4-(trifluoromethyl)-1H-imidazol-1-yl]benzenesulfonamide;

h2) 2-(3-methylphenyl)-1-[4-(methylsulfonyl)phenyl]-4-trifluoromethyl-1H-imidazole;

h3) 4-[2-(3-methylphenyl)-4-trifluoromethyl-1H-imidazol-1-yl]benzenesulfonamide;

h4) 1-[4-(methylsulfonyl)phenyl]-2-(3-chlorophenyl)-4-trifluoromethyl-1H-imidazole;

h5) 4-[2-(3-chlorophenyl)-4-trifluoromethyl-1H-imidazol-1-yl]benzenesulfonamide;

h6) 4-[2-phenyl-4-trifluoromethyl-1H-imidazol-1-yl]benzenesulfonamide;

h7) 4-[2-(4-methoxy-3-chlorophenyl)-4-trifluoromethyl-1H-imidazol-1-yl]benzenesulfonamide;

h8) 1-allyl-4-(4-fluorophenyl)-3-[4-(methylsulfonyl)phenyl]-5-(trifluoromethyl)-1H-pyrazole;

h9) 4-[1-ethyl-4-(4-fluorophenyl)-5-(trifluoromethyl)-1H-pyrazol-3-yl]benzenesulfonamide;

i1) N-phenyl-[4-(4-luorophenyl)-3-[4-(methylsulfonyl)phenyl]-5-(trifluoromethyl)-1H-pyrazol-1-yl]acetamide;

i2) ethyl [4-(4-fluorophenyl)-3-[4-(methylsulfonyl)phenyl]-5-(trifluoromethyl)-1H-pyrazol-1-yl]acetate;

i3) 4-(4-fluorophenyl)-3-[4-(methylsulfonyl)phenyl]-1-(2-phenylethyl)-1H-pyrazole;

i4) 4-(4-fluorophenyl)-3-[4-(methylsulfonyl)phenyl]-1-(2-phenylethyl)-5-(trifluoromethyl)pyrazole;

i5) 1-ethyl-4-(4-fluorophenyl)-3-[4-(methylsulfonyl)phenyl]-5-(trifluoromethyl)-1H-pyrazole;

i6) 5-(4-fluorophenyl)-4-(4-methylsulfonylphenyl)-2-trifluoromethyl-1H-imidazole;

i7) 4-[4-(methylsulfonyl)phenyl]-5-(2-thiophenyl)-2-(trifluoromethyl)-1H-imidazole;

i8) 5-(4-fluorophenyl)-2-methoxy-4-[4-(methylsulfonyl)phenyl]-6-(trifluoromethyl)pyridine;

i9) 2-ethoxy-5-(4-fluorophenyl)-4-[4-(methylsulfonyl)phenyl]-6-(trifluoromethyl)pyridine;

i10) 5-(4-fluorophenyl)-4-[4-(methylsulfonyl)phenyl]-2-(2-propynyloxy)-6-(trifluoromethyl)pyridine;

j1) 2-bromo-5-(4-fluorophenyl)-4-[4-(methylsulfonyl)phenyl]-6-(trifluoromethyl)pyridine;

j2) 4-[2-(3-chloro-4-methoxyphenyl)-4,5-difluorophenyl]benzenesulfonamide;

j3) 1-(4-fluorophenyl)-2-[4-(methylsulfonyl)phenyl]benzene;

j4) 5-difluoromethyl-4-(4-methylsulfonylphenyl)-3-phenylisoxazole;

j5) 4-[3-ethyl-5-phenylisoxazol-4-yl]benzenesulfonamide;

j6) 4-[5-difluoromethyl-3-phenylisoxazol-4-yl]benzenesulfonamide;

j7) 4-[5-hydroxymethyl-3-phenylisoxazol-4-yl]benzenesulfonamide;

j8) 4-[5-methyl-3-phenyl-isoxazol-4-yl]benzenesulfonamide;

j9) 1-[2-(4-fluorophenyl)cyclopenten-1-yl]-4-(methylsulfonyl)benzene;

j10) 1-[2-(4-fluoro-2-methylphenyl)cyclopenten-1-yl]-4-(methylsulfonyl)benzene;

k1) 1-[2-(4-chlorophenyl)cyclopenten-1-yl]-4-(methylsulfonyl)benzene;

k2) 1-[2-(2,4-dichlorophenyl)cyclopenten-1-yl]-4-(methylsulfonyl)benzene;

k3) 1-[2-(4-trifluoromethylphenyl)cyclopenten-1-yl]-4-(methylsulfonyl)benzene;

k4) 1-[2-(4-methylthiophenyl)cyclopenten-1-yl]-4-(methylsulfonyl)benzene;

k5) 1-[2-(4-fluorophenyl)-4,4-dimethylcyclopenten-1-yl]-4-(methylsulfonyl)benzene;

k6) 4-[2-(4-fluorophenyl)-4,4-dimethylcyclopenten-1-yl]benzenesulfonamide;

k7) 1-[2-(4-chlorophenyl)-4,4-dimethylcyclopenten-1-yl]-4-(methylsulfonyl)benzene;

k8) 4-[2-(4-chlorophenyl)-4,4-dimethylcyclopenten-1-yl]benzenesulfonamide;

k9) 4-[2-(4-fluorophenyl)cyclopenten-1-yl]benzenesulfonamide;

k10) 4-[2-(4-chlorophenyl)cyclopenten-1-yl]benzenesulfonamide;

l1) 1-[2-(4-methoxyphenyl)cyclopenten-1-yl]-4-(methylsulfonyl)benzene;

l2) 1-[2-(2,3-difluorophenyl)cyclopenten-1-yl]-4-(methylsulfonyl)benzene;

l3) 4-[2-(3-fluoro-4-methoxyphenyl)cyclopenten-1-yl]benzenesulfonamide;

l4) 1-[2-(3-chloro-4-methoxyphenyl)cyclopenten-1-yl]-4-(methylsulfonyl)benzene;

l5) 4-[2-(3-chloro-4-fluorophenyl)cyclopenten-1-yl]benzenesulfonamide;

l6) 4-[2-(2-methylpyridin-5-yl)cyclopenten-1-yl]benzenesulfonamide;

l7) ethyl 2-[4-(4-fluorophenyl)-5-[4-(methylsulfonyl) phenyl]oxazol-2-yl]-2-benzyl-acetate;

l8) 2-[4-(4-fluorophenyl)-5-[4-(methylsulfonyl)phenyl]oxazol-2-yl]acetic acid;

l9) 2-(tert-butyl)-4-(4-fluorophenyl)-5-[4-(methylsulfonyl)phenyl]oxazole;

l10) 4-(4-fluorophenyl)-5-[4-(methylsulfonyl)phenyl]-2-phenyloxazole;

m1) 4-(4-fluorophenyl)-2-methyl-5-[4-(methylsulfonyl)phenyl]oxazole; and

m2) 4-[5-(3-fluoro-4-methoxyphenyl)-2-trifluoromethyl-4-oxazolyl]benzenesulfonamide.

m3) 6-chloro-2-trifluoromethyl-2H-1-benzopyran-3-carboxylic acid;

m4) 6-chloro-7-methyl-2-trifluoromethyl-2H-1-benzopyran-3-carboxylic acid;

m5) 8-(1-methylethyl)-2-trifluoromethyl-2H-1-benzopyran-3-carboxylic acid;

m6) 6-chloro-7-(1,1-dimethylethyl)-2-trifluoromethyl-2H-1-benzopyran-3-carboxylic acid;

m7) 6-chloro-8-(1-methylethyl)-2-trifluoromethyl-2H-1-benzopyran-3-carboxylic acid;

m8) 2-trifluoromethyl-3H-naphthopyran-3-carboxylic acid

m9) 7-(1,1-dimethylethyl)-2-trifluoromethyl-2H-1-benzopyran-3-carboxylic acid;

m10) 6-bromo-2-trifluoromethyl-2H-1-benzopyran-3-carboxylic acid;

n1) 8-chloro-2-trifluoromethyl-2H-1-benzopyran-3-carboxylic acid;

n2) 6-trifluoromethoxy-2-trifluoromethyl-2H-1-benzopyran-3-carboxylic acid;

n3) 5,7-dichloro-2-trifluoromethyl-2H-1-benzopyran-3-carboxylic acid;

n4) 8-phenyl-2-trifluoromethyl-2H-1-benzopyran-3-carboxylic acid;

n5) 7,8-dimethyl-2-trifluoromethyl-2H-1-benzopyran-3-carboxylic acid;

n6) 6,8-bis(dimethylethyl)-2-trifluoromethyl-2H-1-benzopyran-3-carboxylic acid;

n7) 7-(1-methylethyl)-2-trifluoromethyl-2H-1-benzopyran-3-carboxylic acid;

n8) 7-phenyl-2-trifluoromethyl-2H-1-benzopyran-3-carboxylic acid;

n9) 6-chloro-7-ethyl-2-trifluoromethyl-2H-1-benzopyran-3-carboxylic acid;

n10) 6-chloro-8-ethyl-2-trifluoromethyl-2H-1-benzopyran-3-carboxylic acid;

o1) 6-chloro-7-phenyl-2-trifluoromethyl-2H-1-benzopyran-3-carboxylic acid;

o2) 6,7-dichloro-2-trifluoromethyl-2H-1-benzopyran-3-carboxylic acid;

o3) 6,8-dichloro-2-trifluoromethyl-2H-1-benzopyran-3-carboxylic acid;

o4) 2-trifluoromethyl-3H-naptho[2,1-b]pyran-3-carboxylic acid;

o5) 6-chloro-8-methyl-2-trifluoromethyl-2H-1-benzopyran-3-carboxylic acid;

o6) 8-chloro-6-methyl-2-trifluoromethyl-2H-1-benzopyran-3-carboxylic acid;

o7) 8-chloro-6-methoxy-2-trifluoromethyl-2H-1-benzopyran-3-carboxylic acid;

o8) 6-bromo-8-chloro-2-trifluoromethyl-2H-1-benzopyran-3-carboxylic acid;

o9) 8-bromo-6-fluoro-2-trifluoromethyl-2H-1-benzopyran-3-carboxylic acid;

o10) 8-bromo-6-methyl-2-trifluoromethyl-2H-1-benzopyran-3-carboxylic acid;

p1) 8-bromo-5-fluoro-2-trifluoromethyl-2H-1-benzopyran-3-carboxylic acid;

p2) 6-chloro-8-fluoro-2-trifluoromethyl-2H-1-benzopyran-3-carboxylic acid;

p3) 6-bromo-8-methoxy-2-trifluoromethyl-2H-1-benzopyran-3-carboxylic acid;

p4) 6-[[(phenylmethyl)amino]sulfonyl]-2-trifluoromethyl-2H-1-benzopyran-3-carboxylic acid;

p5) 6-[(dimethylamino)sulfonyl]-2-trifluoromethyl-2H-1-benzopyran-3-carboxylic acid;

p6) 6-[(methylamino)sulfonyl]-2-trifluoromethyl-2H-1-benzopyran-3-carboxylic acid;

p7) 6-[(4-morpholino)sulfonyl]-2-trifluoromethyl-2H-1-benzopyran-3-carboxylic acid;

p8) 6-[(1,1-dimethylethyl)aminosulfonyl]-2-trifluoromethyl-2H-1-benzopyran-3-carboxylic acid;

p9) 6-[(2-methylpropyl)aminosulfonyl]-2-trifluoromethyl-2H-1-benzopyran-3-carboxylic acid;

p10) 6-methylsulfonyl-2-trifluoromethyl-2H-1-benzopyran-3-carboxylic acid;

q1) 8-chloro-6-[[(phenylmethyl)amino]sulfonyl]-2-trifluoromethyl-2H-1-benzopyran-3-carboxylic acid;

q2) 6-phenylacetyl-2-trifluoromethyl-2H-1-benzopyran-3-carboxylic acid;

q3) 6,8-dibromo-2-trifluoromethyl-2H-1-benzopyran-3-carboxylic acid;

q4) 8-chloro-5,6-dimethyl-2-trifluoromethyl-2H-1-benzopyran-3-carboxylic acid;

q5) 6,8-dichloro-(S)-2-trifluoromethyl-2H-1-benzopyran-3-carboxylic acid;

q6) 6-benzylsulfonyl-2-trifluoromethyl-2H-1-benzopyran-3-carboxylic acid;

q7) 6-[[N-(2-furylmethyl)amino]sulfonyl]-2-trifluoromethyl-2H-1-benzopyran-3-carboxylic acid;

q8) 6-[[N-(2-phenylethyl)amino]sulfonyl]-2-trifluoromethyl-2H-1-benzopyran-3-carboxylic acid;

q9) 6-iodo-2-trifluoromethyl-2H-1-benzopyran-3-carboxylic acid;

q10) 7-(1,1-dimethylethyl)-2-pentafluoroethyl-2H-1-benzopyran-3-carboxylic acid;

r1) 5,5-dimethyl-3-(3-fluorophenyl)-4-(4-methyl-sulphonyl-2(5H)-fluranone;

r2) 6-chloro-2-trifluoromethyl-2H-1-benzothiopyran-3-carboxylic acid;

r3) 4-[5-(4-chlorophenyl)-3-(trifluoromethyl)-1H-pyrazol-1-yl]benzenesulfonamide;

r4) 4-[5-(4-methylphenyl)-3-(trifluoromethyl)-1H-pyrazol-1-yl]benzenesulfonamide;

r5) 4-[5-(3-fluoro-4-methoxyphenyl)-3-(difluoromethyl)-1H-pyrazol-1-yl]benzenesulfonamide;

r6) 3-[1-[4-(methylsulfonyl)phenyl]-4-trifluoromethyl-1H-imidazol-2-yl]pyridine;

r7) 2-methyl-5-[1-[4-(methylsulfonyl)phenyl]-4-trifluoromethyl-1H-imidazol-2-yl]pyridine;

r8) 4-[2-(5-methylpyridin-3-yl)-4-(trifluoromethyl)-1H-imidazol-1-yl]benzenesulfonamide;

r9) 4-[5-methyl-3-phenylisoxazol-4-yl]benzenesulfonamide;

r10) 4-[5-hydroxymethyl-3-phenylisoxazol-4-yl]benzenesulfonamide;

s1) [2-trifluoromethyl-5-(3,4-difluorophenyl)-4-oxazolyl]benzenesulfonamide;

s2) 4-[2-methyl-4-phenyl-5-oxazolyl]benzenesulfonamide; or

s3) 4-[5-(3-fluoro-4-methoxyphenyl-2-trifluoromethyl)-4-oxazolyl]benzenesulfonamide;

or a pharmaceutically acceptable salt or prodrug thereof.

In a further preferred embodiment of the invention the Cox-2 inhibitor can be selected from the class of tricyclic Cox-2 selective inhibitors represented by the general structure of formula VII:

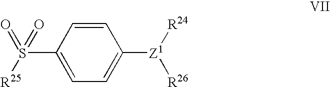

wherein:

Z¹ is selected from the group consisting of partially unsaturated or unsaturated heterocyclyl and partially unsaturated or unsaturated carbocyclic rings;

R²⁴ is selected from the group consisting of heterocyclyl, cycloalkyl, cycloalkenyl and aryl, wherein R²⁴ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R²⁵ is selected from the group consisting of methyl or amino; and

R²⁶ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N- arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;

or a prodrug thereof.

In a preferred embodiment of the invention the Cox-2 selective inhibitor represented by the above Formula VII is selected from the group of compounds, illustrated in Table 2, which includes celecoxib (B-18), valdecoxib (B-19), deracoxib (B-20), rofecoxib (B-21), etoricoxib (MK-663; B-22), JTE-522 (B-23), or a prodrug thereof.

Additional information about selected examples of the Cox-2 selective inhibitors discussed above can be found as follows: celecoxib (CAS RN 169590-42-5, C-2779, SC-58653, and in U.S. Pat. No. 5,466,823); deracoxib (CAS RN 169590-41-4); rofecoxib (CAS RN 162011-90-7); compound B-24 (U.S. Pat. No. 5,840,924); compound B-26 (WO 00/25779); and etoricoxib (CAS RN 202409-33-4, MK-663, SC-86218, and in WO 98/03484).

TABLE 2
Examples of Tricyclic COX-2 Selective Inhibitors

| Compound | |
|---|---|
| Number | Structural Formula |
| B-18 | 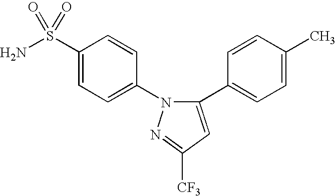 |
| B-19 | 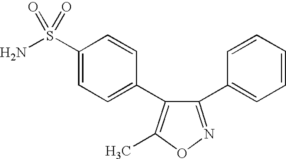 |
| B-20 | 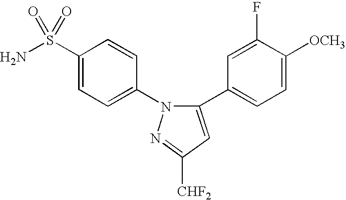 |
| B-21 | 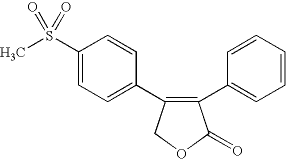 |
| B-22 | 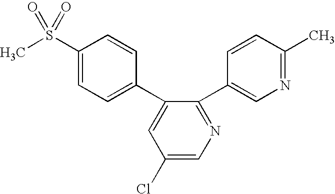 |
| B-23 | 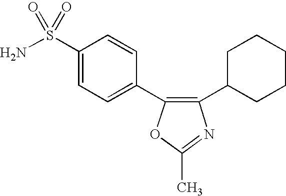 |

In a more preferred embodiment of the invention, the Cox-2 selective inhibitor is selected from the group consisting of celecoxib, rofecoxib and etoricoxib.

In a preferred embodiment of the invention, parecoxib (See, e.g. U.S. Pat. No. 5,932,598), having the structure shown in B-24, which is a therapeutically effective prodrug of the tricyclic Cox-2 selective inhibitor valdecoxib, B-19, (See, e.g., U.S. Pat. No. 5,633,272), may be advantageously employed as a source of a cyclooxygenase inhibitor.

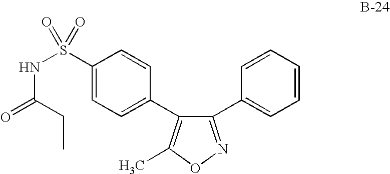

A preferred form of parecoxib is sodium parecoxib.

In another embodiment of the invention, the compound ABT-963 having the formula B-25 that has been previously described in International Publication number WO 00/24719, is another tricyclic Cox-2 selective inhibitor which may be advantageously employed.

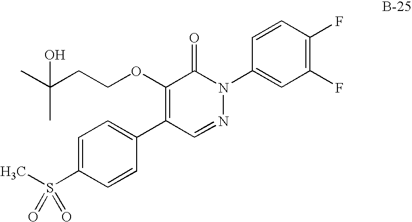

In a further embodiment of the invention, the cyclooxygenase inhibitor can be selected from the class of phenylacetic acid derivative Cox-2 selective inhibitors represented by the general structure of Formula VIII:

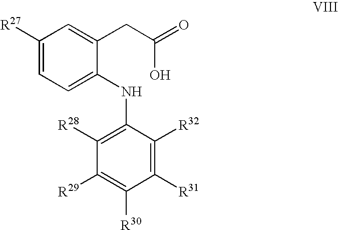

wherein:

R²⁷ is methyl, ethyl, or propyl;

R²⁸ is chloro or fluoro;

R²⁹ is hydrogen, fluoro, or methyl;

R³⁰ is hydrogen, fluoro, chloro, methyl, ethyl, methoxy, ethoxy or hydroxy;

R³¹ is hydrogen, fluoro, or methyl; and

R³² is chloro, fluoro, trifluoromethyl, methyl, or ethyl, provided that R²⁸, R²⁹, R³⁰ and R³¹ are not all fluoro when R²⁷ is ethyl and R³⁰ is H.

A phenylacetic acid derivative Cox-2 selective inhibitor that is described in WO 99/11605 is a compound that has the structure shown in Formula VIII, wherein:

R²⁷ is ethyl;

R²⁸ and R³⁰ are chloro;

R²⁹ and R³¹ are hydrogen; and

R³² is methyl.

Another phenylacetic acid derivative Cox-2 selective inhibitor is a compound that has the structure shown in Formula VIII, wherein:

R²⁷ is propyl;

R²⁸ and R³⁰ are chloro;

R²⁹ and R³¹ are methyl; and

R³² is ethyl.

Another phenylacetic acid derivative Cox-2 selective inhibitor that is described in WO 02/20090 is a compound that is referred to as COX-189 (also termed lumiracoxib), having CAS Reg. No. 220991-20-8, and having the structure shown in Formula VIII, wherein:

R²⁷ is methyl;

R²⁸ is fluoro;

R³² is chloro; and

R²⁹, R³⁰, and R³¹ are hydrogen.

Compounds that have a structure similar to that shown in Formula VIII, which can serve as the Cox-2 selective inhibitor of the present invention, are described in U.S. Pat. Nos. 6,310,099, 6,291,523, and 5,958,978.

Other Cox-2 selective inhibitors that can be used in the present invention have the general structure shown in formula IX, where the J group is a carbocycle or a heterocycle. Preferred embodiments have the structure:

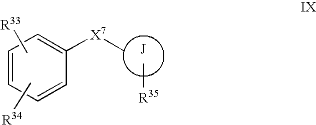

wherein:

X is O; J is 1-phenyl; R³³ is 2-NHSO₂CH₃; R³⁴ is 4-NO₂; and there is no R³⁵ group, (nimesulide), and

X is O; J is 1-oxo-inden-5-yl; R³³ is 2-F; R³⁴ is 4-F; and R³⁵ is 6-NHSO₂CH₃, (flosulide); and

X is O; J is cyclohexyl; R³³ is 2-NHSO₂CH₃; R³⁴ is 5-NO₂; and there is no R³⁵ group, (NS-398); and

X is S; J is 1-oxo-inden-5-yl; R³³ is 2-F; R³⁴ is 4-F; and R³⁵ is 6-N⁻SO₂CH₃ Na⁺,

(L-745337); and

X is S; J is thiophen-2-yl; R³³ is 4-F; there is no R³⁴ group; and R³⁵ is 5-NHSO₂CH₃, (RWJ-63556); and

X is O; J is 2-oxo-5(R)-methyl-5-(2,2,2-trifluoroethyl)furan-(5H)-3-yl; R³³ is 3-F; R³⁴ is 4-F; and R³⁵ is 4-(p-SO₂CH₃)C₆H₄, (L-784512).

Further information on the applications of the Cox-2 selective inhibitor N-(2-cyclohexyloxynitrophenyl) methane sulfonamide (NS-398, CAS RN 123653-11-2), having a structure as shown in formula B-26, have been described by, for example, Yoshimi, N. et al., in Japanese J. Cancer Res., 90(4):406-412 (1999); Falgueyret, J. -P. et al., in Science Spectra, available at: http://www.gbhap.com/Science_Spectra/20-1-article.htm (Jun. 6, 2001); and Iwata, K. et al., in Jpn. J. Pharmacol., 75(2):191-194 (1997).

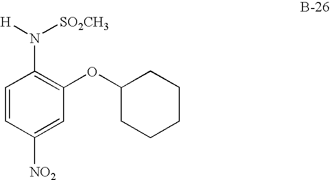

An evaluation of the anti-inflammatory activity of the Cox-2 selective inhibitor, RWJ 63556, in a canine model of inflammation, was described by Kirchner et al., in J Pharmacol Exp Ther 282, 1094-1101 (1997).

Materials that can serve as the Cox-2 selective inhibitor of the present invention include diarylmethylidenefuran derivatives that are described in U.S. Pat. No. 6,180,651. Such diarylmethylidenefuran derivatives have the general formula shown below in formula X:

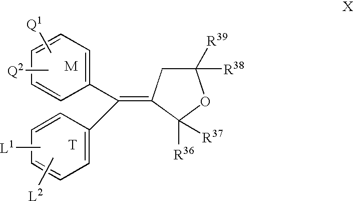

wherein:

the rings T and M independently are:

a phenyl radical,

a naphthyl radical,

a radical derived from a heterocycle comprising 5 to 6 members and possessing from 1 to 4 heteroatoms, or

a radical derived from a saturated hydrocarbon ring having from 3 to 7 carbon atoms;

at least one of the substituents Q¹, Q², L¹ or L² is:

an —S(O)ₙ—R group, in which n is an integer equal to 0, 1 or 2 and R is:

a lower alkyl radical having 1 to 6 carbon atoms or

a lower haloalkyl radical having 1 to 6 carbon atoms, or

an —SO₂NH₂ group;

and is located in the para position,

the others independently being:

a hydrogen atom,

a halogen atom,

a lower alkyl radical having 1 to 6 carbon atoms,

a trifluoromethyl radical, or

a lower O-alkyl radical having 1 to 6 carbon atoms, or

Q¹ and Q² or L¹ and L² are a methylenedioxy group; and

R³⁶, R³⁷, R³⁸ and R³⁹ independently are:

a hydrogen atom,

a halogen atom,

a lower alkyl radical having 1 to 6 carbon atoms,

a lower haloalkyl radical having 1 to 6 carbon atoms, or

an aromatic radical selected from the group consisting of phenyl, naphthyl, thienyl, furyl and pyridyl; or,

R³⁶, R³⁷ or R³⁸, R³⁹ are an oxygen atom, or

R³⁶, R³⁷ or R³⁸, R³⁹, together with the carbon atom to which they are attached, form a saturated hydrocarbon ring having from 3 to 7 carbon atoms;

or an isomer or prodrug thereof.

Particular materials that are included in this family of compounds, and which can serve as the Cox-2 selective inhibitor in the present invention, include N-(2-cyclohexyloxynitrophenyl)methane sulfonamide, and (E)-4-[(4-methylphenyl)(tetrahydro-2-oxo-3-furanylidene) methyl]benzenesulfonamide.

Cox-2 selective inhibitors that are useful in the present invention include darbufelone (Pfizer), CS-502 (Sankyo), LAS 34475 (Almirall Profesfarma), LAS 34555 (Almirall Profesfarma), S-33516 (Servier), SD 8381 (Pharmacia, described in U.S. Pat. No. 6,034,256), BMS-347070 (Bristol-Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis), BF-389 (Biofor/Scherer), GR-253035 (Glaxo Wellcome), 6-dioxo-9H-purin-8-yl-cinnamic acid (Glaxo Wellcome), and S-2474 (Shionogi).

Information about S-33516, mentioned above, can be found in Current Drugs Headline News, at http://www.current-drugs.com/NEWS/Inflam1.htm, Oct. 4, 2001, where it was reported that S-33516 is a tetrahydroisoinde derivative which has IC₅₀ values of 0.1 and 0.001 mM against cyclooxygenase-1 and Cox-2, respectively. In human whole blood, S-33516 was reported to have an ED₅₀=0.39 mg/kg.

Compounds that may act as Cox-2 selective inhibitors include multibinding compounds containing from 2 to 10 ligands covanlently attached to one or more linkers, as described in U.S. Pat. No. 6,395,724.

Compounds that may act as Cox-2 inhibitors include conjugated linoleic acid that is described in U.S. Pat. No. 6,077,868.

Materials that can serve as a Cox-2 selective inhibitor of the present invention include heterocyclic aromatic oxazole compounds that are described in U.S. Pat. Nos. 5,994,381 and 6,362,209. Such heterocyclic aromatic oxazole compounds have the formula shown below in formula XI:

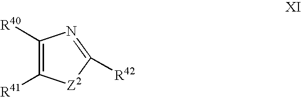

wherein:

Z² is an oxygen atom;

one of R⁴⁰ and R⁴¹ is a group of the formula

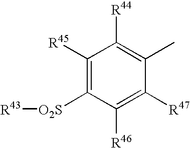

wherein:

R⁴³ is lower alkyl, amino or lower alkylamino; and

R⁴⁴, R⁴⁵, R⁴⁶ and R⁴⁷ are the same or different and each is hydrogen atom, halogen atom, lower alkyl, lower alkoxy, trifluoromethyl, hydroxy or amino, provided that at least one of R⁴⁴, R⁴⁵, R⁴⁶ and R⁴⁷ is not hydrogen atom, and the other is an optionally substituted cycloalkyl, an optionally substituted heterocyclic group or an optionally substituted aryl; and

R³⁰ is a lower alkyl or a halogenated lower alkyl,

and a pharmaceutically acceptable salt thereof.

Cox-2 selective inhibitors that are useful in the subject method and compositions can include compounds that are described in U.S. Pat. Nos. 6,080,876 and 6,133,292, and described by formula XII:

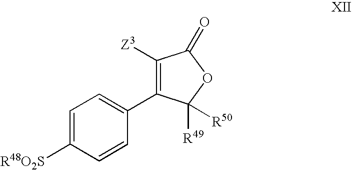

wherein:

Z³ is selected from the group consisting of:

(a) linear or branched C₁₋₆ alkyl,

(b) linear or branched C₁₋₆ alkoxy,

(c) unsubstituted, mono-, di- or tri-substituted phenyl or naphthyl wherein the substituents are selected from the group consisting of:

(1) hydrogen,

(2) halo,

(3) C₁₋₃ alkoxy,

(4) CN,

(5) C₁₋₃ fluoroalkyl

(6) C₁₋₃ alkyl,

(7) —CO₂H;

R⁴⁸ is selected from the group consisting of NH₂ and CH₃,

R⁴⁹ is selected from the group consisting of:

C₁₋₆ alkyl unsubstituted or substituted with C₃₋₆ cycloalkyl, and

C₃₋₆ cycloalkyl;

R⁵⁰ is selected from the group consisting of:

C₁₋₆ alkyl unsubstituted or substituted with one, two or three fluoro atoms; and

C₃₋₆ cycloalkyl;

with the proviso that R⁴⁹ and R⁵⁰ are not the same.

Materials that can serve as Cox-2 selective inhibitors include pyridines that are described in U.S. Pat. Nos. 6,369,275, 6,127,545, 6,130,334, 6,204,387, 6,071,936, 6,001,843 and 6,040,450, and which have the general formula described by formula XIII:

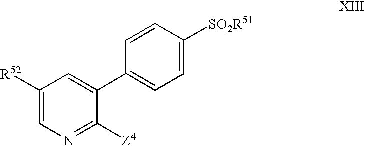

wherein:

R⁵¹ is selected from the group consisting of:

(a) CH₃,

(b) NH₂,

(c) NHC(O)CF₃,

(d) NHCH₃;

Z⁴ is a mono-, di-, or trisubstituted phenyl or pyridinyl (or the N-oxide thereof),

wherein the substituents are chosen from the group consisting of:

(a) hydrogen,

(b) halo,

(c) C₁₋₆ alkoxy,

(d) C₁₋₆ alkylthio,

(e) CN,

(f) C₁₋₆ alkyl,

(g) C₁₋₆ fluoroalkyl,

(h) N₃,

(i) —CO₂R⁵³,

(j) hydroxy,

(k) —C(R⁵⁴)(R⁵⁵)—OH,

(l) —C₁₋₆alkyl-CO₂—R⁵⁶,

(m) C₁₋₆fluoroalkoxy;

R⁵² is chosen from the group consisting of:

(a) halo,

(b) C₁₋₆alkoxy,

(c) C₁₋₆ alkylthio,

(d) CN,

(e) C₁₋₆ alkyl,

(f) C₁₋₆ fluoroalkyl,

(g) N₃,

(h) —CO₂R⁵⁷,

(i) hydroxy,

(j) —C(R⁵⁸)(R⁵⁹)—OH,

(k) —C₁₋₆alkyl-CO₂—R⁶⁰,

(l) C₁₋₆fluoroalkoxy,

(m) NO₂,

(n) NR⁶¹R⁶², and

(o) NHCOR⁶³;

R⁵³, R⁵⁴, R⁵⁵, R⁵⁶, R⁵⁷, R⁵⁸, R⁵⁹, R⁶⁰, R⁶¹, R⁶², R⁶³, are each in chosen from the group consisting of:

(a) hydrogen, and

(b) C₁₋₆alkyl;

or R⁵⁴ and R⁵⁵, R⁵⁸ and R⁵⁹ or R⁶¹ and R⁶² together with the atom to which they are attached form a saturated monocyclic ring of 3, 4, 5, 6, or 7 atoms.

Materials that can serve as the Cox-2 selective inhibitor of the present invention include diarylbenzopyran derivatives that are described in U.S. Pat. No. 6,340,694. Such diarylbenzopyran derivatives have the general formula shown below in formula XIV:

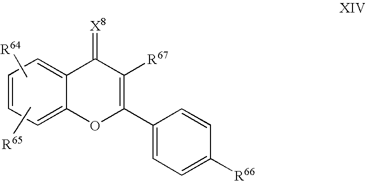

wherein:

X⁸ is an oxygen atom or a sulfur atom;

R⁶⁴ and R⁶⁵, identical to or different from each other, are independently a hydrogen atom, a halogen atom, a C₁-C₆ lower alkyl group, a trifluoromethyl group, an alkoxy group, a hydroxy group, a nitro group, a nitrile group, or a carboxyl group;

R⁶⁶ is a group of a formula: S(O)ₙR⁶⁸ wherein n is an integer of 0˜2, R⁶⁸ is a hydrogen atom, a C₁-C₆ lower alkyl group, or a group of a formula: NR⁶⁹

R⁷⁰ wherein R⁶⁹ and R⁷⁰, identical to or different from each other, are independently a hydrogen atom, or a C₁-C₆ lower alkyl group; and

R⁶⁷ is oxazolyl, benzo[b]thienyl, furanyl, thienyl, naphthyl, thiazolyl, indolyl, pyrolyl, benzofuranyl, pyrazolyl, pyrazolyl substituted with a C₁-C₆ lower alkyl group, indanyl, pyrazinyl, or a substituted group represented by the following structures:

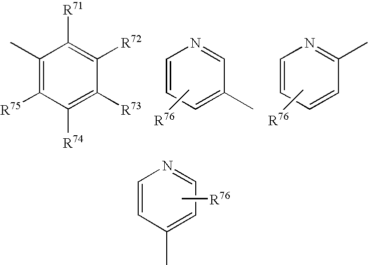

wherein:

R⁷¹ through R⁷⁵, identical to or different from one another, are independently a hydrogen atom, a halogen atom, a C₁-C₆ lower alkyl group, a trifluoromethyl group, an alkoxy group, a hydroxy group, a hydroxyalkyl group, a nitro group, a group of a formula: S(O)ₙR⁶⁸, a group of a formula: NR⁶⁹ R⁷⁰, a trifluoromethoxy group, a nitrile group a carboxyl group, an acetyl group, or a formyl group,

wherein n, R⁶⁸, R⁶⁹ and R⁷⁰ have the same meaning as defined by R⁶⁶ above; and

R⁷⁶ is a hydrogen atom, a halogen atom, a C₁-C₆ lower alkyl group, a trifluoromethyl group, an alkoxy group, a hydroxy group, a trifluoromethoxy group, a carboxyl group, or an acetyl group.

Materials that can serve as the Cox-2 selective inhibitor of the present invention include 1-(4-sulfamylaryl)-3-substituted-5-aryl-2-pyrazolines that are described in U.S. Pat. No. 6,376,519. Such 1-(4-sulfamylaryl)-3-substituted-5-aryl-2-pyrazolines have the formula shown below in formula XV:

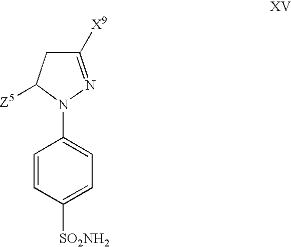

wherein:

X⁹ is selected from the group consisting of C₁-C₆ trihalomethyl, preferably trifluoromethyl; C₁-C₆ alkyl; and an optionally substituted or di-substituted phenyl group of formula XVI:

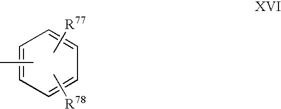

wherein:

R⁷⁷ and R⁷⁸ are independently selected from the group consisting of hydrogen, halogen, preferably chlorine, fluorine and bromine; hydroxyl; nitro; C₁-C₆ alkyl, preferably C₁-C₃ alkyl; C₁-C₆ alkoxy, preferably C₁-C₃ alkoxy; carboxy; C₁-C₆ trihaloalkyl, preferably trihalomethyl, most preferably trifluoromethyl; and cyano;

Z⁵ is selected from the group consisting of substituted and unsubstituted aryl.

Materials that can serve as the Cox-2 selective inhibitor of the present invention include heterocycles that are described in U.S. Pat. No. 6,153,787. Such heterocycles have the general formulas shown below in formulas XVII and XVIII:

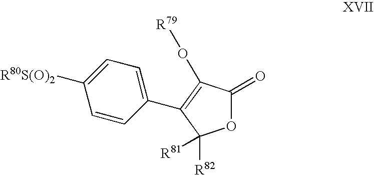

wherein:

R⁷⁹ is a mono-, di-, or tri-substituted C₁₋₁₂ alkyl, or a mono-, or an unsubstituted or mono-, di- or tri-substituted linear or branched C₂₋₁₀ alkenyl, or an unsubstituted or mono-, di- or tri-substituted linear or branched C₂₋₁₀ alkynyl, or an unsubstituted or mono-, di- or tri-substituted C₃₋₁₂ cycloalkenyl, or an unsubstituted or mono-, di- or tri-substituted C₅₋₁₂ cycloalkynyl, wherein the substituents are chosen from the group consisting of:

(a) halo, selected from F, Cl, Br, and I,

(b) OH,

(c) CF₃,

(d) C₃₋₆ cycloalkyl,

(e) ═O,

(f) dioxolane,

(g) CN; and

R⁸⁰ is selected from the group consisting of:

(a) CH₃,

(b) NH₂,

(c) NHC(O)CF₃,

(d) NHCH₃;

R⁸¹ and R⁸² are independently chosen from the group consisting of:

(a) hydrogen,

(b) C₁₋₁₀ alkyl;

or R⁸¹ and R⁸² together with the carbon to which they are attached form a saturated monocyclic carbon ring of 3, 4, 5, 6 or 7 atoms.

Formula XVIII is:

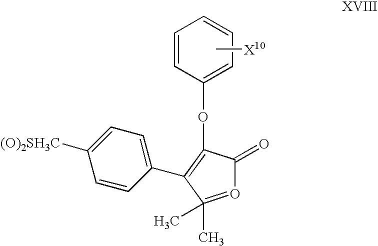

X¹⁰ is fluoro or chloro.

Materials that can serve as the Cox-2 selective inhibitor of the present invention include 2,3,5-trisubstituted pyridines that are described in U.S. Pat. No. 6,046,217. Such pyridines have the general formula shown below in formula XIX:

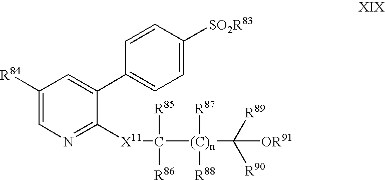

or a pharmaceutically acceptable salt thereof, wherein:

X¹¹ is selected from the group consisting of:

(a) O,

(b) S,

(c) bond;

n is 0 or 1;

R⁸³ is selected from the group consisting of:

(a) CH₃,

(b) NH₂,

(c) NHC(O)CF₃;

R⁸⁴ is chosen from the group consisting of:

(a) halo,

(b) C₁₋₆ alkoxy,

(c) C₁₋₆ alkylthio,

(d) CN,

(e) C₁₋₆ alkyl,

(f) C₁₋₆ fluoroalkyl,

(g) N₃,

(h) —CO₂ R⁹²,

(i) hydroxy,

(j) —C(R⁹³)(R⁹⁴)—OH,

(k) —C₁₋₆ alkyl-CO₂—R⁹⁵,

(l) C₁₋₆ fluoroalkoxy,

(m) NO₂,

(n) NR⁹⁶ R⁹⁷,

(o) NHCOR⁹⁸;

R⁸⁵ to R⁹⁸ are independantly chosen from the group consisting of

(a) hydrogen,

(b) C₁₋₆ alkyl;

or R⁸⁵ and R⁸⁹, or R⁸⁹ and R⁹⁰ together with the atoms to which they are attached form a carbocyclic ring of 3, 4, 5, 6 or 7 atoms, or R⁸⁵ and R⁸⁷ are joined to form a bond.

One preferred embodiment of the Cox-2 selective inhibitor of formula XIX is that wherein X is a bond.

Another preferred embodiment of the Cox-2 selective inhibitor of formula XIX is that wherein X is O.

Another preferred embodiment of the Cox-2 selective inhibitor of formula XIX is that wherein X is S.

Another preferred embodiment of the Cox-2 selective inhibitor of formula XIX is that wherein R⁸³ is CH₃.

Another preferred embodiment of the Cox-2 selective inhibitor of formula XIX is that wherein R⁸⁴ is halo or C₁₋₆ fluoroalkyl.

Materials that can serve as the Cox-2 selective inhibitor of the present invention include diaryl bicyclic heterocycles that are described in U.S. Pat. No. 6,329,421. Such diaryl bicyclic heterocycles have the general formula shown below in formula XX:

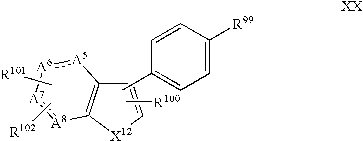

and pharmaceutically acceptable salts thereof wherein:

-A⁵=A⁶-A⁷=A⁸—is selected from the group consisting of:

(a) —CH═CH—CH═CH—,

(b) —CH₂—CH₂—CH₂—C(O)—, —CH₂—CH₂—C(O)—CH₂—, —CH₂—C(O)—CH₂—CH₂, —C(O)—CH₂—CH₂—CH₂,

(c) —CH₂—CH₂—C(O)—, —CH₂—C(O)—CH₂—, —C(O)—CH₂—CH₂—

(d) —CH₂—CH₂—O—C(O)—, CH₂—O—C(O)—CH₂—, —O—C(O)—CH₂—CH₂—,

(e) —CH₂—CH₂—C(O)—O—, —CH₂—C(O)—OCH₂—C(O)—O—CH₂—CH₂—,

(f) —C(R¹⁰⁵)₂—O—C(O)—, —C(O)—O—C(R¹⁰⁵)₂—, —C(O)—C(R¹⁰⁵)₂—, —C(R¹⁰⁵)₂—C(O)—O—,

(g) —N═CH—CH═CH—,

(h) —CH═N—CH═CH—,

(i) —CH═CH—N═CH—,

(j) —CH═CH—CH═N—,

(k) —N═CH—CH═N—,

(l) —N═CH—N═CH—,

(m) —CH═N—CH═N—,

(n) —S—CH═N—,

(o) —S—N═CH—,

(p) —N═N—NH—,

(q) —CH═N—S—, and

(r) —N═CH—S—;

R⁹⁹ is selected from the group consisting of:

(a) S(O)₂ CH₃,

(b) S(O)₂ NH₂,

(c) S(O)₂ NHCOCF₃,

(d) S(O)(NH)CH₃,

(e) S(O)(NH)NH₂,

(f) S(O)(NH)NHCOCF₃,

(g) P(O)(CH₃)OH, and

(h) P(O)(CH₃)NH₂;

R¹⁰⁰ is selected from the group consisting of:

(a) C₁₋₆ alkyl,

(b) C₃₋₇, cycloalkyl,

(c) mono- or di-substituted phenyl or naphthyl wherein the substituent is selected from the group consisting of:

(1) hydrogen,

(2) halo, including F, Cl, Br, I,

(3) C₁₋₆ alkoxy,

(4) C₁₋₆ alkylthio,

(5) CN,

(6) CF₃,

(7) C₁₋₆ alkyl,

(8) N₃,

(9) —CO₂ H,

(10) —CO₂—C₁₋₄ alkyl,

(11) —C(R¹⁰³)(R⁰⁴)—OH,

(12) —C(R¹⁰³)(R¹⁰⁴)—O—C₁₋₄ alkyl, and

(13) —C₁₋₆ alkyl-CO₂—R¹⁰⁶;

(d) mono- or di-substituted heteroaryl wherein the heteroaryl is a monocyclic aromatic ring of 5 atoms, said ring having one hetero atom which is S, O, or N, and optionally 1, 2, or 3 additional N atoms; or the heteroaryl is a monocyclic ring of 6 atoms, said ring having one hetero atom which is N, and optionally 1, 2, 3, or 4 additional N atoms; said substituents are selected from the group consisting of:

(1) hydrogen,

(2) halo, including fluoro, chloro, bromo and iodo,

(3) C₁₋₆ alkyl,

(4) C₁₋₆ alkoxy,

(5) C₁₋₆ alkylthio,

(6) CN,

(7) CF₃,

(8) N₃,

(9) —C(R¹⁰³)(R¹⁰⁴)—OH, and

(10) —C(R¹⁰³)(R¹⁰⁴)—O—C₁₋₄ alkyl;

(e) benzoheteroaryl which includes the benzo fused analogs of (d);

R¹⁰¹ and R¹⁰² are the substituents residing on any position of -A5=A6-A⁷=A8—and are selected independently from the group consisting of:

(a) hydrogen,

(b) CF₃,

(c) CN,

(d) C₁₋₆ alkyl,

(e) -Q³ wherein Q³ is Q⁴, CO₂ H, C(R¹⁰³)(R¹⁰⁴)OH,

(f) —O-Q⁴,

(g) —S-Q⁴, and

(h) optionally substituted:

(1) —C₁₋₅ alkyl-Q³,

(2) —O-C₁₋₅ alkyl-Q³,

(3) —S—C₁₋₅ alkyl-Q³,

(4) —C₁₋₃ alkyl-O—C₁₋₃ alkyl-Q³,

(5) —C₁₋₃ alkyl-S—C₁₋₃ alkyl-Q³,

(6) —C₁₋₅ alkyl-O-Q⁴,

(7) —C₁₋₅ alkyl-S-Q⁴,

wherein the substituent resides on the alkyl chain and the substituent is C₁₋₃ alkyl, and Q³ is Q⁴, CO₂ H, C(R¹⁰³)(R¹⁰⁴)OH Q⁴ is CO₂—C₁₋₄ alkyl, tetrazolyl-5-yl, or C(R¹⁰³)(R¹⁰⁴)O—C₁₋₄ alkyl;

R¹⁰³, R¹⁰⁴ and R¹⁰⁵ are each independently selected from the group consisting of

(a) hydrogen,

(b) C₁₋₆ alkyl; or

R¹⁰³ and R¹⁰⁴ together with the carbon to which they are attached form a saturated monocyclic carbon ring of 3, 4, 5, 6 or 7 atoms, or two R¹⁰⁵ groups on the same carbon form a saturated monocyclic carbon ring of 3, 4, 5, 6 or 7 atoms;

R¹⁰⁶ is hydrogen or C₁₋₆ alkyl;

R¹⁰⁷ is hydrogen, C₁₋₆ alkyl or aryl;

X⁷ is O, S, NR¹⁰⁷, CO, C(R¹⁰⁷)₂, C(R¹⁰⁷)(OH), —C(R¹⁰⁷)═C(R¹⁰⁷)—; —C(R¹⁰⁷)═N—;

—N═C(R¹⁰⁷)—.

Compounds that may act as Cox-2 inhibitors include salts of 5-amino or a substituted amino 1,2,3-triazole compound that are described in U.S. Pat. No. 6,239,137. The salts are of a class of compounds of formula XXI:

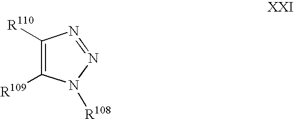

wherein:

R¹⁰⁸ is:

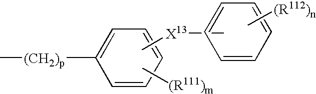

wherein:

p is 0 to 2; m is 0 to 4; and n is 0 to 5; X¹³ is O, S, SO, SO₂, CO, CHCN, CH₂ or C═NR¹¹³ where R¹¹³ is hydrogen, loweralkyl, hydroxy, loweralkoxy, amino, loweralkylamino, diloweralkylamino or cyano; and,

R¹¹¹ and R¹¹² are independently halogen, cyano, trifluoromethyl, loweralkanoyl, nitro, loweralkyl, loweralkoxy, carboxy, lowercarbalkoxy, trifuloromethoxy, acetamido, loweralkylthio, loweralkylsulfinyl, loweralkylsulfonyl, trichlorovinyl, trifluoromethylthio, trifluoromethylsulfinyl, or trifluoromethylsulfonyl; R¹⁰⁹ is amino, mono or diloweralkyl amino, acetamido, acetimido, ureido, formamido, formamido or guanidino; and

R¹¹⁰ is carbamoyl, cyano, carbazoyl, amidino or N-hydroxycarbamoyl; wherein the loweralkyl, loweralkyl containing, loweralkoxy and loweralkanoyl groups contain from 1 to 3 carbon atoms.

Materials that can serve as a Cox-2 selective inhibitor of the present invention include pyrazole derivatives that are described in U.S. Pat. No. 6,136,831. Such pyrazole derivatives have the formula shown below in formula XXII:

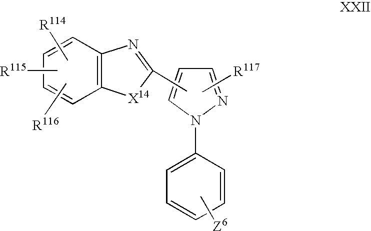

wherein:

R¹¹⁴ is hydrogen or halogen, R¹¹⁵ and R¹¹⁶ are each independently hydrogen, halogen, lower alkyl, lower alkoxy, hydroxy or lower alkanoyloxy;

R¹¹⁷ is lower haloalkyl or lower alkyl;

X¹⁴ is sulfur, oxygen or NH; and

Z⁶ is lower alkylthio, lower alkylsulfonyl or sulfamoyl;

or a pharmaceutically acceptable salt thereof.

Materials that can serve as a Cox-2 selective inhibitor of the present invention include substituted derivatives of benzosulphonamides that are described in U.S. Pat. No. 6,297,282. Such benzosulphonamide derivatives have the formula shown below in formula XXIII:

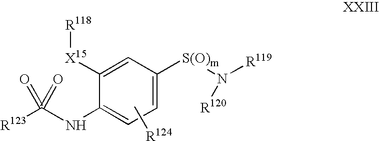

wherein:

X¹⁵ denotes oxygen, sulphur or NH;

R¹¹⁸ is an optionally unsaturated alkyl or alkyloxyalkyl group, optionally mono- or polysubstituted or mixed substituted by halogen, alkoxy, oxo or cyano, a cycloalkyl, aryl or heteroaryl group optionally mono- or polysubstituted or mixed substituted by halogen, alkyl, CF₃, cyano or alkoxy;

R¹¹⁹ and R¹²⁰, independently from one another, denote hydrogen, an optionally polyfluorised alkyl group, an aralkyl, aryl or heteroaryl group or a group (CH₂)ₙ—X¹⁶;

or

R¹¹⁹ and R¹²⁰, together with the N-atom, denote a 3 to 7-membered, saturated, partially or completely unsaturated heterocycle with one or more heteroatoms N, O or S, which can optionally be substituted by oxo, an alkyl, alkylaryl or aryl group, or a group (CH₂)ₙ—X¹⁶;

X¹⁶ denotes halogen, NO₂, —OR¹²¹, —COR¹²¹, —CO₂ R¹²¹, OCO₂ R¹²¹, —CN, —CONR¹²¹OR¹²², —CONR¹²¹ R¹²², —SR¹²¹, —S(O)R¹²¹, —S(O)₂ R¹²¹, —NR¹²¹ R¹²², —NHC(O)R¹²¹, —NHS(O)₂ R¹²¹;

n denotes a whole number from 0 to 6;

R¹²³ denotes a straight-chained or branched alkyl group with 1-10 C-atoms, a cycloalkyl group, an alkylcarboxyl group, an aryl group, aralkyl group, a heteroaryl or heteroaralkyl group which can optionally be mono- or polysubstituted or mixed substituted by halogen or alkoxy;

R¹²⁴ denotes halogen, hydroxy, a straight-chained or branched alkyl, alkoxy, acyloxy or alkyloxycarbonyl group with 1-6 C-atoms, which can optionally be mono- or polysubstituted by halogen, NO₂, —OR¹²¹, —COR¹²¹, CO₂ R¹²¹, —OCO₂ R¹²¹, —CN, —CONR¹²¹ OR¹²², —CONR¹²¹

R¹²¹, —SR¹²¹, —S(O)R¹²¹, —S(O)₂ R¹²¹, —NR¹²¹ R¹²², —NHC(O)R¹²¹, —NHS(O)₂ R¹²¹, or a polyfluoroalkyl group;

R¹²¹ and R¹²² independently from one another, denote hydrogen, alkyl, aralkyl or aryl; and

m denotes a whole number from 0 to 2;

and the pharmaceutically-acceptable salts thereof.

Materials that can serve as a Cox-2 selective inhibitor of the present invention include 3-phenyl-4-(4(methylsulfonyl)phenyl)-2-(5H)-furanones that are described in U.S. Pat. No. 6,239,173. Such 3-phenyl-4-(4(methylsulfonyl)phenyl)-2-(5H)-furanones have the formula shown below in formula XXIV:

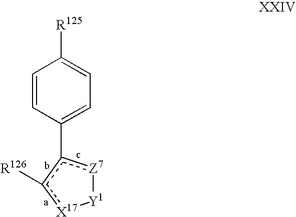

or pharmaceutically acceptable salts thereof wherein:

X¹⁷—Y¹-Z⁷—is selected from the group consisting of:

(a) —CH₂ CH₂ CH₂—,

(b) —C(O)CH₂ CH₂—,

(c) —CH₂ CH₂ C(O)—,

(d) —CR¹²⁹ (R^{129′})—O—C(O)—,

(e) —C(O)—O—CR¹²⁹ (R^{129′})—,

(f) —CH₂—NR¹²⁷—CH₂—,

(g) —CR¹²⁹ (R^{129′})—NR¹²⁷—C(O)—,

(h) —CR¹²⁸═CR¹²⁸—S—,

(i) —S—CR¹²⁸═CR^{128′}—,

(j) —S—N═CH—,

(k) —CH═N—S—,

(l) —N═CR¹²⁸—O—,

(m) —O—CR⁴═N—,

(n) —N═CR¹²⁸—NH—,

(o) —N═CR¹²⁸—S—, and

(p) —S—CR¹²⁸═N—,

(q) —C(O)—NR¹²⁷—CR² (R^{129′})—,

(r) —R¹²⁷ N—CH═CH—provided R₁₂₂ is not —S(O)₂CH₃,

(s) —CH═CH—NR¹²⁷—provided R¹²⁵ is not —S(O)₂CH₃,

when side b is a double bond, and sides a and c are single bonds; and

X¹⁷—Y-Z⁷—is selected from the group consisting of:

(a) ═CH—O—CH═, and

(b) ═CH—NR¹²⁷—CH═,

(c) ═N—S—CH═,

(d) ═CH—S—N═,

(e) ═N—O—CH═,

(f) ═CH—O—N═,

(g) ═N—S—N═,

(h) ═N—O—N═,

when sides a and c are double bonds and side b is a single bond;

R¹²⁵ is selected from the group consisting of:

(a) S(O)₂ CH₃,

(b) S(O)₂ NH₂,

(c) S(O)₂ NHC(O)CF₃,

(d) S(O)(NH)CH₃,

(e) S(O)(NH)NH₂,

(f) S(O)(NH)NHC(O)CF₃,

(g) P(O)(CH₃)OH, and

(h) P(O)(CH₃)NH₂;

R¹²⁶ is selected from the group consisting of

(a) C₁₋₆ alkyl,

(b) C₃, C₄, C₅, C₆, and C₇, cycloalkyl,

(c) mono-, di- or tri-substituted phenyl or naphthyl,

wherein the substituent is selected from the group consisting of:

(1) hydrogen,

(2) halo,

(3) C₁₋₆ alkoxy,

(4) C₁₋₆ alkylthio,

(5) CN,

(6) CF₃,

(7) C₁₋₆ alkyl,

(8) N₃,

(9) —CO₂ H,

(10) —CO₂—C₁₋₄ alkyl,

(11) —C(R¹²⁹)(R¹³⁰)—OH,

(12) —C(R¹²⁹)(R¹³⁰)—O—C₁₋₄ alkyl, and

(13) —C₁₋₆ alkyl-CO₂—R¹²⁹;

(d) mono-, di- or tri-substituted heteroaryl wherein the heteroaryl is a monocyclic aromatic ring of 5 atoms, said ring having one hetero atom which is S, O, or N, and optionally 1, 2, or 3 additionally N atoms; or the heteroaryl is a monocyclic ring of 6 atoms, said ring having one hetero atom which is N, and optionally 1, 2, 3, or 4 additional N atoms; said substituents are selected from the group consisting of:

(1) hydrogen,

(2) halo, including fluoro, chloro, bromo and iodo,

(3) C₁₋₆ alkyl,

(4) C₁₋₆ alkoxy,

(5) C₁₋₆ alkylthio,

(6) CN,

(7) CF₃,

(8) N₃,

(9) —C(R¹²⁹)(R¹³⁰)—OH, and

(10) —C(R¹²⁹)(R¹³⁰)—O—C₁₋₄ alkyl;

(e) benzoheteroaryl which includes the benzo fused analogs of (d);

R¹²⁷ is selected from the group consisting of:

(a) hydrogen,

(b) CF₃,

(c) CN,

(d) C₁₋₆ alkyl,

(e) hydroxyC₁₋₆ alkyl,

(f) —C(O)—C₁₋₆ alkyl,

(g) optionally substituted:

(1) —C₁₋₅ alkyl-Q⁵,

(2) —C₁₋₃ alkyl-O—C₁₋₃ alkyl-Q⁵,

(3) —C₁₋₃ alkyl-S—C₁₋₃ alkyl-Q⁵,

(4) —C₁₋₅ alkyl-O-Q⁵, or

(5) —C₁₋₅ alkyl-S-Q⁵,

wherein the substituent resides on the alkyl and the substituent is C₁₋₃ alkyl;

(h) -Q⁵;

R¹²⁸ and R^{128′} are each independently selected from the group consisting of:

(a) hydrogen,

(b) CF₃,

(c) CN,

(d) C₁₋₆ alkyl,

(e) -Q⁵,

(f) —O-Q⁵;

(g) —S-Q⁵, and

(h) optionally substituted:

(1) —C₁₋₅ alkyl-Q⁵,

(2) —O-C₁₋₅ alkyl-Q⁵,

(3) —S—C₁₋₅ alkyl-Q⁵,

(4) —C₁₋₃ alkyl-O—C₁₋₃ alkyl-Q⁵,

(5) —C₁₋₃ alkyl-S—C₁₋₃ alkyl-Q⁵,

(6) —C₁₋₅ alkyl-O-Q⁵,

(7) —C₁₋₅ alkyl-S-Q⁵,

wherein the substituent resides on the alkyl and the substituent is C₁₋₃ alkyl, and

R¹²⁹, R^{129,} R¹³⁰, R¹³¹ and R¹³² are each independently selected from the group consisting of:

(a) hydrogen,

(b) C₁₋₆ alkyl;

or R¹²⁹ and R¹³⁰ or R¹³¹ and R¹³² together with the carbon to which they are attached form a saturated monocyclic carbon ring of 3, 4, 5, 6 or 7 atoms;

Q⁵ is CO₂ H, CO₂—C₁₋₄ alkyl, tetrazolyl-5-yl, C(R¹³¹)(R¹³²)(OH), or C(R¹³¹)(R¹³²)(O—C₁₋₄ alkyl);

provided that when X—Y-Z is —S—CR¹²⁸═CR^{128′} then R¹²³ and R^{128′} are other than CF₃.

Materials that can serve as a Cox-2 selective inhibitor of the present invention include bicycliccarbonyl indole compounds that are described in U.S. Pat. No. 6,303,628. Such bicycliccarbonyl indole compounds have the formula shown below in formula XXV:

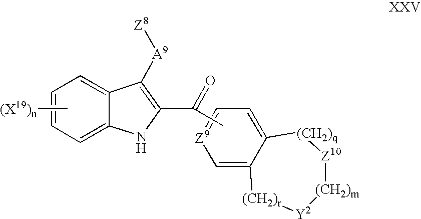

or the pharmaceutically acceptable salts thereof wherein

A⁹ is C₁₋₆ alkylene or —NR¹³³—;

Z⁸ is C(=L³)R¹³⁴, or SO₂ R¹³⁵;

Z⁹ is CH or N;

Z¹⁰ and Y² are independently selected from —CH₂—, O, S and —N—R¹³³;

m is 1 ,2 or 3;

q and r are independently 0, 1 or 2;

X¹⁸ is independently selected from halogen, C₁₋₄ alkyl, halo-substituted C₁₋₄ alkyl, hydroxy, C₁₋₄ alkoxy, halo-substituted C₁₋₄ alkoxy, C₁₋₄ alkylthio, nitro, amino, mono- or di-(C₁₋₄ alkyl)amino and cyano;

n is 0, 1, 2, 3 or 4;

L³ is oxygen or sulfur;

R¹³³ is hydrogen or C₁₋₄ alkyl;

R¹³⁴ is hydroxy, C₁₋₆ alkyl, halo-substituted C₁₋₆ alkyl, C₁₋₆ alkoxy, halo-substituted C₁₋₆ alkoxy, C₃₋₇ cycloalkoxy, C₁₋₄ alkyl(C₃₋₇ cycloalkoxy), —NR¹³⁶ R¹³⁷, C₁₋₄ alkylphenyl-O— or phenyl-O—, said phenyl being optionally substituted with one to five substituents independently selected from halogen, C₁₋₄ alkyl, hydroxy, C₁₋₄ alkoxy and nitro;

R¹³⁵ is C₁₋₆ alkyl or halo-substituted C₁₋₆ alkyl; and

R¹³⁶ and R¹³⁷ are independently selected from hydrogen, C₁₋₆ alkyl and halo-substituted C₁₋₆ alkyl.

Materials that can serve as a Cox-2 selective inhibitor of the present invention include benzimidazole compounds that are described in U.S. Pat. No. 6,310,079. Such benzimidazole compounds have the formula shown below in formula XXVI:

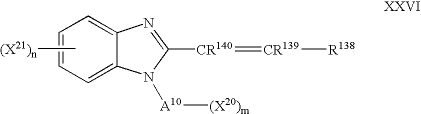

or a pharmaceutically acceptable salt thereof, wherein:

A¹⁰ is heteroaryl selected from

a 5-membered monocyclic aromatic ring having one hetero atom selected from O, S and N and optionally containing one to three N atom(s) in addition to said hetero atom, or

a 6-membered monocyclic aromatic ring having one N atom and optionally containing one to four N atom(s) in addition to said N atom; and

said heteroaryl being connected to the nitrogen atom on the benzimidazole through a carbon atom on the heteroaryl ring;

X²⁰ is independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N—(C₁-C₄ alkyl)amino, N, N-di(C₁-C₄ alkyl)amino, [N—(C₁-C₄ alkyl)amino]C₁-C₄ alkyl, [N, N -di(C₁-C₄ alkyl)amino]C₁-C₄ alkyl, N-(C₁-C₄ alkanoyl)amonio, N—(C₁-C₄ alkyl)(C₁-C₄ alkanoyl)amino, N—[(C₁-C₄ alkyl)sulfonyl]amino, N—[(halo-substituted C₁-C₄ alkyl)sulfonyl]amino, C₁-C₄ alkanoyl, carboxy, (C₁-C₄ alkoxy)carbonyl, carbamoyl, [N—(C₁-C₄ alkyl)amino]carbonyl, [N, N-di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N—(C₁-C₄ alkyl)amino]sulfonyl and [N, N-di(C₁-C₄ alkyl)amino]sulfonyl;

X²¹ is independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N—(C₁-C₄ alkyl)amino, N, N-di(C₁-C₄ alkyl)amino, [N—(C₁-C₄ alkyl)amino]C₁-C₄ alkyl, [N, N-di(C₁-C₄ alkyl)amino]C₁-C₄ alkyl, N—(C₁-C₄ alkanoyl)amino, N—(C₁-C₄ alkyl)-N—(C₁-C₄ alkanoyl) amino, N—[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)cabonyl, cabamoyl, [N—(C₁-C₄ alkyl) amino]carbonyl, [N, N-di(C₁-C₄ alkyl)amino]carbonyl, N-carbomoylamino, cyano, nitro, mercapto, (C₁-C₄ alkyl)thio, (C₁-C₄ alkyl)sulfinyl, (C₁-C₄ alkyl)sulfonyl, aminosulfonyl, [N—(C₁-C₄ alkyl)amino]sulfonyl and [N, N-di(C₁-C₄ alkyl)amino]sulfonyl;

R¹³⁸ is selected from hydrogen,

straight or branched C₁-C₄ alkyl optionally substituted with one to three substituent(s) wherein said substituents are independently selected from halo hydroxy, C₁-C₄ alkoxy, amino, N-(C₁-C₄ alkyl)amino and N, N-di(C₁-C₄ alkyl)amino,

C₃-C₈ cycloalkyl optionally substituted with one to three substituent(s) wherein said substituents are indepently selected from halo, C₁-C₄ alkyl, hydroxy, C₁-C₄ alkoxy, amino, N-(C₁-C₄ alkyl)amino and N, N-di(C₁-C₄ alkyl)amino,

C₄-C₈ cycloalkenyl optionally substituted with one to three substituent(s) wherein said substituents are independently selected from halo, C₁-C₄ alkyl, hydroxy, C₁-C₄ alkoxy, amino, N—(C₁-C₄ alkyl)amino and N, N-di(C₁-C₄ alkyl)amino,

phenyl optionally substituted with one to three substituent(s) wherein said substituents are independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N—(C₁-C₄ alkyl)amino, N, N-di(C₁-C₄ alkyl)amino, [N—(C₁-C₄ alkyl)amino]C₁-C₄ alkyl, [N, N-di(C₁-C₄ alkyl)amino]C₁-C₄ alkyl, N—(C₁-C₄ alkanoyl)amino, N—[C₁-C₄ alkyl)(C₁-C₄ alkanoyl)]amino, N—[(C₁-C₄ alkyl)sulfony]amino, N-[(halo-substituted C₁-C₄ alkyl)sulfonyl]amino, C₁-C₄ alkanoyl, carboxy, (C₁-C₄ alkoxy)carbonyl, carbomoyl, [N—(C₁-C₄ alky)amino]carbonyl, [N, N-di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N—(C₁-C₄ alkyl)amino]sulfonyl and [N, N-di(C₁-C₄ alkyl)amino]sulfonyl; and

heteroaryl selected from:

a 5-membered monocyclic aromatic ring having one hetero atom selected from O, S and N and optionally containing one to three N atom(s) in addition to said hetero atom; or a 6-membered monocyclic aromatic ring having one N atom and optionally containing one to four N atom(s) in addition to said N atom; and

said heteroaryl being optionally substituted with one to three substituent(s) selected from X²⁰;

R¹³⁹ and R¹⁴⁰ are independently selected from:

hydrogen,

halo,

C₁-C₄ alkyl,

phenyl optionally substituted with one to three substituent(s) wherein said substituents are independently selected from halo, C₁-C₄ alkyl, hydroxy, C₁-C₄ alkoxy, amino, N—(C₁-C₄ alkyl)amino and N, N-di(C₁-C₄ alkyl)amino,

or R¹³⁸ and R¹³⁹ can form, together with the carbon atom to which they are attached, a C₃-C₇ cycloalkyl ring;

m is 0, 1, 2, 3, 4 or 5; and

n is 0, 1, 2, 3 or 4.

Materials that can serve as a Cox-2 selective inhibitor of the present invention include indole compounds that are described in U.S. Pat. No. 6,300,363. Such indole compounds have the formula shown below in formula XXVII:

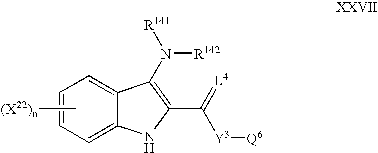

and the pharmaceutically acceptable salts thereof,

wherein:

L⁴ is oxygen or sulfur;

Y³ is a direct bond or C₁₋₄ alkylidene;

Q⁶ is:

(a) C₁₋₆ alkyl or halosubstituted C₁₋₆ alkyl, said alkyl being optionally substituted with up to three substituents independently selected from hydroxy, C₁₋₄ alkoxy, amino and mono- or di-(C₁₋₄ alkyl)amino,

(b) C₃₋₇ cycloalkyl optionally substituted with up to three substituents independently selected from hydroxy, C₁₋₄ alkyl and C₁₋₄ alkoxy,

(c) phenyl or naphthyl, said phenyl or naphthyl being optionally substituted with up to four substituents independently selected from:

(c-1) halo, C₁₋₄ alkyl, halosubstituted C₁₋₄ alkyl, hydroxy, C₁₋₄ alkoxy, halosubstituted C₁₋₄ alkoxy, S(O)ₘ R¹⁴³, SO₂ NH₂, SO₂ N(C₁₋₄ alkyl)₂, amino, mono- or di-(C₁₋₄ alkyl)amino, NHSO₂ R¹⁴³, NHC(O)R¹⁴³, CN, CO₂ H, CO₂ (C₁₋₄ alkyl), C₁₋₄ alkyl-OH, C₁₋₄ alkyl-OR¹⁴³, CONH₂, CONH(C₁₋₄ alkyl), CON(C₁₋₄ alkyl)₂ and —O—Y-phenyl, said phenyl being optionally substituted with one or two substituents independently selected from halo, C₁₋₄ alkyl, CF₃, hydroxy, OR¹⁴³, S(O)ₘR¹⁴³, amino, mono- or di-(C₁₋₄ alkyl)amino and CN;

(d) a monocyclic aromatic group of 5 atoms, said aromatic group having one heteroatom selected from O, S and N and optionally containing up to three N atoms in addition to said heteroatom, and said aromatic group being substituted with up to three substitutents independently selected from:

(d-1) halo, C₁₋₄ alkyl, halosubstituted C₁₋₄ alkyl, hydroxy, C₁₋₄ alkoxy, halosubstituted C₁₋₄ alkoxy, C₁₋₄ alkyl-OH, S(O)ₘ R¹⁴³, SO₂ NH₂, SO₂ N(C₁₋₄ alkyl)₂, amino, mono- or di-(C₁₋₄ alkyl)amino, NHSO₂ R¹⁴³, NHC(O)R¹⁴³, CN, CO₂ H, CO₂ (C₁₋₄ alkyl), C₁₋₄ alkyl-OR¹⁴³, CONH₂, CONH(C₁₋₄ alkyl), CON(C₁₋₄ alkyl)₂, phenyl, and mono-, di- or tri-substituted phenyl wherein the substituent is independently selected from halo, CF₃, C₁₋₄ alkyl, hydroxy, C₁₋₄ alkoxy, OCF₃, SR¹⁴³, SO₂ CH₃, SO₂ NH₂, amino, C₁₋₄ alkylamino and NHSO₂ R¹⁴³;

(e) a monocyclic aromatic group of 6 atoms, said aromatic group having one heteroatom which is N and optionally containing up to three atoms in addition to said heteroatom, and said aromatic group being substituted with up to three substituents independently selected from the above group (d-1);

R¹⁴¹ is hydrogen or C₁₋₆ alkyl optionally substituted with a substituent selected independently from hydroxy, OR¹⁴³, nitro, amino, mono- or di-(C₁₋₄ alkyl)amino, CO₂ H, CO₂ (C₁₋₄ alkyl), CONH₂, CONH(C₁₋₄ alkyl) and CON(C₁₋₄ alkyl)₂;

R¹⁴² is:

(a) hydrogen,

(b) C₁₋₄ alkyl,

(c) C(O)R¹⁴⁵,

wherein R¹⁴⁵ is selected from:

(c-1) C₁₋₂₂ alkyl or C₂₋₂₂ alkenyl, said alkyl or alkenyl being optionally substituted with up to four substituents independently selected from:

(c-1-1) halo, hydroxy, OR¹⁴³, S(O)ₘ R¹⁴³, nitro, amino, mono- or di-(C₁₋₄ alkyl)amino, NHSO₂ R¹⁴³, CO₂ H, CO₂ (C₁₋₄ alkyl), CONH₂, CONH(C₁₋₄ alkyl), CON(C₁₋₄ alkyl)₂, OC(O)R¹⁴³, thienyl, naphthyl and groups of the following formulae:

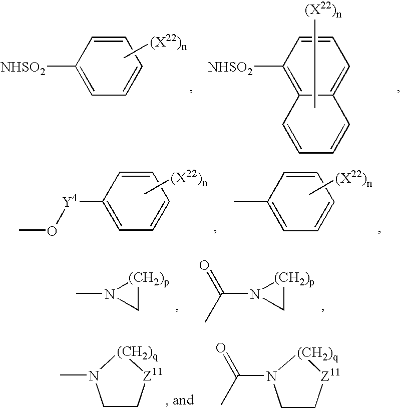

(c-2) C₁₋₂₂ alkyl or C₂₋₂₂ alkenyl, said alkyl or alkenyl being optionally substituted with five to forty-five halogen atoms,

(c-3) —Y⁵—C₃₋₇ cycloalkyl or —Y⁵—C₃₋₇ cycloalkenyl, said cycloalkyl or cycloalkenyl being optionally substituted with up to three substituent independently selected from:

(c-3-1) C₁₋₄ alkyl, hydroxy, OR¹⁴³, S(O)ₘ R¹⁴³, amino, mono- or di-(C₁₋₄ alkyl)amino, CONH₂, CONH(C₁₋₄ alkyl) and CON(C₁₋₄ alkyl)₂,

(c-4) phenyl or naphthyl, said phenyl or naphthyl being optionally substituted with up to seven (preferably up to seven) substituents independently selected from:

(c-4-1) halo, C₁₋₈ alkyl, C₁₋₄ alkyl-OH, hydroxy, C₁₋₈ alkoxy, halosubstituted C₁₋₈ alkyl, halosubstituted C₁₋₈ alkoxy, CN, nitro, S(O)ₘ R¹⁴³, SO₂NH₂, SO₂NH(C₁₋₄ alkyl), SO₂N(C₁₋₄ alkyl)₂, amino, C₁₋₄ alkylamino, di-(C₁₋₄ alkyl)amino, CONH₂, CONH(C₁₋₄ alkyl), CON(C₁₋₄ alkyl)₂, OC(O)R¹⁴³, and phenyl optionally substituted with up to three substituents independently selected from halo, C₁₋₄ alkyl, hydroxy, OCH₃, CF₃, OCF₃, CN, nitro, amino, mono- or di-(C₁₋₄ alkyl)amino, CO₂ H, CO₂(C₁₋₄ alkyl) and CONH₂,

(c-5) a monocyclic aromatic group as defined in (d) and (e) above, said aromatic group being optionally substituted with up to three substituents independently selected from:

(c-5-1) halo, C₁₋₈ alkyl, C₁₋₄ alkyl-OH, hydroxy, C₁₋₈ alkoxy, CF₃, OCF₃, CN, nitro, S(O)ₘR¹⁴³, amino, mono- or di-(C₁₋₄ alkyl)amino, CONH₂, CONH(C₁₋₄ alkyl), CON(C₁₋₄ alkyl)₂, CO₂ H and CO₂(C₁₋₄ alkyl), and —Y-phenyl, said phenyl being optionally substituted with up to three substituents independently selected halogen, C₁₋₄ alkyl, hydroxy, C₁₋₄ alkoxy, CF₃, OCF₃, CN, nitro, S(O)ₘR¹⁴³, amino, mono- or di-(C₁₋₄ alkyl)amino, CO₂H, CO₂(C₁₋₄ alkyl), CONH₂, CONH(C₁₋₄ alkyl) and CON(C₁₋₄ alkyl)₂,

(c-6) a group of the following formula:

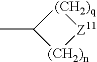

X²² is halo, C₁₋₄ alkyl, hydroxy, C₁₋₄ alkoxy, halosubstitutued C₁₋₄ alkoxy, S(O)ₘR¹⁴³, amino, mono- or di-(C₁₋₄ alkyl)amino, NHSO₂R¹⁴³, nitro, halosubstitutued C₁₋₄ alkyl, CN, CO₂H, CO₂(C₁₋₄ alkyl), C₁₋₄ alkyl-OH, C₁₋₄ alkylOR¹⁴³, CONH₂, CONH(C₁₋₄ alkyl) or CON(C₁₋₄ alkyl)₂;

R¹⁴³ is C₁₋₄ alkyl or halosubstituted C₁₋₄ alkyl;

m is 0, 1 or 2; n is 1, 2 or 3; p is 1, 2, 3, 4 or 5 q is 2 or 3;

Z¹¹ is oxygen, sulfur or NR¹⁴⁴; and

R¹⁴⁴ is hydrogen, C₁₋₆ alkyl, halosubstitutued C₁₋₄ alkyl or —Y⁵-phenyl, said phenyl being optionally substituted with up to two substituents independently selected from halo, C₁₋₄ alkyl, hydroxy, C₁₋₄ alkoxy, S(O)ₘR¹⁴³, amino, mono- or di-(C₁₋₄ alkyl)amino, CF₃, OCF₃, CN and nitro;

with the proviso that a group of formula —Y⁵-Q is not methyl or ethyl when X²² is hydrogen;

L⁴ is oxygen;

R¹⁴¹ is hydrogen; and

R¹⁴² is acetyl.

Materials that can serve as a Cox-2 selective inhibitor of the present invention include aryl phenylhydrazides that are described in U.S. Pat. No. 6,077,869. Such aryl phenylhydrazides have the formula shown below in formula XXVIII:

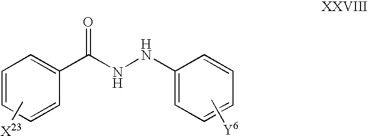

wherein:

X²³ and Y⁶ are selected from hydrogen, halogen, alkyl, nitro, amino or other oxygen and sulfur containing functional groups such as hydroxy, methoxy and methylsulfonyl.

Materials that can serve as a Cox-2 selective inhibitor of the present invention include 2-aryloxy, 4-aryl furan-2-ones that are described in U.S. Pat. No. 6,140,515. Such 2-aryloxy, 4-aryl furan-2-ones have the formula shown below in formula XXIX:

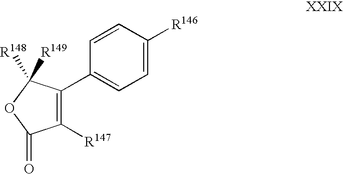

or a pharmaceutical salt thereof, wherein:

R¹⁴⁶ is selected from the group consisting of SCH₃, —SO(O)₂CH₃ and —S(O)₂NH₂;

R¹⁴⁷ is selected from the group consisting of OR¹⁵⁰, mono or di-substituted phenyl or pyridyl wherein the substituents are selected from the group consisting of methyl, chloro and F;

R¹⁵⁰ is unsubstituted or mono or di-substituted phenyl or pyridyl wherein the substituents are selected from the group consisting of methyl, chloro and F;

R¹⁴⁸ is H, C₁₋₄ alkyl optionally substituted with 1 to 3 groups of F, Cl or Br; and

R¹⁴⁹ is H, C₁₋₄ alkyl optionally substituted with 1 to 3 groups of F, Cl or Br, with the proviso that R¹⁴⁸ and R¹⁴⁹ are not the same.

Materials that can serve as a Cox-2 selective inhibitor of the present invention include bisaryl compounds that are described in U.S. Pat. No. 5,994,379. Such bisaryl compounds have the formula shown below in formula XXX:

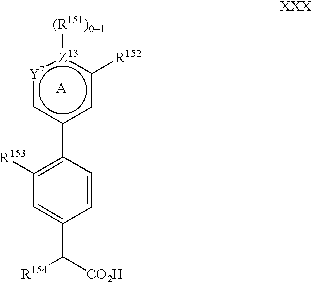

or a pharmaceutically acceptable salt, ester or tautomer thereof, wherein:

Z¹³ is C or N;

when Z¹³ is N, R¹⁵¹ represents H or is absent, or is taken in conjunction with R¹⁵² as described below:

when Z¹³ is C, R¹⁵¹ represents H and R¹⁵² is a moiety which has the following characteristics:

(a) it is a linear chain of 3-4 atoms containing 0-2 double bonds, which can adopt an energetically stable transoid configuration and if a double bond is present, the bond is in the trans configuration,

(b) it is lipophilic except for the atom bonded directly to ring A, which is either lipophilic or non-lipophilic, and

(c) there exists an energetically stable configuration planar with ring A to within about 15 degrees;

or R¹⁵¹ and R¹⁵² are taken in combination and represent a 5- or 6-membered aromatic or non-aromatic ring D fused to ring A, said ring D containing 0-3 heteroatoms selected from O, S and N;

said ring D being lipophilic except for the atoms attached directly to ring A, which are lipophilic or non-lipophilic, and said ring D having available an energetically stable configuration planar with ring A to within about 15 degrees;

said ring D further being substituted with 1 R^{a} group selected from the group consisting of: C₁₋₂ alkyl, —OC₁₋₂ alkyl, —NHC₁₋₂ alkyl, —N(C₁₋₂ alkyl)₂, —C(O)C₁₋₂ alkyl, —S—C₁₋₂ alkyl and —C(S)C₁₋₂ alkyl;

Y⁷ represents N, CH or C—OC₁₋₃ alkyl, and when Z¹³ is N, Y⁷ can also represent a carbonyl group;

R¹⁵³ represents H, Br, Cl or F; and

R¹⁵⁴ represents H or CH₃.

Materials that can serve as a Cox-2 selective inhibitor of the present invention include 1,5-diarylpyrazoles that are described in U.S. Pat. No. 6,028,202. Such 1,5-diarylpyrazoles have the formula shown below in formula XXXI:

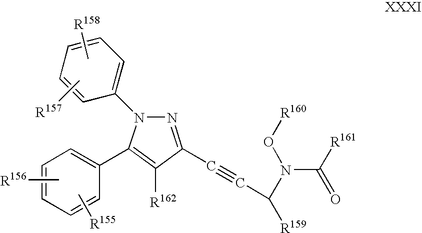

wherein:

R¹⁵⁵ R¹⁵⁶, R¹⁵⁷, and R¹⁵⁸ are independently selected from the groups consisting of hydrogen, C₁₋₅ alkyl, C₁₋₅ alkoxy, phenyl, halo, hydroxy, C₁₋₅ alkylsulfonyl, C₁₋₅ alkylthio, trihaloC₁₋₅ alkyl, amino, nitro and 2-quinolinylmethoxy;

R¹⁵⁹ is hydrogen, C₁₋₅ alkyl, trihaloC₁₋₅ alkyl, phenyl, substituted phenyl where the phenyl substitutents are halogen, C₁₋₅ alkoxy, trihaloC₁₋₅ alkyl or nitro or R¹⁵⁹ is heteroaryl of 5-7 ring members where at least one of the ring members is nitrogen, sulfur or oxygen;

R¹⁶⁰ is hydrogen, C₁₋₅ alkyl, phenyl C₁₋₅ alkyl, substituted phenyl C₁₋₅ alkyl where the phenyl substitutents are halogen, C₁₋₅ alkoxy, trihaloC₁₋₅ alkyl or nitro, or R¹⁶⁰ is C₁₋₅ alkoxycarbonyl, phenoxycarbonyl, substituted phenoxycarbonyl where the phenyl substitutents are halogen, C₁₋₅ alkoxy, trihaloC₁₋₅ alkyl or nitro;

R¹⁶¹ is C₁₋₁₀ alkyl, substituted C₁₋₁₀ alkyl where the substituents are halogen, trihaloC₁₋₅ alkyl, C₁₋₅ alkoxy, carboxy, C₁₋₅ alkoxycarbonyl, amino, C₁₋₅ alkylamino, diC₁₋₅ alkylamino, diC₁₋₅ alkylaminoC₁₋₅ alkylamino, C₁₋₅ alkylaminoC₁₋₅ alkylamino or a heterocycle containing 4-8 ring atoms where one more of the ring atoms is nitrogen, oxygen or sulfur, where said heterocycle may be optionally substituted with C₁₋₅ alkyl; or R¹⁶¹ is phenyl, substituted phenyl (where the phenyl substitutents are one or more of C₁₋₅ alkyl, halogen, C₁₋₅ alkoxy, trihaloC₁₋₅ alkyl or nitro), or R¹⁶¹ is heteroaryl having 5-7 ring atoms where one or more atoms are nitrogen, oxygen or sulfur, fused heteroaryl where one or more 5-7 membered aromatic rings are fused to the heteroaryl; or

R¹⁶¹ is NR¹⁶³R¹⁶⁴ where R¹⁶³ and R¹⁶⁴ are independently selected from hydrogen and C₁₋₅ alkyl or R¹⁶³ and R¹⁶⁴ may be taken together with the depicted nitrogen to form a heteroaryl ring of 5-7 ring members where one or more of the ring members is nitrogen, sulfur or oxygen where said heteroaryl ring may be optionally substituted with C₁₋₅ alkyl;

R¹⁶² is hydrogen, C₁₋₅ alkyl, nitro, amino, and halogen;

and pharmaceutically acceptable salts thereof.

Materials that can serve as a Cox-2 selective inhibitor of the present invention include 2-substituted imidazoles that are described in U.S. Pat. No. 6,040,320. Such 2-substituted imidazoles have the formula shown below in formula XXXII:

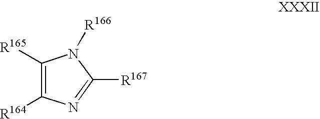

wherein:

R¹⁶⁴ is phenyl, heteroaryl wherein the heteroaryl contains 5 to 6 ring atoms, or substituted phenyl;

wherein the substituents are independently selected from one or members of the group consisting of C₁₋₅ alkyl, halogen, nitro, trifluoromethyl and nitrile;

R¹⁶⁵ is phenyl, heteroaryl wherein the heteroaryl contains 5 to 6 ring atoms, substituted heteroaryl;

wherein the substituents are independently selected from one or more members of the group consisting of C₁₋₅ alkyl and halogen, or substituted phenyl,

wherein the substituents are independently selected from one or members of the group consisting of C₁₋₅ alkyl, halogen, nitro, trifluoromethyl and nitrile;

R¹⁶⁶ is hydrogen, SEM, C₁₋₅ alkoxycarbonyl, aryloxycarbonyl, arylC₁₋₅ alkyloxycarbonyl, arylC₁₋₅ alkyl, phthalimidoC₁₋₅ alkyl, aminoC₁₋₅ alkyl, diaminoC₁₋₅ alkyl, succinimidoC₁₋₅ alkyl, C₁₋₅ alkylcarbonyl, arylcarbonyl, C₁₋₅ alkylcarbonylC₁₋₅ alkyl, aryloxycarbonylC₁₋₅ alkyl, heteroarylC₁₋₅ alkyl where the heteroaryl contains 5 to 6 ring atoms, or substituted arylC₁₋₅ alkyl,

wherein the aryl substituents are independently selected from one or more members of the group consisting of C₁₋₅ alkyl, C₁₋₅ alkoxy, halogen, amino, C₁₋₅ alkylamino, and diC₁₋₅ alkylamino;

R¹⁶⁷ is (A¹¹)ₙ—(CH¹⁶⁵)_{q}—X²⁴ wherein:

A¹¹ is sulfur or carbonyl;

n is 0 or 1;

q is 0-9;

X²⁴ is selected from the group consisting of hydrogen, hydroxy, halogen, vinyl, ethynyl, C₁₋₅ alkyl, C₃₋₇ cycloalkyl, C₁₋₅ alkoxy, phenoxy, phenyl, arylC₁₋₅ alkyl, amino, C₁₋₅ alkylamino, nitrile, phthalimido, amido, phenylcarbonyl, C₁₋₅ alkylaminocarbonyl, phenylaminocarbonyl, arylC₁₋₅ alkylaminocarbonyl, C₁₋₅ alkylthio, C₁₋₅ alkylsulfonyl, phenylsulfonyl, substituted sulfonamido,

wherein the sulfonyl substituent is selected from the group consisting of C₁₋₅ alkyl, phenyl, araC₁₋₅ alkyl, thienyl, furanyl, and naphthyl; substituted vinyl,

wherein the substituents are independently selected from one or members of the group consisting of fluorine, bromine, chlorine and iodine, substituted ethynyl,

wherein the substituents are independently selected from one or more members of the group consisting of fluorine, bromine chlorine and iodine, substituted C₁₋₅ alkyl,

wherein the substituents are selected from the group consisting of one or more C₁₋₅ alkoxy, trihaloalkyl, phthalimido and amino, substituted phenyl,

wherein the phenyl substituents are independently selected from one or more members of the group consisting of C₁₋₅ alkyl, halogen and C₁₋₅ alkoxy, substituted phenoxy,

wherein the phenyl substituents are independently selected from one or more members of the group consisting of C₁₋₅ alkyl, halogen and C₁₋₅ alkoxy, substituted C₁₋₅ alkoxy,

wherein the alkyl substituent is selected from the group consisting of phthalimido and amino, substituted arylC₁₋₅ alkyl,

wherein the alkyl substituent is hydroxyl, substituted arylC₁₋₅ alkyl,

wherein the phenyl substituents are independently selected from one or more members of the group consisting of C₁₋₅ alkyl, halogen and C₁₋₅ alkoxy, substituted amido,

wherein the carbonyl substituent is selected from the group consisting of C₁₋₅ alkyl, phenyl, arylC₁₋₅ alkyl, thienyl, furanyl, and naphthyl, substituted phenylcarbonyl,

wherein the phenyl substituents are independently selected from one or members of the group consisting of C₁₋₅ alkyl, halogen and C₁₋₅ alkoxy, substituted C₁₋₅ alkylthio,

wherein the alkyl substituent is selected from the group consisting of hydroxy and phthalimido, substituted C₁₋₅ alkylsulfonyl,

wherein the alkyl substituent is selected from the group consisting of hydroxy and phthalimido, substituted phenylsulfonyl,

wherein the phenyl substituents are independently selected from one or members of the group consisting of bromine, fluorine, chlorine, C₁₋₅ alkoxy and trifluoromethyl, with the proviso:

if A¹¹ is sulfur and X²⁴ is other than hydrogen, C₁₋₅ alkylaminocarbonyl, phenylaminocarbonyl, arylC₁₋₅ alkylaminocarbonyl, C₁₋₅ alkylsulfonyl or phenylsulfonyl, then q must be equal to or greater than 1;

if A¹¹ is sulfur and q is 1, then X²⁴ cannot be C₁₋₂ alkyl;

if A¹¹ is carbonyl and q is 0, then X²⁴ cannot be vinyl, ethynyl, C₁₋₅ alkylaminocarbonyl, phenylaminocarbonyl, arylC₁₋₅ alkylaminocarbonyl,C₁₋₅ alkylsulfonyl or phenylsulfonyl;

if A¹¹ is carbonyl, q is 0 and X²⁴ is H, then R¹⁶⁶ is not SEM (2-(trimethylsilyl)ethoxymethyl);

if n is 0 and q is 0, then X²⁴ cannot be hydrogen;

and pharmaceutically acceptable salts thereof.

Materials that can serve as a Cox-2 selective inhibitor of the present invention include 1,3- and 2,3-diarylcycloalkano and cycloalkeno pyrazoles that are described in U.S. Pat. No. 6,083,969. Such 1,3- and 2,3-diarylpyrazole compounds have the general formulas shown below in formulas XXXIII and XXXIV:

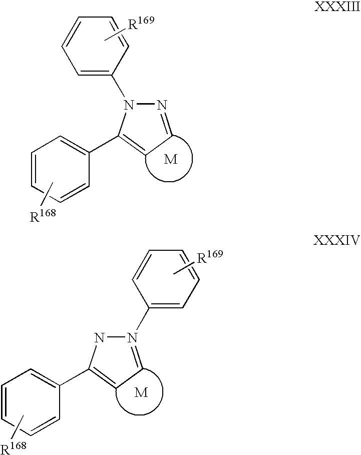

wherein:

R¹⁶⁸ and R¹⁶⁹ are independently selected from the group consisting of hydrogen, halogen, (C₁-C₆)alkyl, (C₁-C₆)alkoxy, nitro, amino, hydroxy, trifluoro, —S(C₁-C₆)alkyl, —SO(C₁-C₆)alkyl and —SO₂(C₁-C₆)alkyl; and the fused moiety M is a group selected from the group consisting of an optionally substituted cyclohexyl and cycloheptyl group having the formulae:

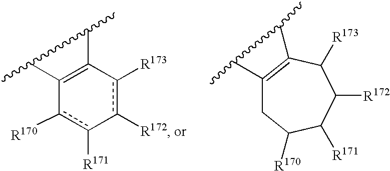

wherein:

R¹⁷⁰ is selected from the group consisting of hydrogen, halogen, hydroxy and carbonyl;

or R¹⁷⁰ and R¹⁷¹ taken together form a moiety selected from the group consisting of —OCOCH₂—, —ONH(CH₃)COCH₂—, —OCOCH.dbd. and —O—;

R¹⁷¹ and R¹⁷² are independently selected from the group consisting of hydrogen, halogen, hydroxy, carbonyl, amino, (C₁-C₆)alkyl, (C₁-C₆)alkoxy, ═NOH, —NR¹⁷⁴R¹⁷⁵, —OCH₃, —OCH₂CH₃, —OSO₂NHCO₂ CH₃, ═CHCO₂CH₂CH₃, —CH₂CO₂H, —CH₂CO₂CH₃, —CH₂CO₂CH₂CH₃, —CH₂CON(CH₃)₂, —CH₂CO₂NHCH₃, —CHCHCO₂CH₂CH₃, —OCON(CH₃)OH, —C(COCH₃)₂, di(C₁-C₆)alkyl and di(C₁-C₆)alkoxy;

R¹⁷³ is selected from the group consisting of hydrogen, halogen, hydroxy, carbonyl, amino, (C₁-C₆)alkyl, (C₁-C₆)alkoxy and optionally substituted carboxyphenyl, wherein substituents on the carboxyphenyl group are selected from the group consisting of halogen, hydroxy, amino, (C₁-C₆)alkyl and (C₁-C₆)alkoxy;

or R¹⁷² and R¹⁷³ taken together form a moiety selected from the group consisting of —O— and

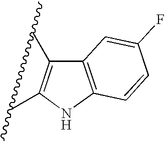

R¹⁷⁴ is selected from the group consisting of hydrogen, OH, —OCOCH₃, —COCH₃ and (C₁-C₆)alkyl; and

R¹⁷⁵ is selected from the group consisting of hydrogen, OH, —OCOCH₃, —COCH₃, (C₁-C₆)alkyl, —CONH₂ and —SO₂CH₃,

with the proviso that

if M is a cyclohexyl group, then R¹⁷⁰ through R¹⁷³ may not all be hydrogen; and

pharmaceutically acceptable salts, esters and pro-drug forms thereof.

Materials that can serve as a Cox-2 selective inhibitor of the present invention include esters derived from indolealkanols and novel amides derived from indolealkylamides that are described in U.S. Pat. No. 6,306,890. Such compounds have the general formula shown below in formula XXXV:

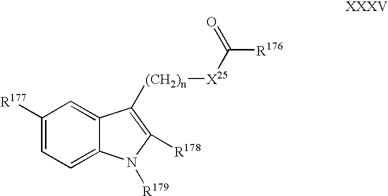

wherein:

R¹⁷⁶ is C₁ to C₆ alkyl, C₁ to C₆ branched alkyl, C₄ to C₈ cycloalkyl, C₁ to C₆ hydroxyalkyl, branched C₁ to C₆ hydroxyalkyl, hydroxy substituted C₄ to C₈ aryl, primary, secondary or tertiary C₁ to C₆ alkylamino, primary, secondary or tertiary branched C₁ to C₆ alkylamino, primary, secondary or tertiary C₄ to C₈ arylamino, C₁ to C₆ alkylcarboxylic acid, branched C₁ to C₆ alkylcarboxylic acid, C₁ to C₆ alkylester, branched C₁ to C₆ alkylester, C₄ to C₈ aryl, C₄ to C₈ arylcarboxylic acid, C₄ to C₈ arylester, C₄ to C₆ aryl substituted C₁ to C₆ alkyl, C₄ to C₈ heterocyclic alkyl or aryl with O, N or S in the ring, alkyl-substituted or aryl-substituted C₄ to C₈ heterocyclic alkyl or aryl with O, N or S in the ring, or halo-substituted versions thereof, where halo is chloro, bromo, fluoro or iodo;

R¹⁷⁷ is C₁ to C₆ alkyl, C₁ to C₆ branched alkyl, C₄ to C₈ cycloalkyl, C₄ to C₈ aryl, C₄ to C₈ aryl-substituted C₁ to C₆ alkyl, C₁ to C₆ alkoxy, C₁ to C₆ branched alkoxy, C₄ to C₈ aryloxy, or halo-substituted versions thereof or

R¹⁷⁷ is halo where halo is chloro, fluoro, bromo, or iodo;

R¹⁷⁸ is hydrogen, C₁ to C₆ alkyl or C₁ to C₆ branched alkyl;

R¹⁷⁹ is C₁ to C₆ alkyl, C₄ to C₈ aroyl, C₄ to C₈ aryl, C₄ to C₈ heterocyclic alkyl or aryl with O, N or S in the ring, C₄ to C₈ aryl-substituted C₁ to C₆ alkyl, alkyl-substituted or aryl-substituted C₄ to C₈ heterocyclic alkyl or aryl with O, N or S in the ring, alkyl-substituted C₄ to C₈ aroyl, or alkyl-substituted C₄ to C₈ aryl, or halo-substituted versions thereof where halo is chloro, bromo, or iodo;

n is 1, 2, 3, or 4; and

X²⁵ is O, NH, or N—R¹⁸⁰, where R¹⁸⁰ is C₁ to C₆ alkyl or C₁ to C₆ branched alkyl.

Materials that can serve as a Cox-2 selective inhibitor of the present invention include pyridazinone compounds that are described in U.S. Pat. No. 6,307,047. Such pyridazinone compounds have the formula shown below in formula XXXVI:

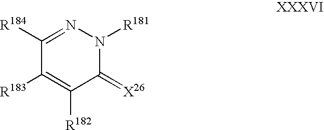

or a pharmaceutically acceptable salt, ester, or prodrug thereof, wherein:

X²⁶ is selected from the group consisting of O, S, —NR¹⁸⁵, —NOR^{a}, and —NNR^{b}R^{c};

R¹⁸⁵ is selected from the group consisting of alkenyl, alkyl, aryl, arylalkyl, cycloalkenyl, cycloalkenylalkyl, cycloalkyl, cycloalkylalkyl, heterocyclic, and heterocyclic alkyl;

R^{a}, R^{b}, and R^{c} are independently selected from the group consisting of alkyl, aryl, arylalkyl, cycloalkyl, and cycloalkylalkyl;

R¹⁸¹ is selected from the group consisting of alkenyl, alkoxy, alkoxyalkyl, alkoxyiminoalkoxy, alkyl, alkylcarbonylalkyl, alkylsulfonylalkyl, alkynyl, aryl, arylalkenyl, arylalkoxy, arylalkyl, arylalkynyl, arylhaloalkyl, arylhydroxyalkyl, aryloxy, aryloxyhaloalkyl, aryloxyhydroxyalkyl, arylcarbonylalkyl, carboxyalkyl, cyanoalkyl, cycloalkenyl, cycloalkenylalkyl, cycloalkyl, cycloalkylalkyl, cycloalkylidenealkyl, haloalkenyl, haloalkoxyhydroxyalkyl, haloalkyl, haloalkynyl, heterocyclic, heterocyclic alkoxy, heterocyclic alkyl, heterocyclic oxy, hydroxyalkyl, hydroxyiminoalkoxy, —(CH₂)ₙC(O)R¹⁸⁶, —(CH₂)ₙCH(OH)R¹⁸⁶, —(CH₂)ₙC(NOR^{d})R¹⁸⁶,—(CH₂)ₙCH(NOR^{d})R¹⁸⁶, —(CH₂)ₙCH(NR^{d}R^{e})R¹⁸⁶, —R¹⁸⁷R¹⁸⁸,—(CH₂)ₙC≡CR¹⁸⁸, —(CH₂)ₙ[CH(CX^{26′}₃)]ₘ(CH₂)ₚR¹⁸⁸, —(CH₂)ₙ(CX^{26′}₂)ₘ(CH₂)ₚR¹⁸⁸, and —(CH₂)ₙ(CHX^{26′})ₘ(CH₂)ₘR¹⁸⁸;

R¹⁸⁶ is selected from the group consisting of hydrogen, alkenyl, alkyl, alkynyl, aryl, arylalkyl, cycloalkenyl, cycloalkyl, haloalkenyl, haloalkyl, haloalkynyl, heterocyclic, and heterocyclic alkyl;

R¹⁸⁷ is selected from the group consisting of alkenylene, alkylene, halo-substituted alkenylene, and halo-substituted alkylene;

R¹⁸⁸ is selected from the group consisting of hydrogen, alkenyl, alkyl, alkynyl, aryl, arylalkyl, cycloalkyl, cycloalkenyl, haloalkyl, heterocyclic, and heterocyclic alkyl;

R^{d} and R^{e} are independently selected from the group consisting of hydrogen, alkenyl, alkyl, alkynyl, aryl, arylalkyl, cycloalkenyl, cycloalkyl, haloalkyl, heterocyclic, and heterocyclic alkyl;

X^{26′} is halogen;

m is an integer from 0-5;

n is an integer from 0-10; and

p is an integer from 0-10; and

R¹⁸², R¹⁸³, and R¹⁸⁴ are independently selected from the group consisting of hydrogen, alkenyl, alkoxyalkyl, alkoxyiminoalkoxy, alkoxyiminoalkyl, alkyl, alkynyl, alkylcarbonylalkoxy, alkylcarbonylamino, alkylcarbonylaminoalkyl, aminoalkoxy, aminoalkylcarbonyloxyalkoxy aminocarbonylalkyl, aryl, arylalkenyl, arylalkyl, arylalkynyl, carboxyalkylcarbonyloxyalkoxy, cyano, cycloalkenyl, cycloalkyl, cycloalkylidenealkyl, haloalkenyloxy, haloalkoxy, haloalkyl, halogen, heterocyclic, hydroxyalkoxy, hydroxyiminoalkoxy, hydroxyiminoalkyl, mercaptoalkoxy, nitro, phosphonatoalkoxy, Y⁸, and Z¹⁴;

provided that one of R¹⁸², R¹⁸³, or R¹⁸⁴ must be Z¹⁴, and further provided that only one of R¹⁸², R¹⁸³, or R¹⁸⁴ is Z¹⁴;

Z¹⁴ is selected from the group consisting of:

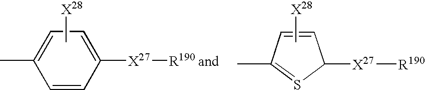

X²⁷ is selected from the group consisting of S(O)₂, S(O)(NR¹⁹¹), S(O), Se(O)₂, P(O)(OR¹⁹²), and P(O)(NR¹⁹³R¹⁹⁴);

X²⁸ is selected from the group consisting of hydrogen, alkenyl, alkyl, alkynyl and halogen;

R¹⁹⁰ is selected from the group consisting of alkenyl, alkoxy, alkyl, alkylamino, alkylcarbonylamino, alkynyl, amino, cycloalkenyl, cycloalkyl, dialkylamino, —NHNH₂, and —NCHN(R¹⁹¹)R¹⁹²;

R¹⁹¹, R¹⁹², R¹⁹³, and R¹⁹⁴ are independently selected from the group consisting of hydrogen, alkyl, and cycloalkyl, or R¹⁹³ and R¹⁹⁴ can be taken together, with the nitrogen to which they are attached, to form a 3-6 membered ring containing 1 or 2 heteroatoms selected from the group consisting of O, S, and NR¹⁸⁸;

Y⁸ is selected from the group consisting of —OR¹⁹⁵, —SR¹⁹⁵, —C(R¹⁹⁷)(R¹⁹⁸)R¹⁹⁵, —C(O)R¹⁹⁵, —C(O)OR¹⁹⁵, —N(R¹⁹⁷)C(O)R¹⁹⁵, —NC(R¹⁹⁷)R¹⁹⁵, and —N(R¹⁹⁷)R¹⁹⁵;

R¹⁹⁵ is selected from the group consisting of hydrogen, alkenyl, alkoxyalkyl, alkyl, alkylthioalkyl, alkynyl, cycloalkenyl, cycloalkenylalkyl, cycloalkyl, cycloalkylalkyl, aryl, arylalkyl, heterocyclic, heterocyclic alkyl, hydroxyalkyl, and NR¹⁹⁹R²⁰⁰; and

R¹⁹⁷, R¹⁹⁸, R¹⁹⁹, and R²⁰⁰ are independently selected from the group consisting of hydrogen, alkenyl, alkoxy, alkyl, cycloalkenyl, cycloalkyl, aryl, arylalkyl, heterocyclic, and heterocyclic alkyl.

Materials that can serve as a Cox-2 selective inhibitor of the present invention include benzosulphonamide derivatives that are described in U.S. Pat. No. 6,004,948. Such benzosulphonamide derivatives have the formula shown below in formula XXXVII:

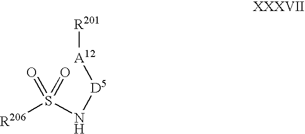

wherein:

A¹² denotes oxygen, sulphur or NH;

R²⁰¹ denotes a cycloalkyl, aryl or heteroaryl group optionally mono- or polysubstituted by halogen, alkyl, CF₃ or alkoxy;

D⁵ denotes a group of formula XXXVIII or XXXIX:

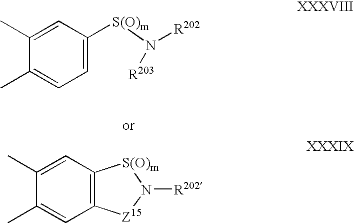

R²⁰² and R²⁰³ independently of each other denote hydrogen, an optionally polyfluorinated alkyl radical, an aralkyl, aryl or heteroaryl radical or a radical (CH₂)ₙ—X²⁹; or

R²⁰² and R²⁰³ together with the N-atom denote a three- to seven-membered, saturated, partially or totally unsaturated heterocycle with one or more heteroatoms N, O, or S, which may optionally be substituted by oxo, an alkyl, alkylaryl or aryl group or a group (CH₂)ₙ—X²⁹, R^{202,} denotes hydrogen, an optionally polyfluorinated alkyl group, an aralkyl, aryl or heteroaryl group or a group (CH₂)ₙ—X²⁹,

wherein:

X²⁹ denotes halogen, NO₂, —OR²⁰⁴, —COR²⁰⁴, —CO₂R²⁰⁴, —OCO₂R²⁰⁴, —CN, —CONR²⁰⁴OR²⁰⁵, —CONR²⁰⁴R²⁰⁵, —SR²⁰⁴, —S(O)R²⁰⁴, —S(O)₂R²⁰⁴, —NR²⁰⁴R²⁰⁵, —NHC(O)R²⁰⁴, —NHS(O)₂R²⁰⁴;

Z¹⁵ denotes —CH₂—, —CH₂—CH₂—, —CH₂—CH₂—CH₂—, —CH₂—CH═CH—, —CH═CH—CH₂—, —CH₂—CO—, —CO—CH₂—, —NHCO—, —CONH—, —NHCH₂—, —CH₂NH—, —N═CH—, —NHCH—, —CH₂—CH₂—NH—, —CH═CH—, >N—R²⁰³, >C═O, >S(O)ₘ;

R²⁰⁴ and R²⁰⁵ independently of each other denote hydrogen, alkyl, aralkyl or aryl;

n is an integer from 0 to 6;

R²⁰⁶ is a straight-chained or branched C₁₋₄-alkyl group which may optionally be mono- or polysubstituted by halogen or alkoxy, or R²⁰⁶ denotes CF₃; and

m denotes an integer from 0 to 2;

with the proviso that A¹² does not represent 0 if R²⁰⁶ denotes CF₃;

and the pharmaceutically acceptable salts thereof.

Cox-2 selective inhibitors that are useful in the subject method and compositions include the compounds that are described in U.S. Pat. Nos. 6,169,188, 6,020,343, 5,981,576 ((methylsulfonyl)phenyl furanones); U.S. Pat. No. 6,222,048 (diaryl-2-(5H)-furanones); U.S. Pat. No. 6,057,319 (3,4-diaryl-2-hydroxy-2,5-dihydrofurans); U.S. Pat. No. 6,046,236 (carbocyclic sulfonamides); U.S. Pat. Nos. 6,002,014 and 5,945,539 (oxazole derivatives); and U.S. Pat. No. 6,359,182 (C-nitroso compounds).

Still other Cox-2 inhibitors that are encompassed by the methods and compositions of the present invention include those compounds described in Table 3.

TABLE 3
Cox-2 Inhibitors

| | Trade | | | | | | Cancer |
|---|---|---|---|---|---|---|---|
| Compound | Name | Company | Mode of Action | Reference | Dosage | Toxicity | Indication |
| Iornoxicam | Safem | Roche | Cyclooxygenase | | | Cynomolgus | |
| | | Holding | inhibitor | | | monkeys: 1-2 | |
| | | AG | | | | mg/kg/day | |
| | | | | | | orally for six | |
| | | | | | | weeks | |
| 1,5-Diphenyl-3- | | Fujisawa | Cyclooxygenase | WO-09713755 | | | |
| substituted pyrazoles | | Phar- | 2 inhibitor | | | | |
| | | maceutical | | | | | |
| | | Co Ltd | | | | | |
| radicicol | | Scripps | Tyrosine kinase | WO-09625928; | | | |
| | | Research | inhibitor, | Kwon et al | | | |
| | | Institute | Cyclooxygenase | (Cancer | | | |
| | | | 2 modulator, IL-1 | Res(1992) | | | |
| | | | antagonist, TNF | 52 6296) | | | |
| | | | alpha antagonist | | | | |
| N-benzyl-3-indoleacetic | | Merck & | Cyclooxygenase | U.S. Pat. No. | | | |
| acids | | Co Inc | inhibitor, | 05510368 | | | |
| | | | Anticancer | | | | |
| GB-02283745 | | Merck & | Cyclooxygenase | | | | |
| | | Co Inc | 2 inhibitor | | | | |
| TP-72 | | Dartmouth | NO synthesis | Cancer | | | |
| | | Medical | inhibitor, | Res 1998 | | | |
| | | School | Cyclooxygenase | 58 4 | | | |
| | | | 2 inhibitor | 717-723 | | | |
| Indene inhibitors of | | American | Cyclooxygenase | WO-09821195 | | | |
| cox-2 | | Home | 2 inhibito | | | | |
| | | Products | | | | | |
| | | Corp | | | | | |
| Iornoxicam | Safem | Roche | Cyclooxygenase | | | Cynomolgus | |
| | | Holding | inhibitor | | | monkeys: 1-2 | |
| | | AG | | | | mg/kg/day | |
| | | | | | | orally for six | |
| | | | | | | weeks | |
| 1,5-Diphenyl-3- | | Fujisawa | Cyclooxygenase | WO-09713755 | | | |
| substituted pyrazoles | | Phar- | 2 inhibitor | | | | |
| | | maceutical | | | | | |
| | | Co Ltd | | | | | |
| radicicol | | Scripps | Tyrosine kinase | WO-09625928; | | | |
| | | Research | inhibitor, | Kwon et al | | | |
| | | Institute | Cyclooxygenase | (Cancer | | | |
| | | | 2 modulator, IL-1 | Res(1992) | | | |
| | | | antagonist, TNF | 52 6296) | | | |
| | | | alpha antagonist | | | | |
| N-benzyl-3-indoleacetic | | Merck & | Cyclooxygenase | U.S. Pat. No. | | | |
| acids | | Co Inc | inhibitor, | 05510368 | | | |
| | | | Anticancer | | | | |
| GB-02283745 | | Merck & | Cyclooxygenase | | | | |
| | | Co Inc | 2 inhibitor | | | | |
| TP-72 | | Dart- | NO synthesis | Cancer | | | |
| | | mouth | inhibitor, | Res 1998 | | | |
| | | Medical | Cyclooxygenase | 58 4 | | | |
| | | School | 2 inhibitor | 717-723 | | | |
| Indene inhibitors of | | American | Cyclooxygenase | WO-09821195 | | | |
| cox-2 | | Home | 2 inhibito | | | | |
| | | Products | | | | | |
| | | Corp | | | | | |
| carbocyclic | | Bristol- | Cyclooxygenase | WO-09805643 | | Rat: >300 | |
| diarylmethylene | | Myers | 2 inhibitor | | | mg/kg po | |
| derivatives | | Squibb | | | | | |
| | | Co | | | | | |
| 1,2-Diarylindole | | Bristol- | Cyclooxygenase | WO-09805639 | | | |
| | | Myers | 2 inhibitor | | | | |
| | | Squibb | | | | | |
| | | Co | | | | | |
| 1,2-Bisarylcyclobutene | | Merck & | Cyclooxygenase | WO-09736863 | | | |
| derivatives | | Co Inc | 2 inhibitor | | | | |
| Novel stilbene | | Merck & | Cyclooxygenase | WO-09728121 | | | |
| derivatives as prodrug | | Co Inc | 2 inhibitor | | | | |
| forms of the | | | | | | | |
| diphenylcyclopentenones | | | | | | | |
| claimed in U.S. Pat. No. | | | | | | | |
| 05474995, WO-09500501 | | | | | | | |
| and WO-09518799. | | | | | | | |
| 2,4-Diphenylbutenoic | | Merck & | | WO-09728120 | | | |
| acid derivatives as | | Co Inc | | | | | |
| prodrugs of COX-2 | | | | | | | |
| inhibitors claimed in | | | | | | | |
| U.S. Pat. No. 05474995, WO- | | | | | | | |
| 09500501 and WO-09518799. | | | | | | | |
| 1-(4-chlorobenzoyl)-3- | A-183827.0 | Abbott | Cyclooxygenase | | | | |
| [4-(4-fluorophenyl) | | | 2 inhibitor | | | | |
| thiazol-2-ylmethyl]-5- | | | | | | | |
| methoxy-2-methy | | | | | | | |
| lindole | | | | | | | |
| | COX-2 | Merck & | Cyclooxygenase | WO 9518799; | | | Colon |
| | inhibitor, | Co | 2 inhibitor | WO 9608482; | | | cancer |
| | Merck | | | WO 9606840; | | | |
| | | | | WO 9621667; | | | |
| | | | | WO 9636623; | | | |
| | | | | WO 9744027 | | | |
| Sulfonamide substi- | CS-179 | Monsanto | Cyclooxygenase | | | | |
| tuted diarylthiazole | | | 2 inhibitor | | | | |
| | GR-253035 | Glaxo | Cyclooxygenase | | | | Chronic |
| | | Wellcome | 2 inhibitor | | | | inflammatory |
| | | | | | | | pain |
| 4-(4-cyclohexyl-2- | JTE-522 | Japan | Cyclooxygenase | | | | Pain |
| methyloxazol-5-yl)-2- | | Tobacco | 2 inhibitor | | | | |
| fluorobenzenesulfonamide | | | | | | | |
| 5,6-diarylthiazolo[3,2- | L-768277 | Merck & | Cyclooxygenase | | | | |
| B][1,2,4]triazolo | | Co | 2 inhibitor | | | | |
| | L-783003 | Merck & | Cyclooxygenase | | | | |
| | | Co | 2 inhibitor | | | | |
| | MK-966 | Merck & | Cyclooxygenase | | 12.5-100 mg | | |
| | | Co | 2 inhibitor | | po | | |
| indometacin-derived | | Merck & | Cyclooxygenase | WO 9637467-9 | 200 | | |
| indolalkanoic acid | | Co | 2 inhibitor | | mg/kg/day | | |
| 1-Methylsulfonyl-4-[1,1- | | Monsanto | Cyclooxygenase | WO 9530656; | | | |
| dimethyl-4-(4- | | | 2 inhibitor | WO 9530652; | | | |
| fluorophenyl)cyclopenta- | | | | WO 9638418; | | | |
| 2,4-dien-3- | | | | WO 9638442 | | | |
| yl]benzene | | | | | | | |
| 4,4-dimethyl-2-phenyl- | | Merck & | Cyclooxygenase | | | | |
| 3-[4- | | Co | 2 inhibitor | | | | |
| (methylsulfonyl)phenyl] | | | | | | | |
| cyclobutenone; 1,2- | | | | | | | |
| diarylcyclobutenes | | | | | | | |
| | | Chugai | Cyclooxygenase | WO 9730030 | | | |
| | | | 2 inhibitor | | | | |
| 2-(4-methoxyphenyl)-4- | | Sankyo | Cyclooxygenase | EP 799823 | | | |
| methyl-1-(4- | | | 2 inhibitor | | | | |
| sulfamoylphenyl)pyrrole; | | | | | | | |
| 1,2-diphenylpyrrole | | | | | | | |
| derivatives | | | | | | | |
| tetrahydrofuranones | | Bristol- | Cyclooxygenase | WO 9737984 | | | |
| | | Myers | 2 inhibitor | | | | |
| | | Squibb | | | | | |
| N-[5-(4- | RWJ-63556 | Johnson | 5 Lipoxygenase | | | | |
| fluoro)phenoxy]thiophene- | | & | inhibitor; | | | | |
| 2-methanesulfonamide | | Johnson | Cyclooxygenase | | | | |
| | | | 2 inhibitor; | | | | |
| | | | Leucotriene B4 | | | | |
| | | | antagonist | | | | |
| 5(E)-(3,5-di-tert-butyl-4- | S-2474 | Shionogi | Prostaglandin E2 | EP 595546 | | | |
| hydroxy)benzylidene-2- | | | antagonist; | | | | |
| ethyl-1,2- | | | Leucotriene B4 | | | | |
| isothiazolidine-1,1- | | | antagonist; | | | | |
| dioxide | | | Cyclooxygenase | | | | |
| | | | 2 inhibitor | | | | |
| | SC-57666 | Monsanto | Cyclooxygenase | | | | |
| | | | 2 inhibitor | | | | |
| 3-formylamino-7- | T-614 | Toyama | Cyclooxygenase | DE 3834204 | | | |
| methylsulfonylamino-6- | | | 2 inhibitor; | | | | |
| phenoxy-4H-1- | | | Interleukin 1b | | | | |
| benzopyran-4-one | | | antagonist; | | | | |
| | | | Interleukin 6 | | | | |
| | | | antagonist | | | | |
| Benzenesulfonamide, | celecoxib; | Monsanto | Cyclooxygenase | | | | |
| 4-(5-(4-methylphenyl)- | Celebra; | | 2 inhibitor | | | | |
| 3-(trifluoromethyl)-1H- | SC- | | | | | | |
| pyrazol-1-yl)- | 58635; | | | | | | |
| | YM-177 | | | | | | |
| 2H-1,2-Benzothiazine- | meloxicam; | Boehringer | Cyclooxygenase | U.S. Pat. No. | 15-30 | | |
| 3-carboxamide, 4- | Mobic; | Ingelheim | 2 inhibitor; | 4233299 | mg/day | | |
| hydroxy-2-methyl-N-(5- | Mobec; | | Prostaglandin | | | | |
| methyl-2-thiazolyl)-, | Moricox; | | synthase inhibitor | | | | |
| 1,1-dioxide- | Mobicox; | | | | | | |
| | Movalis; | | | | | | |
| Methanesulfonamide, | nimesulide | Helsinn | Cyclooxygenase | U.S. Pat. No. | | | |
| N-(4-nitro-2- | | | 2 inhibitor; | 3840597 | | | |
| phenoxyphenyl) | | | Prostaglandin | | | | |
| | | | synthase inhibitor | | | | |
| Methanesulfonamide, | nimesulide, | Poli | Cyclooxygenase | | | | |
| N-(4-nitro-2- | Poli | | 2 inhibitor | | | | |
| phenoxyphentyl) | | | | | | | |

Still other Cox-2 inhibitors that are encompassed by the methods and compositions of the present invention include those compounds described in table 4.

TABLE 4
Cox-2 Inhibitors

| Patent | Publication Date | Oncology Indication | Dosage of Preferred Compounds |
|---|---|---|---|
| U.S. Pat. No. 5776967 A | 980707 | colorectal cancer | |
| WO 9821195 A1 | 980522 | colorectal cancer | |
| WO 9804527 A1 | 980205 | colorectal cancer | 0.01-100 mg/kg/day orally or |
| | | | parenterally |
| WO 9825896 A1 | 980618 | | |
| U.S. Pat. No. 5760068 A | 980602 | | |
| WO 9822101 A2 | 980528 | colorectal cancer | |
| WO 9816227 A1 | 980423 | antiangiogenic | |
| U.S. Pat. No. 5719163 A | 980217 | epithelial cell neoplasia | |
| WO 9806708 A1 | 980219 | | |
| WO 9738986 A1 | 971023 | | |
| U.S. Pat. No. 5663180 A | 970902 | | |
| WO 9729776 A1 | 970821 | | |
| WO 9729774 A1 | 970821 | cancer | 0.1-2000 (preferably 0.5-500, |
| | | | especially 1-100) mg/kg/day orally, |
| | | | intravascularly, intraperitoneally, |
| | | | subcutaneously, intramuscularly, |
| | | | or topically. |
| WO 9729775 A1 | 970821 | cancer | 0.1-2000 (preferably 0.5-500, |
| | | | especially 1-100) mg/kg/day orally, |
| | | | intravascularly, intraperitoneally, |
| | | | subcutaneously, intramuscularly, |
| | | | or topically. |
| WO 9727181 A1 | 970731 | | |
| WO 9714679 A2 | 970424 | | |
| WO 9711704 A1 | 970403 | | |
| U.S. Pat. No. 5616601 A | 970401 | | |
| WO 9641645 A1 | 961227 | | |
| WO 9641625 A1 | 961227 | colorectal cancer | 0.01-100 mg/kg/day oral, topical or |
| | | | parenteral. |
| WO 9641626 A1 | 961227 | | |
| WO 9638442 A1 | 961205 | | |
| WO 9638418 A1 | 961205 | colorectal cancer | 0.1-100 (preferably 0.1-10) mg/kg/day, |
| | | | orally, injection, |
| | | | topically, or transdermally. |
| WO 9625405 A1 | 960822 | | |
| WO 9624585 A1 | 960815 | | |
| WO 9609293 A1 | 960328 | | |
| WO 9603387 A1 | 960208 | | |
| U.S. Pat. No. 5739166 | 980414 | colorectal cancer | 0.01-100 (preferably 0.1-10 mg/kg/day, |
| WO 9616934 A1 | 960606 | | orally, topical or |
| | | | intramuscular |
| WO 9603388 A1 | 960208 | | |
| WO 9603392 A1 | 960208 | | |
| WO 9530652 A1 | 951116 | | |
| WO 9515316 A1 | 950608 | | |
| WO 9515318 A1 | 950608 | | |
| U.S. Pat. No. 5393790 A | 950228 | | |
| U.S. Pat. No. 5380738 | 950110 | colorectal cancer | 0.01-100 (pref. 0.1-50) mg/kg/day, |
| WO 9427980 A1 | 941208 | | oral, parental, or topical |
| U.S. Pat. No. 5719163 | 980217 | colorectal cancer | 0.01-100 (pref. 0.1-50) mg/kg/day, |
| WO 9427980 A1 | 941208 | | oral, parental, or topical |
| U.S. Pat. No. 5420343 A | 950530 | | |
| U.S. Pat. No. 5434178 | 950718 | | |
| U.S. Pat. No. 5466823 | 951114 | | |
| U.S. Pat. No. 5521207 | 960528 | | |
| U.S. Pat. No. 5563165 | 961008 | | |
| U.S. Pat. No. 5508426 | 960416 | | |
| U.S. Pat. No. 5504215 | 960402 | | |
| U.S. Pat. No. 5516907 | 960514 | | |
| U.S. Pat. No. 5510496 | 960423 | | |
| U.S. Pat. No. 5753688 | 980519 | | |
| U.S. Pat. No. 5753688 | 980519 | | |
| U.S. Pat. No. 5736579 | 980407 | colorectal cancer | |
| WO 9521817 A1 | 950817 | | |
| SOFRC 95/1107 | 960424 | | |
| U.S. Pat. No. 5668161 | 970916 | | |
| U.S. Pat. No. 5418254 | 950523 | | |
| U.S. Pat. No. 5576339 | 961119 | | colorectal cancer |
| U.S. Pat. No. 5672626 | 970930 | | |
| U.S. Pat. No. 5670510 | 970923 | | |
| U.S. Pat. No. 5686470 | 971111 | colorectal cancer | 0.01-100 (preferably 0.1-10) mg/kg/day |
| WO 9624584 A1 | 960815 | | |
| U.S. Pat. No. 5580985 | 961203 | | 0.01-100 (preferably 0.1-10) mg/kg/day |
| WO 9603385 A1 | 960208 | | |
| U.S. Pat. No. 5756530 | 980526 | | 0.01-100 (preferably 0.1-10) mg/kg/day |
| WO 9603385 A1 | 960208 | | |
| U.S. Pat. No. 5486534 A | 960123 | | |
| WO 9603385 A1 | 960208 | | |
| U.S. Pat. No. 5620999 | 970415 | colorectal cancer | 0.01-100 (preferably 0.5-20) mg/kg/day, |
| WO 9603387 A1 | 960208 | | oral, intravascular, |
| | | | intraperitoneal, subcutaneous, |
| | | | intramuscular, or topical |
| U.S. Pat. No. 08/765,865 | 970110 | | |
| U.S. Pat. No. 5696143 | 970912 | | |
| WO 960923 A1 | 960328 | | |
| U.S. Pat. No. 5547975 | 960820 | | |
| WO 9609304 A1 | 960328 | | |
| U.S. Pat. No. 08/809475 | 970609 | | |
| U.S. Pat. No. 5565482 | 961015 | | |
| WO 9609304 A1 | 960328 | | |
| U.S. Pat. No. 5670532 | 970923 | | |
| WO 9609304 A1 | 960328 | | |
| U.S. Pat. No. 5596008 | 970121 | | |
| WO 9624585 A1 | 960815 | | |
| U.S. Pat. No. 08/809318 | 970320 | | |
| U.S. Pat. No. 08/849069 | 971117 | | |
| U.S. Pat. No. 08/387680 | 950213 | | |
| U.S. Pat. No. 08/894124 | 970811 | | |
| U.S. Pat. No. 08/702417 | 960814 | | |
| U.S. Pat. No. 08/801768 | 970218 | | |
| U.S. Pat. No. 5643933 | 970701 | | |
| WO 9638442 A1 | 961205 | | |
| U.S. Pat. No. 08/952661 | 960420 | | |
| U.S. Pat. No. 08/945840 | 960531 | | |
| U.S. Pat. No. 08/822528 | 970324 | | |
| U.S. Pat. No. 08/541850 | 951010 | | |
| U.S. Pat. No. 08/540522 | 951010 | | |
| PCT U.S. Pat. No. 97/05497 | 970411 | | |
| U.S. Pat. No. 08/908554 | 970808 | | |
| U.S. Pat. No. 09/005610 | 980112 | | |
| U.S. Pat. No. 08/987356 | 971209 | | |
| U.S. Pat. No. 60/032688 | 961210 | | |
| PCT U.S. Pat. No. 98/07677 | 980418 | | |
| U.S. Pat. No. 09/062537 | 980417 | | |
| U.S. Pat. No. 60/044485 | 970421 | | |
| U.S. Pat. No. 08/004/822 | 930115 | | |
| U.S. Pat. No. 08/464722 | 950624 | | |
| U.S. Pat. No. 08/425022 | 950413 | | |
| U.S. Pat. No. 08/425029 | 950419 | | |
| U.S. Pat. No. 08/424979 | 950419 | | |
| U.S. Pat. No. 08/969953 | 971125 | | |
| U.S. Pat. No. 5380738 | 950110 | | |
| U.S. Pat. No. 08/952156 | 971111 | | |
| U.S. Pat. No. 08/647911 | 960530 | | |
| U.S. Pat. No. 08/457902 | 950601 | | |
| U.S. Pat. No. 08/957345 | 971024 | | |
| EPO 95909447.5 | 950207 | | |
| U.S. Pat. No. 08/776358 | 970124 | | |
| U.S. Pat. No. 08/237739 | 940504 | | |
| U.S. Pat. No. 08/894102 | 970808 | | |
| EPO 95928164.3 | 950727 | | |
| U.S. Pat. No. 09/101493 | 980709 | | |
| U.S. Pat. No. 08/992327 | 971217 | | |
| U.S. Pat. No. 08/776090 | 970609 | | |
| U.S. Pat. No. 08/765865 | 970110 | | |
| AT 9700165 A | 980415 | | |
| AU 9719132 A | 970814 | | |
| CA 2164559 AA | 960610 | | |
| DE 19518421 A1 | 961121 | | |
| DE 19533643 A1 | 970313 | | 0.01-1000 mg/day orally or |
| | | | parenterally |
| DE 19533644 A1 | 970313 | | 0.01-1000 mg/day orally or |
| | | | parenterally |
| EP 714895 A1 | 960605 | | 0.001-150 (preferably 5-20) mg/kg/day |
| EP 799823 A1 | 971008 | | |
| EP 832652 A1 | 980401 | adenocarcinoma | |
| EP 846689 A1 | 980610 | metastasis inhibitors | |
| EP 850894 A1 | 980701 | | |
| EP 850895 A1 | 980701 | | |
| FR 2751966 Al | 980206 | | Oral or parenteral 0.1-100 mg/kg/day. |
| GB 2283745 A1 | 950517 | | |
| GB 2294879 A1 | 960515 | | |
| GB 2319772 A | 980603 | cancer | 50 mg to 5 g/day (preferably 100-500 mg/day |
| DE 19753463 A1 | 980604 | | in 1 to 3 doses) |
| GB 2320715 A | 980701 | | |
| JP 08157361 A2 | 960618 | | |
| JP 09048769 A2 | 970218 | | |
| JP 09071656 A2 | 970318 | | |
| JP 09071657 A2 | 970318 | | |
| JP 09077664 A2 | 970325 | | |
| JP 09194354 A2 | 970729 | ulcerative colitis | |
| JP 09221422 A2 | 970826 | | |
| JP 10175861 A2 | 980630 | metastasis inhibitors | |
| U.S. Pat. No. 5474995 A | 951212 | | |
| U.S. Pat. No. 5510368 A | 960423 | | 0.1-140 mg/kg/day or 0.5-7g/ |
| | | | patient, oral, topical, perenteral, |
| | | | inhalation, rectal |
| U.S. Pat. No. 5604260 A | 970218 | | |
| U.S. Pat. No. 5616458 A | 970401 | | |
| U.S. Pat. No. 5633272 A | 970527 | | |
| U.S. Pat. No. 5663195 A | 970902 | | 0.01-100 mg/kg/day; 0.5 mg-6 g/day |
| U.S. Pat. No. 5677318 A | 971014 | inhibitor of cellular neoplastic | |
| | | transformations and metastatic | |
| | | tumor growth; treatment of | |
| | | proliferative disorders, e.g., | |
| | | tumor angiogenesis | |
| U.S. Pat. No. 5677318 A | 971014 | | |
| U.S. Pat. No. 5681842 A | 971028 | | |
| U.S. Pat. No. 5686460 A | 971111 | | |
| U.S. Pat. No. 5733909 A | 980331 | | |
| U.S. Pat. No. 5783597 A | 980721 | | |
| WO 9413635 Al | 940623 | | |
| WO 9414977 A1 | 940707 | | |
| WO 9420480 A1 | 940915 | Inhibition of neoplastic | 0.01-140 mg/kg/day adminstered |
| | | transformations and metastatic | orally. |
| | | tumor growth | |
| WO 9426731 A1 | 941124 | | |
| WO 9500501 A2 | 950105 | | |
| WO 9511883 A1 | 950504 | colorectal cancer | |
| WO 9606840 A1 | 960307 | | |
| WO 9608482 A1 | 960321 | | |
| WO 9611676 A1 | 960425 | | 0.01-140 mg/kg/day |
| WO 9612483 A1 | 960502 | inhibition of nitric oxide | |
| | | formation | |
| WO 9613483 A1 | 960509 | Inhibition of neoplastic | 0.01-140 mg/kg/day |
| | | transformation and metastatic | |
| | | tumor growth | |
| WO 9619462 A1 | 960627 | | 0.01-1000 (preferably 0.1-300) mg/day |
| | | | p.o. or parenterally |
| WO 9619462 A1 | 960627 | | |
| WO 9619463 A1 | 960627 | | |
| WO 9619463 A1 | 960627 | | 0.1-1000 (preferably 1-300) mg/day |
| | | | p.o. or parenterally |
| WO 9619469 A1 | 960627 | | |
| WO 9621667 A1 | 960718 | | |
| WO 9623786 A1 | 960808 | osteosarcoma | 0.01-140 mg/kg/day, orally, rectal, |
| | | | injection, topical. |
| WO 9624604 A1 | 960815 | | |
| WO 9625405 A1 | 960822 | | |
| WO 9625928 A1 | 960829 | | |
| WO 9626921 A1 | 960906 | | |
| WO 9631509 A1 | 961010 | | |
| WO 9636617 A1 | 961121 | colorectal cancer | |
| WO 9636623 A1 | 961121 | | |
| WO 9637467 A1 | 961128 | | 0.01-140 mg/kg/day, orally, |
| | | | topical, parenteral, rectal or |
| | | | inhalation. |
| WO 9637469 A1 | 961128 | | |
| WO 9639144 A1 | 961212 | | |
| WO 9640143 A1 | 961219 | | |
| WO 9641626 A1 | 961227 | colorectal cancer | |
| WO 9703667 Al | 970206 | colonic adenomas; colonic | |
| | | adenocarcinomas | |
| WO 9703953 A1 | 970206 | | 0.01-1000 mg p.o or i.p. (oral, |
| | | | parenteral, rectal, topical or |
| | | | transdermal) |
| WO 9709977 A1 | 970320 | | |
| WO 9710840 A1 | 970327 | | |
| WO 9711701 A1 | 970403 | cancer | |
| WO 9711701 A1 | 970403 | | |
| WO 9713755 A1 | 970417 | cancer | |
| WO 9713767 A1 | 970417 | | |
| WO 9714691 A1 | 970424 | | |
| WO 9716435 A1 | 970509 | | |
| WO 9725045 A1 | 970717 | | 0.1-80 mg/kg/day orally or |
| | | | parenterally |
| WO 9725046 A1 | 970717 | | |
| WO 9725047 A1 | 970717 | | 0.1-80 mg/kg/day oral or |
| | | | parenteral |
| WO 9725048 A1 | 970717 | pulmonary sarcoisosis | 0.1-80 mg/kg/day oral or |
| | | | parenteral |
| WO 9727181 A1 | 970731 | colorectal cancer | |
| WO 9728120 A1 | 970807 | | |
| WO 9728121 A1 | 970807 | | 0.01-140 mg/kg/day |
| WO 9730030 A1 | 970821 | | 3-150 mg/hg p.o. or 1-50 mg/hg |
| | | | parenterally |
| WO 9731631 A1 | 970904 | | |
| WO 9734882 A1 | 970925 | colorectal cancer | |
| WO 9736497 A2 | 971009 | antineoplastic; prostate, renal, | |
| | | colon, breast, or cervical | |
| | | cancer | |
| WO 9736863 A1 | 971009 | | 0.01-140 mg/kg/day (oral, topical, |
| | | | rectal, parenteral, inhalation) |
| WO 9737984 A1 | 971016 | | Orally 300 mg/kg/day |
| WO 9738686 A1 | 971023 | regulation of COX-II | |
| | | expression | |
| WO 9740012 A1 | 971030 | | |
| WO 9744027 A1 | 971127 | | Orally 2.5-250 mg/day (preferably |
| | | | 12.5-20 mg/day) |
| WO 9744028 A1 | 971127 | | |
| WO 9745420 A1 | 971204 | | |
| WO 9746524 A1 | 971211 | | |
| WO 9746532 A1 | 971211 | | 0.08-15.0 mg/kg/day (preferably |
| | | | 0.16-3.0 mg/kg/day) |
| WO 9800416 A1 | 980108 | | |
| WO 9803484 A1 | 980129 | Inhibit neoplastic formation | Orally 0.01-140 mg/kg/day |
| | | and metastic tumor growth | (preferably 0.5-7 mg/kg/day) |
| WO 9805639 A1 | 980212 | | |
| WO 9806715 A1 | 980219 | | |
| WO 9807425 A1 | 980226 | | 0.01-80 mg/kg/day oral or |
| | | | parenteral; topical 0.1-150 mg/day |
| | | | in 1-4 doses. |
| WO 9807714 A1 | 980226 | | |
| WO 9811080 A1 | 980319 | | 1-1000 mg/day (oral, rectal, |
| | | | topical); 0.1-500 mg/day |
| | | | parenteral. |
| WO 9815528 A1 | 980416 | | |
| WO 9816227 A1 | 980423 | | |
| WO 9817292 A1 | 980430 | | |
| WO 9821195 A1 | 980522 | tumor angiogenesis; colorectal | |
| | | cancers | |
| WO 9822101 A2 | 980528 | metastasis | |
| WO 9822104 A2 | 980528 | | |
| WO 9822442 A2 | 980528 | | |
| WO 9822457 A1 | 980528 | | |
| WO 9824782 A2 | 980611 | | |
| ZA 9704806 A | 980325 | colon cancer | 0.1-500 mg/kg/day administered |
| | | | orally |

Cox-2 inhibitors that are useful in the present invention can be supplied by any source as long as the Cox-2 inhibitor is pharmaceutically acceptable. Cox-2 inhibitors can be isolated and purified from natural sources or can be synthesized. Cox-2 inhibitors should be of a quality and purity that is conventional in the trade for use in pharmaceutical products.

The celecoxib used in the therapeutic combinations of the present invention can be prepared in the manner set forth in U.S. Pat. No. 5,466,823.

The valdecoxib used in the therapeutic combinations of the present invention can be prepared in the manner set forth in U.S. Pat. No. 5,633,272.

The parecoxib used in the therapeutic combinations of the present invention can be prepared in the manner set forth in U.S. Pat. No. 5,932,598.

The rofecoxib used in the therapeutic combinations of the present invention can be prepared in the manner set forth in U.S. Pat. No. 5,968,974.

The Japan Tobacco JTE-522 used in the therapeutic combinations of the present invention can be prepared in the manner set forth in JP 90/52,882.

Pyrazoles can be prepared by methods described in WO 95/15,316. Pyrozoles can further be prepared by methods described in WO 95/15315. Pyrozoles can also be prepared by methods described in WO 96/03385.

Thiophene analogs can be prepared by methods described in WO 95/00501. Preparation of thiophene analogs is also described in WO 94/15932.

Oxazoles can be prepared by the methods described in WO 95/00501. Preparation of oxazoles is also described in WO 94/27980.

Isoxazoles can be prepared by the methods described in WO 96/25405.

Imidazoles can be prepared by the methods described in WO 96/03388. Preparation of imidazoles is also described in WO 96/03387.

Cyclopentene Cox-2 inhibitors can be prepared by the methods described in U.S. Pat. No. 5,344,991. Preparation of cyclopentane Cox-2 inhibitors is also described in WO 95/00501.

Terphenyl compounds can be prepared by the methods described in WO 96/16934.

Thiazole compounds can be prepared by the methods described in WO 96/03,392.

Pyridine compounds can be prepared by the methods described in WO 96/03392. Preparation of pyridine compounds is also described in WO 96/24,585.

The major categories that some preferred antineoplastic agents fall into include antimetabolite agents, alkylating agents, antibiotic-type agents, hormonal anticancer agents, immunological agents, interferon-type agents, and a category of miscellaneous antineoplastic agents. Some antineoplastic agents operate through multiple or unknown mechanisms and can thus be classified into more than one category.

A first family of antineoplastic agents which may be used in combination with the present invention consists of antimetabolite-type antineoplastic agents. Antimetabolites are typically reversible or irreversible enzyme inhibitors, or compounds that otherwise interfere with the replication, translation or transcription of nucleic acids. Suitable antimetabolite antineoplastic agents that may be used in the present invention include, but are not limited to acanthifolic acid, aminothiadiazole, anastrozole, bicalutamide, brequinar sodium, capecitabine, carmofur, Ciba-Geigy CGP-30694, cladribine, cyclopentyl cytosine, cytarabine phosphate stearate, cytarabine conjugates, cytarabine ocfosfate, Lilly DATHF, Merrel Dow DDFC, dezaguanine, dideoxycytidine, dideoxyguanosine, didox, Yoshitomi DMDC, doxifluridine, Wellcome EHNA, Merck & Co. EX-015, fazarabine, finasteride, floxuridine, fludarabine phosphate, N-(2′-furanidyl)-5-fluorouracil, Daiichi Seiyaku FO-152, fluorouracil (5-FU), 5-FU-fibrinogen, isopropyl pyrrolizine, Lilly LY-188011, Lilly LY-264618, methobenzaprim, methotrexate, Wellcome MZPES, nafarelin, norspermidine, nolvadex, NCI NSC-127716, NCI NSC-264880, NCI NSC-39661, NCI NSC-612567, Warner-Lambert PALA, pentostatin, piritrexim, plicamycin, Asahi Chemical PL-AC, stearate; Takeda TAC-788, thioguanine, tiazofurin, Erbamont TIF, trimetrexate, tyrosine kinase inhibitors, tyrosine protein kinase inhibitors, Taiho UFT, toremifene, and uricytin.

A second family of antineoplastic agents which may be used in combination with the present invention consists of alkylating-type antineoplastic agents. The alkylating agents are believed to act by alkylating and cross-linking guanine and possibly other bases in DNA, arresting cell division. Typical alkylating agents include nitrogen mustards, ethyleneimine compounds, alkyl sulfates, cisplatin, and various nitrosoureas. A disadvantage with these compounds is that they not only attack malignant cells, but also other cells which are naturally dividing, such as those of bone marrow, skin, gastro-intestinal mucosa, and fetal tissue. Suitable alkylating-type antineoplastic agents that may be used in the present invention include, but are not limited to, Shionogi 254-S, aldo-phosphamide analogues, altretamine, anaxirone, Boehringer Mannheim BBR-2207, bestrabucil, budotitane, Wakunaga CA-102, carboplatin, carmustine (BiCNU), Chinoin-139, Chinoin-153, chlorambucil, cisplatin, cyclophosphamide, American Cyanamid CL-286558, Sanofi CY-233, cyplatate, dacarbazine, Degussa D-19-384, Sumimoto DACHP(Myr)2, diphenylspiromustine, diplatinum cytostatic, Erba distamycin derivatives, Chugai DWA-2114R, ITI E09, elmustine, Erbamont FCE-24517, estramustine phosphate sodium, etoposide phosphate, fotemustine, Unimed G-6-M, Chinoin GYKI-17230, hepsul-fam, ifosfamide, iproplatin, lomustine, mafosfamide, mitolactol, mycophenolate, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, thiotepa, Yakult Honsha SN-22, spiromus-tine, Tanabe Seiyaku TA-077, tauromustine, temozolomide, teroxirone, tetraplatin and trimelamol.

A third family of antineoplastic agents which may be used in combination with the present invention consists of antibiotic-type antineoplastic agents. Suitable antibiotic-type antineoplastic agents that may be used in the present invention include, but are not limited to Taiho 4181-A, aclarubicin, actinomycin D, actinoplanone, Erbamont ADR-456, aeroplysinin derivative, Ajinomoto AN-201-II, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, doxorubicin-fibrinogen, elsamicin-A, epirubicin, erbstatin, esorubicin, esperamicin-A1, esperamicin-Alb, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SRI International NSC-357704, oxalysine, oxaunomycin, peplomycin, pilatin, pirarubicin, porothramycin, pyrindamycin A, Tobishi RA-I, rapamycin, rhizoxin, rodorubicin, sibanomicin, siwenmycin, Sumitomo SM-5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024 and zorubicin.

A fourth family of antineoplastic agents which may be used in combination with the present invention consists of synthetic nucleosides. Several synthetic nucleosides have been identified that exhibit anticancer activity. A well-known nucleoside derivative with strong anticancer activity is 5-fluorouracil (5-FU). 5-Fluorouracil has been used clinically in the treatment of malignant tumors, including, for example, carcinomas, sarcomas, skin cancer, cancer of the digestive organs, and breast cancer. 5-Fluorouracil, however, causes serious adverse reactions such as nausea, alopecia, diarrhea, stomatitis, leukocytic thrombocytopenia, anorexia, pigmentation, and edema. Derivatives of 5-fluorouracil with anti-cancer activity have been described in U.S. Pat. No. 4,336,381.

U.S. Pat. No. 4,000,137 discloses that the peroxidate oxidation product of inosine, adenosine, or cytidine with methanol or ethanol has activity against lymphocytic leukemia. Cytosine arabinoside (also referred to as Cytarabin, araC, and Cytosar) is a nucleoside analog of deoxycytidine that was first synthesized in 1950 and introduced into clinical medicine in 1963. It is currently an important drug in the treatment of acute myeloid leukemia. It is also active against acute lymphocytic leukemia, and to a lesser extent, is useful in chronic myelocytic leukemia and non-Hodgkin's lymphoma. The primary action of araC is inhibition of nuclear DNA synthesis. Handschumacher, R. and Cheng, Y., “Purine and Pyrimidine Antimetabolites”, Cancer Medicine, Chapter XV-1, 3 rd Edition, Edited by J. Holland, et al., Lea and Febigol, publishers.

5-Azacytidine is a cytidine analog that is primarily used in the treatment of acute myelocytic leukemia and myelodysplastic syndrome.

2-Fluoroadenosine-5′-phosphate (Fludara, also referred to as FaraA) is one of the most active agents in the treatment of chronic lymphocytic leukemia. The compound acts by inhibiting DNA synthesis. Treatment of cells with F-araA is associated with the accumulation of cells at the G1/S phase boundary and in S phase; thus, it is a cell cycle S phase-specific drug. InCorp of the active metabolite, F-araATP, retards DNA chain elongation. F-araA is also a potent inhibitor of ribonucleotide reductase, the key enzyme responsible for the formation of dATP. 2-Chlorodeoxyadenosine is useful in the treatment of low grade B-cell neoplasms such as chronic lymphocytic leukemia, non-Hodgkins' lymphoma, and hairy-cell leukemia. The spectrum of activity is similar to that of Fludara. The compound inhibits DNA synthesis in growing cells and inhibits DNA repair in resting cells.

A fifth family of antineoplastic agents which may be used in combination with the present invention consists of hormonal agents. Suitable hormonal-type antineoplastic agents that may be used in the present invention include, but are not limited to Abarelix, Abbott A-84861, Abiraterone acetate, Aminoglutethimide, anastrozole, Asta Medica AN-207, Antide, Chugai AG-041 R, Avorelin, aseranox, Sensus B2036-PEG, Bicalutamide, buserelin, BTG CB-7598, BTG CB-7630, Casodex, cetrolix, clastroban, clodronate disodium, Cosudex, Rotta Research CR-1505, cytadren, crinone, deslorelin, droloxifene, dutasteride, Elimina, Laval University EM-800, Laval University EM-652, epitiostanol, epristeride, Mediolanum EP-23904, EntreMed 2-ME, exemestane, fadrozole, finasteride, flutamide, formestane, Pharmacia & Upjohn FCE-24304, ganirelix, goserelin, Shire gonadorelin agonist, Glaxo Wellcome GW-5638, Hoechst Marion Roussel Hoe-766, NCI hCG, idoxifene, isocordoin, Zeneca ICI-182780, Zeneca ICI-118630, Tulane University J015X, Schering Ag J96, ketanserin, lanreotide, Milkhaus LDI-200, letrozol, leuprolide, leuprorelin, liarozole, lisuride hydrogen maleate, loxiglumide, mepitostane, Leuprorelin, Ligand Pharmaceuticals LG-1127, LG-1447, LG-2293, LG-2527, LG-2716, Bone Care International LR-103, Lilly LY-326315, Lilly LY-353381-HCl, Lilly LY-326391, Lilly LY-353381, Lilly LY-357489, miproxifene phosphate, Orion Pharma MPV-2213ad, Tulane University MZ-4-71, nafarelin, nilutamide, Snow Brand NKS01, octreotide, Azko Nobel ORG-31710, Azko Nobel ORG-31806, orimeten, orimetene, orimetine, ormeloxifene, osaterone, Smithkline Beecham SKB-105657, Tokyo University OSW-1, Peptech PTL-03001, Pharmacia & Upjohn PNU-156765, quinagolide, ramorelix, Raloxifene, statin, sandostatin LAR, Shionogi S-10364, Novartis SMT-487, somavert, somatostatin, tamoxifen, tamoxifen methiodide, teverelix, toremifene, triptorelin, TT-232, vapreotide, vorozole, Yamanouchi YM-116, Yamanouchi YM-511, Yamanouchi YM-55208, Yamanouchi YM-53789, Schering AG ZK-1911703, Schering AG ZK-230211, and Zeneca ZD-182780.

A sixth family of antineoplastic agents which may be used in combination with the present invention consists of a miscellaneous family of antineoplastic agents including, but not limited to alpha-carotene, alpha-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluron, benzotript, Ipsen-Beaufour BIM-23015, bisantrene, Bristo-Myers BMY-40481, Vestar boron-10, bromofosfamide, Wellcome BW-502, Wellcome BW-773, calcium carbonate, Calcet, Calci-Chew, Calci-Mix, Roxane calcium carbonate tablets, caracemide, carmethizole hydrochloride, Ajinomoto CDAF, chlorsulfaquinoxalone, Chemes CHX-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Cell Pathways CP-461, Yakult Honsha CPT-11, crisnatol, curaderm, cytochalasin B, cytarabine, cytocytin, Merz D-609, DABIS maleate, dacarbazine, datelliptinium, DFMO, didemnin-B, dihaematoporphyrin ether, dihydrolenperone, dinaline, distamycin, Toyo Pharmar DM-341, Toyo Pharmar DM-75, Daiichi Seiyaku DN-9693, docetaxel, Encore Pharmaceuticals E7869, elliprabin, elliptinium acetate, Tsumura EPMTC, ergotamine, etoposide, etretinate, Eulexin®, Cell Pathways Exisulind® (sulindac sulphone or CP-246), fenretinide, Merck Research Labs Finasteride, Florical, Fujisawa FR-57704, gallium nitrate, gemcitabine, genkwadaphnin, Gerimed, Chugai GLA-43, Glaxo GR-63178, grifolan NMF-5N, hexadecylphosphocholine, Green Cross HO-221, homoharringtonine, hydroxyurea, BTG ICRF-187, ilmofosine, irinotecan, isoglutamine, isotretinoin, Otsuka JI-36, Ramot K-477, ketoconazole, Otsuak K-76COONa, Kureha Chemical K-AM, MECT Corp KI-8110, American Cyanamid L-623, leucovorin, levamisole, leukoregulin, Ionidamine, Lundbeck LU-23-112, Lilly LY-186641, Materna, NCI (US) MAP, marycin, Merrel Dow MDL-27048, Medco MEDR-340, megestrol, merbarone, merocyanine derivatives, methylanilinoacridine, Molecular Genetics MGI-136, minactivin, mitonafide, mitoquidone, Monocal, mopidamol, motretinide, Zenyaku Kogyo MST-16, Mylanta, N-(retinoyl)amino acids, Nilandron; Nisshin Flour Milling N-021, N-acylated-dehydroalanines, nafazatrom, Taisho NCU-190, Nephro-Calci tablets, nocodazole derivative, Normosang, NCI NSC-145813, NCI NSC-361456, NCI NSC-604782, NCI NSC-95580, octreotide, Ono ONO-1 12, oquizanocine, Akzo Org-10172, paclitaxel, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, retinoids, Encore Pharmaceuticals R-flurbiprofen, Sandostatin; Sapporo Breweries RBS, restrictin-P, retelliptine, retinoic acid, Rhone-Poulenc RP-49532, Rhone-Poulenc RP-56976, Scherring-Plough SC-57050, Scherring-Plough SC-57068, selenium(selenite and selenomethionine), SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, Sugen SU-101, Sugen SU-5416, Sugen SU-6668, sulindac, sulindac sulfone; superoxide dismutase, Toyama T-506, Toyama T-680, taxol, Teijin TEI-0303, teniposide, thaliblastine, Eastman Kodak TJB-29, tocotrienol, Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, withanolides, Yamanouchi YM-534, Zileuton, ursodeoxycholic acid, and Zanosar.

Table No. 5 provides illustrative examples of median dosages for selected cancer agents that may be used in combination with an antiangiogenic agent. It should be noted that specific dose regimen for the chemotherapeutic agents below depends upon dosing considerations based upon a variety of factors including the type of neoplasia; the stage of the neoplasm; the age, weight, sex, and medical condition of the patient; the route of administration; the renal and hepatic function of the patient; and the particular combination employed.

TABLE No. 5
Median dosages for selected cancer agents.

| NAME OF | |
|---|---|
| CHEMOTHERAPEUTIC AGENT | MEDIAN DOSAGE |
| Asparaginase | 10,000 units |
| Bleomycin Sulfate | 15 units |
| Carboplatin | 50-450 mg. |
| Carmustine. | 100 mg. |
| Cisplatin. | 10-50 mg. |
| Cladribine | 10 mg. |
| Cyclophosphamide. (lyophilized) | 100 mg.-2 gm. |
| Cyclophosphamide. (non-lyophilized) | 100 mg.-2 gm. |
| Cytarabine (lyophilized. powder) | 100 mg.-2 gm. |
| Dacarbazine. | 100 mg.-200 mg. |
| Dactinomycin | 0.5 mg. |
| Daunorubicin. | 20 mg. |
| Diethylstilbestrol. | 250 mg. |
| Doxorubicin. | 10-150 mg. |
| Etidronate. | 300 mg. |
| Etoposide | 100 mg. |
| Floxuridine | 500 mg. |
| Fludarabine Phosphate. | 50 mg. |
| Fluorouracil. | 500 mg.-5 gm. |
| Goserelin | 3.6 mg. |
| Granisetron Hydrochloride. | 1 mg. |
| Idarubicin | 5-10 mg. |
| Ifosfamide. | 1-3 gm. |
| Leucovorin Calcium. | 50-350 mg. |
| Leuprolide. | 3.75-7.5 mg. |
| Mechlorethamine. | 10 mg. |
| Medroxyprogesterone. | 1 gm. |
| Melphalan. | 50 gm. |
| Methotrexate | 20 mg.-1 gm. |
| Mitomycin. | 5-40 mg. |
| Mitoxantrone. | 20-30 mg. |
| Ondansetron Hydrochloride. | 40 mg. |
| Paclitaxel. | 30 mg. |
| Pamidronate Disodium. | 30-90 mg. |
| Pegaspargase | 750 units |
| Plicamycin. | 2,500 mcgm. |
| Streptozocin | 1 gm. |
| Thiotepa. | 15 mg. |
| Teniposide. | 50 mg. |
| Vinblastine | 10 mg. |
| Vincristine. | 1-5 mg. |
| Aldesleukin | 22 million units |
| Epoetin Alfa | 2,000-10,000 units |
| Filgrastim. | 300-480 mcgm. |
| Immune Globulin. | 500 mg.-10 gm. |
| Interferon Alpha-2a | 3-36 million units |
| Interferon Alpha-2b | 3-50 million units |
| Levamisole. | 50 mg. |
| Octreotide. | 1,000-5,000 mcgm. |
| Sargramostim | 250-500 mcgm. |

Still more preferred antineoplastic agents include: anastrozole, calcium carbonate, capecitabine, carboplatin, cisplatin, Cell Pathways CP-461, cyclophosphamide, docetaxel, doxorubicin, etoposide, Exisulind®, fluorouracil (5-FU), fluoxymestrine, gemcitabine, goserelin, irinotecan, ketoconazole, letrozol, leucovorin, levamisole, megestrol, mitoxantrone, paclitaxel, raloxifene, retinoic acid, tamoxifen, thiotepa, topotecan, toremifene, vinorelbine, vinblastine, vincristine, selenium (selenomethionine), ursodeoxycholic acid, sulindac sulfone and eflornithine (DFMO).

The phrase “taxane” includes a family of diterpene alkaloids all of which contain a particular eight (8)-member “taxane” ring structure. Taxanes such as paclitaxel prevent the normal post division breakdown of microtubules, which form to pull and separate the newly duplicated chromosome pairs to opposite poles of the cell prior to cell division. In cancer cells, which are rapidly dividing, taxane therapy causes the microtubules to accumulate which ultimately prevents further division of the cancer cell. Taxane therapy also affects other cell processes dependant on microtubules such as cell motility, cell shape and intracellular transport. The major adverse side-effects associated with taxane therapy can be classified into cardiac effects, neurotoxicity, haematological toxicity, and hypersensitivity reactions. (See Exp. Opin. Thera. Patents (1998) 8(5); hereby incorporated by reference). Specific adverse side-effects include neutropenia, alopecia, bradycardia, cardiac conduction defects, acute hypersensitivity reactions, neuropathy, mucositis, dermatitis, extravascular fluid accumulation, arthralgias, and myalgias. Various treatment regimens have been developed in an effort to minimize the side effects of taxane therapy, but adverse side effects remain the limiting factor in taxane therapy.

Taxane derivatives have been found to be useful in treating refractory ovarian carcinoma, urothelial cancer, breast carcinoma, melanoma, non-small-cell lung carcinoma, gastric, and colon carcinomas, squamous carcinoma of the head and neck, lymphoblastic, myeloblastic leukemia, and carcinoma of the esophagus.

Paclitaxel is typically administered in a 15-420 mg/m² dose over a 6 to 24 hour infusion. For renal cell carcinoma, squamous carcinoma of head and neck, carcinoma of esophagus, small and non-small cell lung cancer, and breast cancer, paclitaxel is typically administered as a 250 mg/m² 24 hour infusion every 3 weeks. For refractory ovarian cancer paclitaxel is typically dose escalated starting at 110 mg/m². Docetaxel is typically administered in a 60-100 mg/M² i.v. over 1 hour, every three weeks. It should be noted, however, that specific dose regimen depends upon dosing considerations based upon a variety of factors including the type of neoplasia; the stage of the neoplasm; the age, weight, sex, and medical condition of the patient; the route of administration; the renal and hepatic function of the patient; and the particular agents and combination employed.

In one embodiment, paclitaxel is used in the present invention in combination with an integrin antagonist and with cisplatin, cyclophosphamide, or doxorubicin for the treatment of breast cancer. In another embodiment paciltaxel is used in combination with an integrin antagonist, cisplatin or carboplatin, and ifosfamide for the treatment of ovarian cancer. In still another embodiment, a taxane such as paclitaxel is used in combination with a Cox-2 inhibitor and an EGF receptor antagonist.

In another embodiment, docetaxal is used in the present invention in combination with an integrin antagonist and in combination with cisplatin, cyclophosphamide, or doxorubicin for the treatment of ovary and breast cancer and for patients with locally advanced or metastatic breast cancer who have progressed during anthracycline based therapy.

U.S. Pat. No. 5,019,504 describes the isolation of paclitaxel and related alkaloids from culture grown Taxus brevifolia cells.

U.S. Pat. No. 5,675,025 describes methods for synthesis of Taxol®, Taxol® analogues and intermediates from baccatin III.

U.S. Pat. No. 5,688,977 describes the synthesis of Docetaxel from 10-deacetyl baccatin III.

U.S. Pat. No. 5,202,488 describes the conversion of partially purified taxane mixture to baccatin III.

U.S. Pat. No. 5,869,680 describes the process of preparing taxane derivatives.

U.S. Pat. No. 5,856,532 describes the process of the production of Taxol®.

U.S. Pat. No. 5,750,737 describes the method for paclitaxel synthesis.

U.S. Pat. No. 6,688,977 describes methods for docetaxel synthesis.

U.S. Pat. No. 5,677,462 describes the process of preparing taxane derivatives.

U.S. Pat. No. 5,594,157 describes the process of making Taxol® derivatives.

The phrase “retinoid” includes compounds, which are natural and synthetic analogues of retinol (Vitamin A). The retinoids bind to one or more retinoic acid receptors to initiate diverse processes such as reproduction, development, bone formation, cellular proliferation and differentiation, apoptosis, hematopoiesis, immune function and vision. Retinoids are required to maintain normal differentiation and proliferation of almost all cells and have been shown to reverse/suppress carcinogenesis in a variety of in vitro and in vivo experimental models of cancer, See Moon, et al., Ch. 14 Retinoids and cancer. In The Retinoids, Vol. 2. Academic Press, Inc. (1984) and Roberts, et al. Cellular biology and biochemistry of the retinoids. In The Retinoids, Vol. 2. Academic Press, Inc. 1984, both of which are hereby incorporated by reference, which also shows that vesanoid (tretinoid trans retinoic acid) is indicated for induction of remission in patients with acute promyelocytic leukemia (APL).

A synthetic description of retinoid compounds, hereby incorporated by reference, is described in: Dawson M I and Hobbs P D. The synthetic chemistry of retinoids: in The retinoids, 2^{nd} edition. M B Sporn, A B Roberts, and D S Goodman(eds). New York: Raven Press, 1994, pp 5-178.

Lingen et al. describe the use of retinoic acid and interferon alpha against head and neck squamous cell carcinoma (Lingen, M W, et al., Retinoic acid and interferon alpha act synergistically as antiangiogenic and antitumor agents against human head and neck squamous cell carcinoma. See Cancer Research 58(23):5551-5558 (1998), hereby incorporated by reference.)

lurlaro et al. describe the use of beta interferon and 13-cis retinoic acid to inhibit angiogenesis. (Lurlaro, M, et al., Beta interferon inhibits HIV-1 Tat-induced angiogenesis: synergism with 13-cis retinoic acid. European Journal of Cancer 34 (4):570-576 (1998), hereby incorporated by reference.)

Majewski, et al. describe Vitamin D3 and retinoids in the inhibition of tumor cell-induced angiogenesis. See Majewski, S, et al., Vitamin D3 is a potent inhibitor of tumor cell-induced angiogenesis. J. Invest. Dermatology. Symposium Proceedings, 1(1):97-101 (1996), hereby incorporated by reference.

Majewski et al. describe the role of retinoids and other factors in tumor angiogenesis. (Majewski, S, et al., Role of cytokines, retinoids and other factors in tumor angiogenesis. Central-European journal of Immunology 21(4):281-289 (1996), hereby incorporated by reference.)

Bollag describes retinoids and alpha-interferon in the prevention and treatment of neoplastic disease. (Bollag W. Retinoids and alpha-interferon in the prevention and treatment of preneoplastic and neoplastic diseases. Chemotherapie Journal, (Suppl) 5 (10):55-64 (1996), hereby incorporated by reference. )

Bigg, H F et al. describe all-trans retinoic acid with basic fibroblast growth factor and epidermal growth factor to stimulate tissue inhibitor of metalloproteinases from fibroblasts. (Bigg, H F, et al., All-trans-retoic acid interacts synergystically with basic fibroblast growth factor and epidermal growth factor to stimulate the production of tissue inhibitor of metalloproteinases from fibroblasts. Arch. Biochem. Biophys. 319(1):74-83 (1995), hereby incorporated by reference.)

Some preferred retinoids include Accutane, Adapalene, Allergan AGN-193174, Allergan AGN-193676, Allergan AGN-193836, Allergan AGN-193109, Aronex AR-623, BMS-181162, Galderma CD-437, Eisai ER-34617, Etrinate, Fenretinide, Ligand LGD-1550, lexacalcitol, Maxia Pharmaceuticals MX-781, mofarotene, Molecular Design MDI-101, Molecular Design MDI-301, Molecular Design MDI-403, Motretinide, Eisai 4-(2-[5-(4-methyl-7-ethylbenzofuran-2-yl)pyrrolyl]) benzoic acid, Johnson & Johnson N-[4-[2-thyl-1-(1H-imidazol-1-yl)butyl]phenyl]-2-benzothiazolamine, Soriatane, Roche SR-11262, Tocoretinate, Advanced Polymer Systems trans-retinoic acid, UAB Research Foundation UAB-8, Tazorac, TopiCare, Taiho TAC-101, and Vesanoid.

cGMP phosphodiesterase inhibitors, including Sulindac sulfone (Exisuland®) and CP-461 for example, are apoptosis inducers and do not inhibit the cyclooxygenase pathways. cGMP phosphodiesterase inhibitors increase apoptosis in tumor cells without arresting the normal cycle of cell division or altering the cell's expression of the p53 gene.

Ornithine decarboxylase is a key enzyme in the polyamine synthesis pathway that is elevated in most tumors and premalignant lesions. Induction of cell growth and proliferation is associated with dramatic increases in ornithine decarboxylase activity and subsequent polyamine synthesis. Further, blocking the formation of polyamines slows or arrests growth in transformed cells. Consequently, polyamines are thought to play a role in tumor growth. Difluoromethylornithine (DFMO) is a potent inhibitor of ornithine decarboxylase that has been shown to inhibit carcinogen-induced cancer development in a variety of rodent models (Meyskens, et al., Development of Difluoromethylornithine (DFMO) as a chemoprevention agent. Clin. Cancer Res. 1999 May, 5(%):945-951, hereby incorporated by reference, herein). DFMO is also known as 2-difluoromethyl-2,5-diaminopentanoic acid, or 2-difluoromethyl-2,5-diaminovaleric acid, or a-(difluoromethyl) ornithine; DFMO is marketed under the tradename Elfornithine®. Therefore, the use of DFMO in combination with Cox-2 inhibitors is contemplated to treat or prevent cancer, including but not limited to colon cancer or colonic polyps.

Populations with high levels of dietary calcium have been reported to be protected from colon cancer. In vivo, calcium carbonate has been shown to inhibit colon cancer via a mechanism of action independent from Cox-2 inhibition. Further, calcium carbonate is well tolerated. A combination therapy including an integrin antagonist, calcium carbonate and a selective Cox-2 inhibitor is contemplated to treat or prevent cancer, including but not limited to colon cancer or colonic polyps.

Several studies have focused attention on bile acids as a potential mediator of the dietary influence on colorectal cancer risk. Bile acids are important detergents for fat solubilization and digestion in the proximal intestine. Specific transprot processes in the apical domain of the terminal ileal enterocyte and basolateral domain of the hepatocyte account for the efficient conservation in the enterohepatic circulation. Only a small fraction of bile acids enter the colon; however, perturbations of the cycling rate of bile acids by diet (e.g. fat) or surgery may increase the fecal bile load and perhaps account for the associated increased risk of colon cancer. (Hill, M J, Bile flow and colon cancer. 238 Mutation Review, 313 (1990).) Ursodeoxycholate (URSO), the hydrophilic 7-beta epimer of chenodeoxycholate, is non-cytotoxic in a variety of cell model systems including colonic epithelia. URSO is also virtually free of side effects. URSO, at doses of 15 mg/kg/day used primarily in biliary cirrhosis trials were extremely well tolerated and without toxicity. (Pourpon, et al., A multicenter, controlled trial of ursodiol for the treatment of primary biliary cirrhosis. 324 New Engl. J. Med. 1548 (1991)). While the precise mechanism of URSO action is unknown, beneficial effects of URSO therapy are related to the enrichment of the hepatic bile acid pool with this hydrophilic bile acid. It has thus been hypothesized that bile acids more hydrophilic than URSO will have even greater beneficial effects than URSO. For example, tauroursodeoxycholate (TURSO) the taurine conjugate of URSO. Non-steroidal anti-inflammatory drugs (NSAIDs) can inhibit the neoplastic transformation of colorectal epithelium. The likely mechanism to explain this chemopreventive effect is inhibition of prostaglandin synthesis. NSAIDs inhibit cyclooxygenase, the enzyme that converts arachidonic acid to prostaglandins and thromboxanes. However, the potential chemopreventive benefits of NSAIDs such as sulindac or mesalamine are tempered by their well-known toxicities and moderately high risk of intolerance. Abdominal pain, dispepsia, nausea, diarrhea, constipation, rash, dizziness, or headaches have been reported in up to 9% of patients. The elderly appear to be particularly vulnerable as the incidence of NSAID-induced gastroduodenal ulcer disease, including gastrointestinal bleeding, is higher in those over the age of 60; this is also the age group most likely to develop colon cancer, and therefore most likely to benefit from chemoprevention. The gastrointestinal side effects associated with NSAID use result from the inhibition of cyclooxygenase-1, an enzyme responsible for maintenance of the gastric mucosa. Therefore, the use of Cox-2 inhibitors in combination with URSO is contemplated to treat or prevent cancer, including but not limited to colon cancer or colonic polyps; it is contemplated that this treatment will result in lower gastrointestinal side effects than the combination of standard NSAIDs and URSO.

An additional class of antineoplastic agents that may be used in the present invention include nonsteroidal antiinflammatory drugs (NSAIDs). NSAIDs have been found to prevent the production of prostaglandins by inhibiting enzymes in the human arachidonic acid/prostaglandin pathway, including the enzyme cyclooxygenase (Cox). However, for the purposes of the present invention the definition of an NSAID does not include the “Cox-2 inhibitors” described herein. Thus the phrase “nonsteroidal antiinflammatory drug” or “NSAID” includes agents that specifically inhibit Cox-1, without significant inhibition of Cox-2; or inhibit Cox-1 and Cox-2 at substantially the same potency; or inhibit neither Cox-1 or Cox-2. The potency and selectivity for the enzyme Cox-1 and Cox-2 can be determined by assays well known in the art, See for example, Cromlish and Kennedy, Biochemical Pharmacology, Vol. 52, pp 1777-1785 (1996).

Examples of NSAIDs that can be used in the combinations of the present invention include ibuprofen, naproxen, benoxaprofen, flurbiprofen, fenoprofen, fenbufen, ketoprofen, indoprofen, pirprofen, carprofen, oxaprozin, prapoprofen, miroprofen, tioxaprofen, suprofen, alminoprofen, tiaprofenic acid, fluprofen, bucloxic acid, indomethacin, sulindac, tolmetin, zomepirac, diclofenac, fenclofenec, alclofenac, ibufenac, isoxepac, furofenac, tiopinac, zidometacin, acetyl salicylic acid, indometacin, piroxicam, tenoxicam, nabumetone, ketorolac, azapropazone, mefenamic acid, tolfenamic acid, diflunisal, podophyllotoxin derivatives, acemetacin, droxicam, floctafenine, oxyphenbutazone, phenylbutazone, proglumetacin, acemetacin, fentiazac, clidanac, oxipinac, mefenamic acid, meclofenamic acid, flufenamic acid, niflumic acid, flufenisal, sudoxicam, etodolac, piprofen, salicylic acid, choline magnesium trisalicylate, salicylate, benorylate, fentiazac, clopinac, feprazone, isoxicam and 2-fluoro-a-methyl[1,1′-biphenyl]-4-acetic acid, 4-(nitrooxy)butyl ester.

Another component of the present invention is an antineoplastic agent such as an EGF receptor antagonist. In one embodiment of the present invention, an EGF antagonist is administered in combination with an antiangiogenesis agent such as a Cox-2 inhibitor to a subject that is in need of the prevention or treatment of a neoplasia disorder.

By “epidermal growth factor receptor” or “EGFR” or “EGF receptor” is meant a protein a portion thereof capable of binding the EGF ligand or protein or a portion thereof. Exemplary is the human epidermal growth factor receptor (Ullrich, et al., Nature 309:418-425 (1984); Genbank accession number NM_{—}005228). Preferably, the binding of the EGF ligand activates the EGF receptor (e.g. resulting in activation of intracellular mitogenic signaling, autophosphorylation of EGFR). One of skill in the art will appreciate that other ligands, in addition to EGF, may bind to the EGF receptor and activate the EGF receptor. Examples of such ligands include, but are not limited to, TGF-.alpha., betacellulin, amphiregulin, heparin-binding EGF (HB-EGF) and neuregulin (also known as hergulin) (Strawn and Shawver (1998) Exp. -Opin. Invest Drugs 7(4)553-573, and “The Protein Kinase Facts Book: Protein Tyrosine Kinases” (1995) Hardie, et al. (eds.), Academic Press, NY, N.Y.).

By “EGFR antagonist” or “EGF receptor antagonist” is meant any agent capable of directly or indirectly inhibiting the effect of the EGF receptor, particularly the effect of the EGF receptor on neoplasia or neoplasm growth and proliferation. The EGF receptor can be activated through ligand-dependent and ligand-independent mechanisms, resulting in either autophosphorylation or trans-phosphorylation, respectively. EGF receptor antagonists of interest may inhibit either or both of these mechanisms. For example, binding of TNF-.alpha. to the EGF receptor results in a ligand-dependent phosphorylation, which may be blocked by an antibody that binds EGF receptor, thereby preventing the interaction of the EGF receptor with a ligand that would activate the EGF receptor. Examples of such antibodies are described by Goldstein, et al., Clin. Cancer Res. 1:1311-1318 (1995); Lorimer, et al., Clin. Cancer Res. 1:859-864 (1995); Schmidt and Wels, Br. J. Cancer 74:853-862 (1996). Small molecule tyrosine kinase inhibitors are also effective as EGF receptor antagonists.

The EGF receptor antagonist administered in the therapeutic method may be in any form. By way of example, the EGF receptor antagonist may be in the form of a small molecule (i.e., antisense oligonucleotide, tyrosine kinase inhibitor, EGFR inhbitor, etc.), antibodies or portion of antibodies that bind to the EGF ligand or the EGF receptor.

Tyrosine kinase inhibitors that act on the EGF receptor, and that are selective for the EGF receptor, are known in the art, and may be used in the subject methods and compositions. Examples are described above, and of such may include BIBX1522 (Boehringer Ingelheim, Inc., Ingelheim, Germany); CGP59326B (Novartis Corporation, Basel, Switzerland); 4-aminoquinazoline EGF receptor inhibitors (described in U.S. Pat. No. 5,760,041); substituted styrene compounds which can also be a naphthalene, an indane or a benzoxazine; including nitrile and molononitrile compounds (described in U.S. Pat. No. 5,217,999); the inhibitors disclosed in U.S. Pat. No. 5,773,476; potato carboxypeptidase inhibitor (PCI), a 39-amino acid protease inhibitor with three disulfide bridges, (Blanco-Aparicio, et al., J. Biol Chem 273(20):12370-12377, 1998); bombesin antagonist RC-3095 (Szepeshazi, et al., Proc Natl Acad Sci USA 94:10913-10918,1997) etc. Other tyrosine kinase inhibitors include quinazolines, such as PD 153035, 4-(3-chloroanilino)quinazoline, or CP-358,774, pyridopyrimidines, pyrimidopyrimidines, pyrrolopyrimidines, such as CGP 59326, CGP 60261 and CGP 62706, and pyrazolopyrimidines (Shawn and Shawver, supra.), 4-(phenylamino)-7H-pyrrolo[2,3-d]pyrimidines (Traxler, et al., J. Med. Chem 39:2285-2292, 1996), curcumin (Korutia, et al., Biochim Biophys Acta 1224:597-600, 1994); (Laxmin arayana, Carcinogen 16:1741-1745,1995); etc.

Preferred tyrosine kinase inhibitors are selective for the EGF receptor, i.e. the EGF receptor is inhibited to a greater degree than other cell surface receptors having tyrosine kinase activity. In one embodiment, the EGF receptor antagonist is an inhibitor of the tyrosine kinase activity of the EGF receptor, particularly small molecule inhibitors having selective action on the EGF receptor as compared to other tyrosine kinases—preferred small molecules block the natural EGF receptor in a mammal, and preferably a human.

Inhibitors of EGF and the EGF receptor include, but are not limited to, tyrosine kinase inhibitors such as quinazolines, such as PD 153035, 4-(3-chloroanilino) quinazoline, or CP-358,774, pyridopyrimidines, pyrimidopyrimidines, pyrrolopyrimidines, such as CGP 59326, CGP 60261 and CGP 62706, and pyrazolopyrimidines (Shawn and Shawver, supra.), 4-(phenylamino)-7H-pyrrolo[2,3-d]pyrimidines (Traxler, et al., J. Med. Chem 39:2285-2292, 1996), curcumin (diferuloyl methane) (Laxmin, arayana, et al., Carcinogen 16:1741-1745, 1995), 4,5-bis (4-fluoroanilino)phthalimide (Buchdunger, et al., Clin. Cancer Res. 1:813-821,1995; Dinney, et al., Clin. Cancer Res. 3:161-168,1997); tyrphostins containing nitrothiophene moieties (Brunton, et al., Anti Cancer Drug Design 11:265-295, 1996); the protein kinase inhibitor ZD-1839 (AstraZeneca); CP-358774 (Pfizer, inc.); PD-0183805 (Warner-Lambert); or as described in International patent application WO99/09016 (American Cyanamid); WO98/43960 (American Cyanamid); WO97/38983 (Warener Labert); WO99/06378 (Warner Lambert); WO99/06396 (Warner Lambert); WO96/30347 (Pfizer, Inc.); WO96/33978 (Zeneca); WO96/33977 (Zeneca); and WO96/33980) Zeneca; all herein incorporated by reference; or antisense molecules.

The present invention encompasses those antineoplastic agents that are EGF receptor antagonists mentioned above, and in addition, those antineoplastic agents that are EGF receptor antagonists or that can function as EGF receptor antagonists, which are described in Tables 10, 11 and 12 below.

TABLE 10
Antineoplastic Agents

| | Trade | | Mode of | | | | Oncology |
|---|---|---|---|---|---|---|---|
| Compound | Name | Company | Action | Reference | Dosage | Toxicity | Indication |
| Butanedioic acid, | reveromycin | Snow | Epidermal | EP 491956, | | | anticancer, |
| mono[3-butyl-8-(9- | A | Brand | growth factor | J. Antibiot, | | | antibiotic |
| carboxy-6-hydroxy- | | | antagonist | 1991, 44, | | | |
| 3,7-dimethyl-2,4,8- | | | | 259 | | | |
| nonatrienyl)-2-(4- | | | | | | | |
| carboxy-3-methyl- | | | | | | | |
| 1,3-butadienyl)-9- | | | | | | | |
| methyl-1,7- | | | | | | | |
| dioxaspiro[5.5]undec- | | | | | | | |
| 3-yl] ester | | | | | | | |
| Carbamic acid, | AGM-1470; | Takeda | Angiogenesis | EP 359036, | | tolerated @ | anticancer, |
| (chloroacetyl)-,5- | TNP-470; | | inhibitor; | Clin Cancer | | 60 mg/m2 iv | antibiotic, |
| methoxy-4-[2-methyl- | | | Endothelial | Res, 1997, | | over 60 min | symptomatic |
| 3-(3-methyl-2- | | | growth factor | 3, 1501 | | this is the | anti-diabetic, |
| butenyl)oxiranyl]-1- | | | antagonist; | | | recommended | ophthalmological, |
| oxaspiro[2.5]oct-6-yl | | | Fibroblast | | | dose for | anti- |
| ester, [3R- | | | growth factor | | | Phase II | psoriasis, |
| [3alpha,4alpha(2R*, | antagonist | studies | anti-arthritic | | | | |
| 3R*),5beta,6beta]]- | | | | | | | |
| | PD-171026; | Warner- | Tyrosine | | | | |
| | PD-089828; | Lambert | kinase | | | | |
| | PD-090560; | | inhibitor | | | | |
| 6-amino-4-(3- | ZM-254530; | Zeneca | Epidermal | | | | |
| methylphenylamino)- | ZM-105180; | | growth factor | | | | |
| quinazoline | | | receptor | | | | |
| | | | kinase | | | | |
| | | | inhibitor; | | | | |
| | | | Endothelial | | | | |
| | | | growth factor | | | | |
| | | | antagonist; | | | | |
| | | | Tyrosine | | | | |
| | | | kinase | | | | |
| | | | inhibitor | | | | |
| Cinnoline Derivatives | | Zeneca | Tyrosine | WO | | | |
| WO 097/34876 | | Group Plc | kinase | 97/34876, | | | |
| | | | inhibitor, | EP 050722, | | | |
| | | | VEGF | EP | | | |
| | | | antagonist | 0566226, | | | |
| | | | | EP | | | |
| | | | | 0602851, | | | |
| | | | | EP | | | |
| | | | | 0635498 | | | |
| | EGF- | | Epidermal | | | | |
| | genistein, | | growth factor | | | | |
| | Wayne | | antagonist; | | | | |
| | | | Tyrosine | | | | |
| | | | kinase | | | | |
| | | | inhibitor | | | | |
| | anti-EGER | ImClone | Epidermal | | | | |
| | Mabs; C225; | Systems | growth factor | | | | |
| | MAb C225 | | receptor | | | | |
| | | | kinase | | | | |
| | | | inhibitor | | | | |
| | anti-VEGF | Genen-tech | Endothelial | | | | |
| | mono-clonal, | | growth factor | | | | |
| | Genen | | antagonist; | | | | |
| | | | Angiogenesis | | | | |
| | | | | inhibitor | | | |
| | EMD-72000; | Merck | Epidermal | | | | |
| | anti-EGFR | KGaA | growth factor | | | | |
| | Mab; EMD- | | receptor | | | | |
| | 6200 | | kinase | | | | |
| | | | inhibitor | | | | |
| | MDX-447; | Medarex | Epidermal | | | | |
| | BAB-447; | | growth factor | | | | |
| | EMD-82633; | | antagonist; | | | | |
| | H-447 | | CD8 agonist | | | | |
| | ABX-EGF | Cell | Epidermal | | | | |
| | anti-EGFr | Genesys | growth factor | | | | |
| | MAb, | | antagonist | | | | |
| | Abgenix; | | | | | | |
| | anti-EG Fr | | | | | | |
| | MAb, Cell | | | | | | |
| | Genesys | | | | | | |
| | anti-EGFR- | ImmunoGen | Epidermal | | | | |
| | DM1 Ab, | | growth factor | | | | |
| | ImmunoGen | | agonist; | | | | |
| | anti-EGFR | | Epidermal | | | | |
| | conjugate, | | growth factor | | | | |
| | ImmunoGen; | | antagonist | | | | |
| | EGFR | | | | | | |
| | conjugate, | | | | | | |
| | ImmunoGen | | | | | | |
| | anti-flk-1 | ImClone | Endothelial | U.S. Pat. No. | | | |
| | MAb, | Systems | growth factor | 5747651 | | | |
| | ImClone DC- | | antagonist; | | | | |
| | 101 | | Tyrosine | | | | |
| | | | kinase | | | | |
| | | | inhibitor; | | | | |
| | | | Angiogenesis | | | | |
| | | | inhibitor | | | | |
| | CELLCOM | Cortecs | MAP kinase | | | | |
| | molecules, | | inhibitor; | | | | |
| | Cortecs | | Tyrosine | | | | |
| | bromelain | | kinase | | | | |
| | molecules, | | inhibitor; T | | | | |
| | Cortecs; | | cell inhibitor | | | | |
| | CCX, | | | | | | |
| | Cortecs; | | | | | | |
| | CCZ, Cortecs | | | | | | |
| Erlotinib (OSI-774) 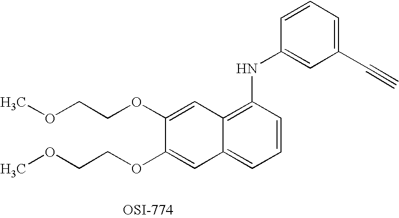 | Tarceva ™ | Genentech; Hoffmann- La Roche; OSI Pharmaceuticals | EGF receptor inhibitor; small molecule tyrosine kinase inhibitor; quinazoline structural class | Kim, T., et al., Curr. Opin. Investig. Drugs. 3(9): 1385-95 (2002). | Phase II dose of 150 mg/day | generally well tolerated at the Phase II dose with | |
| #a generally reversible acneiform rash and occasional diarrhea that responds to therapy being the most common side-effects reported to date | variety of cancers including ovarian, pancreatic, nonsmall cell lung, breast and head and neck | | | | | | |
| Tecogalan sodium | | | Inhibition of | | 390 | | |
| (sulfated | | | binding of | | mg/m² | | |
| polysaccharide from | | | bFGF to its | | infusion | | |
| Arthrobacter) | | | receptor. | | for 24 hours | | |
| | | | Inhibits | | every 3 weeks. | | |
| | | | angiogenesis | | | | |
| Platelet factor 4 | | | Blocks | J Natl | | | |
| | | | response of | Cancer Inst | | | |
| | | | endothelial | 1995, | | | |
| | | | cell to bFGF; | 87: 304-309 | | | |
| | | | antiangiogenic | | | | |
| | | | properties. | | | | |
| Inhibitors of vascular | | | Endothelial | Nature | | | |
| endothelial growth | | | cell-specific | 1993, | | | |
| factor antagonist | | | mitogen | 362: 841- | | | |
| (VEGF) and its | | | | 844; J Clin | | | |
| receptor flk-1 | | | | Invest | | | |
| | | | | 1995: 1789-1797. | | | |
| | anti-EGFR | York | Epidermal | | | | |
| | MAb, York | Medical | growth factor | | | | |
| | Medical anti- | | antagonist | | | | |
| | EGFR MAb, | | | | | | |
| | CIMYM; | | | | | | |
| | DiaCIM; ior | | | | | | |
| | egfr/r3; | | | | | | |
| | TheraCIM | | | | | | |
| | BIBX-1382 | Boehringer | Epidermal | | | | |
| | | Ingeiheim | growth factor | | | | |
| | | | receptor | | | | |
| | | | kinase inhibitor | | | | |
| Benzenesulfonamide, | celecoxib | Monsanto | Cyclooxygense 2 | | | | |
| 4-(5-(4- | Celebra; SC- | | inhibitor | | | | |
| methylphenyl)-3- | 58635; YM- | | | | | | |
| (trifluoromethyl)-1H- | 177 | | | | | | |
| pyrazol-1-yl)- | | | | | | | |
| | CEP-2563 | Cephalon | Tyrosine | | | | |
| | dihydrochloride | Incorporate | kinase | | | | |
| | CEP- | | inhibitor | | | | |
| | 2563; CEP- | | | | | | |
| | 701; CEP- | | | | | | |
| | 751; KT-8391 | | | | | | |
| 4-(3- | CP-358774 | OSI | Epidermal | | | | |
| Ethynylphenylamino)- | EFGR | Pharmaceuticals | growth factor | | | | |
| 6,7-bis(2- | inhibitor, OSI; | | receptor | | | | |
| methoxyethoxy)- | EFGR | | kinase | | | | |
| quinazoline | inhibitor, | | inhibitor | | | | |
| hydrochloride | Pfizer; | | | | | | |
| | tumour growth | | | | | | |
| | inhibitors, | | | | | | |
| | OSI, tumour | | | | | | |
| | growth inhibitors, | | | | | | |
| | Pfiz | | | | | | |
| | growth factor | Institute for | Epidermal | | | | |
| | complex, IPR | Pharmaceutical | growth factor | | | | |
| | GFC, IPR | Research | agonist; | | | | |
| | | | Transforming | | | | |
| | | | growth factor | | | | |
| | | | alpha agonist; | | | | |
| | | | Transforming | | | | |
| | | | growth factor | | | | |
| | | | beta agonist | | | | |
| | PN-355 | Paracelsian | Tyrosine | | | | |
| | AndroVir; | | kinase | | | | |
| | AndroVir-DS | | inhibitor | | | | |
| | ZD-1838 | Zeneca | Epidermal | | | | |
| | | | growth factor | | | | |
| | | | receptor kinase | | | | |
| | | | inhibitor | | | | |
| 4-(3-Chloro-4- | Iressa ® | Zeneca | Epidermal growth | U.S. Pat. No. | 250-500 | | |
| fluorophenylamino)- | | | factor receptor | 5,616,582; | mg daily. | | |
| 7-methoxy-6-(3-(4- | | | kinase inhibitor; | U.S. Pat. No. | | | |
| morpholinyl)propoxy) | | | quinazoline structural | 5,457,105 | | | |
| quinazoline (ZD-1839 | | | class | | | | |
| 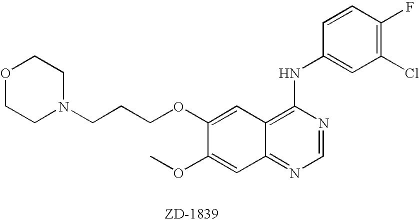 | | | | | | | |
| 4-(4-methylpiperazin- | CGP-57148 | Novartis | Tyrosine | | | | |
| 1-ylmethyl)-N-[4- | | | kinase | | | | |
| methyl-3-[4-(3- | | | inhibitor; | | | | |
| pyridyl)pyrimidin-2- | | | Platelet | | | | |
| ylamino] | | | growth factor | | | | |
| phenyl]benzamide | | | antagonist | | | | |
| | CGP-59326; | Novartis | Epidermal | | | | |
| | CGP- | | growth factor | | | | |
| | 59326B; | | receptor | | | | |
| | CGP-62706; | | kinase | | | | |
| | CGP-74321; | | inhibitor | | | | |
| | CGP-75166; | | | | | | |
| | CGP-76627 | | | | | | |
| | CGP-79787 | Novartis | Epidermal | | | | |
| | | | growth factor | | | | |
| | | | receptor | | | | |
| | | | kinase inhibitor | | | | |
| | DWP-408 | Daewoong | Epidermal | | | | |
| | | | growth factor | | | | |
| | | | agonist | | | | |
| | EGF- | | Epidermal | | | | |
| | genistein, | | growth factor | | | | |
| | Wayne | | antagonist; | | | | |
| | genistein, | | Tyrosine | | | | |
| | Wayne | | kinase | | | | |
| | | | inhibitor | | | | |
| Muellerian-inhibiting | mullerian | | Epidermal | | | | |
| hormone | inhibiting | | growth factor | | | | |
| | subst, Ma | | receptor | | | | |
| MIS, | | kinase | | | | | |
| | Massachusetts | | inhibitor | | | | |
| 4-(m-chloro)-5,6- | | Novartis | Epidermal | EP 682027 | | | |
| dimethyl-7H- | | | growth factor | | | | |
| pyrrolo[2,3- | | | receptor | | | | |
| d]pyrimidine | | | kinase | | | | |
| | | | inhibitor; | | | | |
| | | | Tyrosine | | | | |
| | | | kinase inhibitor | | | | |
| 5-[3-[3-methoxy-4-[2- | | Takeda | Tyrosine | WO | | | |
| [(E)-2-phenylethenyl]-4- | | | kinase | 9700249 | | | |
| oxazolylmethoxyl]phenyl] | | | inhibitor | | | | |
| propyl]-3-[2-[(E)- | | | | | | | |
| 2-phenylethenyl]-4- | | | | | | | |
| oxazolylmethyl]-2,4- | | | | | | | |
| oxazolidinedione | | | | | | | |
| N-(6-Benzothiazolyl)- | | Celltech | ZAP-70 | WO | | | |
| 4-(2-(1 - | | | protein | 9811095 | | | |
| piperazinyl)pyrid-5- | | | tyrosine | | | | |
| yl)-2-pyrimidineamine | | | kinase | | | | |
| | | | inhibitor; | | | | |
| | | | Protein | | | | |
| | | | kinase C | | | | |
| | | | inhibitor | | | | |
| | PI-88 | Progen | Heparanase | | | | |
| | | | inhibitor; | | | | |
| | | | Fibroblast | | | | |
| | | | growth factor | | | | |
| | | | antagonist; | | | | |
| | | | Epidermal | | | | |
| | | | growth factor | | | | |
| | | | antagonist | | | | |
| | PJ3505 | PowderJect | | | | | |
| | | Pharmaceuticals | | | | | |
| | S-96-8045 | Hoechst | Endothelial | | | | |
| | antisense | Marion | growth factor | | | | |
| | oligonucleotides, | Roussel | antagonist; | | | | |
| | Ho; VEGF | | Protein synthesis | | | | |
| | oligonucleotides, | | antagonist; | | | | |
| | Hoechst | | RNA antagonist | | | | |
| | VEGF | Chugai | Endothelial | WO | | | |
| | inhibitor, | | growth factor | 9803663 | | | |
| | Chugai | | antagonist | | | | |
| 4-(4-chloro-2-fluoro- | VEGF | Zeneca | Endothelial | WO | | | |
| 5-hydroxyanilino)-6- | inhibitors, | | growth factor | 9734876 | | | |
| methoxy-7-(2- | Zeneca | | antagonist | | | | |
| methoxyethoxy)cinnoline | | | | | | | |
| N-[4-[(3-chloro-4- | | Pfizer | tyrosine | | well | No signs of | Suppresses |
| fluorophenyl) amino]- | | | kinase | | tolerated | toxicity | tumor growth |
| 7-[3-(4-morpholinyl) | | | inhibitor; | | at doses | | |
| propoxy]-6- | | | EGF receptor | | of 50-650 | | |
| quinazolinyl]-2- | | | inhibitor | | mg/d | | |
| propenamide | | | | | | | |
| EKB-569 | | Wyeth- | Irreversible | | | | |
| | | Ayerst | EGFR | | | | |
| | | Pharmaceuticals | tyrosine | | | | |
| | | kinase inhibitor | | | | | |
| PTK787 | | Novartis | Inhibits | | | | Advanced |
| | | Pharmaceuticals | vascualar | | | | cancers |
| | | | endothelial GFR, | | | | |
| | | | tyrosine kinases; | | | | |
| | | | impairs vascular | | | | |
| | | | endothelial | | | | |
| | | | growth factor- | | | | |
| | | | induced responses | | | | |
| | | | and tumor growth | | | | |
| HER-2/neu protein | Herceptin | UCLA; | Humanized | | | | non- |
| antibody | | Genetech | Mab against | | | | Hodgkin's |
| | | | Her-2 growth | | | | lymphoma, |
| | | | factor receptor | | | | breast and |
| | | | | | | | colon cancer, |
| | | | | | | | and melanoma |
| EGF receptor | | M.D. | | | | | |
| antibody | | Anderson | | | | | |
| | | Cancer | | | | | |
| | | Center in | | | | | |
| | | Houston | | | | | |
| Trastuzumab | Herceptin ® | NCl; | HER-2 | U.S. Pat. No. | | | Breast, |
| | | Genentech | blocker; | 6,165,464 | | | colon, |
| | | | epidermal | | | | bladder, lung, |
| | | | growth factor | | | | pancreatic |
| | | | inhibitor, | | | | cancers |
| | | | antibody | | | | |
| Bevacizumab; anti- | | Genentech; | Monoclonal | | | | |
| VEGF humanized | | National | antibody; | | | | |
| monoclonal antibody | | Cancer | neutralizes | | | | |
| | | Institute | the vascular | | | | |
| | | | endothelial | | | | |
| | | | growth factor | | | | |
| | | | (VEGF) protein; | | | | |
| | | | inhibits tumor | | | | |
| | | | growth | | | | |
| Butanedioic acid, | reveromycin | Snow | Epidermal | EP 491956, | | | anticancer, |
| mono[3-butyl-8-(9- | A | Brand | growth factor | J. Antibiot, | | | antibiotic |
| carboxy-6-hydroxy- | | | antagonist | 1991, 44, | | | |
| 3,7-dimethyl-2,4,8- | | | | 259 | | | |
| nonatrienyl)-2-(4- | | | | | | | |
| carboxy-3-methyl- | | | | | | | |
| 1,3-butadienyl)-9- | | | | | | | |
| methyl-1,7- | | | | | | | |
| dioxaspiro[5.5]undec- | | | | | | | |
| 3-yl]ester | | | | | | | |
| Carbamic acid, | AGM-1470; | Takeda | Angiogenesis | EP 359036, | | tolerated @ | anticancer, |
| (chloroacetyl)-,5- | TNP-470; | | inhibitor; | Clin Cancer | | 60 mg/m2 iv | antibiotic, |
| methoxy-4-[2-methyl- | | | Endothelial | Res, 1997, | | over 60 min | symptomatic |
| 3-(3-methyl-2- | | | growth factor | 3, 1501 | | this is the | anti-diabetic |
| butenyl)oxiranyl]-1- | | | antagonist; | | | recommended | ophthalmological, |
| oxaspiro[2.5]oct-6-yl | | | Fibroblast | | | dose for | anti- |
| ester, [3R- | | | growth factor | | | Phase II | psoriasis, |
| [3alpha, 4alpha(2R*, | | | antagonist | | | studies | anti-arthritic |
| 3R*), 5beta, 6beta]]- | | | | | | | |
| | PD-171026; | Warner- | Tyrosine | | | | |
| | PD-089828; | Lambert | kinase | | | | |
| | PD-090560; | | inhibitor | | | | |
| 6-amino-4-(3- | ZM-254530; | Zeneca | Epidermal | | | | |
| methylphenylamino)- | ZM-105180; | | growth factor | | | | |
| quinazoline | | | receptor | | | | |
| | | | kinase inhibitor; | | | | |
| | | | Endothelial | | | | |
| | | | growth factor | | | | |
| | | | antagonist; | | | | |
| | | | Tyrosine kinase | | | | |
| | | | inhibitor | | | | |
| Cinnoline Derivatives | | Zeneca | Tyrosine | WO | | | |
| WO 097/34876 | | Group Plc | kinase | 97/34876, | | | |
| | | | inhibitor, | EP 050722, | | | |
| | | | VEGF | EP 0566226, | | | |
| | | | antagonist | EP 0602851, | | | |
| | | | | EP 0635498 | | | |
| | EGF- | | Epidermal | | | | |
| | genistein, | | growth factor | | | | |
| | Wayne | | antagonist; | | | | |
| | | | Tyrosine | | | | |
| | | | kinase | | | | |
| | | | inhibitor | | | | |
| | anti-EGFR | ImClone | Epidermal | | | | |
| | Mabs; C225; | Systems | growth factor | | | | |
| | MAb C225 | | receptor | | | | |
| | | | kinase inhibitor | | | | |
| | anti-VEGF | Genen-tech | Endothelial | | | | |
| | mono-clonal, | | growth factor | | | | |
| | Genen | | antagonist; | | | | |
| | | | Angiogenesis | | | | |
| | | | inhibitor | | | | |
| | EMD-72000; | Epidermal | | | | | |
| | Merck anti- | KGaA | growth factor | | | | |
| | EGFR Mab; | | receptor | | | | |
| | EMD-6200 | | kinase inhibitor | | | | |
| | MDX-447; | Medarex | Epidermal | | | | |
| | BAB-447; | | growth factor | | | | |
| | EMD-82633; | | antagonist; | | | | |
| | H-447 | | CD8 agonist | | | | |
| | ABX-EGF | Cell | Epidermal | | | | |
| | anti-EGFr | Genesys | growth factor | | | | |
| | MAb, | | antagonist | | | | |
| | Abgenix; | | | | | | |
| | anti-EGFr | | | | | | |
| | MAb, Cell | | | | | | |
| | Genesys | | | | | | |
| | anti-EGFR- | ImmunoGen | Epidermal | | | | |
| | DM1 Ab, | | growth factor | | | | |
| | ImmunoGen | | agonist; | | | | |
| | anti-EGFR | | Epidermal | | | | |
| | conjugate, | | growth factor | | | | |
| | ImmunoGen; | | antagonist | | | | |
| | EGFR | | | | | | |
| | conjugate, | | | | | | |
| | ImmunoGen | | | | | | |
| | anti-flk-1 | ImClone | Endothelial | U.S. Pat. No. | | | |
| | MAb, | Systems | growth factor | 5747651 | | | |
| | ImClone DC- | | antagonist; | | | | |
| | 101 | | Tyrosine | | | | |
| | | | kinase | | | | |
| | | | inhibitor; | | | | |
| | | | Angiogenesis | | | | |
| | | | inhibitor | | | | |
| | CELLCOM | Cortecs | MAP kinase | | | | |
| | molecules, | | inhibitor; | | | | |
| | Cortecs | | Tyrosine | | | | |
| | bromelain | | kinase | | | | |
| | molecules, | | inhibitor; T | | | | |
| | Cortecs; | | cell inhibitor | | | | |
| | CCX, | | | | | | |
| | Cortecs; | | | | | | |
| | CCZ, Cortecs | | | | | | |
| Tecogalan sodium | | | Inhibition of | | 390 | | |
| (sulfated | | | binding of | | mg/m² | | |
| polysaccharide from | | | bFGF to its | | infusion | | |
| Arthrobacter) | | | receptor. | | for 24 | | |
| | | | Inhibits | | hours | | |
| | | | angiogenesis | | every 3 weeks. | | |
| Platelet factor 4 | | | Blocks | J Natl | | | |
| | | | response of | Cancer Inst | | | |
| | | | endothelial | 1995, | | | |
| | | | cell to bFGF; | 87: 304-309 | | | |
| | | | antiangiogenic | | | | |
| | | | properties. | | | | |
| Inhibitors of vascular | | | Endothelial | Nature | | | |
| endothelial growth | | | cell-specific | 1993, | | | |
| factor antagonist | | | mitogen | 362: 841- | | | |
| (VEGF) and its | | | | 844; J Clin | | | |
| receptor flk-1 | | | | Invest | | | |
| | | | | 1995: 1789-1797. | | | |
| | anti-EGFR | York | Epidermal | | | | |
| | MAb, York | Medical | growth factor | | | | |
| | Medical anti- | | antagonist | | | | |
| | EGFR MAb, | | | | | | |
| | CIMYM; | | | | | | |
| | DiaCIM; ior | | | | | | |
| | egfr/r3; | | | | | | |
| | TheraCiM | | | | | | |
| | BIBX-1382 | Boehringer | Epidermal | | | | |
| | | Ingelheim | growth factor | | | | |
| | | | receptor | | | | |
| | | | kinase inhibitor | | | | |
| Benzenesulfonamide, | celecoxib | Monsanto | Cyclooxygenase 2 | | | | |
| 4-(5-(4- | Celebra; SC- | | inhibitor | | | | |
| methylphenyl)-3- | 58635; YM- | | | | | | |
| (trifluoromethyl)-1H- | 177 | | | | | | |
| pyrazol-1-yl)- | | | | | | | |
| | CEP-2563 | Cephalon | Tyrosine | | | | |
| | dihydrochioride | Incorporate | kinase | | | | |
| | CEP- | | inhibitor | | | | |
| | 2563; CEP- | | | | | | |
| | 701; CEP- | | | | | | |
| | 751; KT-8391 | | | | | | |
| 4-(3- | CP-358774 | OSI | Epidermal | | | | |
| Ethynylphenylamino)- | EFGR | Pharmaceuticals | growth factor | | | | |
| 6,7-bis(2- | inhibitor, OSI; | | receptor | | | | |
| methoxyethoxy)- | EFGR | | kinase inhibitor | | | | |
| quinazoline | inhibitor, | | | | | | |
| hydrochloride | Pfizer; | | | | | | |
| | tumour growth | | | | | | |
| | inhibitors, | | | | | | |
| | OSI; tumour | | | | | | |
| | growth inhibitors, | | | | | | |
| | Pfiz | | | | | | |
| | growth factor | Institute for | Epidermal | | | | |
| | complex, IPR | Pharmaceutical | growth factor | | | | |
| | GFC, IPR | Research | agonist; | | | | |
| | | | Transforming | | | | |
| | | | growth factor | | | | |
| | | | alpha agonist; | | | | |
| | | | Transforming | | | | |
| | | | growth factor | | | | |
| | | | beta agonist | | | | |
| | PN-355 | Paracelsian | Tyrosine | | | | |
| | AndroVir; | | kinase | | | | |
| | AndroVir-DS | | inhibitor | | | | |
| | ZD-1838 | Zeneca | Epidermal | | | | |
| | | | growth factor | | | | |
| | | | receptor | | | | |
| | | | kinase | | | | |
| | | | inhibitor | | | | |
| 4-(3-Chloro-4- | Iressa ® | Zeneca | Epidermal growth | U.S. Pat. No. | 250-500 | | |
| fluorophenylamino)- | | | factor receptor | 5,616,582; | mg daily. | | |
| 7-methoxy-6-(3-(4- | | | kinase inhibitor; | U.S. Pat. No. | | | |
| morpholinyl)propoxy) | | | quinazoline structural | 5,457,105 | | | |
| quinazoline (ZD-1839) | | | class | | | | |
| 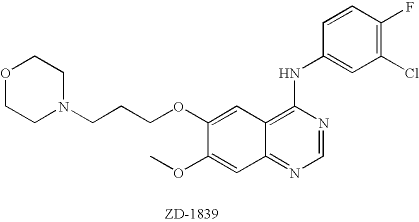 | | | | | | | |
| 4-(4-methylpiperazin- | CGP-57148 | Novartis | Tyrosine | | | | |
| 1-ylmethyl)-N-[4- | | | kinase | | | | |
| methyl-3-[4-(3- | | | inhibitor; | | | | |
| pyridyl)pyrimidin-2- | | | Platelet | | | | |
| ylamino]phenyl] | | | growth factor | | | | |
| benzamide | | | antagonist | | | | |
| | CGP-59326; | Novartis | Epidermal | | | | |
| | CGP- | | growth factor | | | | |
| | 59326B; | | receptor | | | | |
| | CGP-62706; | | kinase | | | | |
| | CGP-74321; | | inhibitor | | | | |
| | CGP-75166; | | | | | | |
| | CGP-76627 | | | | | | |
| | CGP-79787 | Novartis | Epidermal | | | | |
| | | | growth factor | | | | |
| | | | receptor | | | | |
| | | | kinase inhibitor | | | | |
| | DWP-408 | Daewoong | Epidermal | | | | |
| | | | growth factor | | | | |
| | | | agonist | | | | |
| | EGF- | | Epidermal | | | | |
| | genistein, | | growth factor | | | | |
| | Wayne | | antagonist; | | | | |
| | genistein, | | Tyrosine | | | | |
| | Wayne | | kinase | | | | |
| | | | inhibitor | | | | |
| Muellerian-inhibiting | mullerian | | Epidermal | | | | |
| hormone | inhibiting | | growth factor | | | | |
| | subst, Ma | | receptor | | | | |
| | MIS, | | kinase | | | | |
| | Massachusetts | | inhibitor | | | | |
| 4-(m-chloro)-5,6- | | Novartis | Epidermal | EP 682027 | | | |
| dimethyl-7H- | | | growth factor | | | | |
| pyrrolo[2,3- | | | receptor | | | | |
| d]pyrimidine | | | kinase inhibitor; | | | | |
| | | | Tyrosine kinase | | | | |
| | | | inhibitor | | | | |
| 5-[3-[3-methoxy-4-[2- | | Takeda | Tyrosine | WO | | | |
| [(E)-2-phenylethenyl]- | | | kinase | 9700249 | | | |
| 4- | | | inhibitor | | | | |
| oxazolylmethoxy] | | | | | | | |
| phenyl]propyl]-3-[2-[(E)- | | | | | | | |
| 2-phenylethenyl]-4- | | | | | | | |
| oxazolylmethyl]-2,4- | | | | | | | |
| oxazolidinedione | | | | | | | |
| N-(6-Benzothiazolyl)- | | Celltech | ZAP-70 | WO | | | |
| 4-(2-(1- | | | protein | 9811095 | | | |
| piperazinyl)pyrid-5- | | | tyrosine | | | | |
| yl)-2-pyrimidineamine | | | kinase inhibitor; | | | | |
| | | | Protein kinase C | | | | |
| | | | inhibitor | | | | |
| | PI-88 | Progen | Heparanase | | | | |
| | | | inhibitor; | | | | |
| | | | Fibroblast | | | | |
| | | | growth factor | | | | |
| | | | antagonist; | | | | |
| | | | Epidermal | | | | |
| | | | growth factor | | | | |
| | | | antagonist | | | | |
| | PJ3505 | PowderJect | | | | | |
| | | Pharmaceuticals | | | | | |
| | S-96-8045 | Hoechst | Endothelial | | | | |
| | antisense | Marion | growth factor | | | | |
| | oligonucleotides, | Roussel | antagonist; | | | | |
| | Ho; VEGF | | Protein synthesis | | | | |
| | oligonucleotides, | | antagonist; | | | | |
| | Hoechst | | RNA antagonist | | | | |
| | VEGF | Chugai | Endothelial | WO | | | |
| | inhibitor, | | growth factor | 9803663 | | | |
| | Chugai | | antagonist | | | | |
| 4-(4-chloro-2-fluoro- | VEGF | Zeneca | Endothelial | WO | | | |
| 5-hydroxyanilino)-6- | inhibitors, | | growth factor | 9734876 | | | |
| methoxy-7-(2- | Zeneca | | antagonist | | | | |
| methoxyethoxy)cinnoline | | | | | | | |
| N-[4-[(3-chloro-4- | | Pfizer | tyrosine | | well | No signs of | Suppresses |
| fluorophenyl)amino]- | | | kinase | | tolerated | toxicity | tumor growth |
| 7-[3-(4-morpholinyl) | | | inhibitor; | | at doses | | |
| propoxy]-6- | | | EGF receptor | | of 50-650 | | |
| quinazolinyl]-2- | | | inhibitor | | mg/d | | |
| propenamide | | | | | | | |
| EKB-569 | | Wyeth- | EGFR | | | | |
| | | Ayerst | tyrosine | | | | |
| | | Pharmaceuticals | kinase inhibitor | | | | |
| PTK787 | | Novartis | Inhibits | | | | Advanced |
| | | Pharmaceuticals | vascualar | | | | cancers |
| | | | endothelial GFR, | | | | |
| | | | tyrosine kinases; | | | | |
| | | | impairs vascular | | | | |
| | | | endothelial | | | | |
| | | | growth factor- | | | | |
| | | | induced | | | | |
| | | | responses and | | | | |
| tumor growth | | | | | | | |
| HER-2/neu protein | Herceptin | UCLA; | Humanized | | | | non- |
| antibody | | Genetech | Mab against | | | | Hodgkin's |
| | | | Her-2 growth | | | | lymphoma, |
| | | | factor | | | | breast and |
| | | | receptor | | | | colon cancer, |
| | | | | | | | and melanoma |
| EGF receptor | | M.D. | | | | | |
| antibody | | Anderson | | | | | |
| | | Cancer | | | | | |
| | | Center in | | | | | |
| | | Houston | ______________ | | | | |
| Bevacizumab; anti- | | Genentech; | Monoclonal | | | | |
| VEGF humanized | | National | antibody; | | | | |
| monoclonal antibody | | Cancer | neutralizes | | | | |
| | | Institute | the vascular | | | | |
| | | endothelial | | | | | |
| | | growth factor | | | | | |
| | | (VEGF) protein; | | | | | |
| | | inhibits tumor | | | | | |
| | | growth | | | | | |

TABLE 11
Antineoplastic Agents

| Name | Company | Patent | Oncology Indication | Mode of Action |
|---|---|---|---|---|
| VEGF inhibitor, Agouron | Agouron | | Angiogenesis disorders, | EGF antagonist |
| | Pharmaceuticals Inc | | Carcinoma | |
| GEM-220 | Hybridon Inc | WO-09627006 | Neoplasm | EGF antagonist |
| AR-639 | Aronex Pharmaceuticals | | Liver tumor, Neoplasm, | EGF antagonist |
| | Inc | | Renal tumor | |
| MDX-447 | Merck KGaA | | Carcinoma, Head & | EGF antagonist |
| | | | neck tumor, Prostate | |
| | | | tumor | |
| MDX-260 | Medarex Inc | | Glioma, Melanoma, | EGF antagonist |
| | | | Nervous system tumor | |
| DAB-720 | Mitsubishi Chemical | | Neoplasm | EGF binding agent |
| | Corp | | | |
| HER-2 antagonist, | Sugen Inc | | Breast tumor, Lung | EGF binding agent |
| Sugen/Asta | | | tumor, Ovary tumor, | |
| | | | Prostate tumor, | |
| | | | Stomach tumor | |
| VRCTC-310 | Ventech Research | | Neoplasm | EGF binding agent |
| MR1scFvPE38KDEL, | National Cancer Institute | | Neoplasm | EGF binding agent |
| NCI | | | | |
| ABX-EGF | Abgenix Inc | | Neoplasm | EGF binding agent |
| EMD-55900 | Merck KGaA | | Carcinoma, Glioma | EGF binding agent |
| EMD-72000 | Merck KGaA | | Carcinoma | EGF binding agent |
| EGF fusion toxin, | Seragen Inc | | Solid tumor, Psoriasis, | EGF binding agent |
| Seragen | | | Restenosis, Carcinoma, | |
| | | | Lung tumor | |
| OLX-103 | Merck & Co Inc | | Bladder tumor | EGF binding agent |
| SELEX | NeXstar | US-05270163 | Neoplasm | Elastase inhibitor |
| | Pharmaceuticals Inc | | | |
| CGP-62706 | Novartis AG | | Neoplasm | Endothelial growth factor |
| | | | | antagonist |
| SU-5271 | Zeneca Group Plc | | Psoriasis, Neoplasm | Endothelial growth factor |
| | | | | antagonist |
| NX-278-L | NeXstar | WO-09627604 | Angiogenesis disorder, | Endothelial growth factor |
| | Pharmaceuticals Inc | | Kaposis sarcoma | antagonist |
| metalloprotease | Glycomed Inc | | Neoplasm | Endothelin converting |
| inhibitor, Glycomed | | | | enzyme inhibitor |
| EGF fusion protein, | Seragen Inc | | Solid tumor | Epidermal growth factor |
| Seragen | | | | |
| Amphiregulin | Bristol-Myers Squibb Co | | Carcinoma | Epidermal growth factor |
| SU-5271 | Zeneca Group Plc | | Neoplasm | Epidermal growth factor |
| | | | | antagonist |
| CGP-52411 | Novartis AG | EP-00516588 | Neoplasm | Epidermal growth factor |
| | | | | antagonist |
| AG-1478 | University of California- | | Neoplasm | Epidermal growth factor |
| | San Diego Medical | | | antagonist |
| | Center | | | |
| RC-3940-ll | Pharmacia & Upjohn Inc | | Breast tumor, Neoplasm | Epidermal growth factor |
| | | | | antagonist |
| argos | Medical Research | | Carcinoma | Epidermal growth factor |
| | Council (MRC) | | | antagonist |
| CP-358774 | OSI Pharmaceuticals Inc | | Carcinoma, | Epidermal growth factor |
| | | | Angiogenesis disorder, | antagonist |
| | | | Non-Hodgkin lymphoma, | |
| | | | Head & neck tumor, | |
| | | | Breast tumor, Bladder | |
| | | | tumor | |
| C225 | Imclone Systems Inc | | Breast tumor, Head & | Epidermal growth factor |
| | | | neck tumor, Lung tumor, | antagonist |
| | | | Prostate tumor, Renal | |
| | | | tumor | |
| hbEGF-toxin, Prizm | Prizm Pharmaceuticals | | Bladder tumor, | Epidermal growth factor |
| | Inc | | Carcinoma, Ovary tumor | antagonist |
| MAb 4D5 | Genentech Inc | | Breast tumor | Epidermal growth factor |
| | | | | antagonist |
| BBR-1611 | Boehringer Mannheim | | Carcinoma | Epidermal growth factor |
| | GmbH | | | antagonist |
| PD-169450 | Parke-Davis & Co | | Neoplasm | Epidermal growth factor |
| | | | | antagonist |
| reveromycin-A | Snow Brand Milk | | Carcinoma, Neoplasm | Epidermal growth factor |
| | Products Co Ltd | | | antagonist |
| QX-101 | Taiho Pharmaceutical | | Neoplasm | Tyrosinase inhibitor |
| | Co Ltd | | | |
| SU-5271 | Zeneca Group Plc | | Neoplasm | Tyrosine kinase inhibitor |
| flavopiridol | Hoechst AG | | Breast tumor, Lung | Tyrosine kinase inhibitor |
| | | | tumor, Digestive system | |
| | | | tumor, Neoplasma, | |
| | | | Lymphoma | |
| SU-101 | Sugen Inc | WO-09633745 | Neoplasm, Solid tumor, | Tyrosine kinase inhibitor |
| | | | Ovary tumor, Glioma, | |
| | | | Kaposis sarcoma, | |
| | | | Prostate tumor, Lung | |
| | | | tumor | |
| celastrol | Schering AG | | Neoplasm | Tyrosine kinase inhibitor |
| CGP-52411 | Novartis AG | EP-00516588 | Neoplasm | Tyrosine kinase inhibitor |
| anti-flk-1, ImClone | Imclone Systems Inc | WO-09521868 | Angiogenesis disorder, | Tyrosine kinase inhibitor |
| systems Inc | | | Carcinoma | |
| CEP-2563 | Cephalon Inc | WO-09631515 | Prostate tumor | Tyrosine kinase inhibitor |
| HER-2 antagonist, | Sugen Inc | | Breast tumor, Lung | Tyrosine kinase inhibitor |
| Sugen/Asta | | | tumor, Ovary tumor, | |
| | | | Prostate tumor, | |
| | | | Stomach tumor | |
| NSC-675967 | National Cancer Institute | | Carcinoma | Tyrosine kinase inhibitor |
| SU-5416 | Sugen Inc | | Angiogenesis disorder, | Tyrosine kinase inhibitor |
| | | | Diabetic retinopathy, | |
| | | | Neoplasm, Solid tumor | |
| FCE-26806 | Pharmacia & Upjohn | | Neoplasm | Tyrosine kinase inhibitor |
| | SpA | | | |
| DAB-720 | Mitsubishi Chemical | | Neoplasm | Tyrosine kinase inhibitor |
| | Corp | | | |
| CEP-751 | Cephalon Inc | | Prostate tumor | Tyrosine kinase inhibitor |
| ZD-1838 | Zeneca Group Plc | WO-09615118 | Breast tumor, Lung | Tyrosine kinase inhibitor |
| | | | tumor | |
| tyrosine kinase inhibitor, | Pfizer Inc | | Neoplasm | Tyrosine kinase inhibitor |
| Pfizer | | | | |
| CGP-60261 | Novartis AG | | Carcinoma | Tyrosine kinase inhibitor |
| EGF-RTK antagonist, | Sugen Inc | | Brain tumor, Breast | Tyrosine kinase inhibitor |
| Sugen | | | tumor, Head & neck | |
| | | | tumor, Lung tumor, | |
| | | | Stomach tumor | |
| ALL-TK antagonists, | Sugen Inc | | Lymphoma, Leukemia | Tyrosine kinase inhibitor |
| Sugen | | | | |
| GRB2 antagonists, | Sugen Inc | | Leukemia, Neoplasm | Tyrosine kinase inhibitor |
| Sugen | | | | |
| CGP-57148 | Novartis AG | | Bone marrow | Tyrosine kinase inhibitor |
| | | | transplantation, Myeloid | |
| | | | leukemia, Neoplasm | |
| ZD-1839 | Zeneca Group Plc | WO-09633980 | Carcinoma, Solid tumor | Tyrosine kinase inhibitor |
| erbB-2 receptor | Southern Research Inst | | Neoplasm | Tyrosine kinase inhibitor |
| inhibitors, SRI | | | | |
| PD-158780 | Parke-Davis & Co Ltd | | Carcinoma, Neoplasm, | Tyrosine kinase inhibitor |
| | | | Breast tumor | |
| benzothiazoles | University of Nottingham | | Breast tumor | Tyrosine kinase inhibitor |
| PD-171026 | Parke-Davis & Co | | Neoplasm | Tyrosine kinase inhibitor |
| BE-23372M derivatives, | Banyu Pharmaceutical | | Neoplasm | Tyrosine kinase inhibitor |
| Banyu | Co Ltd | | | |
| Met TK antagonist, | Sugen Inc | | Stomach tumor, | Tyrosine kinase inhibitor |
| Sugen | | | Colorectal tumor, Lung | |
| | | | tumor | |
| PD-159973 | Parke-Davis & Co | | Carcinoma | Tyrosine kinase inhibitor |
| GW-282974 | Glaxo Wellcome plc | | Breast tumor, Lung | Tyrosine kinase inhibitor |
| | | | tumor | |
| CP-292597 | Pfizer Central Research | | Neoplasm | Tyrosine kinase inhibitor |
| ZM-105180 | Zeneca Pharmaceuticals | WO-09615118 | Neoplasm | Tyrosine kinase inhibitor |
| GW-7072X | Glaxo Wellcome plc | | Neoplasm | Tyrosine kinase inhibitor |
| Lck tyrosine kinase | Bristol-Myers Squibb Co | | Carcinoma | Tyrosine kinase inhibitor |
| inhibitors, BMS | | | | |
| PD-168393 | Parke-Davis & Co | | Neoplasm | Tyrosine kinase inhibitor |
| PD-173956 | Parke-Davis & Co | | Neoplasm | Tyrosine kinase inhibitor |
| tyrosine kinase | Novartis AG | | Neoplasm | Tyrosine kinase inhibitor |
| inhibitors, Novartis | | | | |
| RG-14620 | Rhone-Poulenc Rorer | WO-09116051 | Psoriasis, Squamous | Tyrosine kinase inhibitor |
| | Inc | | cell carcinoma | |
| CGP-59326 | Novartis AG | WO-09610028 | Neoplasm | Tyrosine kinase inhibitor |
| genistein | Yamanouchi | | Carcinoma | Tyrosine kinase inhibitor |
| | Pharmaceutical Co Ltd | | | |
| FCE-27119 | Pharmacia & Upjohn | | Neoplasm | Tyrosine kinase inhibitor |
| | SpA | | | |
| RG-13022 | Rhone-Poulenc Rorer | WO-09116051 | Breast tumor, | Tyrosine kinase inhibitor |
| | Inc | | Squamous cell | |
| | | | carcinoma | |
| RG-50864 | Rhone-Poulenc SA | WO-09116892 | Neoplasm | Tyrosine kinase inhibitor |
| PD-154233 | Parke-Davis & Co | | Neoplasm | Tyrosine kinase inhibitor |
| TT-232 | BioSignal Inc | | Neoplasm | Tyrosine kinase inhibitor |
| AG-514 | Agouron | | Neoplasm | Tyrosine kinase inhibitor |
| | Pharmaceuticals Inc | | | |
| AG-568 | Agouron | | Neoplasm | Tyrosine kinase inhibitor |
| | Pharmaceuticals Inc | | | |
| PD-151514 | Parke-Davis & Co | | Neoplasm | Tyrosine kinase inhibitor |
| BE-23372M | Banyu Pharmaceutical | JP-04275284 | Neoplasm | Tyrosine kinase inhibitor |
| | Co Ltd | | | |
| KW-6151 | Kyowa Hakko Kogyo Co | | Prostate tumor | Tyrosine kinase inhibitor |
| | Ltd | | | |
| paeciloquinones | Novartis AG | | Neoplasm | Tyrosine kinase inhibitor |
| PDGFrTK inhibitors, | Sterling Winthrop Group | | Carcinoma | Tyrosine kinase inhibitor |
| Sterling Winthrop | Ltd | | | |
| SDZ-LAP-977 | Novartis AG | | Melanoma, Neoplasm | Tyrosine kinase inhibitor |
| CGP-53716 | Novartis AG | | Neoplasm | Tyrosine kinase inhibitor |
| CGP-79787 | Novartis AG | | Carcinoma | Tyrosine kinase inhibitor |
| B43-genistein | University of Minnesota | WO-09606116 | Leukemia | Tyrosine kinase inhibitor |
| tyrosine kinase | Sugen Inc | | Carcinoma | Tyrosine kinase inhibitor |
| inhibitors, Sugen | | | | |
| CGP-62706 | Novartis AG | | Neoplasm | Tyrosine kinase inhibitor, |
| | | | | Anticancer |
| AG-957 | National Cancer Institute | | Myeloid leukemia | Tyrosine kinase |
| | | | | modulator |
| VEGF inhibitor, Agouron | Agouron | | Angiogenesis disorders, | EGF antagonist |
| | Pharmaceuticals Inc | | Carcinoma | |
| GEM-220 | Hybridon Inc | WO-09627006 | Neoplasm | EGF antagonist |
| AR-639 | Aronex Pharmaceuticals | | Liver tumor, Neoplasm, | EGF antagonist |
| | Inc | | Renal tumor | |
| MDX-447 | Merck KGaA | | Carcinoma, Head & | EGF antagonist |
| | | | neck tumor, Prostate | |
| | | | tumor | |
| MDX-260 | Medarex Inc | | Glioma, Melanoma, | EGF antagonist |
| | | | Nervous system tumor | |
| DAB-720 | Mitsubishi Chemical | | Neoplasm | EGF binding agent |
| | Corp | | | |
| HER-2 antagonist, | Sugen Inc | | Breast tumor, Lung | EGF binding agent |
| Sugen/Asta | | | tumor, Ovary tumor, | |
| | | | Prostate tumor, | |
| | | | Stomach tumor | |
| VRCTC-310 | Ventech Research | | Neoplasm | EGF binding agent |
| MR1scFvPE38KDEL, | National Cancer Institute | | Neoplasm | EGF binding agent |
| NCI | | | | |
| ABX-EGF | Abgenix Inc | | Neoplasm | EGF binding agent |
| EMD-55900 | Merck KGaA | | Carcinoma, Glioma | EGF binding agent |
| EMD-72000 | Merck KGaA | | Carcinoma | EGF binding agent |
| EGF fusion toxin, | Seragen Inc | | Solid tumor, Psoriasis, | EGF binding agent |
| Seragen | | | Restenosis, Carcinoma, | |
| | | | Lung tumor | |
| OLX-103 | Merck & Co Inc | | Bladder tumor | EGF binding agent |
| SELEX | NeXstar | US-05270163 | Neoplasm | Elastase inhibitor |
| | Pharmaceuticals Inc | | | |
| CGP-62706 | Novartis AG | | Neoplasm | Endothelial growth factor |
| | | | | antagonist |
| SU-5271 | Zeneca Group Plc | | Psoriasis, Neoplasm | Endothelial growth factor |
| | | | | antagonist |
| NX-278-L | NeXstar | WO-09627604 | Angiogenesis disorder, | Endothelial growth factor |
| | Pharmaceuticals Inc | | Kaposis sarcoma | antagonist |
| EGF fusion protein, | Seragen Inc | | Solid tumor | Epidermal growth factor |
| Seragen | | | | |
| Amphiregulin | Bristol-Myers Squibb Co | | Carcinoma | Epidermal growth factor |
| SU-5271 | Zeneca Group Plc | | Neoplasm | Epidermal growth factor |
| | | | | antagonist |
| CGP-52411 | Novartis AG | EP-00516588 | Neoplasm | Epidermal growth factor |
| | | | | antagonist |
| AG-1478 | University of California- | | Neoplasm | Epidermal growth factor |
| | San Diego Medical | | | antagonist |
| | Center | | | |
| RC-3940-II | Pharmacia & Upjohn Inc | | Breast tumor, Neoplasm | Epidermal growth factor |
| | | | | antagonist |
| argos | Medical Research | | Carcinoma | Epidermal growth factor |
| | Council (MRC) | | | antagonist |
| CP-358774 | OSI Pharmaceuticals Inc | | Carcinoma, | Epidermal growth factor |
| | | | Angiogenesis disorder, | antagonist |
| | | | Non-Hodgkin lymphoma, | |
| | | | Head & neck tumor, | |
| | | | Breast tumor, Bladder | |
| | | | tumor | |
| C225 | Imclone Systems Inc | | Breast tumor, Head & | Epidermal growth factor |
| | | | neck tumor, Lung tumor, | antagonist |
| | | | Prostate tumor, Renal | |
| | | | tumor | |
| hbEGF-toxin, Prizm | Prizm Pharmaceuticals | | Bladder tumor, | Epidermal growth factor |
| | Inc | | Carcinoma, Ovary tumor | antagonist |
| MAb 4D5 | Genentech Inc | | Breast tumor | Epidermal growth factor |
| | | | | antagonist |
| BBR-1611 | Boehringer Mannheim | | Carcinoma | Epidermal growth factor |
| | GmbH | | | antagonist |
| PD-169450 | Parke-Davis & Co | | Neoplasm | Epidermal growth factor |
| | | | | antagonist |
| reveromycin-A | Snow Brand Milk | | Carcinoma, Neoplasm | Epidermal growth factor |
| | Products Co Ltd | | | antagonist |
| QX-101 | Taiho Pharmaceutical | | Neoplasm | Tyrosinase inhibitor |
| | Co Ltd | | | |
| SU-5271 | Zeneca Group Plc | | Neoplasm | Tyrosine kinase inhibitor |
| flavopiridol | Hoechst AG | | Breast tumor, Lung | Tyrosine kinase inhibitor |
| | | | tumor, Digestive system | |
| | | | tumor, Neoplasma, | |
| | | | Lymphoma | |
| SU-101 | Sugen Inc | WO-09633745 | Neoplasm, Solid tumor, | Tyrosine kinase inhibitor |
| | | | Ovary tumor, Glioma, | |
| | | | Kaposis sarcoma, | |
| | | | Prostate tumor, Lung | |
| | | | tumor | |
| celastrol | Schering AG | | Neoplasm | Tyrosine kinase inhibitor |
| CGP-52411 | Novartis AG | EP-00516588 | Neoplasm | Tyrosine kinase inhibitor |
| anti-flk-1, ImClone | Imclone Systems Inc | WO-09521868 | Angiogenesis disorder, | Tyrosine kinase inhibitor |
| systems Inc | | | Carcinoma | |
| CEP-2563 | Cephalon Inc | WO-09631515 | Prostate tumor | Tyrosine kinase inhibitor |
| HER-2 antagonist, | Sugen Inc | | Breast tumor, Lung | Tyrosine kinase inhibitor |
| Sugen/Asta | | | tumor, Ovary tumor, | |
| | | | Prostate tumor, | |
| | | | Stomach tumor | |
| NSC-675967 | National Cancer Institute | | Carcinoma | Tyrosine kinase inhibitor |
| SU-5416 | Sugen Inc | | Angiogenesis disorder, | Tyrosine kinase inhibitor |
| | | | Diabetic retinopathy, | |
| | | | Neoplasm, Solid tumor | |
| FCE-26806 | Pharmacia & Upjohn | | Neoplasm | Tyrosine kinase inhibitor |
| | SpA | | | |
| DAB-720 | Mitsubishi Chemical | | Neoplasm | Tyrosine kinase inhibitor |
| | Corp | | | |
| CEP-751 | Cephalon Inc | | Prostate tumor | Tyrosine kinase inhibitor |
| ZD-1838 | Zeneca Group Plc | WO-09615118 | Breast tumor, Lung | Tyrosine kinase inhibitor |
| | | | tumor | |
| tyrosine kinase inhibitor, | Pfizer Inc | | Neoplasm | Tyrosine kinase inhibitor |
| Pfizer | | | | |
| CGP-60261 | Novartis AG | | Carcinoma | Tyrosine kinase inhibitor |
| EGF-RTK antagonist, | Sugen Inc | | Brain tumor, Breast | Tyrosine kinase inhibitor |
| Sugen | | | tumor, Head & neck | |
| | | | tumor, Lung tumor, | |
| | | | Stomach tumor | |
| ALL-TK antagonists, | Sugen Inc | | Lymphoma, Leukemia | Tyrosine kinase inhibitor |
| Sugen | | | | |
| GRB2 antagonists, | Sugen Inc | | Leukemia, Neoplasm | Tyrosine kinase inhibitor |
| Sugen | | | | |
| CGP-57148 | Novartis AG | | Bone marrow | Tyrosine kinase inhibitor |
| | | | transplantation, Myeloid | |
| | | | leukemia, Neoplasm | |
| ZD-1839 | Zeneca Group Plc | WO-09633980 | Carcinoma, Solid tumor | Tyrosine kinase inhibitor |
| erbB-2 receptor | Southern Research Inst | | Neoplasm | Tyrosine kinase inhibitor |
| inhibitors, SRI | | | | |
| PD-158780 | Parke-Davis & Co Ltd | | Carcinoma, Neoplasm, | Tyrosine kinase inhibitor |
| | | | Breast tumor | |
| benzothiazoles | University of Nottingham | | Breast tumor | Tyrosine kinase inhibitor |
| PD-171026 | Parke-Davis & Co | | Neoplasm | Tyrosine kinase inhibitor |
| BE-23372M derivatives, | Banyu Pharmaceutical | | Neoplasm | Tyrosine kinase inhibitor |
| Banyu | Co Ltd | | | |
| Met TK antagonist, | Sugen Inc | | Stomach tumor, | Tyrosine kinase inhibitor |
| Sugen | | | Colorectal tumor, Lung | |
| | | | tumor | |
| PD-159973 | Parke-Davis & Co | | Carcinoma | Tyrosine kinase inhibitor |
| GW-282974 | Glaxo Wellcome plc | | Breast tumor, Lung | Tyrosine kinase inhibitor |
| | | | tumor | |
| CP-292597 | Pfizer Central Research | | Neoplasm | Tyrosine kinase inhibitor |
| ZM-105180 | Zeneca Pharmaceuticals | WO-09615118 | Neoplasm | Tyrosine kinase inhibitor |
| GW-7072X | Glaxo Wellcome plc | | Neoplasm | Tyrosine kinase inhibitor |
| Lck tyrosine kinase | Bristol-Myers Squibb Co | | Carcinoma | Tyrosine kinase inhibitor |
| inhibitors, BMS | | | | |
| PD-168393 | Parke-Davis & Co | | Neoplasm | Tyrosine kinase inhibitor |
| PD-173956 | Parke-Davis & Co | | Neoplasm | Tyrosine kinase inhibitor |
| tyrosine kinase | Novartis AG | | Neoplasm | Tyrosine kinase inhibitor |
| inhibitors, Novartis | | | | |
| RG-14620 | Rhone-Poulenc Rorer | WO-09116051 | Psoriasis, Squamous | Tyrosine kinase inhibitor |
| | Inc | | cell carcinoma | |
| CGP-59326 | Novartis AG | WO-09610028 | Neoplasm | Tyrosine kinase inhibitor |
| genistein | Yamanouchi | | Carcinoma | Tyrosine kinase inhibitor |
| | Pharmaceutical Co Ltd | | | |
| FCE-27119 | Pharmacia & Upjohn | | Neoplasm | Tyrosine kinase inhibitor |
| | SpA | | | |
| RG-13022 | Rhone-Poulenc Rorer | WO-09116051 | Breast tumor, | Tyrosine kinase inhibitor |
| | Inc | | Squamous cell | |
| | | | carcinoma | |
| RG-50864 | Rhone-Poulenc SA | WO-09116892 | Neoplasm | Tyrosine kinase inhibitor |
| PD-154233 | Parke-Davis & Co | | Neoplasm | Tyrosine kinase inhibitor |
| TT-232 | BioSignal Inc | | Neoplasm | Tyrosine kinase inhibitor |
| AG-514 | Agouron | | Neoplasm | Tyrosine kinase inhibitor |
| | Pharmaceuticals Inc | | | |
| AG-568 | Agouron | | Neoplasm | Tyrosine kinase inhibitor |
| | Pharmaceuticals Inc | | | |
| PD-151514 | Parke-Davis & Co | | Neoplasm | Tyrosine kinase inhibitor |
| BE-23372M | Banyu Pharmaceutical | JP-04275284 | Neoplasm | Tyrosine kinase inhibitor |
| | Co Ltd | | | |
| KW-6151 | Kyowa Hakko Kogyo Co | | Prostate tumor | Tyrosine kinase inhibitor |
| | Ltd | | | |
| paeciloquinones | Novartis AG | | Neoplasm | Tyrosine kinase inhibitor |
| PDGFrTK inhibitors, | Sterling Winthrop Group | | Carcinoma | Tyrosine kinase inhibitor |
| Sterling Winthrop | Ltd | | | |
| SDZ-LAP-977 | Novartis AG | | Melanoma, Neoplasm | Tyrosine kinase inhibitor |
| CGP-53716 | Novartis AG | | Neoplasm | Tyrosine kinase inhibitor |
| CGP-79787 | Novartis AG | | Carcinoma | Tyrosine kinase inhibitor |
| B43-genistein | University of Minnesota | WO-09606116 | Leukemia | Tyrosine kinase inhibitor |
| tyrosine kinase | Sugen Inc | | Carcinoma | Tyrosine kinase inhibitor |
| inhibitors, Sugen | | | | |
| CGP-62706 | Novartis AG | | Neoplasm | Tyrosine kinase inhibitor, |
| | | | | Anticancer |
| AG-957 | National Cancer Institute | | Myeloid leukemia | Tyrosine kinase |
| | | | | modulator |

TABLE 12
Epidermal Growth Factor Antagonists

| | | Trade | | | | |
|---|---|---|---|---|---|---|
| No. | Compound Name | Name(s) | Drug Class | Dose | Manufacturer | Reference |
| A1 | Cetuximab (IMC-225) | Erbitux ™ | EGFR | | ImClone | |
| | | | inhibitor; | | Systems | |
| | | | Monoclonal | | | |
| | | | antibody | | | |
| A2 | Imatinib mesylate (STI-571) | Gleevec ™ | | | Novartis | |
| | | | | | Pharmaceuticals | |
| A3 | 4-(3-Chloroanilino)-6,7- | Tyrphostin | Selective | | AG | Takeyama K, et al., |
| | dimethoxyquinazoline (AG-1478) | | EGFR | | Scientific | Oxidative |
| | | | tyrosine | | | stress causes mucin |
| | | | kinase | | | synthesis |
| | | | inhibitor | | | via transactivation of |
| | | | | | | epidermal |
| | | | | | | growth factor receptor: |
| | | | | | | role |
| | | | | | | of neutrophils, J. |
| | | | | | | Immunol. |
| | | | | | | 2000 Feb 1; 164(3): 1546- |
| | | | | | | 52 |
| A4 | [(dimethylamino)methyl]acrylo- | | Dianilinopth | | Ciba Geigy | |
| | para-[(hydroxy-benzoylsulfonyl)- | | alimide | | (Novartis) | |
| | oxy]phenone | | | | | |
| A5 | OSI-774 in combination with | | Anti-EGFR | | Genentech; | |
| | Taxotere | | | | Hoffmann-La | |
| | | | | | Roche; OSI | |
| | | | | | Pharmaceuticals | |
| A6 | C1033 | | EGFR | | Pfizer | |
| | | | antagonist | | | |
| A7 | Sulindac in combination with | | single | | The Johns | |
| | EKB-569 | | agent | | Hopkins | |
| | | | antibody | | University | |
| | | | directed | | Oncology Center | |
| | | | against the | | and Wyeth- | |
| | | | epidermal | | Ayerst Research | |
| | | | growth | | | |
| | | | factor | | | |
| | | | receptor in | | | |
| | | | combination | | | |
| | | | with | | | |
| | | | NSAIDs | | | |
| A8 | ior-egf/r3 | | Epidermal | | Center of | EP 586002 |
| | | | growth | | Molecular | |
| | | | factor | | Immunology | |
| | | | receptor | | | |
| | | | kinase | | | |
| | | | inhibitor; | | | |
| | | | DNA | | | |
| | | | antagonist | | | |
| A9 | CI-1033 | | HER | | | Marinus W. Lobbezoo, |
| | | | receptor | | | PhD., et al., Signal |
| | | | inhibitor | | | transduction modulators |
| | | | (inhibits all | | | for cancer therapy: from |
| | | | 4 | | | promise to practice? The |
| | | | receptors) | | | Oncologist' ( © AlphaMed |
| | | | | | | Press). |
| A10 | GW-211 | | Inhibits | | | Marinus W. Lobbezoo, |
| | | | both | | | PhD., et al., Signal |
| | | | EGER and | | | transduction modulators |
| | | | HER2 | | | for cancer therapy: from |
| | | | equally | | | promise to practice? The |
| | | | well. | | | Oncologist' ( © AlphaMed |
| | | | | | | Press). |
| A11 | PKI-166 | | EGFR | | Novartis | |
| | | | inhibitor | | | |
| A12 | Combination of paclitaxel and | | | | University of | |
| | the tyrosine kinase inhibitors | | | | Texas M. D. | |
| | PKI166 and STI571 | | | | Anderson | |
| | | | | | Cancer Center | |
| A13 | BIBX1522 | | Selective | | | Takeyama K, et al., |
| | | | EGFR | | | Oxidative stress causes |
| | | | tyrosine | | | mucin synthesis via |
| | | | kinase | | | transactivation of |
| | | | inhibitor | | | epidermal growth factor |
| | | | | | | receptor: role of |
| | | | | | | neutrophils, J. Immunol. |
| | | | | | | 2000 Feb 1; 164(3): 1546- |
| | | | | | | 52 |
| A14 | A synthetic oligonucleotide | | EGFR | | | U.S. Pat. App. |
| | complementary to a nucleic acid | | blocker | | | 20030045494 |
| | encoding epidermal growth | | | | | |
| | factor receptor (EGFR), the | | | | | |
| | oligonucleotide being | | | | | |
| | complemetary to a region of | | | | | |
| | EGFR mRNA selected from the | | | | | |
| | group consisting of location 245- | | | | | |
| | 1117, 2407-3201, 3786-4102, | | | | | |
| | and 4574-45633 | | | | | |
| A15 | 4-(3-bromoanilino)-6,7- | | inhibitor of | | | Bridges A. J., et al. |
| | dimethoxyquinazoline analogues | | the | | | (1996) |
| | (PD 153035) | | epidermal | | | Tyrosine kinase |
| | | | growth | | | inhibitors. 8. An unusually |
| | | | factor | | | steep structure-activity |
| | | | receptor | | | relationship for analogues |
| | | | | | | of 4-(3-bromoanilino)-6,7- |
| | | | | | | dimethoxyquinazoline |
| | | | | | | (PD 153035), a potent |
| | | | | | | inhibitor of the epidermal |
| | | | | | | growth factor receptor. J. |
| | | | | | | Med. Chem. 39, 267- |
| | | | | | | 276. |
| A16 | 4- | | Anilinoquin | | Pfizer | PD158780 |
| | [ar(alk)ylamino]pyridopyrimidines | | azoline | | | Parke-Davis (now |
| | (4-(phenylamino)quinazolines) | | | | | Pfizer) |
| | | | | | | (Fry et al., 1997¹). |
| | 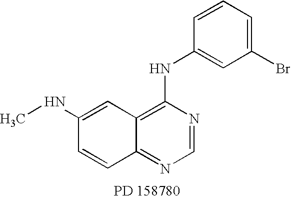 | | | | | PD69896 Parke-Davis (now Pfizer) (Fry et al., 1997). PD153717 Parke-Davis (now Pfizer) (Fry et al., 1997). Parke-Davis (now Pfizer) |
| | 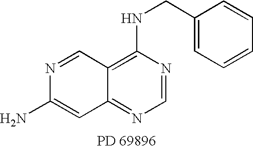 | | | | | |
| | 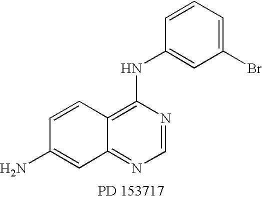 | | | | | |
| A17 | GW2974 | | | | GlaxoSmithKline | WO9828009 |
| A18 | GW9263 | | | | GlaxoSmithKline | WO9828009 |
| A19 | GW4263 | | | | GlaxoSmithKline | UK GB 2345486 |
| A20 | GW0277 | | | | GlaxoSmithKline | WO9713771 |
| A21 | GW5289 | | | | GlaxoSmithKline | WO9703069 |
| A22 | GW5949 | | | | GlaxoSmithKline | WO9935132 |
| A23 | GW9525 | | | | GlaxoSmithKline | WO9935146 |
| A24 | GW572016 | | | | GlaxoSmithKline | Phase I |
| A25 | PD13530 | | | | Pfizer | |
| A26 | 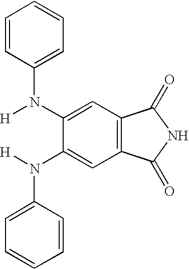 | | | | | |
| A27 | CGP5211 | | | | Novartis | |
| A28 | CGP53353 | | | | Novartis | |
| A29 | CGP 75166/PKI166 | | | | Novartis | |
| A30 | BIBX 1382 | | | | Boehringer | |
| | | | | | Ingeiheim | |
| A31 | GW-2016 | | | | GlaxoSmithKline | |
| A32 | MDX-210 | | | | Medarex | |
| A33 | 2C4 | | | | Genentech | |
| A34 | TgDCC-E1A | | | | Targeted | |
| | | | | | Genetics | |
| A35 | Butanedioic acid, mono[3-butyl- | Reveromycin | Epidermal | | Snow Brand | EP 491956, J. Antibiot, |
| | 8-(9-carboxy-6-hydroxy-3,7- | A | growth | | | 1991, 44, 259 |
| | dimethyl-2,4,8-nonatrienyl)-2-(4- | | factor | | | |
| | carboxy-3-methyl-1,3- | | antagonist | | | |
| | butadienyl)-9-methyl-1,7- | | | | | |
| | dioxaspiro[5.5]undec-3-yl]ester | | | | | |
| A36 | Imatinib mesylate (STI-571) | Gleevec ™ | | | Novartis | |
| | | | | | Pharmaceuticals | |
| A37 | 4-(3-Chloroanilino)-6,7- | Tyrphostin | Selective | | AG | Takeyama K, et al., |
| | dimethoxyquinazoline (AG-1478) | | EGER | | Scientific | Oxidative |
| | | | tyrosine | | | stress causes mucin |
| | | | kinase | | | synthesis |
| | | | inhibitor | | | via transactivation of |
| | | | | | | epidermal |
| | | | | | | growth factor receptor: |
| | | | | | | role |
| | | | | | | of neutrophils, J. |
| | | | | | | Immunol. |
| | | | | | | 2000 Feb 1; 164(3): 1546- |
| | | | | | | 52 |
| A38 | [(dimethylamino)methyl]acrylo- | | Dianilinopth | | Ciba Geigy | |
| | para-[(hydroxy-benzoylsulfonyl)- | | alimide | | (Novartis) | |
| | oxy]phenone | | | | | |
| A39 | TheraCIM h-R3 | | EGRF | | YM Biosciences | |
| | | | Inhibitor; | | | |
| | | | MAb | | | |
| | | | labeled with | | | |
| | | | rhenium- | | | |
| | | | 188 | | | |
| A40 | OSI-774 in combination with | | Anti-EGFR | | Genentech; | |
| | Taxotere | | | | Hoffmann-La | |
| | | | | | Roche; OSI | |
| | | | | | Pharmaceuticals | |
| A41 | C1033 | | EGFR | | Pfizer | |
| | | | antagonist | | | |
| A42 | EKB-569 | | Irreversible | | Wyeth | |
| | | | inhibitor of | | | |
| | | | epidermal | | | |
| | | | growth | | | |
| | | | factor | | | |
| | | | receptor | | | |
| A43 | ABX-EGF | | single | Preferred | Wyeth-Ayerst | |
| | | | agent | doses: 1 | Research | |
| | | | antibody | mg/kg, | | |
| | | | directed | 1.5 | | |
| | | | against the | mg/kg, | | |
| | | | epidermal | 2.0 | | |
| | | | growth | mg/kg, | | |
| | | | factor | and 2.5 | | |
| | | | receptor | mg/kg | | |
| A44 | Sulindac in combination with | | single | | The Johns | |
| | EKB-569 | | agent | | Hopkins | |
| | | | antibody | | University | |
| | | | directed | | Oncology Center | |
| | | | against the | | and Wyeth- | |
| | | | epidermal | | Ayerst Research | |
| | | | growth | | | |
| | | | factor | | | |
| | | | receptor in | | | |
| | | | combination | | | |
| | | | with | | | |
| | | | NSAIDs | | | |
| A45 | anti-EGFR Mabs; C225; MAb | | Epidermal | | ImClone | |
| | C225 | | growth | | Systems | c |
| | | | factor | | | |
| | | | receptor | | | |
| | | | kinase | | | |
| | | | inhibitor | | | |
| A46 | EMD-72000; anti-EGFR Mab; | | Epidermal | | Merck KGaA | |
| | EMD-6200 | | growth | | | |
| | | | factor | | | |
| | | | receptor | | | |
| | | | kinase | | | |
| | | | inhibitor | | | |
| A47 | MDX-447; BAB-447; EMD- | | Epidermal | | Medarex | |
| | 82633; H-447 | | growth | | | |
| | | | factor | | | |
| | | | antagonist; | | | |
| | | | CD8 | | | |
| | | | agonist | | | |
| A48 | ior-egf/r3 | | Epidermal | | Center of | EP 586002 |
| | | | growth | | Molecular | |
| | | | factor | | Immunology | |
| | | | receptor | | | |
| | | | kinase | | | |
| | | | inhibitor; | | | |
| | | | DNA | | | |
| | | | antagonist | | | |
| A49 | EGF-genistein, Wayne | | Epidermal | | | |
| | | | growth | | | |
| | | | factor | | | |
| | | | antagonist; | | | |
| | | | Tyrosine | | | |
| | | | kinase | | | |
| | | | inhibitor | | | |
| A50 | ABX-EGF anti-EGFr MAb, | | Epidermal | | Cell Genesys | |
| | Abgenix; anti-EGFr MAb, Cell | | growth | | | |
| | Genesys | | factor | | | |
| | | | antagonist | | | |
| A51 | anti-EGFR-DM1 Ab, | | Epidermal | | ImmunoGen | |
| | ImmunoGen anti-EGFR | | growth | | | |
| | conjugate, ImmunoGen; EGFR | | factor | | | |
| | conjugate, ImmunoGen | | agonist; | | | |
| | | | Epidermal | | | |
| | | | growth | | | |
| | | | factor | | | |
| | | | antagonist | | | |
| A52 | C1-1033 | | HER | | | Marinus W. Lobbezoo, |
| | | | receptor | | | PhD., et al., Signal |
| | | | inhibitor | | | transduction modulators |
| | | | (inhibits all | | | for cancer therapy: from |
| | | | 4 | | | promise to practice? The |
| | | | receptors) | | | Oncologist’ |
| A53 | GW-211 | | Inhibits | | | Marinus W. Lobbezoo, |
| | | | both | | | PhD., et al., Signal |
| | | | EGFR and | | | transduction modulators |
| | | | HER2 | | | for cancer therapy: from |
| | | | equally | | | promise to practice? The |
| | | | well. | | | Oncologist’ |
| A54 | PKI-166 | | EGFR | | Novartis | |
| | | | inhibitor | | | |
| A55 | Combination of paclitaxel and | | | | University of | |
| | the tyrosine kinase inhibitors | | | | Texas M. D. | |
| | PK1166 and STI571 | | | | Anderson | |
| | | | | | Cancer Center | |
| A56 | BIBX1522 | | Selective | | | Takeyama K, et al., |
| | | | EGFR | | | Oxidative stress causes |
| | | | tyrosine | | | mucin synthesis via |
| | | | kinase | | | transactivation of |
| | | | inhibitor | | | epidermal growth factor |
| | | | | | | receptor: role of |
| | | | | | | neutrophils, J. Immunol. |
| | | | | | | 2000 Feb 1; 164(3): 1546- |
| | | | | | | 52 |
| A57 | 4-(phenylamino)quinazolines | | selectively | | | |
| | | | inhibit | | | |
| | | | EGFR-TK | | | |
| | | | activity | | | |
| A60 | A synthetic oligonucleotide | | EGER | | | U.S. Pat. App. |
| | complementary to a nucleic acid | | blocker | | | 20030045494 |
| | encoding epidermal growth | | | | | |
| | factor receptor (EGFR), the | | | | | |
| | oligonucleotide being | | | | | |
| | complemetary to a region of | | | | | |
| | EGFR mRNA selected from the | | | | | |
| | group consisting of location 245- | | | | | |
| | 1117, 2407-3201, 3786-4102, | | | | | |
| | and 4574-45633 | | | | | |
| A61 | RC-3940-II | | Epidermal | | Pharmacia & | |
| | | | growth | | Upjohn Inc | |
| | | | factor | | | |
| | | | antagonist | | | |
| A62 | argos | | Epidermal | | Medical | |
| | | | growth | | Research | |
| | | | factor | | Council (MRC) | |
| | | | antagonist | | | |
| A63 | CP-358774 | | Epidermal | | OSI | |
| | | | growth | | Pharmaceuticals | |
| | | | factor | | Inc | |
| | | | antagonist | | | |
| A64 | C225 | | Epidermal | | lmclone Systems | |
| | | | growth | | Inc | |
| | | | factor | | | |
| | | | antagonist | | | |
| A65 | hbEGF-toxin, Prizm | | Epidermal | | Prizm | |
| | | | growth | | Pharmaceuticals | |
| | | | factor | | Inc | |
| | | | antagonist | | | |
| A66 | MAb 4D5 | | Epidermal | | Genentech Inc | |
| | | | growth | | | |
| | | | factor | | | |
| | | | antagonist | | | |
| A67 | BBR-1611 | | Epidermal | | Boehringer | |
| | | | growth | | Mannheim | |
| | | | factor | | GmbH | |
| | | | antagonist | | | |
| A68 | PD-169450 | | Epidermal | | Parke-Davis & | |
| | | | growth | | Co | |
| | | | factor | | | |
| | | | antagonist | | | |
| A69 | CGP-52411 | | Epidermal | | Novartis AG | |
| | | | growth | | | |
| | | | factor | | | |
| | | | antagonist | | | |
| A70 | SU-5271 | | Epidermal | | Zeneca Group | |
| | | | growth | | Plc | |
| | | | factor | | | |
| | | | antagonist | | | |
| A71 | Amphiregulin | | Epidermal | | Bristol-Myers | |
| | | | growth | | Squibb Co | |
| | | | factor | | | |
| A72 | EGF fusion protein, Seragen | | Epidermal | | Seragen Inc | |
| | | | growth | | | |
| | | | factor | | | |
| A73 | 4-(3-bromoanilino)-6,7- | | inhibitor of | | | Bridges A. J., et al. |
| | dimethoxyquinazoline analogues | | the | | | (1996) |
| | (PD 153035) | | epidermal | | | Tyrosine kinase |
| | | | growth | | | inhibitors. 8. An unusually |
| | | | factor | | | steep structure-activity |
| | | | receptor | | | relationship for analogues |
| | | | | | | of 4-(3-bromoanilino)-6,7- |
| | | | | | | dimethoxyquinazoline |
| | | | | | | (PD 153035), a potent |
| | | | | | | inhibitor of the epidermal |
| | | | | | | growth factor receptor. J. |
| | | | | | | Med. Chem. 39: 267-276. |
| A74 | 4- | | Anilinoquin | | Pfizer | PD158780 |
| | [ar(alk)ylamino]pyridopyrimidines; | | azoline | | | Parke-Davis (now |
| | (4-(phenylamino)quinazolines) | | | | | Pfizer) |
| | | | | | | (Fry, et al., 1997³). |
| | 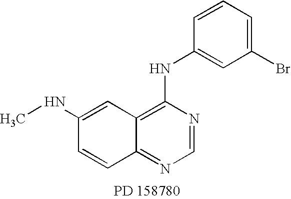 | | | | | PD69896 Parke-Davis (now Pfizer) (Fry, et al., 1997). PD153717 Parke-Davis (now Pfizer (Fry, et al., 1997). Parke-Davis (now Pfizer) |
| | 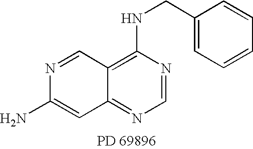 | | | | | |
| | 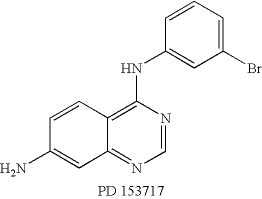 | | | | | |
| A75 | GW2974 | | | | GlaxoSmithKline | WO9828009 |
| A76 | GW9263 | | | | GlaxoSmithKline | WO9828009 |
| A77 | GW4263 | | | | GlaxoSmithKline | UK GB 2345486 |
| A78 | GW0277 | | | | GlaxoSmithKline | WO971 3771 |
| A79 | GW5289 | | | | GlaxoSmithKline | WO09703069 |
| A80 | GW5949 | | | | GlaxoSmithKline | WO099351 32 |
| A81 | GW9525 | | | | GlaxoSmithKline | WO09935146 |
| A82 | GW572016 | | | | GlaxoSmithKline | Phase I |
| A83 | PD13530 | | | | Pfizer | |
| A84 | 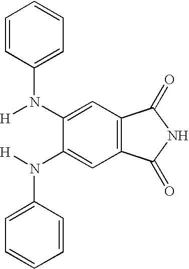 | | | | | |
| A85 | CGP5211 | | | | Novartis | |
| A86 | CGP53353 | | | | Novartis | |
| A87 | CGP 75166/PKI166 | | | | Novartis | |
| A88 | BIBX 1382 | | | | Boehringer | |
| | | | | | Ingeiheim | |
| A89 | EKB-569 | | | | Wyeth-Ayerst | |
| A90 | PKI-166 | | | | | |
| A91 | CI-1033 | | | | Pfizer | |
| A92 | GW-2016 | | | | GlaxoSmithKline | |
| A93 | EMD-72000 | | | | Merck | |
| A94 | MDX-210 | | | | Medarex | |
| A95 | 2C4 | | | | Genentech | |
| A96 | TgDCC-E1A | | | | Targeted | |
| | | | | | Genetics | |

Also included in the combination of an antiangiogenesis agent and antineoplastic agent for the present invention are the isomeric forms, prodrugs and tautomers of the compounds described herein and the pharmaceutically-acceptable salts, isomers, prodrugs, enantiomers, and stereoisomers thereof. Therefore, also included in the combination of a Cox-2 inhbitor and EGF receptor antagonist for the present invention are the isomeric forms, prodrugs and tautomers of the compounds described herein and the pharmaceutically-acceptable salts, isomers, prodrugs, enantiomers, and stereoisomers thereof.

In one embodiment, the present invention encompasses a novel therapeutic composition comprising a Cox-2 inhibitor and an EGF receptor antagonist.

In yet another embodiment, the present invention encompasses a pharmaceutical composition for preventing or treating a neoplasia-related disorder in a subject that is in need of such prevention and treatment, the pharmaceutical composition comprising a Cox-2 inhibitor, an EGF receptor antagonist, and a pharmaceutically acceptable carrier.

Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, b-hydroxybutyric, galactaric and galacturonic acids.

Suitable pharmaceutically-acceptable base addition salts of compounds of the present invention include metallic ion salts and organic ion salts. More preferred metallic ion salts include, but are not limited to appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

When used as a therapeutic the compounds described herein are preferably administered with a physiologically acceptable carrier. A physiologically acceptable carrier is a formulation to which the compound can be added to dissolve it or otherwise facilitate its administration. Examples of physiologically acceptable carriers include, but are not limited to, water, saline, physiologically buffered saline. Additional examples are provided below.

The phrase “pharmaceutically acceptable” is used adjectivally herein to mean that the modified noun is appropriate for use in a pharmaceutical product. Pharmaceutically acceptable cations include metallic ions and organic ions. More preferred metallic ions include, but are not limited to appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

A compound of the present invention can be formulated as a pharmaceutical composition. Such a composition can then be administered orally, parenterally, by inhalation spray, rectally, or topically in dosage unit formulations containing conventional nontoxic pharmaceutically acceptable carriers, adjuvants, and vehicles as desired. Topical administration can also involve the use of transdermal administration such as transdermal patches or iontophoresis devices. The term parenteral as used herein includes subcutaneous injections, intravenous, intramuscular, intrasternal injection, or infusion techniques. Formulation of drugs is discussed in, for example, Hoover, John E., Remington's Pharmaceutical Sciences, Mack Publishing Co., Easton, Pa.; 1975 and Liberman, H. A. and Lachman, L., Eds., Pharmaceutical Dosage Forms, Marcel Decker, New York, N.Y., 1980.

Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables. Dimethyl acetamide, surfactants including ionic and non-ionic detergents, polyethylene glycols can be used. Mixtures of solvents and wetting agents such as those discussed above are also useful.

Suppositories for rectal administration of the drug can be prepared by mixing the drug with a suitable nonirritating excipient such as cocoa butter, synthetic mono- di- or triglycerides, fatty acids and polyethylene glycols that are solid at ordinary temperatures but liquid at the rectal temperature and will therefore melt in the rectum and release the drug.

Solid dosage forms for oral administration can include capsules, tablets, pills, powders, and granules. In such solid dosage forms, the compounds of this invention are ordinarily combined with one or more adjuvants appropriate to the indicated route of administration. If administered per os, a contemplated aromatic sulfone hydroximate inhibitor compound can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. A contemplated aromatic sulfone hydroximate inhibitor compound can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage form varies depending upon the mammalian host treated and the particular mode of administration.

Dosage of Antiangiogenic Agents

Dosage levels of antiangiogenic inhibitors on the order of about 0.1 mg to about 10,000 mg of the active antiangiogenic ingredient compound are useful in the treatment of the above conditions, with preferred levels of about 1.0 mg to about 1,000 mg. The amount of active ingredient that may be combined with other anticancer agents to produce a single dosage form will vary depending upon the host treated and the particular mode of administration.

It is understood, however, that a specific dose level for any particular patient will depend upon a variety of factors including the activity of the specific compound employed, the age, body weight, general health, sex, diet, time of administration, rate of excretion, drug combination, and the severity of the particular disease being treated and form of administration.

Treatment dosages generally may be titrated to optimize safety and efficacy. Typically, dosage-effect relationships from in vitro initially can provide useful guidance on the proper doses for patient administration. Studies in animal models also generally may be used for guidance regarding effective dosages for treatment of cancers in accordance with the present invention. In terms of treatment protocols, it should be appreciated that the dosage to be administered will depend on several factors, including the particular agent that is administered, the route administered, the condition of the particular patient, etc. Generally speaking, one will desire to administer an amount of the compound that is effective to achieve a serum level commensurate with the concentrations found to be effective in vitro. Thus, where a compound is found to demonstrate in vitro activity at, e.g., 10 μM, one will desire to administer an amount of the drug that is effective to provide about a 10 μM concentration in vivo. Determination of these parameters are well within the skill of the art. These considerations, as well as effective formulations and administration procedures are well known in the art and are described in standard textbooks.

A combination therapy comprising a Cox-2 inhibitor and an EGF receptor antagonist will have an appropriate dosage level of the Cox-2 inhibitor that will generally be from about 0.01 mg per kg to about 140 mg per kg subject body weight per day, which may be administered in single or multiple doses. Preferably, the dosage level will be about 0.1 mg/kg to about 25 mg/kg per day; more preferably about 0.5 mg/kg to about 10 mg/kg per day.

In larger mammals, for example humans, a typical indicated dose is about 0.5 mg to 7 grams orally per day. A compound may be administered on a regimen of several times per day, for example 1 to 4 times per day, preferably once or twice per day.

The amount of the Cox-2 inhibitor that may be combined with the carrier materials to produce a single dosage form will vary depending upon the host treated and the particular mode of administration. For example, a formulation intended for the oral administration of humans may contain from 0.5 mg to 7 g of active agent compounded optionally with an appropriate and convenient amount of carrier material, which may vary from about 5 to about 95 percent of the total composition. Dosage unit forms for the Cox-2 inhibitor will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg.

Preferably, the dosage level of the Cox-2 inhibitor will be about 0.001 mg per kg to about 100 mg per kg per day; more preferably about 0.01 mg per kg to about 50 mg per kg per day; even more preferably about 0.1 mg per kg to about 10 mg per kg subject body weight.

A combination therapy comprising a Cox-2 inhibitor and an EGF receptor antagonist will have an appropriate dosage level of the EGF receptor antagonist that will generally be from about 10 mg per day for an adult human to about 5000 mg per day for an adult human, which may be administered in single or multiple doses. Preferably, the dosage level will be about 50 mg to about 1000 mg per day; more preferably about 100 mg to about 750 mg per day for an adult human.

The exact dosage and regimen for administering an EGF receptor antagonist alone and in combination with a Cox-2 inhibitor will necessarily depend upon the potency and duration of action of the compounds used, the nature and severity of the illness to be treated, as well as the sex, age, weight, general health and individual responsiveness of the patient to be treated, and other relevant circumstances. Those skilled in the art will appreciate that dosages may also be determined with guidance from Goodman & Goldman's The Pharmacological Basis of Therapeutics, Ninth Edition (1996), Appendix II, pp.1707-1711.

Methods for the extrapolation of effective dosages in mice, and other animals, to humans are known to the art; for example, See U.S. Pat. No. 4,938,949.

To determine the effectiveness of a particular dosage of an EGF receptor antagonist alone and in combination with a Cox-2 inhibitor is to monitor the effect of a given dosage on the progression of the disorder or prevention of a neoplasia disorder.

In one embodiment, the effectiveness of a particular dosage of EGF receptor antagonist alone and in combination with a Cox-2 inhibitor is determined by staging the disorder at multiple points during a subject's treatment. For example, once a histologic diagnosis is made, staging (i.e., determination of the extent of disease) helps determine treatment decisions and prognosis. Clinical staging uses data from the patient's history, physical examination, and noninvasive studies. Pathologic staging requires tissue specimens.

Pathological staging is performed by obtaining a biopsy of the neoplasm or tumor. A biopsy is performed by obtaining a tissue specimen of the tumor and examining the cells microscopically. A bone marrow biopsy is especially useful in determining metastases from malignant lymphoma and small cell lung cancer. Marrow biopsy will be positive in 50 to 70% of patients with malignant lymphoma (low and intermediate grade) and in 15 to 18% of patients with small cell lung cancer at diagnosis. See The Merck Manual of Diagnosis & Therapy, Beers & Brakow, 17^{th} edition, Published by Merck Research Labs, Sec. 11, Chapter 84, Hematology and Oncology, Overview of Cancer (1999).

Determination of serum chemistries and enzyme levels may also help staging. Elevation of liver enzymes (alkaline phosphatase, LDH, and ALT) suggests the presence of liver metastases. Elevated alkaline phosphatase and serum calcium may be the first evidence of bone metastases. Elevated acid phosphatase (tartrate inhibited) suggests extracapsular extension of prostate cancer. Fasting hypoglycemia may indicate an insulinoma, hepatocellular carcinoma, or retroperitoneal sarcoma. Elevated BUN or creatinine levels may indicate an obstructive uropathy secondary to a pelvic mass, intrarenal obstruction from tubular precipitation of myeloma protein, or uric acid nephropathy from lymphoma or other cancers. Elevated uric acid levels often occur in myeloproliferative and lymphoproliferative disorders. α-Fetoprotein may be elevated in hepatocellular carcinoma and testicular carcinomas, carcinoembryonic antigen-S in colon cancer, human chorionic gonadotropin in choriocarcinoma and testicular carcinoma, serum immunoglobulins in multiple myeloma, and DNA probes (bcr probe to identify the chromosome 22 change) in CML.

Tumors may synthesize proteins that produce no clinical symptoms, e.g., human chorionic gonadotropin, α-fetoprotein, carcinoembryonic antigen, CA 125, and CA 153. These protein products may be used as tumor markers in the serial evaluation of patients for determining disease recurrence or response to therapy. Thus, monitering a subject for these tumor markers is indicitive of the progress of a neoplasia disorder. Such monitering is also indicative of how well the methods and compositions of the present invention are treating or preventing a neoplasia disorder. Likewise, tumor marker monitering is effective to determine the approporiate dosages of the compositions of the present invention for treating neoplasia.

The term “clinical tumor” or “tumor” includes neoplasms that are identifiable through clinical screening or diagnostic procedures including, but not limited to, palpation, biopsy, cell proliferation index, endoscopy, mammagraphy, digital mammography, ultrasonography, computed tomagraphy (CT), magnetic resonance imaging (MRI), positron emmission tomaagraphy (PET), radiography, radionuclide evaluation, CT- or MRI-guided aspiration cytology, and imaging-guided needle biopsy, among others. Such diagnostic techniques are well known to those skilled in the art and are described in Cancer Medicine 4^{th} Edition, Volume One. J. F. Holland, R. C. Bast, D. L. Morton, E. Frei III, D. W. Kufe, and R. R. Weichselbaum (Editors). Williams & Wilkins, Baltimore (1997).

The term “tumor marker” or “tumor biomarker” encompasses a wide variety of molecules with divergent characteristics that appear in body fluids or tissue in association with a clinical tumor and also includes tumor-associated chromosomal changes. Tumor markers fall primarily into three categories: molecular or cellular markers, chromosomal markers, and serological or serum markers. Molecular and chromosomal markers complement standard parameters used to describe a tumor (i.e. histopathology, grade, tumor size) and are used primarily in refining disease diagnosis and prognosis after clinical manifestation. Serum markers can often be measured many months before clinical tumor detection and are thus useful as an early diagnostic test, in patient monitoring, and in therapy evaluation.

Molecular Tumor Markers

Molecular markers of cancer are products of cancer cells or molecular changes that take place in cells because of activation of cell division or inhibition of apoptosis. Expression of these markers can predict a cell's malignant potential. Because cellular markers are not secreted, tumor tissue samples are generally required for their detection. Non-limiting examples of molecular tumor markers that can be used in the present invention are listed in Table No. 13, below.

TABLE NO. 13
Non-limiting Examples of Molecular Tumor Markers

| Tumor | Marker |
|---|---|
| Breast | p53 |
| Breast, Ovarian | ErbB-2/Her-2 |
| Breast | S phase and ploidy |
| Breast | pS2 |
| Breast | MDR2 |
| Breast | urokinase plasminogen activator |
| Breast, Colon, | myc family |
| Lung | |

Chromosomal Tumor Markers

Somatic mutations and chromosomal aberrations have been associated with a variety of tumors. Since the identification of the Philadelphia Chromosome by Nowel and Hungerford, a wide effort to identify tumor-specific chromosomal alterations has ensued. Chromosomal cancer markers, like cellular markers, are can be used in the diagnosis and prognosis of cancer. In addition to the diagnostic and prognostic implications of chromosomal alterations, it is hypothesized that germ-line mutations can be used to predict the likelihood that a particular person will develop a given type of tumor. Non-limiting examples of chromosomal tumor markers that can be used in the present invention are listed in Table No. 14, below.

TABLE NO. 14
Non-limiting Examples of Chromosomal Tumor Markers

| Tumor | Marker |
|---|---|
| Breast | 1p36 loss |
| Breast | 6q24-27 loss |
| Breast | 11q22-23 loss |
| Breast | 11q13 amplification |
| Breast | TP53 mutation |
| Colon | Gain of chromosome 13 |
| Colon | Deletion of short arm of chromosome 1 |
| Lung | Loss of 3p |
| Lung | Loss of 13q |
| Lung | Loss of 17p |
| Lung | Loss of 9p |

Serological Tumor Markers

Serum markers including soluble antigens, enzymes and hormones comprise a third category of tumor markers. Monitoring serum tumor marker concentrations during therapy provides an early indication of tumor recurrence and of therapy efficacy. Serum markers are advantageous for patient surveillance compared to chromosomal and cellular markers because serum samples are more easily obtainable than tissue samples, and because serum assays can be performed serially and more rapidly. Serum tumor markers can be used to determine appropriate therapeutic doses within individual patients. For example, the efficacy of a combination regimen consisting of chemotherapeutic and antiangiogenic agents can be measured by monitoring the relevant serum cancer marker levels. Moreover, an efficacious therapy dose can be achieved by modulating the therapeutic dose so as to keep the particular serum tumor marker concentration stable or within the reference range, which may vary depending upon the indication. The amount of therapy can then be modulated specifically for each patient so as to minimize side effects while still maintaining stable, reference range tumor marker levels. Table No. 15 provides non-limiting examples of serological tumor markers that can be used in the present invention.

TABLE NO. 15
Non-limiting Examples of Serum Tumor Markers

| Cancer Type | Marker |
|---|---|
| Germ Cell Tumors | a-fetoprotein (AFP) |
| Germ Cell Tumors | human chorionic gonadotrophin (hCG) |
| Germ Cell Tumors | placental alkaline phosphatase (PLAP) |
| Germ Cell Tumors | lactate dehydrogenase (LDH) |
| Prostate | prostate specific antigen (PSA) |
| Breast | carcinoembryonic antigen (CEA) |
| Breast | MUC-1 antigen (CA15-3) |
| Breast | tissue polypeptide antigen (TPA) |
| Breast | tissue polypeptide specific antigen |
| | (TPS) |
| Breast | CYFRA 21.1 |
| Breast | soluble erb-B-2 |
| Ovarian | CA125 |
| Ovarian | OVX1 |
| Ovarian | cancer antigen CA72-4 |
| Ovarian | TPA |
| Ovarian | TPS |
| Gastrointestinal | CD44v6 |
| Gastrointestinal | CEA |
| Gastrointestinal | cancer antigen CA19-9 |
| Gastrointestinal | NCC-ST-439 antigen (Dukes C) |
| Gastrointestinal | cancer antigen CA242 |
| Gastrointestinal | soluble erb-B-2 |
| Gastrointestinal | cancer antigen CA195 |
| Gastrointestinal | TPA |
| Gastrointestinal | YKL-40 |
| Gastrointestinal | TPS |
| Esophageal | CYFRA 21-1 |
| Esophageal | TPA |
| Esophageal | TPS |
| Esophageal | cancer antigen CA19-9 |
| Gastric Cancer | CEA |
| Gastric Cancer | cancer antigen CA19-9 |
| Gastric Cancer | cancer antigen CA72-4 |
| Lung | neruon specific enolase (NSE) |
| Lung | CEA |
| \Lung | CYFRA 21-1 |
| Lung | cancer antigen CA 125 |
| Lung | TPA |
| Lung | squamous cell carcinoma antigen (SCC) |
| Pancreatic cancer | ca19-9 |
| Pancreatic cancer | ca50 |
| Pancreatic cancer | ca119 |
| Pancreatic cancer | ca125 |
| Pancreatic cancer | CEA |
| Pancreatic cancer | |
| Renal Cancer | CD44v6 |
| Renal Cancer | E-cadherin |
| Renal Cancer | PCNA (proliferating cell nuclear antigen) |

Germ Cell Cancers

Non-limiting examples of tumor markers useful in the present invention for the detection of germ cell cancers include, but are not limited to, a-fetoprotein (AFP), human chorionic gonadotrophin (hCG) and its beta subunit (hCGb), lactate dehydrogenase (LDH), and placental alkaline phosphatase (PLAP).

AFP has an upper reference limit of approximately −10 kU/L after the first year of life and may be elevated in germ cell tumors, hepatocellular carcinoma and also in gastric, colon, biliary, pancreatic and lung cancers. AFP serum half-life is approximately five days after orchidectomy. According to EGTM recommendations, AFP serum levels less than 1,000 kU/L correlate with a good prognosis, AFP levels between 1,000 and 10,000 kU/L, inclusive, correlate with intermediate prognosis, and AFP levels greater than 10,000 U/L correlate with a poor prognosis.

HCG is synthesized in the placenta and is also produced by malignant cells. Serum hCG concentrations may be increased in pancreatic adenocarcinomas, islet cell tumors, tumors of the small and large bowel, hepatoma, stomach, lung, ovaries, breast and kidney. Because some tumors only hCGb, measurement of both hCG and hCGb is recommended. Normally, serum hCG in men and pre-menopausal women is as high as −5 U/L while post-menopausal women have levels up to −10 U/L. Serum half life of hCG ranges from 16-24 hours. According to the EGTM, hCG serum levels under 5000 U/L correlate with a good prognosis, levels between 5000 and 50000 U/L, inclusively correlate with an intermediate prognosis, and hCG serum levels greater than 50000 U/L correlate with a poor prognosis. Further, normal hCG half lives correlate with good prognosis while prolonged half lives correlate with poor prognosis.

LDH is an enzyme expressed in cardiac and skeletal muscle as well as in other organs. The LDH-1 isoenzyme is most commonly found in testicular germ cell tumors but can also occur in a variety of benign conditions such as skeletal muscle disease and myocardial infarction. Total LDH is used to measure independent prognostic value in patients with advanced germ cell tumors. LDH levels less than 1.5×the reference range are associated with a good prognosis, levels between 1.5 and 10×the reference range, inclusive, are associated with an intermediate prognosis, and levels more than 10×the reference range are associated with a poor prognosis.

PLAP is an enzyme of alkaline phosphatase normally expressed by placental syncytiotrophoblasts. Elevated serum concentrations of PLAP are found in seminomas, non-seminomatous tumors, and ovarian tumors, and may also provide a marker for testicular tumors. PLAP has a normal half-life after surgical resection of between 0.6 and 2.8 days.

Prostate Cancer

A nonlimiting example of a tumor marker useful in the present invention for the detection of prostate cancer is prostate specific antigen (PSA). PSA is a glycoprotein that is almost exclusively produced in the prostate. In human serum, uncomplexed f-PSA and a complex of f-PSA with a1-anthichymotrypsin make up total PSA (t-PSA). T-PSA is useful in determining prognosis in patients that are not currently undergoing anti-androgen treatment. Rising t-PSA levels via serial measurement indicate the presence of residual disease.

Breast Cancer

Non-limiting examples of serum tumor markers useful in the present invention for the detection of breast cancer include, but is not limited to carcinoembryonic antigen (CEA) and MUC-1 (CA 15.3). Serum CEA and CA15.3 levels are elevated in patients with node involvement compared to patients without node involvement, and in patients with larger tumors compared to smaller tumors. Normal range cutoff points (upper limit) are 5-10 mg/L for CEA and 35-60 u/ml for CA15.3. Additional specificity (99.3%) is gained by confirming serum levels with two serial increases of more than 15%.

Ovarian Cancer

A non-limiting example of a tumor marker useful in the present invention for the detection of ovarian cancer is CA125. Normally, women have serum CA125 levels between 0-35 kU/L; 99% of post-menopausal women have levels below 20 kU/L. Serum concentration of CA125 after chemotherapy is a strong predictor of outcome as elevated CA125 levels are found in roughly 80% of all patients with epithelial ovarian cancer. Further, prolonged CA125 half-life or a less than 7-fold decrease during early treatment is also a predictor of poor disease prognosis.

Gastrointestinal Cancers

A non-limiting example of a tumor marker useful in the present invention for the detection of colon cancer is carcinoembryonic antigen (CEA). CEA is a glycoprotein produced during embryonal and fetal development and has a high sensitivity for advanced carcinomas including those of the colon, breast, stomach and lung. High pre- or postoperative concentrations (>2.5 ng/ml) of CEA are associated with worse prognosis than are low concentrations. Further, some studies in the literature report that slow rising CEA levels indicates local recurrence while rapidly increasing levels suggests hepatic metastasis.

Lung Cancer

Examples of serum markers useful in the present invention to monitor lung cancer therapy include, but are not limited to, CEA, cytokeratin 19 fragments (CYFRA 21-1), and Neuron Specific Enolase (NSE).

NSE is a glycolytic isoenzyme of enolase produced in central and peripheral neurons and malignant tumors of neuroectodermal origin. At diagnosis, NSE concentrations greater than 25 ng/mL are suggestive of malignancy and lung cancer while concentrations greater than 100 ng/mL are suggestive of small cell lung cancer.

CYFRA 21-1 is a tumor marker test, which uses two specific monoclonal antibodies against a cytokeratin 19 fragment. At diagnosis, CYFRA 21-1 concentrations greater than 10 ng/mL are suggestive of malignancy while concentrations greater than 30 ng/mL are suggestive of lung cancer.

Accordingly, dosing of the antiangiongenic agnents and optionally an antineoplastic agent may be determined and adjusted based on measurement of tumor markers in body fluids or tissues, particularly based on tumor markers in serum. For example, a decrease in serum marker level relative to baseline serum marker prior to administration of the antiangiongenic agnents and optionally the antineoplastic agent indicates a decrease in cancer-associated changes and provides a correlation with inhibition of the cancer. In one embodiment, therefore, the method of the present invention comprises administering the Cox-2 inhibitor, matrix metalloproteinase inhibitor, integrin antagonist and antineoplastic agent at doses that in combination result in a decrease in one or more tumor markers, particularly a decrease in one or more serum tumor markers, in the mammal relative to baseline tumor marker levels.

Similarly, decreasing tumor marker concentrations or serum half lives after administration of the combination indicates a good prognosis, while tumor marker concentrations which decline slowly and do not reach the normal reference range predict residual tumor and poor prognosis. Further, during follow-up therapy, increases in tumor marker concentration predict recurrent disease many months before clinical manifestation.

In addition to the above examples, Table No. 16, below, lists several references, hereby individually incorporated by reference herein, that describe tumor markers and their use in detecting and monitoring tumor growth and progression.

TABLE NO. 16
Tumor marker references.

European Group on Tumor Markers Publications Committee. Consensus
Recommendations. Anticancer Research 19: 2785-2820 (1999)
Human Cytogenetic Cancer Markers. Sandra R. Wolman and Stewart
Sell (eds.). Totowa, New Jersey: Humana Press. 1997
Cellular Markers of Cancer. Carleton Garrett and Stewart Sell (eds.).
Totowa, New Jersey: Human Press. 1995

Other techniques include mediastinoscopy, which is especially valuable in the staging of non-small cell lung cancer. If mediastinoscopy shows mediastinal lymph node involvement, then the subject would not usually benefit from a thoracotomy and lung resection. Imaging studies, especially CT and MRI, can detect metastases to brain, lung, spinal cord, or abdominal viscera, including the adrenal glands, retroperitoneal lymph nodes, liver, and spleen. MRI (with gadolinium) is the procedure of choice for recognition and evaluation of brain tumors.

Ultrasonography can be used to study orbital, thyroid, cardiac, pericardial, hepatic, pancreatic, renal, and retroperitoneal areas. It may guide percutaneous biopsies and differentiate renal cell carcinoma from a benign renal cyst. Lymphangiography reveals enlarged pelvic and low lumbar lymph nodes and is useful in the clinical staging of patients with Hodgkin's disease, but it has generally been replaced by CT.

Liver-spleen scans can identify liver metastases and splenomegaly. Bone scans are sensitive in identifying metastases before they are evident on x-ray. Because a positive scan requires new bony formation (i.e., osteoblastic activity), this technique is useless in neoplasms that are purely lytic (e.g., multiple myeloma); routine bone x-rays are the study of choice in such diseases. Gallium scans can help in staging lymphoid neoplasms. Radiolabeled monoclonal antibodies (e.g., to carcinoembryonic antigen, small cell lung cancer cells) provide important staging data in various neoplasms (e.g., colon cancer, small cell lung cancer). See The Merck Manual of Diagnosis & Therapy, Beers & Brakow, 17^{th} edition, Published by Merck Research Labs, Sec. 11, Chapter 84, Hematology and Oncology, Overview of Cancer (1999).

As used herein, the term “subject” for purposes of treatment includes any subject, and preferably is a subject who is in need of the treatment of neoplasia or a neoplasia-related disorder. For purposes of prevention, the subject is any subject, and preferably is a subject that is at risk for, or is predisposed to, developing neoplasia or a neoplasia-related disorder.

As used herein, the terms “subject in need of” refer to any subject who is suffering from or is predisposed to neoplasia or any neoplasia-related disorder described herein. The terms “subject in need of” also refer to any subject that requires a lower dose of conventional neoplasia treatment agents. In addition, the terms “subject in need of” means any subject who requires a reduction in the side effects of a conventional treatment agent. Furthermore, the terms “subject in need of” means any subject who requires improved tolerability to any conventional treatment agent for a neoplasia disorder therapy.

The subject is typically an animal, and yet more typically is a mammal. “Mammal”, as that term is used herein, refers to any animal classified as a mammal, including humans, domestic and farm animals, zoo, sports, or pet animals, such as dogs, horses, cats, cattle, etc. The subject may also be a human subject who is at risk for developing neoplasia or at risk for a relapse of a neoplasia disorder.

The methods and compositions of the present invention may be used for the treatment or prevention of several neoplasia disorders and neoplasia-related disorders and complications including, but are not limited to, acral lentiginous melanoma, actinic keratoses, adenocarcinoma, adenoid cycstic carcinoma, adenomas, adenosarcoma, adenosquamous carcinoma, adrenocortical carcinoma, AIDS-related lymphoma, anal cancer, astrocytic tumors, bartholin gland carcinoma, basal cell carcinoma, bile duct cancer, bladder cancer, brain stem glioma, brain tumors, breast cancer, bronchial gland carcinomas, capillary carcinoma, carcinoids, carcinoma, carcinosarcoma, cavernous, central nervous system lymphoma, cerebral astrocytoma, cholangiocarcinoma, chondosarcoma, choriod plexus papilloma/carcinoma, clear cell carcinoma, colon cancer, colorectal cancer, cutaneous T-cell lymphoma, cystadenoma, endodermal sinus tumor, endometrial hyperplasia, endometrial stromal sarcoma, endometrioid adenocarcinoma, ependymal, epitheloid, esophageal cancer, Ewing's sarcoma, extragonadal germ cell tumor, fibrolamellar, focal nodular hyperplasia, gallbladder cancer, gastrinoma, germ cell tumors, gestational trophoblastic tumor, glioblastoma, glioma, glucagonoma, hemangiblastomas, hemangioendothelioma, hemangiomas, hepatic adenoma, hepatic adenomatosis, hepatocellular carcinoma, Hodgkin's lymphoma, hypopharyngeal cancer, hypothalamic and visual pathway glioma, childhood, insulinoma, intaepithelial neoplasia, interepithelial squamous cell neoplasia, intraocular melanoma, invasive squamous cell carcinoma, large cell carcinoma, islet cell carcinoma, Kaposi's sarcoma, kidney cancer, laryngeal cancer, leiomyosarcoma, lentigo maligna melanomas, leukemia-related disorders, lip and oral cavity cancer, liver cancer, lung cancer, lymphoma, malignant mesothelial tumors, malignant thymoma, medulloblastoma, medulloepithelioma, melanoma, meningeal, merkel cell carcinoma, mesothelial, metastatic carcinoma, mucoepidermoid carcinoma, multiple myeloma/plasma cell neoplasm, mycosis fungoides, myelodysplastic syndrome, myeloproliferative disorders, nasal cavity and paranasal sinus cancer, nasopharyngeal cancer, neuroblastoma, neuroepithelial adenocarcinoma nodular melanoma, non-Hodgkin's lymphoma, non-small cell lung cancer, oat cell carcinoma, oligodendroglial, oral cancer, oropharyngeal cancer, osteosarcoma, pancreatic polypeptide, ovarian cancer, ovarian germ cell tumor, pancreatic cancer, papillary serous adenocarcinoma, pineal cell, pituitary tumors, plasmacytoma, pseudosarcoma, pulmonary blastoma, parathyroid cancer, penile cancer, pheochromocytoma, pineal and supratentorial primitive neuroectodermal tumors, pituitary tumor, plasma cell neoplasm, pleuropulmonary blastoma, prostate cancer, rectal cancer, renal cell carcinoma, retinoblastoma, rhabdomyosarcoma, sarcoma, serous carcinoma, small cell carcinoma, small intestine cancer, soft tissue carcinomas, somatostatin-secreting tumor, squamous carcinoma, squamous cell carcinoma, submesothelial, superficial spreading melanoma, supratentorial primitive neuroectodermal tumors, thyroid cancer, undifferentiatied carcinoma, urethral cancer, uterine sarcoma, uveal melanoma, verrucous carcinoma, vaginal cancer, vipoma, vulvar cancer, Waldenstrom's macroglobulinemia, well differentiated carcinoma, and Wilm's tumor.

### EXAMPLES

### Example 1

Cancer cells were implanted subcutaneously in genetically engineered mice and grew large-volume tumors (>1,500 mm³). Subsequent administration of S836 reduced tumor growth by as much as 85 percent in a dose dependent manner. (Nickols A, et al., Inhibition of tumor growth and metastasis by an ανβ3 integrin antagonist. Presented at the 89^{th} Annual Meeting of the American Association for Cancer Research, March, 1998.) In another mouse model, scientists engineered lung tumors of volumes greater than 2,000 mm. They then separated the mice into four groups, including a control group and three treatment groups: S386 alone; S386 with cisplatin (a cytotoxic drug); or cisplatin alone. Compared to the control groups, the mice treated with combination S386/cisplatin therapy experienced more than an 80 percent reduction in tumor size. In comparison, the group receiving cisplatin alone experienced 50 percent reductions in tumor size and the S386 group experienced 20-30 percent reductions. These studies indicate that S836 has prominent anti-tumor activity due to antiangiogenic properties.

### Example 2

Lung Cancer

In many countries including Japan, Europe and America, the number of patients with lung cancer is large and continues to increase year after year and is the most frequent cause of cancer death in both men and women. Although there are many potential causes for lung cancer, tobacco use, and particularly cigarette smoking, is the most important. Additionally, etiologic factors such as exposure to asbestos, especially in smokers, or radon are contributory factors. Also occupational hazards such as exposure to uranium have been identified as an important factor. Finally, genetic factors have also been identified as another factor that increase the risk of cancer.

Lung cancers can be histologically classified into non-small cell lung cancers (e.g. squamous cell carcinoma (epidermoid), adenocarcinoma, large cell carcinoma (large cell anaplastic), etc.) and small cell lung cancer (oat cell). Non-small cell lung cancer (NSCLC) has different biological properties and responses to chemotherapeutics from those of small cell lung cancer (SCLC). Thus, chemotherapeutic formulas and radiation therapy are different between these two types of lung cancer.

Non-Small Cell Lung Cancer

Where the location of the non-small cell lung cancer tumor can be easily excised (stage I and II disease) surgery is the first line of therapy and offers a relatively good chance for a cure. However, in more advanced disease (stage IIIa and greater), where the tumor has extended to tissue beyond the bronchopulmonary lymph nodes, surgery may not lead to complete excision of the tumor. In such cases, the patient's chance for a cure by surgery alone is greatly diminished. Where surgery will not provide complete removal of the NSCLC tumor, other types of therapies must be utilized.

Today radiation therapy is the standard treatment to control unresectable or inoperable NSCLC. Improved results have been seen when radiation therapy has been combined with chemotherapy, but gains have been modest and the search continues for improved methods of combining modalities.

Radiation therapy is based on the principle that high-dose radiation delivered to a target area will result in the death of reproductive cells in both tumor and normal tissues. The radiation dosage regimen is generally defined in terms of radiation absorbed dose (rad), time and fractionation, and must be carefully defined by the oncologist. The amount of radiation a patient receives will depend on various consideration but the two most important considerations are the location of the tumor in relation to other critical structures or organs of the body, and the extent to which the tumor has spread. A prefered course of treatment for a patient undergoing radiation therapy for NSCLC will be a treatment schedule over a 5 to 6 week period, with a total dose of 50 to 60 Gy administered to the patient in a single daily fraction of 1.8 to 2.0 Gy, 5 days a week. A Gy is an abbreviation for Gray and refers to 100 rad of dose.

However, as NSCLC is a systemic disease, and radiation therapy is a local modality, radiation therapy as a single line of therapy is unlikely to provide a cure for NSCLC, at least for those tumors that have metastasized distantly outside the zone of treatment. Thus, the use of radiation therapy with other modality regimens have important beneficial effects for the treatment of NSCLC.

Generally, radiation therapy has been combined temporally with chemotherapy to improve the outcome of treatment. There are various terms to describe the temporal relationship of administering radiation therapy and chemotherapy, and the following examples are the preferred treatment regimens and are generally known by those skilled in the art and are provided for illustration only and are not intended to limit the use of other combinations. “Sequential” radiation therapy and chemotherapy refers to the administration of chemotherapy and radiation therapy separately in time in order to allow the separate administration of either chemotherapy or radiation therapy. “Concomitant” radiation therapy and chemotherapy refers to the administration of chemotherapy and radiation therapy on the same day. Finally, “alternating” radiation therapy and chemotherapy refers to the administration of radiation therapy on the days in which chemotherapy would not have been administered if it was given alone.

It is reported that advanced non-small cell lung cancers do not respond favorably to single-agent chemotherapy and useful therapies for advanced inoperable cancers have been limited. (Journal of Clinical Oncology, vol.10, pp. 829-838 (1992)).

Japanese Patent Kokai 5-163293 refers to some specified antibiotics of 16-membered-ring macrolides as a drug delivery carrier capable of transporting anthoracycline-type anticancer drugs into the lungs for the treatment of lung cancers. However, the macrolide antibiotics specified herein are disclosed to be only a drug carrier, and there is no reference to the therapeutic use of macrolides against non-small cell lung cancers.

WO 93/18652 refers to the effectiveness of the specified 16-membered-ring macrolides such as bafilomycin, etc. in treating non-small cell lung cancers, but they have not yet been clinically practicable.

Pharmacology, vol. 41, pp.177-183 (1990) describes that a long-term use of erythromycin increases productions of interleukins 1, 2 and 4, all of which contribute to host immune responses, but there is no reference to the effect of this drug on non-small cell lung cancers.

Tetragenesis, Carcinogenesis, and Mutagenesis, vol. 10, pp. 477-501 (1990) describes that some of antimicrobial drugs can be used as an anticancer agent, but does not refer to their application to non-small cell lung cancers.

In addition, interleukins are known to have an antitumor effect, but have not been reported to be effective against non-small cell lung cancers.

Any 14- or 15-membered-ring macrolides have not been reported to be effective against non-small cell lung cancers.

However, several chemotherapeutic agents have been shown to be efficacious against NSCLC. Preferred chemotherapeutic agents against NSCLC include etoposide, carboplatin, methotrexate, 5-Fluorouracil, epirubicin, doxorubicin, and cyclophosphamide. The most preferred chemotherapeutic agents active against NSCLC include cisplatin, ifosfamide, mitomycin C, epirubicin, vinblastine, and vindesine.

Other agents that are under investigation for use against NSCLC include: camptothecins, a topoisomerase 1 inhibitor; navelbine (vinorelbine), a microtubule assebly inhibitor; taxol, inhibitor of normal mitotic activity; gemcitabine, a deoxycytidine analogue; fotemustine, a nitrosourea compound; and edatrexate, a antifol.

The overall and complete response rates for NSCLC has been shown to increase with use of combination chemotherapy as compared to single-agent treatment. See Haskel, C M, Chest. 99:1325, 1991; Bakowski, M T, Cancer Treat Rev 10:159 (1983), and Joss, R A, Cancer Treat Rev 11:205 (1984).

The most preferred therapy for the treatment of NSCLC is a combination of therapeutically effective amounts of one or more antiangiogenesis agents selected from the group consisting of a matrix metalloproteinase inhibitor (MMP), a cyclooxygenase-II inhibitor (COX-II), an alpha v beta 3 inhibitor, an angiostatin, an endostatin, or a pBATT; in combination with 1) itosfamide, cisplatin, etoposide; 2) cyclophoshamide, doxorubicin, cisplatin; 3) isofamide, carboplatin, etoposide; 4) bleomycin, etoposide, cisplatin; 5) isofamide, mitomycin, cisplatin; 6) cisplatin, vinblastine; 7) cisplatin, vindesine; 8) mitomycin C, vinblastine, cisplatin; 9) mitomycin C, vindesine, cisplatin; 10) isofamide, etoposide; 11) etoposide, cisplatin; 12) isofamide, mitomycin C; 13) flurouracil, cisplatin, vinblastine; 14) carboplatin, etoposide; or 15) radiation therapy.

Accordingly, apart from the conventional concept of anticancer therapy, there is a strong need for the development of therapies practicably effective for the treatment of non-small cell lung cancers.

Small Cell Lung Cancer

Approximately 15 to 20 percent of all cases of lung cancer reported worldwide is small cell lung cancer (SCLC). Ihde, D C, Cancer 54:2722 (1984). Currently, treatment of SCLC incorporates multi-modal therapy, including chemotherapy, radiation therapy and surgery. Response rates of localized or disseminated SCLC remain high to systemic chemotherapy, however, persistence of the primary tumor and persistence of the tumor in the associated lymph nodes has led to the integration of several therapeutic modalities in the treatment of SCLC.

The most preferred chemotherapeutic agents against SCLC include vincristine, cisplatin, carboplatin, cyclophosphamide, epirubicin (high dose), etoposide (VP-16) I.V., etoposide (VP-16) oral, isofamide, teniposide (VM-26), and doxorubicin. Preferred single-agents chemotherapeutic agents include BCNU (carmustine), vindesine, hexamethylmelamine (altretamine), methotrexate, nitrogen mustard, and CCNU (lomustine). Other chemotherapeutic agents under investigation that have shown activity againe SCLC include iroplatin, gemcitabine, lonidamine, and taxol. Single-agent chemotherapeutic agents that have not shown activity against SCLC include mitoguazone, mitomycin C, aclarubicin, diaziquone, bisantrene, cytarabine, idarubicin, mitomxantrone, vinblastine, PCNU and esorubicin.

The poor results reported from single-agent chemotherapy has led to use of combination chemotherapy.

The most preferred therapy for the treatment of SCLC is a combination of therapeutically effective amounts of one or more antiangiogenesis agents selected from the group consisting of a matrix metalloproteinase inhibitor (MMP), a cyclooxygenase-II inhibitor (COX-II), an alpha v beta 3 inhibitor, an angiostatin, an endostatin, or a pBATT; in combination with 1) etoposide (VP-16), cisplatin; 2) cyclophosphamide, adrianmycin [(doxorubicin), vincristine, etoposide (VP-16)]; 3) Cyclophosphamide, adrianmycin(doxorubicin), vincristine; 4) Etoposide (VP-16), ifosfamide, cisplatin; 5) etoposide (VP-16), carboplatin; 6) cisplatin, vincristine (Oncovin), doxorubicin, etoposide.

Additionally, radiation therapy in conjunction with the preferred combinations of angiogenesis inhibitors and systemic chemotherapy is contemplated to be effective at increasing the response rate for SCLC patients. The typical dosage regimen for radiation therapy ranges from 40 to 55 Gy, in 15 to 30 fractions, 3 to 7 times week. The tissue volume to be irradiated is determined by several factors and generally, the hilum and subcarnial nodes, and bialteral mdiastinal nodes up to the thoraic inlet are treated, as well as the primary tumor up to 1.5 to 2.0 cm of the margins.

### Example 3

Colorectal Cancer

Survival from colorectal cancer depends on the stage and grade of the tumor, for example precursor adenomas to metastatic adenocarcinoma. Generally, colorectal cancer can be treated by surgically removing the tumor, but overall survival rates remain between 45 and 60 percent. Colonic excision morbidity rates are fairly low and is generally associated with the anastomosis and not the extent of the removal of the tumor and local tissue. In patients with a high risk of reoccurrence, however, chemotherapy has been incorporated into the treatment regimen in order to improve survival rates.

Tumor metastasis prior to surgery is generally believed to be the cause of surgical intervention failure and up to one year of chemotherapy is required to kill the non-excised tumor cells. As severe toxicity is associated with the chemotherapeutic agents, only patients at high risk of recurrence are placed on chemotherapy following surgery. Thus, the incorporation of an antiangiogenesis inhibitor into the management of colorectal cancer will play an important role in the treatment of colorectal cancer and lead to overall improved survival rates for patients diagnosed with colorectal cancer.

The preferred combination therapy for the treatment of colorectal cancer is surgery, followed by a regimen of one or more chemotherapeutic agents and one or more antiangiogenic agents, cycled over a one year time period. Another preferred combination therapy for the treatment of colorectal cancer is a regimen of one or more antiangiogenic agents, followed by surgical removal of the tumor from the colon or rectum and then followed be a regimen of one or more chemotherapeutic agents and one or more antiangiogenic agents, cycled over a one year time period.

Preferred chemotherapeutic agents include fluorouracil, and Levamisole. Preferably, fluorouracil and Levamisole are used in combination.

### Example 4

Breast Cancer

Today, among women in the United States, breast cancer remains the most frequent diagnoses cancer. One in 8 women in the United States at risk of developing breast cancer in their lifetime. Age, family history, diet, and genetic factors have been identified as risk factors for breast cancer. Breast cancer is the second leading cause of death among women.

Different chemotherapeutic agents are known in art for treating breast cancer. Cytoxic agents used for treating breast cancer include doxorubicin,cyclophosphamide, methotrexate, 5-fluorouracil, mitomycin C, mitoxantrone, taxol, and epirubicin. CANCER SURVEYS, Breast Cancer volume 18, Cold Spring Harbor Laboratory Press, 1993.

In the treatment of locally advanced noninflammatory breast cancer, antiangiogenic agents can be used to treat the disease in combination with other antiangiogenic agents, or in combination with surgery, radiation therapy or with chemotherapeutic agents. Preferred combinations of chemotherapeutic agents, radiation therapy and surgery that can be used in combination with the angiogenesis inhibitors include, but are not limited to: 1) doxorubicin, vincristine, radical mastectomy; 2) doxorubicin, vincristine, radiation therapy; 3) cyclophosphamide, doxorubicin, 5-flourouracil, vincristine, prednisone, mastecomy; 4) cyclophosphamide, doxorubicin, 5-flourouracil, vincristine, prednisone, radiation therapy; 5) cyclophosphamide, doxorubicin, 5-flourouracil, premarin, tamoxifen, radiation therapy for pathologic complete response; 6) cyclophosphamide, doxorubicin, 5-flourouracil, premarin, tamoxifen, mastectomy, radiation therapy for pathologic partial response; 7) mastectomy, radiation therapy, levamisole; 8) mastectomy, radiation therapy; 9) mastectomy, vincristine, doxorubicin, cyclophosphamide, levamisole; 10) mastectomy, vincristine, doxorubicin, cyclophosphamide; 11) mastecomy, cyclophosphamide, doxorubicin, 5-fluorouracil, tamoxifen, halotestin, radiation therapy; 12) mastecomy, cyclophosphamide, doxorubicin, 5-fluorouracil, tamoxifen, halotestin.

In the treatment of locally advanced inflammatory breast cancer, antiangiogenic agents can be used to treat the disease in combination with other antiangiogenic agents, or in combination with surgery, radiation therapy or with chemotherapeutic agents. Preferred combinations of chemotherapeutic agents, radiation therapy and surgery that can be used in combination with the angiogenesis inhibitors include, but or not limited to: 1) cyclophosphamide, doxorubicin, 5-fluorouracil, radiation therapy; 2) cyclophosphamide, doxorubicin, 5-fluorouracil, mastectomy, radiation therapy; 3) 5-flurouracil, doxorubicin, clyclophosphamide, vincristine, prednisone, mastectomy, radiation therapy; 4) 5-flurouracil, doxorubicin, clyclophosphamide, vincristine, mastectomy, radiation therapy; 5) cyclophosphamide, doxorubicin, 5-fluorouracil, vincristine, radiation therapy; 6) cyclophosphamide, doxorubicin, 5-fluorouracil, vincristine, mastectomy, radiation therapy; 7) doxorubicin, vincristine, methotrexate, radiation therapy, followed by vincristine, cyclophosphamide, 5-florouracil; 8) doxorubicin, vincristine, cyclophosphamide, methotrexate, 5-florouracil, radiation therapy, followed by vincristine, cyclophosphamide, 5-florouracil; 9) surgery, followed by cyclophosphamide, methotrexate, 5-fluorouracil, predinsone, tamoxifen, followed by radiation therapy, followed by cyclophosphamide, methotrexate, 5-fluorouracil, predinsone, tamoxifen, doxorubicin, vincristine, tamoxifen; 10) surgery, followed by cyclophosphamide, methotrexate, 5-fluorouracil, followed by radiation therapy, followed by cyclophosphamide, methotrexate, 5-fluorouracil, predinsone, tamoxifen, doxorubicin, vincristine, tamoxifen; 11) surgery, followed by cyclophosphamide, methotrexate, 5-fluorouracil, predinsone, tamoxifen, followed by radiation therapy, followed by cyclophosphamide, methotrexate, 5-fluorouracil, doxorubicin, vincristine, tamoxifen; 12) surgery, followed by cyclophosphamide, methotrexate, 5-fluorouracil, followed by radiation therapy, followed by cyclophosphamide, methotrexate, 5-fluorouracil, predinsone, tamoxifen, doxorubicin, vincristine; 13) surgery, followed by cyclophosphamide, methotrexate, 5-fluorouracil, predinsone, tamoxifen, followed by radiation therapy, followed by cyclophosphamide, methotrexate, 5-fluorouracil, predinsone, tamoxifen, doxorubicin, vincristine, tamoxifen; 14) surgery, followed by cyclophosphamide, methotrexate, 5-fluorouracil, followed by radiation therapy, followed by cyclophosphamide, methotrexate, 5-fluorouracil, predinsone, tamoxifen, doxorubicin, vincristine; 15) surgery, followed by cyclophosphamide, methotrexate, 5-fluorouracil, predinsone, tamoxifen, followed by radiation therapy, followed by cyclophosphamide, methotrexate, 5-fluorouracil, doxorubicin, vincristine; 16) 5-florouracil, doxorubicin, cyclophosphamide followed by mastectomy, followed by 5-florouracil, doxorubicin, cyclophosphamide, followed by radtiation therapy.

In the treatment of metastatic breast cancer, antiangiogenic agents can be used to treat the disease in combination with other antiangiogenic agents, or in combination with surgery, radiation therapy or with chemotherapeutic agents. Preferred combinations of chemotherapeutic agents, radiation therapy and surgery that can be used in combination with the angiogenesis inhibitors include, but are not limited to: 1) cyclosphosphamide, methotrexate, 5-fluorouracil; 2) cyclophosphamide, adriamycin, 5-fluorouracil; 3) cyclosphosphamide, methotrexate, 5-flurouracil, vincristine, prednisone; 4) adriamycin, vincristine; 5) thiotepa, adriamycin, vinblastine; 6) mitomycin, vinblastine; 7) cisplatin, etoposide.

### Example 5

Prostate Cancer

Prostate cancer is now the leading form of cancer among men and the second most frequent cause of death from cancer in men. It is estimated that more than 165,000 new cases of prostate cancer were diagnosed in 1993, and more than 35,000 men died from prostate cancer in that year. Additionally, the incidence of prostate cancer has increased by 50% since 1981, and mortality from this disease has continued to increase. Previously, most men died of other illnesses or diseases before dying from their prostate cancer. We now face increasing morbidity from prostate cancer as men live longer and the disease has the opportunity to progress.

Current therapies for prostate cancer focus exclusively upon reducing levels of dihydrotestosterone to decrease or prevent growth of prostate cancer.

In addition to the use of digital rectal examination and transrectal ultrasonography, prostate-specific antigen (PSA) concentration is frequently used in the diagnosis of prostate cancer.

U.S. Pat. No. 4,472,382 discloses treatment of benign prostatic hyperplasia (BPH) with an antiandrogen and certain peptides which act as LH-RH agonists.

U.S. Pat. No. 4,596,797 discloses aromatase inhibitors as a method of prophylaxis and/or treatment of prostatic hyperplasia.

U.S. Pat. No. 4,760,053 describes a treatment of certain cancers which combines an LHRH agonist with an antiandrogen and/or an antiestrogen and/or at least one inhibitor of sex steroid biosynthesis.

U.S. Pat. No. 4,775,660 discloses a method of treating breast cancer with a combination therapy which may include surgical or chemical prevention of ovarian secretions and administering an antiandrogen and an antiestrogen.

U.S. Pat. No. 4,659,695 discloses a method of treatment of prostate cancer in susceptible male animals including humans whose testicular hormonal secretions are blocked by surgical or chemical means, e.g. by use of an LHRH agonist, which comprises administering an antiandrogen, e.g. flutamide, in association with at least one inhibitor of sex steroid biosynthesis, e.g. aminoglutethimide and/or ketoconazole.

Prostate Specific Antigen

One well known prostate cancer marker is Prostate Specific Antigen (PSA). PSA is a protein produced by prostate cells and is frequently present at elevated levels in the blood of men who have prostate cancer. PSA has been shown to correlate with tumor burden, serve as an indicator of metastatic involvement, and provide a parameter for following the response to surgery, irradiation, and androgen replacement therapy in prostate cancer patients. It should be noted that Prostate Specific Antigen (PSA) is a completely different protein from Prostate Specific Membrane Antigen (PSMA). The two proteins have different structures and functions and should not be confused because of their similar nomenclature.

Prostate Specific Membrane Antigen (PSMA)

In 1993, the molecular cloning of a prostate-specific membrane antigen (PSMA) was reported as a potential prostate carcinoma marker and hypothesized to serve as a target for imaging and cytotoxic treatment modalities for prostate cancer. Antibodies against PSMA have been described and examined clinically for diagnosis and treatment of prostate cancer. In particular, Indium-111 labelled PSMA antibodies have been described and examined for diagnosis of prostate cancer and itrium-labelled PSMA antibodies have been described and examined for the treatment of prostate cancer.

### Example 6

Bladder Cancer

The classification of bladder cancer is divided into three main classes: 1) superficial disease, 2) muscle-invasive disease, and 3) metastatic disease.

Currently, transurethral resection (TUR), or segmental resection, account for first line therapy of superficial bladder cancer, i.e., disease confined to the mucosa or the lamina propria. However, intravesical therapies are necessary, for example, for the treatment of high-grade tumors, carcinoma in situ, incomplete resections, recurrences, and multifocal papillary. Recurrence rates range from up to 30 to 80 percent, depending on stage of cancer.

Therapies that are currently used as intravesical therapies include chemotherapy, immuontherapy, bacille Calmette-Guerin (BCG) and photodynamic therapy. The main objective of intravesical therapy is twofold: to prevent recurrence in high-risk patients and to treat disease that cannot by resected. The use of intravesical therapies must be balanced with its potentially toxic side effects. Additionally, BCG requires an unimpaired immune system to induce an antitumor effect. Chemotherapeutic agents that are known to be inactive against superficial bladder cancer include Cisplatin, actinomycin D, 5-fluorouracil, bleomycin, and cyclophosphamide methotrxate.

In the treatment of superficial bladder cancer, antiangiogenic agents can be used to treat the disease in combination with other antiangiogenic agents, or in combination with surgery (TUR), and intravesical therapies.

Preferred combinations of chemotherapeutic agents are selected from the group consisting of thiotepa (30 to 60 mg/day), mitomycin C (20 to 60 mg/day), and doxorubicin (20 to 80 mg/day).

The preferred intravesicle immunotherapuetic agent that may be used in the present invention is BCG. The preferred daily dose ranges from 60 to 120 mg, depending on the strain of the live attenuated tuberculosis organism used.

The preferred photodynamic therapuetic agent that may be used with the present invention is Photofrin I, a photosensitizing agent, administered intravenously. It is taken up by the low-density lipoprotein receptors of the tumor cells and is activated by exposure to visible light. Additionally, neomydium YAG laser activation generates large amounts of cytotoxic free radicals and singlet oxygen.

In the treatment of muscle-invasive bladder cancer, antiangiogenic agents can be used to treat the disease in combination with other antiangiogenic agents, or in combination with surgery (TUR), intravesical chemotherapy, radiation therapy, and radical cystectomy with pelvic lymph node dissection.

The preferred radiation dose is between 5,000 to 7,000 cGY in fractions of 180 to 200 cGY to the tumor. Additionally, 3,500 to 4,700 cGY total dose is administered to the normal bladder and pelvic contents in a four-field technique. Radiation therapy should be considered only if the patient is not a surgical candidate, but may be considered as preoperative therapy.

The preferred combination of surgery and chemotherapeutic agents that can be used in combination with the angiogenesis inhibitors is cystectomy in conjunction with five cycles of cisplatin (70 to 100 mg/m(square)); doxorubicin (50 to 60 mg/m(square); and cyclophosphamide (500 to 600 mg/m(square).

The preferred combinations of chemotherapeutic agents that can be used in combination with the angiogenesis inhibitors is include: 1) cisplatin, doxorubicin, cyclophosphamide; and 2) cisplatin, 5-fluorouracil. The preferred combination of chemotherapeutic agents that can be used in combination with radiation therapy and the angiogenesis inhibitors is cisplatin, methotrexate, vinblastine.

Currently no curative therapy exists for metastatic bladder cancer. The present invention contemplates an effective treatment of bladder cancer leading to improved tumor inhibition or regression, as compared to current therapies.

In the treatment of metastatic bladder cancer, antiangiogenic agents can be used to treat the disease in combination with other antiangiogenic agents, or in combination with surgery, radiation therapy or with chemotherapeutic agents.

Preferred combinations of chemotherapeutic agents include, but are not limited to: 1) cisplatin and methotrexate; 2) doxorubicin, vinblastine, cyclophoshamide, and 5-fluorouracil; 3) vinblastine, doxorubicin, cisplatin, methotrexate; 4) vinblastine, cisplatin, methotrexate; 5) cyclophosphamide, doxorubicin, cisplatin; 6) 5-fluorouracil, cisplatin.

### Example 7

Pancreas Cancer

Approximately 2% of new cancer cases diagnoses in the United States is pancreatic cancer. Pancreatic cancer is generally classified into two clinical types: 1) adenocarcinoma (metastatic and non-metastatic), and 2) cystic neoplasms (serous cystadenomas, mucinous cystic neoplasms, papilary cystic neoplasms, acinar cell systadenocarcinoma, cystic choriocarcinoma, cystic teratomas, angiomatous neoplasms).

Preferred combinations of therapy for the treatment of non-metastatic adenocarcinoma include the use of an antiangiogenic agent along with preoperative bilary tract decompression (patients presenting with obstructive jaundice); surgical resection, including standard resection, extended or radial resection and distal pancreatectomy (tumors of body and tail); adjuvant radiation; and chemotherapy.

For the treatment of metastatic adenocarcinoma, the preferred combination therapy consists of an antiangiogenesis inhibitor in combination with continuous treatment of 5-fluorouracil, followed weekly cisplatin therapy.

The preferred combination of therapy for the treatment of cystic neoplasms is the use of an antiangiogenic agent along with resection.

### Example 8

Ovary Cancer

Celomic epithelial carcinoma accounts for approximately 90% of ovarian cancer cases. Preferred single agents that can be used in combination with an antiangiogenesis agent include: alkylating agents, ifosfamide, cisplatin, carboplatin, taxol, doxorubicin, 5-fluorouracil, methotrexate, mitomycin, hexamethylmelamine, progestins, antiestrogens, prednimustine, dihydroxybusulfan, galactitol, interferon alpha, and interferon gama.

Preferred combinations that can be used along with an antiangiogenesis agent for the treatment of celomic epithelial carcinoma include: 1) cisplatin, doxorubicin, cyclophosphamide; 2) hexamthylmelamine, cyclosphamide, doxorubicin, cisplatin; 3) cyclophosphamide, hexamehtylmelamine, 5-flurouracil, cisplatin; 4) melphalan, hexamethylmelamine, cyclophosphamide; 5) melphalan, doxorubicin, cyclophosphamide; 6) cyclophosphamide, cisplatin, carboplatin; 7) cyclophosphamide, doxorubicin, hexamethylmelamine, cisplatin; 8) cyclophosphamide, doxorubicin, hexamethylmelamine, carboplatin; 9) cyclophosphamide, cisplatin; 10) hexamethylmelamine, doxorubicin, carboplatin; 11) cyclophosphamide, hexamethimelamine, doxorubicin, cisplatin; 12) carboplatin, cyclophosphamide; 13) cisplatin, cyclophosphamide.

Germ cell ovarian cancer accounts for approximately 5% of ovarian cancer cases. Germ cell ovarian carcinomas are classified into two main groups: 1) dysgerminoma, and nondysgerminoma. Nondysgerminoma is further classified into teratoma, endodermal sinus tumor, embryonal carcinoma, chloricarcinoma, polyembryoma, and mixed cell tumors.

Preferred combinations that can be used along with an antiangiogenesis agent for the treatment of germ cell ovarian carcinoms include: 1) vincristine, actinomycin D, cyclophosphamide; 2) bleomycin, etoposide, cisplatin; 3) vinblastine, bleomycin, cisplatin.

Cancer of the fallopian tube is the least common type of ovarian cancer, accounting for approximately 400 new cancer cases per year in the United States. Papillary serous adenocarcinoma accounts for approximately 90% of all malignancies of the ovarian tube.

Preferred single agents that can be used in combination with an antiangiogenesis agent for the treatment of papillary serous adenocarcinoma include: alkylating agents, ifosfamide, cisplatin, carboplatin, taxol, doxorubicin, 5-fluorouracil, methotrexate, mitomycin, hexamethylmelamine, progestins, antiestrogens, prednimustine, dihydroxybusulfan, galactitol, interferon alpha, and interferon gama.

Preferred combinations that can be used along with an antiangiogenesis agent for the treatment of papillary serous adenocarcinoma include: 1) cisplatin, doxorubicin, cyclophosphamide; 2) hexamthylmelamine, cyclosphamide, doxorubicin, cisplatin; 3) cyclophosphamide, hexamehtylmelamine, 5-flurouracil, cisplatin; 4) melphalan, hexamethylmelamine, cyclophosphamide; 5) melphalan, doxorubicin, cyclophosphamide; 6) cyclophosphamide, cisplatin, carboplatin; 7) cyclophosphamide, doxorubicin, hexamethylmelamine, cisplatin; 8) cyclophosphamide, doxorubicin, hexamethylmelamine, carboplatin; 9) cyclophosphamide, cisplatin; 10) hexamethylmelamine, doxorubicin, carboplatin; 11) cyclophosphamide, hexamethimelamine, doxorubicin, cisplatin; 12) carboplatin, cyclophosphamide; 13) cisplatin, cyclophosphamide.

### Example 9

Central Nervous System Cancers

Central nervous system cancer accounts for approximately 2% of new cancer cases in the United States. Common intracranial neoplasms include glioma, meninigioma, neurinoma, and adenoma.

Preferred combinations that can be used along with an antiangiogenesis agent for the treatment of malignant glioma include: 1) radiation therapy, BCNU (carmustine); 2) radiation therapy, methyl CCNU (lomustine); 3) radiation therapy, medol; 4) radiation therapy, procarbazine; 5) radiation therapy, BCNU, medrol; 6) hyperfraction radiation therapy, BCNU; 7) radiation therapy, misonidazole, BCNU; 8) radiation therapy, streptozotocin; 9) radiation therapy, BCNU, procarbazine; 10) radiation therapy, BCNU, hydroxyurea, procarbazine, VM-26; 11) radiation therapy, BNCU, 5-flourouacil; 12) radiation therapy, Methyl CCNU, dacarbazine; 13) radiation therapy, misonidazole, BCNU; 14) diaziquone; 15) radiation therapy, PCNU; 16) procarbazine (matulane), CCNU, vincristine. The preferred dose of radiation therapy is about 5,500 to about 6,000 cGY. Preferred radiosensitizers include misonidazole, intra-arterial Budr and intravenous iododeoxyuridine (IUdR). It is also contemplated that radiosurgery may be used in combinations with antiangiogenesis agents.

### Biological Evaluation

MMP Inhibitors

1. Pancreatic Cell (PC-3) Model:

In this study, the test groups were a vehicle control, Compound M14, Compound M14 with cisplatin and cisplatin alone with n=10 for each group. The tumors were measured with a caliper and the volume calculated using the formula for the volume of an elipsoid. The cisplatin dose was 10 mpk administered by the intraperitonal route on day 8 post injecion of tumor cells Compound M14, 50 mpk, was first administered about 6:00 pm the evening of the same day that the tumor cells were injected in the morning. The same dose of Compound M14 was administered bid for each following day. Tumor volume (mm³) was measured on day 25. The data below clearly show an improved response with the combination of the MMP inhibitor and cisplatin.

PC3 Model MMP Inhibitor Combination Study Results

| Agent Administered | Tumor Volume at Day 25 |
|---|---|
| PC3 Model | (mm³) |
| vehicle | 860 |
| cisplatin | 630 |
| Compound M14 | 480 |
| Compound M14 | 110 |
| with cisplatin | |

2. Breast Tumor Model:

This study was carried out essentially as PC-3 model. MX-1 breast tumor pieces were implanted (with a trocar) into nude mice with n=10 per group. Dosing with Compound M14(10 mpk or 50 mpk, PO bid) was initiated when the tumors reached a size of 60-120 mg. Dosing was continued for 26 days. Taxol was administered at a dose of 9 mpk for the first five days following the start of dosing by the interperitonal route. The tumors were measured using a caliper and the volume calculated using the formula for the volume of an elipsoid. The results tabulated below clearly show an improved response with combination therapy. An improved response is obtained with lower doses Compound M14.

MX-1 Model MMP Inhibitor Combination Study Results

| | Tumor Volume at Day 25 |
|---|---|
| Agent Administered | (mm³) |
| vehicle | 1920 |
| taxol | 1280 |
| Compound M14 | 960 |
| @ 10 mpk | |
| Compound M14 | 1260 |
| @ 50 mpk | |
| Compound M14 @ 50 mpk + | 480 |
| taxol @ 9 mpk | |
| Compound M14 @ 10 mpk + | 240 |
| taxol @ 9 mpk | |

3. MX-1 Adjuvant Model:

Mice were implanted with MX-1 tumors and allowed to grow to 50-100 mm3. The animals were dosed with cyclophosphamide (100 or 80 mpk). This was considered Day 1. Two weeks later the animals were pair matched after tumor regression and dosing BID with the MMPI was begun until the end of the experiment. Tumors were measured weekly. The endpoint for the study was a final tumor size of 1.5 g.

| | Cyclo- | | | | |
|---|---|---|---|---|---|
| | phos- | | | | |
| | phamide | | MMPI | | |
| | Dose | | Dose | | |
| | (mpk) | MMPI | (mpk) | MDS | sem |
| saline | | | | 23.9 | 1.3 |
| cyclophosphamide | 100 | | | 39.5 | 1.2 |
| cyclophosphamide | 80 | | | 37.2 | 1.5 |
| cyclophosphamide | 100 | Compound | 200 | 52.7 | 2.9 |
| | | M14 | | | |
| cyclophosphamide | 100 | Compound | 50 | 43.7 | 1.6 |
| | | M14 | | | |
| cyclophosphamide | 0 | Compound | 200 | 53.9 | 2.9 |
| | | M14 | | | |
| cyclophosphamide | 80 | Compound | 50 | 44.2 | 1.8 |
| | | M14 | | | |

MDS = mean days to tumor weight of 1.5 g

4. MX-1 Breast Tumor with Taxol:

Mice were implanted with MX-1 tumors and allowed to grow to 50-100 mg. The animals were pair matched and this was considered Day 1. Treatment with MMPI was begun BID on Day 1 until the end of the experiment. Taxol was injected IP (15 or 9 mpk) QD for 5 days (days 1-5). Tumors were measured weekly until an endpoint of 1.5 g was reached.

| | Taxol | | MMPI | | |
|---|---|---|---|---|---|
| | Dose | | Dose | | |
| | (mpk) | MMPI | (mpk) | MDS | sem |
| vehicle | | | | 25.3 | 0.8 |
| mmpi | | Compound | 100 | 32.2 | 2.8 |
| | | M14 | | | |
| mmpi | | Compound | 20 | 34.7 | 3 |
| | | M14 | | | |
| taxol + mmpi | 18 | Compound | | 56 | 11 |
| | | M14 | | | |
| taxol + mmpi | 9 | Compound | | 30.1 | 1.8 |
| | | M14 | | | |
| taxol + mmpi | 18 | Compound | 100 | 61 | |
| | | M14 | | | |
| taxol + mmpi | 9 | Compound | 100 | 46.7 | 3.7 |
| | | M14 | | | |
| taxol + mmpi | 18 | Compound | 20 | 59.3 | 7 |
| | | M14 | | | |
| taxol + mmpi | 9 | Compound | 20 | 39.3 | 1.9 |
| | | M14 | | | |

MDS = 1.5 g

5. SK-Mes Tumor with Taxol

Mice were implanted with SK-mes tumors and allowed to grow to 50-100 mg. The animals were pair matched and this was considered Day 1. Treatment with MMPI was begun BID on Day 1 until the end of the experiment. Taxol was injected IP (18 or 9 mpk) QD for 5 days (days 1-5). Tumors were measured weekly until an endpoint of 1.0 g was reached.

| | | | MMPI | | |
|---|---|---|---|---|---|
| | Taxol Dose | | Dose | | |
| | (mpk) | MMPI | (mpk) | MDS | sem |
| vehicle | | | | 21.2 | 2.1 |
| mmpi | | Compound | 100 | 24.7 | 1.6 |
| | | M14 | | | |
| mmpi | | Compound | 20 | 18 | 1.1 |
| | | M14 | | | |
| taxol | 18 | | | 31.5 | 2.4 |
| taxol | 9 | | | 26.1 | 2.3 |
| taxol + mmpi | 18 | Compound | 100 | 43 | 4 |
| | | M14 | | | |
| taxol + mmpi | 9 | Compound | 100 | 34.8 | 1.9 |
| | | M14 | | | |
| taxol + mmpi | 18 | Compound | 20 | 39.5 | 3.6 |
| | | M14 | | | |
| taxol + mmpi | 9 | Compound | 20 | 34.1 | 5.7 |
| | | M14 | | | |

MDS = 1.0 g

6. HT-29 Tumor with Irinotecan

Mice were implanted with HT-29 tumors and allowed to grow to 50-100 mg. The animals were pair matched and this was considered Day 1. Treatment with MMPI was begun BID on Day 1 until the end of the experiment. Irinotecan was injected IP (100 or 50 mpk) QD for 5 days (days 1-5). Tumors were measured weekly until an endpoint of 1.0 g was reached.

| | | | MMPI | | |
|---|---|---|---|---|---|
| | Irinotecan | | Dose | | |
| | Dose (mpk) | MMPI | (mpk) | MDS | SEM |
| vehicle | | | | 36.4 | 4.3 |
| mmpi | | Compound | 100 | 37.9 | 5.0 |
| | | M14 | | | |
| mmpi | | Compound | 20 | 36 | 4.2 |
| | | M14 | | | |
| Irinotecan | 100 | | | 36.7 | 2.6 |
| Irinotecan | 50 | | | 38.1 | 3.0 |
| Irinotecan + | 100 | Compound | 100 | 51.4 | 4.4 |
| mmpi | | M14 | | | |
| Irinotecan + | 50 | Compound | 100 | 44.4 | 4.0 |
| mmpi | | M14 | | | |
| Irinotecan + | 100 | Compound | 20 | 40.6 | 4.7 |
| mmpi | | M14 | | | |
| Irinotecan + | 50 | Compound | 20 | 36.1 | 3.0 |
| mmpi | | M14 | | | |

MDS = 1.0 g

COX-2 Inhibitors

1. Lewis Lung Model:

Mice were injected subcutaneously in the left paw (1×10⁶ tumor cells suspended in 30% Matrigel) and tumor volume was evaluated using a phlethysmometer twice a week for 30-60 days. Blood was drawn twice during the experiment in a 24 h protocol to assess plasma concentration and total exposure by AUC analysis. The data are expressed as the mean±SEM. Student's and Mann-Whitney tests were used to assess differences between means using the InStat software package. Celecoxib given in the diet at doses between 160-3200 ppm retarded the growth of these tumors. The inhibitory effect of celecoxib was dose-dependent and ranged from 48% to 85% as compared with the control tumors. Analysis of lung metastasis was done in all the animals by counting metastasis in a stereomicroscope and by histochemical analysis of consecutive lung sections. Celecoxib did not affect lung metastasis at the lower dose of 160 ppm, however surface metastasis was reduced by more than 50% when given at doses between 480-3200 ppm. In addition, histopathological analysis revealed that celecoxib dose-dependently reduced the size of the metastasic lesions in the lung.

2. HT-29 Model:

Mice were injected subcutaneously in the left paw (1×10⁶ tumor cells suspended in 30% Matrigel) and tumor volume was evaluated using a phlethysmometer twice a week for 30-60 days. Implantation of human colon cancer cells (HT-29) into nude mice produces tumors that will reach 0.6-2 ml between 30-50 days. Blood was drawn twice during the experiment in a 24 h protocol to assess plasma concentration and total exposure by AUC analysis. The data are expressed as the mean±SEM. Student's and Mann-Whitney tests were used to assess differences between means using the InStat software package.

A. Mice injected with HT-29 cancer cells were treated with cytoxin i.p at doses of 50 mg/kg on days 5,7 and 9 in the presence or absence of celecoxib in the diet. The efficacy of both agents were determined by measuring tumor volume. Treatment using a celecoxib related Cox-2 inhibitor (SC-58236) reduced tumor volume by 89%. In the same assay, indomethacin given at near the maximum tolerated dose of 2 mg/kg/day in the drinking water inhibited tumor formation by 77%. Moreover, the Cox-2 selective inhibitor completely inhibited the formation of lung metastasis while the non-selective NSAID indomethacin was ineffective. The results from these studies demonstrate that celecoxib administered in the diet to tumor bearing mice can delay the growth of tumors and metastasis when administered as sole therapy. Moreover, a positive benefit is observed when celecoxib is administered in combination with a cytotoxic agent such as cyclophosphamide.

B. In a second assay, mice injected with HT-29 cancer cells were treated with 5-FU on days 12 through 15. Mice injected with HT-29 cancer cells were treated with 5-FU i.p at doses of 50 mg/kg on days 12, 13, 14, and 15 in the presence or absence of celecoxib in the diet. The efficacy of both agents were determined by measuring tumor volume. Treatment using a celecoxib reduced tumor volume by 68%. In the same assay, 5-FU decreased tumor volume by 61%. Further, the combination of celecoxib and 5-FU decreased tumor volume by 83%.

C. In a third assay, mice injected with HT-29 colon cancer cells were treated with 5-FU i.p 50 mg/kg on days 14 through 17 in the presence or absence of celecoxib (1600 ppm) and valdecoxib (160 ppm) in the diet. The efficacy of both agents were determined by measuring tumor volume. Treatment with 5-FU resulted in a 35% reduction in tumor voloume. Treatment with celecoxib and valdecoxib reduced tumor volume by 52% and 69%, respectively. In the same assay, the combination of 5-FU and celecoxib decreased tumor volume by 72% while the combination of 5-FU and valdecoxib decreased tumor volume by 74b % (Table 17).

TABLE 17
Tumor Volume Effect of Celecoxib and Valdecoxib
alone and in combination with 5-Fluorouracil.

| | | | | celecoxib | | valdecoxib |
|---|---|---|---|---|---|---|
| | | | | 160 ppm/ | | 160 ppm/ |
| | | 5FU | celecoxib | 5FU | valdecoxib | 5FU |
| Days | Vehicle | 50 mpk | 160 ppm | 50 mpk | 160 ppm | 50 mpk |
| 11 | 0.04 | 0.05 | 0.05 | 0.05 | 0.06 | 0.06 |
| 14 | 0.13 | 0.12 | 0.13 | 0.13 | 0.13 | 0.13 |
| 18 | 0.19 | 0.16 | 0.17 | 0.14 | 0.17 | 0.16 |
| 21 | 0.23 | 0.21 | 0.2 | 0.17 | 0.2 | 0.19 |
| 28 | 0.38 | 0.3 | 0.25 | 0.22 | 0.25 | 0.21 |
| 35 | 0.62 | 0.46 | 0.35 | 0.28 | 0.32 | 0.29 |
| 42 | 1.01 | 0.68 | 0.52 | 0.32 | 0.36 | 0.31 |

Volume (ml)

D. In a fourth assay, mice injected with HT-29 colon cancer cells were treated with celecoxib (10, 40 or 160 ppm) in the diet beginning 20 at day 10. An approximate dose dependent effect was observed. (Table 18).

TABLE 18
Celecoxib Inhibitis HT-29 Human Colon Carcinoma

| Days | vehicle | 10 ppm | 40 ppm | 160 ppm |
|---|---|---|---|---|
| 14 | 0.114 | 0.124 | 0.125 | 0.120 |
| 22 | 0.25 | 0.25 | 0.19 | 0.14 |
| 28 | 0.45 | 0.36 | 0.27 | 0.21 |
| 35 | 0.79 | 0.57 | 0.4 | 0.3 |
| 42 | 1.38 | 0.89 | 0.68 | 0.49 |
| 50 | 1.9 | 1.49 | 1.04 | 0.8 |

Volume (ml)

Integrin Antagonists

1. Cancer cells were implanted subcutaneously in genetically engineered mice and grew large-volume tumors (>1,500 mm³). Subsequent administration of compound I7 reduced tumor growth by as much as 85 percent in a dose dependent manner. (Nickols A, et al. Inhibition of tumor growth and metastasis by an ανβ3 integrin antagonist. Presented at the 89^{th} Annual Meeting of the American Association for Cancer Research, March, 1998.)

2. In an additional experiment, tumor cells were implanted into mice; lung tumors of volumes greater than 2,000 mm³ were developed. The mice were then separated into four groups, including a control group and three treatment groups: compound I7 alone; compound I7 with cisplatin (a cytotoxic drug); or cisplatin alone. Compared to the control groups, the mice treated with combination compound I7/cisplatin therapy experienced more than an 80 percent reduction in tumor size. In comparison, the group receiving cisplatin alone experienced 50 percent reductions in tumor size and the compound I7 group experienced 20-30 percent reductions. These studies indicate that compound I7 has prominent anti-tumor activity.

3. M21 human melanoma, rat Leydig testicular carcinoma, Lewis Lung and human xenograft models:

To test the utility of aᵥb₃ antagonists as single agents and in combination chemotherapy, the M21 human melanoma, rat Leydig testicular carcinoma, and the Lewis Lung carcinoma (LLC) model as well as other human tumor xenograft models were utilized. Tumor cells for implantation were taken from cells either grown in tissue culture (Leydig, M21) or serially passaged as tumors in mice and prepared as tumor brei (LLC). Mice were injected subcutaneously in the proximal dorsal midline with 5×10⁶ tumor cells and administration of test compound or vehicle was initiated the evening of the same day. Tumor volumes were measured at intervals over the course of the experiments. Tumors were measured with a vernier caliper and volumes were determined using the formula for the volume of a cylinder: tumor volume=width²×length×0.52. Blood was routinely drawn for plasma drug concentration 6 hours post-dosing on day 4 or 5 and again 12 hours post-dosing on the day of sacrifice. On the final day of the experiment, tumors were dissected free and weighed. The data are expressed as the mean±SEM. Student's and Mann-Whitney tests were used to assess differences between means or medians using the InStat software package.

In the LLC model, compound I7 was administered continuously beginning on day 1 after implantation of the tumor cells, and the chemotherapeutic, cisplatin, was administered as a single intraperitoneal dose of 10 mg/kg on day 5. In this study, cisplatin alone significantly retarded the growth of the LLC tumor (p<0.05). Compound I7 (1 and 10 mg/kg, BID, PO) did not affect the growth of the primary tumor mass. However, the combination of compound I7 together with cisplatin resulted in an additive effect and a significant tumor growth delay (time to develop a tumor>500 mm³ was: vehicle=18.1 days; cisplatin=22.4 days; cisplatin+compound I7 (10 mg/kg)=27.3 days). The final tumor volume was also significantly reduced with the combination of cisplatin and compound I7 producing a reduction of final tumor volume of 68% in combination (p<0.05). Moreover, the combination of cisplatin and compound I7 resulted in a 39% improvement in median survival time over vehicle controls and an enhancement over either agent alone (28 days for the vehicle group; 33 days for the cisplatin group; 33 days for the compound I7 at 10 mg/kg group; 38 days for the combination group). Similarly, compound I7 reduced tumor volume when given with cisplatin in a dose-sequencing protocol. The combination of aᵥb₃ antagonist and chemotherapeutic agent was more efficacious than cisplatin alone, particularly when therapy with compound I7 (po, BID) was begun at the same time as cisplatin (once, IP on day 5) or 5 days later (p<0.05 or less for all).

In the M21 model, M21 human melanoma cells implanted subcutaneously into SCID mice developed tumors, which grew to approximately 400 mm³ within 30 days. Oral administration of compound compound I7 (BID) dose-dependently retarded the growth of these tumors when administered at the time of tumor implantation or beginning up to 21 days after implantation. Time to develop a tumor mass>200 mm³ was significantly lengthened in the group treated with the aᵥb₃ antagonist (time to tumor volume>200 mm³ was: vehicle=15 days; compound I7, 10 mg/kg=27 days). These data clearly demonstrate the utility of compound compound I7 to inhibit the growth of pre-existing and established tumors. Moreover, compound compound I7 increased the antitumor efficacy of cisplatin when treatment with the aᵥb₃ antagonist was begun on day 1, prophylactically, or therapeutically, on day 14 or 17 (all combinations significantly less than cisplatin alone, p<0.05). Cisplatin was administered once by ip injection (10 mg/kg) on day 14. Final tumor weights were nearly identical in the combination treated groups, with clear enhancement of the effect of cisplatin treatment alone. The results of this dose sequencing experiment establish the efficacy of compound I7 in combination therapy with cisplatin when administered before, concurrent with, or after cisplatin dosing.

The Rice 500 rat Leydig testicular tumor grows very quickly when implanted into the flank of SCID mice. Compound I7 inhibited tumor growth dose-dependently when given in the drinking water at concentrations of 0.02 to 2 mg/ml. Tumor growth was reduced by about 50% at the 2 mg/ml dose in this aggressive model. Since the tumor does not express the aᵥb₃ integrin, the antitumor effects were likely to be produced by the inhibition of angiogenesis. Similar to the results seen in the M21 tumor model, compound I7 increased the effects of cisplatin in the Leydig tumor model. Indeed, the combination of cisplatin and compound I7 was almost 100% effective in preventing tumor growth over the 11 day course of the study. Dose-related inhibition of tumor growth by compound I7 (10 or 100 mg/kg, BID, PO) was also seen when the compound was given as monotherapy or in combination with cisplatin (10 mg/kg, ip once on day 5) (p<0.01 vs control). Therapeutic treatment with the aᵥb₃ antagonist was begun at the same time as cisplatin on day 5, with tumor volumes of about 200 mm³ at the initiation of therapy. In a similar experiment, the effects of compound I7, cisplatin and the combination were evaluated for potentiation of overall survival in the Leydig tumor mice. Survival was increased by either compound I7 or cisplatin alone when compared to vehicle treated controls (p<0.05). More importantly, the combination of the two agents almost doubled overall survival (from 17 to 29 days) (p<0.01 combination vs. cisplatin, p<0.001 combination vs. control). Thus, the ability of compound I7 to work alone or in combination therapy to prevent tumor growth clearly correlates with enhanced survival.

4. U251 Glioblastoma Model:

Compound I7 was evaluated in the human U251 glioblastoma model. The tumors were implanted onto the flanks of SCID mice and the mean tumor volume with time was calculated. In this model, at the dose tested (10 mg/kg, BID, PO), compound I7 produced little inhibition of tumor growth by itself when administered from day 14 through 44. The chemotherapeutic agent, BCNU (12 mg/kg) administered once a day on days 14, 18 and 22, induced a regression of the tumors to the limit of detectability, but the tumors grew back. Combination treatment with BCNU and compound I7 regressed tumors to the limit of dectability throughout the period of treatment (compound 17 administered from day 14-44) and almost through the rest of the study. When the data are examined as time to tumor progression (days to 2 tumor doublings), there is clear enhancement by the drug combination over the antitumor effects of either agent alone (p<0.01). Moreover, the response rate (responders to BCNU) is markedly enhanced and the duration of the response is increased 5-fold from 5 days to 25 days (p<0.01). These clinically relevant measurements of antitumor efficacy establish the antitumor efficacy of compound 17, especially when combined with standard of care chemotherapeutic agents. 5. A2780 Mouse Model:

Compound I7 prevents the growth of human ovarian carcinoma in SCID mice. The A2780 tumor line is another aggressive tumor model characterized by rapid growth. Compound I7 treatment (10 mg/kg, BID, PO) was equally effective as cisplatin (10 mg/kg, ip once on day 20) in decreasing tumor growth. However, as seen in the other tumor models, compound I7 potentiated the effects of cisplatin, resulting in an 80% reduction vs control on day 30. Survival studies are now underway to characterize the survival benefit of combination therapy in this model.

6. Corneal Micropocket Assay:

In this model, an intrastromal pocket is surgically created in the normally avascular cornea of female C57BL6 mice 1 mm distance from the corneal-scleral junction. A slow release hydron polymer pellet containing an angiogenic growth factor (bFGF or VEGF) is inserted into the corneal pocket. The pocket is self sealing and antibiotic ointment is placed in the eye. Five days later the eyes are examined under a slit lamp and the neovascular response is quantitated by measuring the average vessel length (VL) and the contiguous circumferential zone (CH=clock hours where 1 CH=30 degrees) and plugged into the formula of half an ellipse; Area (mm2)=0.5×3.1416×VL×CH×0.4. compound I7 administered BID is a potent inhibitor of angiogenesis in the mouse corneal micropocket model. compound I7 dose-dependently inhibited the angiogenic response up to 42% with maximal inhibitory activity observed at doses of 10 mg/kg, BID orally. Moreover, compound I7 inhibited angiogenesis induced by either bFGF or VEGF, the two predominant growth factors known to be produced by tumor cells in vivo. These data confirm the mechanism of action of compound I7 as direct inhibition of angiogenesis in vivo.

7. Metastasis

Accurate quantitation of early-stage metastasis in animal models is typically hampered by the lack of sensitive and convenient assays to detect low numbers of tumor cells in a background of normal tissue. Quantitation of late-stage metastasis by counting of visible foci or comparison of organ weights requires substantial tumor burden which can take 3-4 months to develop in conventional models of breast cancer, and generally cannot detect subtle differences. To develop a more quantitative metastasis model in which the effect of inhibitors on multiple stages of the metastatic process could be dissected, we have produced stable MDA-MB-435 breast carcinoma cell lines expressing a synthetic variant of green fluorescent protein (GFP). The GFP-transfected cells are easily detected by flow cytometry, and fixation of the cells or the addition of antibodies or exogenous substrates is not required. A highly aggressive clone was isolated from the lung of a SCID mouse implanted in the mammary fat pad with several GFP-expressing clones. This line, designated 435/GFP HAL-1, consistently generates substantial tumor burden in the lungs by 8-9 weeks compared with 12-16 weeks for the parent line. As few as 1 tumor cell in 200,000 host cells can be detected by flow cytometry, and fluorescent cells are detected in the lungs and blood as early as one week post-orthotopic implantation. compound I7 was administered at doses of 1, 10, and 30 mg/kg, BID, orally following orthotopic surgical implantation of 435/GFP HAL-1 cells into the mammary fat pad of SCID mice. Eight weeks later, lungs were removed and weighed. Metastasis was quantitated using a semi-quantitative visible scoring method of gross metastases under a dissecting scope or, following dissection and disaggregation of lung tissue, by flow cytometry of GFP expressing cells. Compound I7 administration dose-dependently reduced the spontaneous metastasis of 435 breast carcinoma cells to the lungs as determined either by direct visual counting or quantitation by flow cytometry. Doses of 10 and 30 mg/kg resulted in a 55% and 69% reduction in lung metastatic burden, respectively. However, compound I7 did not delay the growth of the primary tumor mass in this model. Histological examination of lung sections from these studies revealed a dramatic reduction in the number of large macroscopic metastases and an increase in the presence of microscopic foci of metastases in the compound I7 treated animals.

Radiatoin Therapy

Solitary tumors are generated in the right hind legs of mice by the injection of 3×10⁵ viable NFSA tumor cells. Treatment with an integrin antagonist (6 mg/kg body weight) or vehicle (0.05% Tween 20 and 0.95% polyethylene glycol) given in the drinking water is started when tumors are approximately 6 mm in diameter and the treatment is continued for 10 consecutive days. Water bottles are changed every 3 days. Tumor irradiation is performed 3-8 days after initiation of the treatment with an integrin antagonist. The end points of the treatment are tumor growth delay (days) and TCD₅₀ (tumor control dose 50, defined as the radiation dose yielding local tumor cure in 50% of irradiated mice 120 days after irradiation). To obtain tumor growth curves, three mutually orthogonal diameters of tumors are measured daily with a vernier caliper, and the mean values are calculated.

Local tumor irradiation with single γ-ray doses of 30, 40, or 50 Gy is given when these tumors reach 8 mm in diameter. Irradiation to the tumor is delivered from a dual-source ¹³⁷Cs irradiator at a dose rate of 6.31 Gy/minute. During irradiation, unanesthetized mice are immobolized on a jig and the tumor is centered in a circular radiation field 3 cm in diameter. Regression and regrowth of tumors are followed at 1-3 day intervals until the tumor diameter reaches approximately 14 mm.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description shall be interpreted as illustrative and not in a limiting sense.

What is claimed is:

1. A method of preventing or treating neoplasia disorders and neoplasia disorder-related complications in a subject that is in need of such prevention or treatment comprising administering to the subject a Cox-2 inhibitor in combination with an EGF receptor antagonist.

2. The method according to claim 1, wherein the Cox-2 inhibitor and EGF receptor antagonist is administered to the subject in combination with one or more antineoplastic agents.

3. The method according to claim 1, wherein the Cox-2 inhibitor comprises a non-steroidal anti-inflammatory drug.

4. The method according to claim 1, wherein the Cox-2 inhibitor is selected from the group consisting of ibuprofen, naproxen, benoxaprofen, flurbiprofen, fenoprofen, fenbufen, ketoprofen, indoprofen, pirprofen, carprofen, oxaprozin, prapoprofen, miroprofen, tioxaprofen, suprofen, alminoprofen, tiaprofenic acid, fluprofen, bucloxic acid, indomethacin, sulindac, tolmetin, zomepirac, diclofenac, fenclofenec, alclofenac, ibufenac, isoxepac, furofenac, tiopinac, zidometacin, acetyl salicylic acid, indometacin, piroxicam, tenoxicam, nabumetone, ketorolac, azapropazone, mefenamic acid, tolfenamic acid, diflunisal, podophyllotoxin derivatives, acemetacin, droxicam, floctafenine, oxyphenbutazone, phenylbutazone, proglumetacin, acemetacin, fentiazac, clidanac, oxipinac, mefenamic acid, meclofenamic acid, flufenamic acid, niflumic acid, flufenisal, sudoxicam, etodolac, piprofen, salicylic acid, choline magnesium trisalicylate, salicylate, benorylate, fentiazac, clopinac, feprazone, isoxicam and 2-fluoro-a-methyl[1,1 ′-biphenyl]-4-acetic acid, 4-(nitrooxy)butyl ester.

5. The method according to claim 1, wherein the Cox-2 inhibitor comprises a Cox-2 selective inhibitor.

6. The method according to claim 5, wherein the Cox-2 selective inhibitor is selected from the group consisting of celecoxib, parecoxib, deracoxib, valdecoxib, etoricoxib, meloxicam, rofecoxib, lumiracoxib, RS 57067, T-614, BMS-347070, JTE-522, S-2474, SVT-2016, CT-3, ABT-963, SC-58125, nimesulide, flosulide, NS-398, L-745337, RWJ-63556, L-784512, darbufelone, CS-502, LAS-34475, LAS-34555, S-33516, SD-8381, prodrugs of any of them, and mixtures thereof.

7. The method according to claim 5, wherein the Cox-2 selective inhibitor is selected from the group consisting of celecoxib, parecoxib, deracoxib, valdecoxib, etoricoxib, meloxicam, rofecoxib, lumiracoxib, prodrugs of any of them, and mixtures thereof.

8. The method according to claim 5, wherein the Cox-2 selective inhibitor comprises celecoxib.

9. The method according to claim 2, wherein the antineoplastic agent is selected from the group consisting of antimetabolite agents, alkylating agents, antibiotic-type agents, hormonal anticancer agents, immunological agents, interferon-type agents, prodrugs of any of them, and mixtures thereof.

10. The method according to claim 2, wherein the antineoplastic agent is a taxane derivative.

11. The method according to claim 10, wherein the taxane derivative is paclitaxel.

12. The method according to claim 1, wherein the EGF receptor antagonist is selected from the group consisting of 4-aminoquinazolines, potato carboxypeptidase inhibitor, tyrosine kinase inhibitor, bombesin antagonist RC-3095, quinazolines, pyridopyrimidines, pyrimidopyrimidines, pyrrolopyrimidines, pyrazolopyrimidines, 4-(phenylamino)-7H-pyrrolo[2,3-d]pyrimidines, curcumin, 4,5-bis (4-fluoroanilino)phthalimide, tyrphostins containing nitrothiophene moieties, benzothiazoles, paeciloquinones, cinnoline derivatives, EGF receptor antisense molecules, substituted styrenes, anti-EGF receptor antibodies and dianilinopthalimides, prodrugs of any of them, and mixtures thereof.

13. The method according to claim 1, wherein the EGF receptor antagonist is selected from the group consisting of AGM-1470, reveromycin A, TNP-470, PD-171026, PD-089828, PD-090560, ZM-254530, ZM-105180, EGF-genistein, wayne anti-EGFR Mabs, anti-VEGF monoclonal, Genen EMD-72000, anti-EGFR Mab, EMD-6200, MDX-447, BAB-447, EMD-82633, H-447, ABX-EGF, anti-EGFr MAb, anti-EGFR-DM1 Ab, anti-EGFR conjugate, anti-flk-1 MAb DC-101, bromelain molecules, CCX, CCZ, tecogalan sodium, platelet factor 4, Inhibitors of vascular endothelial growth factor antagonist (VEGF) and its receptor flk-1, 4-(3-Ethynylphenylamino)-6,7-bis(2-methoxyethoxy)-quinazoline hydrochloride, ZD-1838, ZD-1839, 4-(4-methylpiperazin-1-ylmethyl)-N-[4-methyl-3-[4-(3-pyridyl)pyrimidin-2-ylamino]phenyl]benzamide, CGP-59326, CGP-79787 CGP-59326B, CGP-62706, CGP-74321, CGP-75166, CGP-76627, DWP-408, muellerian-inhibiting hormone, 4-(m-chloro)-5,6-dimethyl-7H-pyrrolo[2,3-d]pyrimidine, 5-[3-[3-methoxy-4-[2-[(E)-2-phenylethenyl]-4-oxazolylmethoxy]phenyl]propyl]-3-[2-[(E)-2-phenylethenyl]-4-oxazolylmethyl]-2,4-oxazolidinedione, N-(6-Benzothiazolyl)-4-(2-(1-piperazinyl)pyrid-5-yl)-2-pyrimidineamine, PI-88, PJ3505, S-96-8045, 4-(4-chloro-2-fluoro-5-hydroxyanilino)-6-methoxy-7-(2-methoxyethoxy)cinnoline, N-[4-[(3-chloro-4-fluorophenyl)amino]-7-[3-(4-morpholinyl)propoxy]-6-quinazolinyl]-2-propenamide, EKB-569, PTK787, HER-2/neu protein antibody, EGF receptor antibody, trastuzumab, GEM-220 AR-639, MDX-447, MDX-260, DAB-720, HER-2 antagonist, VRCTC-310, MR1scFvPE38KDEL, EMD-55900, EGF fusion toxin, OLX-103, Selex, CGP-62706, SU-5271, NX-278-L, metalloprotease inhibitor, EGF fusion protein, Amphiregulin, CGP-52411, AG-1478, RC-3940-II, CP-358774, C225, hbEGF-toxin, MAb 4D5, BBR-1611, PD-169450, QX-101, SU-5271, flavopiridol, SU-101, celastrol, CGP-52411, anti-flk-1, CEP-2563, HER-2 antagonist, NSC-675967, SU-5416, FCE-26806, DAB-720, CEP-751, ZD-1838, CGP-60261, EGF-RTK antagonist, ALL-TK antagonists, GRB2 antagonists, CGP-57148, ZD-1839, erbB-2 receptor inhibitors, PD-1 58780, benzothiazoles, PD-171026, BE-23372M derivatives, Met TK antagonist, PD-1 59973, GW-282974, CP-292597, ZM-105180, GW-7072X, Lck tyrosine kinase inhibitors, PD-168393, PD-173956, RG-14620, CGP-59326, genistein, FCE-27119, RG-13022, RG-50864, PD-154233, TT-232, AG-514, AG-568, PD-151514, BE-23372M, KW-6151, paeciloquinones, PDGFrTK inhibitors, SDZ-LAP-977, CGP-53716, CGP-79787, B43-genistein, CGP-62706, AG-957, erlotinib, iressa, cetuximab, trastuzumab-DM1, mono[3-butyl-8-(9-carboxy-6-hydroxy-3,7-dimethyl-2,4,8-nonatrienyl)-2-(4-carboxy-3-methyl-1,3-butadienyl)-9-methyl-1,7-dioxaspiro[5.5]undec-3-yl]ester, Imatinib mesylate, trastuzumab, 4-(3-chloroanilino)-6,7-dimethoxyquinazoline, [(dimethylamino)methyl]acrylo-para-[(hydroxy-benzoylsulfonyl)oxy]phenone, cetuximab, theraCIM h-R3, erlotinib in combination with taxotere, C1033, EKB-569, ior-egf/r3, EGF-genistein, anti-EGFr MAb, anti-EGFR-DM1 Ab, anti-EGFR conjugate, EGFR conjugate, CI-1033, GW-211, PKI-166, STI571, BIBX1522, 4-(phenylamino)quinazolines, EGF receptor antisense oligonucleotide, amphiregulin, EGF fusion protein, 4-(3-bromoanilino)-6,7-dimethoxyquinazoline analogues, 4-[ar(alk)ylamino]pyridopyrimidines, GW2974, GW9263, GW4263, GW0277, GW5289, GW5949, GW9525, GW572016, PD13530, CGP5211, CGP53353, CGP 75166/PKI166, BIBX 1382, EKB-569, CI-1033, GW-2016, EMD- 72000, MDX-210, 2C4, TgDCC-E1A, prodrugs of any of them, and mixtures thereof.

14. The method according to claim 1, wherein the EGF receptor antagonist is selected from the group consisting of AGM-1470, reveromycin A, TNP-470, PD-171026, PD-089828, PD-090560, ZM-254530, ZM-105180, EGF-genistein, wayne anti-EGFR Mabs, anti-VEGF monoclonal, Genen EMD-72000, anti-EGFR Mab, EMD-6200, MDX-447, BAB-447, EMD-82633, H-447, ABX-EGF, anti-EGFr MAb, anti-EGFr Mab, anti-EGFR-DM1 Ab, anti-EGFR conjugate, anti-flk-1 MAb DC-101, bromelain molecules, CCX, CCZ, tecogalan sodium, platelet factor 4, Inhibitors of vascular endothelial growth factor antagonist (VEGF) and its receptor flk-1, 4-(3-Ethynylphenylamino)-6,7-bis(2-methoxyethoxy)-quinazoline hydrochloride, ZD-1838, ZD-1839, 4-(4-methylpiperazin-1-ylmethyl)-N-[4-methyl-3-[4-(3-pyridyl)pyrimidin-2-ylamino]phenyl]benzamide, CGP-59326, CGP-79787 CGP-59326B, CGP-62706, CGP-74321, CGP-75166, CGP-76627, DWP-408, EGF-genistein, muellerian-inhibiting hormone, 4-(m-chloro)-5,6-dimethyl-7H-pyrrolo[2,3-d]pyrimidine, 5-[3-[3-methoxy-4-[2-[(E)-2-phenylethenyl]-4-oxazolylmethoxy]phenyl]propyl]-3-[2-[(E)-2-phenylethenyl]-4-oxazolylmethyl]-2,4-oxazolidinedione, N-(6-Benzothiazolyl)-4-(2-(1-piperazinyl)pyrid-5-yl)-2-pyrimidineamine, PI-88, PJ3505, S-96-8045, 4-(4-chloro-2-fluoro-5-hydroxyanilino)-6-methoxy-7-(2-methoxyethoxy)cinnoline, N-[4-[(3-chloro-4-fluorophenyl)amino]-7-[3-(4-morpholinyl)propoxy]-6-quinazolinyl]-2-propenamide, EKB-569, PTK787, HER-2/neu protein antibody, EGF receptor antibody, trastuzumab, GEM-220 AR-639, MDX-447, MDX-260, DAB-720, HER-2 antagonist, VRCTC-310, MR1scFvPE38KDEL, ABX-EGF, EMD-55900, EMD-72000, EGF fusion toxin, OLX-103, Selex, CGP-62706, SU-5271, NX-278-L, metalloprotease inhibitor, EGF fusion protein, Amphiregulin, SU-5271, CGP-52411, AG-1478, RC-3940-II, CP-358774, C225, hbEGF-toxin, MAb 4D5, BBR-1611, PD-169450, QX-101, SU-5271, flavopiridol, SU-101, celastrol, CGP-52411, anti-flk-1, CEP-2563, HER-2 antagonist, NSC-675967, SU-5416, FCE-26806, DAB-720, CEP-751, ZD-1838, CGP-60261, EGF-RTK antagonist, ALL-TK antagonists, GRB2 antagonists, CGP-57148, ZD-1839, erbB-2 receptor inhibitors, PD-158780, benzothiazoles, PD-171026, BE-23372M derivatives, Met TK antagonist, PD-159973, GW-282974, CP-292597, ZM-105180, GW-7072X, Lck tyrosine kinase inhibitors, PD-168393, PD-173956, RG-14620, CGP-59326, genistein, FCE-27119, RG-13022, RG-50864, PD-154233, TT-232, AG-514, AG-568, PD-151514, BE-23372M, KW-6151, paeciloquinones, PDGFrTK inhibitors, SDZ-LAP-977, CGP-53716, CGP-79787, B43-genistein, CGP-62706, AG-957, erlotinib, iressa, cetuximab, trastuzumab-DM1, mono[3-butyl-8-(9-carboxy-6-hydroxy-3,7-dimethyl-2,4,8-nonatrienyl)-2-(4-carboxy-3-methyl-1,3-butadienyl)-9-methyl-1,7-dioxaspiro[5.5]undec-3-yl]ester, Imatinib mesylate, trastuzumab, 4-(3-chloroanilino)-6,7-dimethoxyquinazoline, [(dimethylamino)methyl]acrylo-para-[(hydroxy-benzoylsulfonyl)-oxy]phenone, cetuximab, theraCIM h-R3, erlotinib in combination with taxotere, C1033, ABX-EGF, EKB-569, anti-EGFR Mabs, EMD-72000; anti-EGFR Mab, EMD-6200, MDX-447, BAB-447, EMD-82633, H-447, ior-egf/r3, EGF-genistein, ABX-EGF, anti-EGFr MAb, anti-EGFr MAb, anti-EGFR-DM1 Ab, anti-EGFR conjugate, EGFR conjugate, CI-1033, GW-211, PKI-166, STI571, BIBX1522, 4-(phenylamino)quinazolines, EGF receptor antisense oligonucleotide, RC-3940-II, CP-358774, C225, hbEGF-toxin, MAb 4D5, BBR-1611, PD-169450, CGP-52411, SU-5271, amphiregulin, EGF fusion protein, 4-(3-bromoanilino)-6,7-dimethoxyquinazoline analogues, 4-[ar(alk)ylamino]pyridopyrimidines, (4-(phenylamino)quinazolines), GW2974, GW9263, GW4263, GW0277, GW5289, GW5949, GW9525, GW572016, PD13530, CGP5211, CGP53353, CGP 75166/PKI166, BIBX 1382, EKB-569, PKI-166, CI-1033, GW-2016, EMD-72000, MDX-210, 2C4, TgDCC-E1A, prodrugs of any of them, and mixtures thereof.

15. The method according to claim 1, wherein the EGF receptor antagonist is selected from the group consisting of erlotinib, iressa, cetuximab, ABX-EGF, prodrugs of any of them, and mixtures thereof.

16. The method according to claim 1, wherein the EGF receptor antagonist is erlotinib.

17. The method according to claim 1, wherein the subject suffers from or is predisposed to one or more neoplasia disorders selected from the group consisting of acral lentiginous melanoma, actinic keratoses, adenocarcinoma, adenoid cycstic carcinoma, adenomas, adenosarcoma, adenosquamous carcinoma, adrenocortical carcinoma, AIDS-related lymphoma, anal cancer, astrocytic tumors, bartholin gland carcinoma, basal cell carcinoma, bile duct cancer, bladder cancer, brain stem glioma, brain tumors, breast cancer, bronchial gland carcinomas, capillary carcinoma, carcinoids, carcinoma, carcinosarcoma, cavernous, central nervous system lymphoma, cerebral astrocytoma, cholangiocarcinoma, chondosarcoma, choriod plexus papilloma/carcinoma, clear cell carcinoma, colon cancer, colorectal cancer, cutaneous T-cell lymphoma, cystadenoma, endodermal sinus tumor, endometrial hyperplasia, endometrial stromal sarcoma, endometrioid adenocarcinoma, ependymal, epitheloid, esophageal cancer, Ewing's sarcoma, extragonadal germ cell tumor, fibrolamellar, focal nodular hyperplasia, gallbladder cancer, gastrinoma, germ cell tumors, gestational trophoblastic tumor, glioblastoma, glioma, glucagonoma, hemangiblastomas, hemangioendothelioma, hemangiomas, hepatic adenoma, hepatic adenomatosis, hepatocellular carcinoma, Hodgkin's lymphoma, hypopharyngeal cancer, hypothalamic and visual pathway glioma, childhood, insulinoma, intaepithelial neoplasia, interepithelial squamous cell neoplasia, intraocular melanoma, invasive squamous cell carcinoma, large cell carcinoma, islet cell carcinoma, Kaposi's sarcoma, kidney cancer, laryngeal cancer, leiomyosarcoma, lentigo maligna melanomas, leukemia-related disorders, lip and oral cavity cancer, liver cancer, lung cancer, lymphoma, malignant mesothelial tumors, malignant thymoma, medulloblastoma, medulloepithelioma, melanoma, meningeal, merkel cell carcinoma, mesothelial, metastatic carcinoma, mucoepidermoid carcinoma, multiple myeloma/plasma cell neoplasm, mycosis fungoides, myelodysplastic syndrome, myeloproliferative disorders, nasal cavity and paranasal sinus cancer, nasopharyngeal cancer, neuroblastoma, neuroepithelial adenocarcinoma nodular melanoma, non-Hodgkin's lymphoma, non-small cell lung cancer, oat cell carcinoma, oligodendroglial, oral cancer, oropharyngeal cancer, osteosarcoma, pancreatic polypeptide, ovarian cancer, ovarian germ cell tumor, pancreatic cancer, papillary serous adenocarcinoma, pineal cell, pituitary tumors, plasmacytoma, pseudosarcoma, pulmonary blastoma, parathyroid cancer, penile cancer, pheochromocytoma, pineal and supratentorial primitive neuroectodermal tumors, pituitary tumor, plasma cell neoplasm, pleuropulmonary blastoma, prostate cancer, rectal cancer, renal cell carcinoma, retinoblastoma, rhabdomyosarcoma, sarcoma, serous carcinoma, small cell carcinoma, small intestine cancer, soft tissue carcinomas, somatostatin-secreting tumor, squamous carcinoma, squamous cell carcinoma, submesothelial, superficial spreading melanoma, supratentorial primitive neuroectodermal tumors, thyroid cancer, undifferentiatied carcinoma, urethral cancer, uterine sarcoma, uveal melanoma, verrucous carcinoma, vaginal cancer, vipoma, vulvar cancer, Waldenstrom's macroglobulinemia, well differentiated carcinoma, and Wilm's tumor.

18. The method according to claim 1, wherein the subject suffers from or is predisposed to colon cancer.

19. The method according to claim 1, further comprising administering an amount of a Cox-2 inhibitor and an amount of an EGF receptor antagonist wherein the amount of the Cox-2 inhibitor and the amount of the EGF receptor antagonist together comprises a therapeutically effective amount.

20. The method according to claim 1, wherein the Cox-2 inhibitor and EGF receptor antagonist is administered to the subject in combination with one or more antineoplastic agents and the antineoplastic agent is other than a Cox-2 inhibitor and other than a EGF receptor antagonist.

21. A therapeutic composition comprising at least one Cox-2 inhibitor and one or more EGF receptor antagonists.

22. The therapeutic composition according to claim 21, wherein the Cox-2 inhibitor comprises a Cox-2 selective inhibitor.

23. The therapeutic composition according to claim 22, wherein the Cox-2 selective inhibitor is selected from the group consisting of celecoxib, parecoxib, deracoxib, valdecoxib, etoricoxib, meloxicam, rofecoxib, lumiracoxib, RS 57067, T-614, BMS-347070, JTE-522, S-2474, SVT-2016, CT-3, ABT-963, SC-58125, nimesulide, flosulide, NS-398, L-745337, RWJ-63556, L-784512, darbufelone, CS-502, LAS-34475, LAS-34555, S-33516, SD-8381, prodrugs of any of them, and mixtures thereof.

24. The therapeutic composition according to claim 21, further comprising one or more antineoplastic agents.

25. The therapeutic composition according to claim 21, wherein the EGF receptor antagonist is selected from the group consisting of AGM-1470, reveromycin A, TNP-470, PD-171026, PD-089828, PD-090560, ZM-254530, ZM-105180, EGF-genistein, wayne anti-EGFR Mabs, anti-VEGF monoclonal, Genen EMD-72000, anti-EGFR Mab, EMD-6200, MDX-447, BAB-447, EMD-82633, H-447, ABX-EGF, anti-EGFr MAb, anti-EGFr Mab, anti-EGFR-DM1 Ab, anti-EGFR conjugate, anti-flk-1 MAb DC-101, bromelain molecules, CCX, CCZ, tecogalan sodium, platelet factor 4, inhibitors of vascular endothelial growth factor antagonist (VEGF) and its receptor flk-1, 4-(3-Ethynylphenylamino)-6,7-bis(2-methoxyethoxy)quinazoline hydrochloride, ZD-1838, ZD-1839, 4-(4-methylpiperazin-1-ylmethyl)-N-[4-methyl-3-[4-(3-pyridyl)pyrimidin-2-ylamino]phenyl]benzamide, CGP-59326, CGP-79787 CGP-59326B, CGP-62706, CGP-74321, CGP-75166, CGP-76627, DWP-408, EGF-genistein, muellerian-inhibiting hormone, 4-(m-chloro)-5,6-dimethyl-7H-pyrrolo[2,3-d]pyrimidine, 5-[3-[3-methoxy-4-[2-[(E)-2-phenylethenyl]-4-oxazolylmethoxy]phenyl]propyl]-3-[2-[(E)-2-phenylethenyl]-4-oxazolylmethyl]-2,4-oxazolidinedione, N-(6-Benzothiazolyl)-4-(2-(1-piperazinyl)pyrid-5-yl)-2-pyrimidineamine, PI-88, PJ3505, S-96-8045, 4-(4-chloro-2-fluoro-5-hydroxyanilino)-6-methoxy-7-(2-methoxyethoxy)cinnoline, N-[4-[(3-chloro-4-fluorophenyl)amino]-7-[3-(4-morpholinyl)propoxy]-6-quinazolinyl]-2-propenamide, EKB-569, PTK787, HER-2/neu protein antibody, EGF receptor antibody, trastuzumab, GEM-220 AR-639, MDX-447, MDX-260, DAB-720, HER-2 antagonist, VRCTC-310, MR1scFvPE38KDEL, EMD-55900, EGF fusion toxin, OLX-103, Selex, CGP-62706, SU-5271, NX-278-L, metalloprotease inhibitor, EGF fusion protein, Amphiregulin, AG-1478, RC-3940-II, CP-358774, C225, hbEGF-toxin, MAb 4D5, BBR-1611, PD-169450, QX-101, SU-5271, flavopiridol, SU-101, celastrol, CGP-52411, anti-flk-1, CEP-2563, HER-2 antagonist, NSC-675967, SU-5416, FCE-26806, DAB-720, CEP-751, ZD-1838, CGP-60261, EGF-RTK antagonist, ALL-TK antagonists, GRB2 antagonists, CGP-57148, ZD-1839, erbB-2 receptor inhibitors, PD-158780, benzothiazoles, PD-171026, BE-23372M derivatives, Met TK antagonist, PD-159973, GW-282974, CP-292597, ZM-105180, GW-7072X, Lck tyrosine kinase inhibitors, PD-168393, PD-173956, RG-14620, CGP-59326, genistein, FCE-27119, RG-13022, RG-50864, PD-154233, TT-232, AG-514, AG-568, PD-151514, BE-23372M, KW-6151, paeciloquinones, PDGFrTK inhibitors, SDZ-LAP-977, CGP-53716, CGP-79787, B43-genistein, CGP-62706, AG-957, erlotinib, iressa, cetuximab, trastuzumab-DM1, mono[3-butyl-8-(9-carboxy-6-hydroxy-3,7-dimethyl-2,4,8-nonatrienyl)-2-(4-carboxy-3-methyl-1,3-butadienyl)-9-methyl-1,7-dioxaspiro[5.5]undec-3-yl]ester, imatinib mesylate, trastuzumab, 4-(3-chloroanilino)-6,7-dimethoxyquinazoline, [(dimethylamino)methyl]acrylo-para-[(hydroxy-benzoylsulfonyl)-oxy]phenone, cetuximab, theraCIM h-R3, erlotinib in combination with taxotere, C1033, EKB-569, ior-egf/r3, CI-1033, GW-211, PKI-166, ST1571, BIBX1522, 4-(phenylamino)quinazolines, EGF receptor antisense oligonucleotide, 4-(3-bromoanilino)-6,7-dimethoxyquinazoline analogues, 4-[ar(alk)ylamino]pyridopyrimidines, GW2974, GW9263, GW4263, GW0277, GW5289, GW5949, GW9525, GW572016, PD13530, CGP5211, CGP53353, CGP 75166/PKI166, BIBX 1382, CI-1033, GW-2016, EMD-72000, MDX-210, 2C4, TgDCC-E1A, prodrugs of any of them, and mixtures thereof.

26. The therapeutic composition according to claim 21, wherein the EGF receptor antagonist is selected from the group consisting of AGM-1470, reveromycin A, TNP-470, PD-171026, PD-089828, PD-090560, ZM-254530, ZM-105180, EGF-genistein, wayne anti-EGFR Mabs, anti-VEGF monoclonal, Genen EMD-72000, anti-EGFR Mab, EMD-6200, MDX-447, BAB-447, EMD-82633, H-447, ABX-EGF, anti-EGFr MAb, anti-EGFr Mab, anti-EGFR-DM1 Ab, anti-EGFR conjugate, anti-flk-1 MAb DC-101, bromelain molecules, CCX, CCZ, tecogalan sodium, platelet factor 4, Inhibitors of vascular endothelial growth factor antagonist (VEGF) and its receptor flk-1, 4-(3-Ethynylphenylamino)-6,7-bis(2-methoxyethoxy)-quinazoline hydrochloride, ZD-1838, ZD-1839, 4-(4-methylpiperazin-1-ylmethyl)-N-[4-methyl-3-[4-(3-pyridyl)pyrimidin-2-ylamino]phenyl]benzamide, CGP-59326, CGP-79787 CGP-59326B, CGP-62706, CGP-74321, CGP-75166, CGP-76627, DWP-408, EGF-genistein, muellerian-inhibiting hormone, 4-(m-chloro)-5,6-dimethyl-7H-pyrrolo[2,3-d]pyrimidine, 5-[3-[3-methoxy-4-[2-[(E)-2-phenylethenyl]-4-oxazolylmethoxy]phenyl]propyl]-3-[2-[(E)-2-phenylethenyl]-4-oxazolylmethyl]-2,4-oxazolidinedione, N-(6-Benzothiazolyl)-4-(2-(1-piperazinyl)pyrid-5-yl)-2-pyrimidineamine, PI-88, PJ3505, S-96-8045, 4-(4-chloro-2-fluoro-5-hydroxyanilino)-6-methoxy-7-(2-methoxyethoxy)cinnoline, N-[4-[(3-chloro-4-fluorophenyl)amino]-7-[3-(4-morpholinyl)propoxy]-6-quinazolinyl]-2-propenamide, EKB-569, PTK787, HER-2/neu protein antibody, EGF receptor antibody, trastuzumab, GEM-220 AR-639, MDX-447, MDX-260, DAB-720, HER-2 antagonist, VRCTC-310, MR1scFvPE38KDEL, ABX-EGF, EMD-55900, EMD-72000, EGF fusion toxin, OLX-103, Selex, CGP-62706, SU-5271, NX-278-L, metalloprotease inhibitor, EGF fusion protein, Amphiregulin, SU-5271, CGP-52411, AG-1478, RC-3940-II, CP-358774, C225, hbEGF-toxin, MAb 4D5, BBR-1611, PD-169450, reveromycin-A, QX-101, SU-5271, flavopiridol, SU-101, celastrol, CGP-52411, anti-flk-1, CEP-2563, HER-2 antagonist, NSC-675967, SU-5416, FCE-26806, DAB-720, CEP-751, ZD-1838, CGP-60261, EGF-RTK antagonist, ALL-TK antagonists, GRB2 antagonists, CGP-57148, ZD-1839, erbB-2 receptor inhibitors, PD-158780, benzothiazoles, PD-171026, BE-23372M derivatives, Met TK antagonist, PD-159973, GW-282974, CP-292597, ZM-105180, GW-7072X, Lck tyrosine kinase inhibitors, PD-168393, PD-173956, RG-14620, CGP-59326, genistein, FCE-27119, RG-13022, RG-50864, PD-154233, TT-232, AG-514, AG-568, PD-151514, BE-23372M, KW-6151, paeciloquinones, PDGFrTK inhibitors, SDZ-LAP-977, CGP-53716, CGP-79787, B43-genistein, CGP-62706, AG-957, erlotinib, iressa, cetuximab, trastuzumab-DM1, mono[3-butyl-8-(9-carboxy-6-hydroxy-3,7-dimethyl-2,4,8-nonatrienyl)-2-(4-carboxy-3-methyl-1,3-butadienyl)-9-methyl-1,7-dioxaspiro[5.5]undec-3-yl]ester, Imatinib mesylate, trastuzumab, 4-(3-chloroanilino)-6,7-dimethoxyquinazoline, [(dimethylamino)methyl]acrylo-para-[(hydroxy-benzoylsulfonyl)-oxy]phenone, cetuximab, theraCIM h-R3, erlotinib in combination with taxotere, C1033, ABX-EGF, EKB-569, anti-EGFR Mabs, EMD-72000; anti-EGFR Mab, EMD-6200, MDX-447, BAB-447, EMD-82633, H-447, ior-egf/r3, EGF-genistein, ABX-EGF, anti-EGFr MAb, anti-EGFr MAb, anti-EGFR-DM1 Ab, anti-EGFR conjugate, EGFR conjugate, CI-1033, GW-211, PKI-166, ST1571, BIBX1522, 4-(phenylamino)quinazolines, EGF receptor antisense oligonucleotide, RC-3940-II, CP-358774, C225, hbEGF-toxin, MAb 4D5, BBR-1611, PD-169450, CGP-52411, SU-5271, amphiregulin, EGF fusion protein, 4-(3-bromoanilino)-6,7-dimethoxyquinazoline analogues, 4-[ar(alk)ylamino]pyridopyrimidines, (4-(phenylamino)quinazolines), GW2974, GW9263, GW4263, GW0277, GW5289, GW5949, GW9525, GW572016, PD13530, CGP5211, CGP53353, CGP 75166/PKI166, BIBX 1382, EKB-569, PKI-166, CI-1033, GW-2016, EMD- 72000, MDX-210, 2C4, TgDCC-E1A, prodrugs of any of them, and mixtures thereof.

27. The therapeutic composition according to claim 21, wherein the EGF receptor antagonist is erlotinib.

28. A pharmaceutical composition comprising at least one Cox-2 inhibitor, one or more EGF receptor antagonists, and a pharmaceutically acceptable carrier.

29. The pharmaceutical composition according to claim 28, further comprising one or more antineoplastic agents.

30. The pharmaceutical composition according to claim 28, wherein the Cox-2 inhibitor comprises a Cox-2 selective inhibitor.

31. The pharmaceutical composition according to claim 28, wherein the Cox-2 selective inhibitor is selected from the group consisting of celecoxib, parecoxib, deracoxib, valdecoxib, etoricoxib, meloxicam, rofecoxib, lumiracoxib, RS 57067, T-614, BMS-347070, JTE-522, S-2474, SVT-2016, CT-3, ABT-963, SC-58125, nimesulide, flosulide, NS-398, L-745337, RWJ-63556, L-784512, darbufelone, CS-502, LAS-34475, LAS-34555, S-33516, SD-8381, prodrugs of any of them, and mixtures thereof.

32. The pharmaceutical composition according to claim 28, wherein the EGF receptor antagonist is selected from the group consisting of AGM-1470, reveromycin A, TNP-470, PD-171026, PD-089828, PD-090560, ZM-254530, ZM-105180, EGF-genistein, wayne anti-EGFR Mabs, anti-VEGF monoclonal, Genen EMD-72000, anti-EGFR Mab, EMD-6200, MDX-447, BAB-447, EMD-82633, H-447, ABX-EGF, anti-EGFr MAb, anti-EGFr Mab, anti-EGFR-DM1 Ab, anti-EGFR conjugate, anti-flk-1 MAb DC-101, bromelain molecules, CCX, CCZ, tecogalan sodium, platelet factor 4, Inhibitors of vascular endothelial growth factor antagonist (VEGF) and its receptor flk-1, 4-(3-Ethynylphenylamino)-6,7-bis(2-methoxyethoxy)quinazoline hydrochloride, ZD-1838, ZD-1839, 4-(4-methylpiperazin-1-ylmethyl)-N-[4-methyl-3-[4-(3-pyridyl)pyrimidin-2-ylamino]phenyl]benzamide, CGP-59326, CGP-79787 CGP-59326B, CGP-62706, CGP-74321, CGP-75166, CGP-76627, DWP-408, EGF-genistein, muellerian-inhibiting hormone, 4-(m-chloro)-5,6-dimethyl-7H-pyrrolo[2,3-d]pyrimidine, 5-[3-[3-methoxy-4-[2-[(E)-2-phenylethenyl]-4-oxazolylmethoxy]phenyl]propyl]-3-[2-[(E)-2-phenylethenyl]-4-oxazolylmethyl]-2,4-oxazolidinedione, N-(6-Benzothiazolyl)-4-(2-(1-piperazinyl)pyrid-5-yl)-2-pyrimidineamine, PI-88, PJ3505, S-96-8045, 4-(4-chloro-2-fluoro-5-hydroxyanilino)-6-methoxy-7-(2-methoxyethoxy)cinnoline, N-[4-[(3-chloro-4-fluorophenyl)amino]-7-[3-(4-morpholinyl)propoxy]-6-quinazolinyl]-2-propenamide, EKB-569, PTK787, HER-2/neu protein antibody, EGF receptor antibody, trastuzumab, GEM-220 AR-639, MDX-447, MDX-260, DAB-720, HER-2 antagonist, VRCTC-310, MR1 scFvPE38KDEL, EMD-55900, EGF fusion toxin, OLX-103, SELEX, CGP-62706, SU-5271, NX-278-L, metalloprotease inhibitor, EGF fusion protein, Amphiregulin, SU-5271, CGP-52411, AG-1478, RC-3940-II, CP-358774, C225, hbEGF-toxin, MAb 4D5, BBR-1611, PD-169450, QX-101, SU-5271, flavopiridol, SU-101, celastrol, CGP-52411, anti-flk-1, CEP-2563, HER-2 antagonist, NSC-675967, SU-5416, FCE-26806, DAB-720, CEP-751, ZD-1838, CGP-60261, EGF-RTK antagonist, ALL-TK antagonists, GRB2 antagonists, CGP-57148, ZD-1839, erbB-2 receptor inhibitors, PD-158780, benzothiazoles, PD-171026, BE-23372M derivatives, Met TK antagonist, PD-159973, GW-282974, CP-292597, ZM-105180, GW-7072X, Lck tyrosine kinase inhibitors, PD-168393, PD-173956, RG-14620, CGP-59326, genistein, FCE-27119, RG-13022, RG-50864, PD-154233, TT-232, AG-514, AG-568, PD-151514, BE-23372M, KW-6151, paeciloquinones, PDGFrTK inhibitors, SDZ-LAP-977, CGP-53716, CGP-79787, B43-genistein, CGP-62706, AG-957, erlotinib, iressa, cetuximab, trastuzumab-DM1, mono[3-butyl-8-(9-carboxy-6-hydroxy-3,7-dimethyl-2,4,8-nonatrienyl)-2-(4-carboxy-3-methyl-1,3-butadienyl)-9-methyl-1,7-dioxaspiro[5.5]undec-3-yl]ester, Imatinib mesylate, trastuzumab, 4-(3-chloroanilino)-6,7-dimethoxyquinazoline, [(dimethylamino)methyl]acrylo-para-[(hydroxy-benzoylsulfonyl)oxy]phenone, cetuximab, theraCIM h-R3, erlotinib in combination with taxotere, C1033, EKB-569, ior-egf/r3, CI-1033, GW-211, PKI-166, STI571, BIBX1522, 4-(phenylamino)quinazolines, EGF receptor antisense oligonucleotide, 4-(3-bromoanilino)-6,7-dimethoxyquinazoline analogues, 4-[ar(alk)ylamino]pyridopyrimidines, GW2974, GW9263, GW4263, GW0277, GW5289, GW5949, GW9525, GW572016, PD13530, CGP5211, CGP53353, CGP 75166/PKI166, BIBX 1382, CI-1033, GW-2016, EMD-72000, MDX-210, 2C4, TgDCC-E1A, prodrugs of any of them, and mixtures thereof.

33. The pharmaceutical composition according to claim 28, wherein the EGF receptor antagonist is selected from the group consisting of AGM-1470, reveromycin A, TNP-470, PD-171026, PD-089828, PD-090560, ZM-254530, ZM-105180, EGF-genistein, wayne anti-EGFR Mabs, anti-VEGF monoclonal, Genen EMD-72000, anti-EGFR Mab, EMD-6200, MDX-447, BAB-447, EMD-82633, H-447, ABX-EGF, anti-EGFr MAb, anti-EGFr Mab, anti-EGFR-DM1 Ab, anti-EGFR conjugate, anti-flk-1 MAb DC-101, bromelain molecules, CCX, CCZ, tecogalan sodium, platelet factor 4, Inhibitors of vascular endothelial growth factor antagonist (VEGF) and its receptor flk-1, 4-(3-Ethynylphenylamino)-6,7-bis(2-methoxyethoxy)quinazoline hydrochloride, ZD-1838, ZD-1839, 4-(4-methylpiperazin-1-ylmethyl)-N-[4-methyl-3-[4-(3-pyridyl)pyrimidin-2-ylamino]phenyl]benzamide, CGP-59326, CGP-79787 CGP-59326B, CGP-62706, CGP-74321, CGP-75166, CGP-76627, DWP-408, EGF-genistein, muellerian-inhibiting hormone, 4-(m-chloro)-5,6-dimethyl-7H-pyrrolo[2,3-d]pyrimidine, 5-[3-[3-methoxy-4-[2-[(E)-2-phenylethenyl]-4-oxazolylmethoxy]phenyl]propyl]-3-[2-[(E)-2-phenylethenyl]-4-oxazolylmethyl]-2,4-oxazolidinedione, N-(6-Benzothiazolyl)-4-(2-(1-piperazinyl)pyrid-5-yl)-2-pyrimidineamine, PI-88, PJ3505, S-96-8045, 4-(4-chloro-2-fluoro-5-hydroxyanilino)-6-methoxy-7-(2-methoxyethoxy)cinnoline, N-[4-[(3-chloro-4-fluorophenyl)amino]-7-[3-(4-morpholinyl)propoxy]-6-quinazolinyl]-2-propenamide, EKB-569, PTK787, HER-2/neu protein antibody, EGF receptor antibody, trastuzumab, GEM-220 AR-639, MDX-447, MDX-260, DAB-720, HER-2 antagonist, VRCTC-310, MR1scFvPE38KDEL, ABX-EGF, EMD-55900, EMD-72000, EGF fusion toxin, OLX-103, Selex, CGP-62706, SU-5271, NX-278-L, metalloprotease inhibitor, EGF fusion protein, Amphiregulin, SU-5271, CGP-52411, AG-1478, RC-3940-II, CP-358774, C225, hbEGF-toxin, MAb 4D5, BBR-1611, PD-169450, QX-101, SU-5271, flavopiridol, SU-101, celastrol, CGP-52411, anti-flk-1, CEP-2563, HER-2 antagonist, NSC-675967, SU-5416, FCE-26806, DAB-720, CEP-751, ZD-1838, CGP-60261, EGF-RTK antagonist, ALL-TK antagonists, GRB2 antagonists, CGP-57148, ZD-1839, erbB-2 receptor inhibitors, PD-158780, benzothiazoles, PD-171026, BE-23372M derivatives, Met TK antagonist, PD-159973, GW-282974, CP-292597, ZM-105180, GW-7072X, Lck tyrosine kinase inhibitors, PD-168393, PD-173956, RG-14620, CGP-59326, genistein, FCE-27119, RG-13022, RG-50864, PD-154233, TT-232, AG-514, AG-568, PD-151514, BE-23372M, KW-6151, paeciloquinones, PDGFrTK inhibitors, SDZ-LAP-977, CGP-53716, CGP-79787, B43-genistein, CGP-62706, AG-957, erlotinib, iressa, cetuximab, trastuzumab-DM1, mono[3-butyl-8-(9-carboxy-6-hydroxy-3,7-dimethyl-2,4,8-nonatrienyl)-2-(4-carboxy-3-methyl-1,3-butadienyl)-9-methyl-1,7-dioxaspiro[5.5]undec-3-yl]ester, Imatinib mesylate, trastuzumab, 4-(3-chloroanilino)-6,7-dimethoxyquinazoline, [(dimethylamino)methyl]acrylo-para-[(hydroxy-benzoylsulfonyl)-oxy]phenone, cetuximab, theraCIM h-R3, erlotinib in combination with taxotere, C1033, ABX-EGF, EKB-569, anti-EGFR Mabs, EMD-72000; anti-EGFR Mab, EMD-6200, MDX-447, BAB-447, EMD-82633, H-447, ior-egf/r3, EGF-genistein, ABX-EGF, anti-EGFr MAb, anti-EGFr MAb, anti-EGFR-DM1 Ab, anti-EGFR conjugate, EGFR conjugate, CI-1033, GW-211, PKI-166, ST1571, BIBX1522, 4-(phenylamino)quinazolines, EGF receptor antisense oligonucleotide, RC-3940-II, CP-358774, C225, hbEGF-toxin, MAb 4D5, BBR-1611, PD-169450, CGP-52411, SU-5271, amphiregulin, EGF fusion protein, 4-(3-bromoanilino)-6,7-dimethoxyquinazoline analogues, 4-[ar(alk)ylamino]pyridopyrimidines, (4-(phenylamino)quinazolines), GW2974, GW9263, GW4263, GW0277, GW5289, GW5949, GW9525, GW572016, PD13530, CGP5211, CGP53353, CGP 75166/PKI166, BIBX 1382, EKB-569, PKI-166, CI-1033, GW-2016, EMD-72000, MDX-210, 2C4, TgDCC-E1A, prodrugs of any of them, and mixtures thereof.

34. A kit for preventing or treating neoplasia disorders and neoplasia disorder-related complications in a subject that is in need of such prevention or treatment, the kit comprising one dosage form comprising a Cox-2 inhibitor and a second dosage form comprising an EGF receptor antagonist.
